(19) United States
(12) Patent Application Publication
(10) Pub. No.: US 2014/0018788 A1
(43) Pub. Date: Jan. 16, 2014

(54) Devices and Systems for Carotid Body Ablation

(71) Applicants: Zoar Jacob Engelman, Salt Lake City, UT (US); Martin M. Grasse, Boston, MA (US); Mark Gelfand, New York, NY (US); Charles Lennox, Hudson, NH (US); Marwan Abboud, Pierrefonds (CA); Mark Leung, Shawnigan Lake (CA); Howard Levin, Teaneck, NJ (US); Marat Fudim, Duesseldorf (DE); Michael Brick Markham, Sunnyvale, CA (US); Miriam H. Taimisto, San Jose, CA (US); Kenneth M. Martin, Woodside, CA (US); Robert Brommer, Fremont, CA (US); Scott H. West, Livermore, CA (US)

(72) Inventors: Zoar Jacob Engelman, Salt Lake City, UT (US); Martin M. Grasse, Boston, MA (US); Mark Gelfand, New York, NY (US); Charles Lennox, Hudson, NH (US); Marwan Abboud, Pierrefonds (CA); Mark Leung, Shawnigan Lake (CA); Howard Levin, Teaneck, NJ (US); Marat Fudim, Duesseldorf (DE); Michael Brick Markham, Sunnyvale, CA (US); Miriam H. Taimisto, San Jose, CA (US); Kenneth M. Martin, Woodside, CA (US); Robert Brommer, Fremont, CA (US); Scott H. West, Livermore, CA (US)

(21) Appl. No.: 13/936,121

(22) Filed: Jul. 5, 2013

Related U.S. Application Data
(60) Provisional application No. 61/667,991, filed on Jul. 4, 2012.
(60) Provisional application No. 61/667,996, filed on Jul. 4, 2012.
(60) Provisional application No. 61/667,998, filed on Jul. 4, 2012.
(60) Provisional application No. 61/682,034, filed on Aug. 10, 2012.
(60) Provisional application No. 61/768,101, filed on Feb. 22, 2013.
(60) Provisional application No. 61/791,769, filed on Mar. 15, 2013.
(60) Provisional application No. 61/791,420, filed on Mar. 15, 2013.
(60) Provisional application No. 61/792,214, filed on Mar. 15, 2013.
(60) Provisional application No. 61/792,741, filed on Mar. 15, 2013.
(60) Provisional application No. 61/793,267, filed on Mar. 15, 2013.
(60) Provisional application No. 61/794,667, filed on Mar. 15, 2013.
(60) Provisional application No. 61/810,639, filed on Apr. 10, 2013.
(60) Provisional application No. 61/836,100, filed on Jun. 17, 2013.

(51) Int. Cl. A61B 18/18

(52) U.S. Cl. CPC A61B 18/18
USPC 606/33

(57) ABSTRACT

Methods and endovascular catheters for assessing, and treating patients having sympathetically mediated disease, involving augmented peripheral chemoreflex and heightened sympathetic tone by reducing chemosensor input to the nervous system via transmural carotid body ablation.

### CROSS REFERENCE TO RELATED APPLICATIONS

This application claims priority to the following U.S. Provisional Applications, the disclosures of which are incorporated by reference herein in their entireties: U.S. Prov. App. No. 61/667,991, filed Jul. 4, 2012; U.S. Prov. App. No. 61/667,996, filed Jul. 4, 2012; U.S. Prov. App. No. 61/667,998, filed Jul. 4, 2012; U.S. Prov. App. No. 61/682,034, filed Aug. 10, 2012; U.S. Prov. App. No. 61/768,101, filed Feb. 22, 2013; U.S. Prov. App. No. 61/791,769, filed Mar. 15, 2013; U.S. Prov. App. No. 61/791,420, filed Mar. 15, 2013; U.S. Prov. App. No. 61/792,214, filed Mar. 15, 2013; U.S. Prov. App. No. 61/792,741, filed Mar. 15, 2013; U.S. Prov. App. No. 61/793,267, filed Mar. 15, 2013; U.S. Prov. App. No. 61/794,667, filed Mar. 15, 2013; U.S. Prov. App. No. 61/810,639, filed Apr. 10, 2013; and U.S. Prov. App. No. 61/836,100, filed Jun. 17, 2013.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

The present disclosure is directed generally to devices, systems and methods for treating patients having sympathetically mediated disease associated at least in part with augmented peripheral chemoreflex, heightened sympathetic activation, or autonomic imbalance by ablating at least one peripheral chemoreceptor (e.g., a carotid body) or an associated nerve.

### BACKGROUND

It is known that an imbalance of the autonomic nervous system is associated with several disease states. Restoration of autonomic balance has been a target of several medical treatments including modalities such as pharmacological, device-based, and electrical stimulation. For example, beta blockers are a class of drugs used to reduce sympathetic activity to treat cardiac arrhythmias and hypertension; Gelfand and Levin (U.S. Pat. No. 7,162,303) describe a device-based treatment used to decrease renal sympathetic activity to treat heart failure, hypertension, and renal failure; Yun and Yuarn-Bor (U.S. Pat. No. 7,149,574; U.S. Pat. No. 7,363,076; U.S. Pat. No. 7,738,952) describe a method of restoring autonomic balance by increasing parasympathetic activity to treat disease associated with parasympathetic attrition; Kieval, Burns and Serdar (U.S. Pat. No. 8,060,206) describe an electrical pulse generator that stimulates a baroreceptor, increasing parasympathetic activity, in response to high blood pressure; Hlavka and Elliott (US 2010/0070004) describe an implantable electrical stimulator in communication with an afferent neural pathway of a carotid body chemoreceptor to control dyspnea via electrical neuromodulation. US 2012/0172680 describes carotid body ablation for treating sympathetically mediated diseases.

Ablating a carotid body in a human patient is risky and difficult. A carotid body is typically about the size of a grain of rice, located near other glands, nerves, muscles and other organs, and moves with movement of the jaw and neck, respiration and blood pulsation. Conventional open surgical techniques to access the carotid body directly through the neck that are referred to as open surgery are challenging due to the nerves, muscles, arteries, veins and other organs near the carotid body. In the modern medicine open surgery is only used to access a carotid body for removal of carotid body tumors that are immediately life threatening.

### SUMMARY

There is a desire for minimally invasive surgical techniques and instruments configured to ablate at least a portion of the carotid body. Endovascular catheter assemblies are known for performing minimally invasive procedures and surgeries, including endovascular ablation of nerves, on the heart, kidney, pulmonary artery, renal artery and other body organs typically located below the neck. These catheter assemblies tend to be too short, too large, lack necessary features needed for retention and targeting of energy delivery and otherwise not suited to reaching the neck and, particularly, the narrow blood vessels in the neck. Endovascular catheter assemblies are also known for treating arteries in the neck such as to treat aneurysms in the wall of a blood vessel.

It is not conventional to use traditional minimally invasive surgical ablation instruments and techniques to treat organs in the neck, particularly at and near the bifurcation of carotid artery where the carotid body is located. One difficulty with applying endovascular catheter ablation techniques to an organ in the neck, other than an artery or vein in the torso or abdomen, is the long and tortuous path through the vascular system that a catheter is generally advanced to reach the neck. Another difficulty can be properly positioning the distal end of the catheter in an artery to act on the target organ that is external to the artery. Another difficulty is avoiding damage to carotid endothelium that can lead to formation of thrombus, avoiding excessive heating and scarring of blood vessel walls that can lead to stenosis, or disturbing atherosclerotic plaque that can cause embolization of brain arteries and stroke. The organ may move with respect to the artery, the narrow arteries in the neck and the complex geometries of these arteries present challenges to a minimally invasive technique to reach the carotid body. Ablation procedures may take tens of seconds and even minutes and in the highly mobile are of the neck catheter can be displaced during energy application.

While catheter probes with stimulation electrodes have been proposed for electrically stimulating the carotid body, these approaches do not describe ablating or otherwise permanently changing the carotid body. Nor do they describe devices and systems that are used to accomplish the same. Ablating, modulating or otherwise permanently changing the carotid body or its function requires the application of energy, chemicals or other forces sufficient to damage the carotid body or its associated nerves and potentially tissue and blood vessel walls near the carotid body. Damaging the carotid body, nerves and nearby tissue is not necessary or desired if the object of a treatment is to electrically stimulate the carotid body. Applying a relatively low level of energy to electrically stimulate the carotid body will unlikely damage a blood vessel or surrounding tissue, even if the energy is applied to a broader area than the carotid body. The level of energy and force or the chemicals needed to ablate the carotid body is substantially higher than the levels needed for stimulation. Applying energy, chemicals and forces (e.g., thermal energy) sufficient to damage the carotid body raises concerns that the damage could extend to nearby non-target nerves and other organs, rupture the wall of the blood vessel, disturb and dislodge plaque or create blood clots that could flow to the brain.

In view of the need to damage the carotid body, there are strict requirements for positioning and retaining the tip of an ablating catheter in a carotid artery for the duration of the procedure, and for narrowly targeting the delivery of the energy, chemicals or force to the carotid body. Recognizing and identifying the requirements for positioning an ablating tip, or energy application element, of a catheter was a first step for an endovascular catheter assembly for ablating the carotid body. A second step included the invention of endovascular catheter assemblies that satisfied the requirements. Then parameters for energy application were developed that preserve the blood vessel and surrounding non-target tissues but substantially ablate the carotid body or an associated nerve.

Methods, devices, and systems have been conceived for endovascular transmural ablation of a carotid body with a catheter having two arms to facilitate positioning and apposition of ablation elements on an intercarotid septum. Endovascular transmural ablation of a carotid body herein generally refers to delivering a device through a patient's vasculature to a blood vessel proximate to a target ablation site (carotid body, intercarotid plexus, carotid body nerves) of the patient and placing an ablation element associated with the device against the internal wall of the vessel adjacent to the peripheral chemosensor and activating the ablation element to ablate the peripheral chemosensor.

A system has been conceived comprising a catheter having a means for coupling with a carotid bifurcation for transmural carotid body ablation and an ablation energy console. The system may additionally comprise a connector cable for connecting the ablation energy console with the catheter, a computer controlled software algorithm for controlling delivery of ablation energy, a delivery sheath, or a guide wire. Ablation energy can be thermal energy such as heating (e.g., RF, ultrasound, laser) or freezing (e.g., cryogenic element).

A carotid body may be ablated by placing an ablation element within and against the wall of a carotid artery adjacent to the carotid body of interest, then delivering ablation energy from the ablation element causing a change in temperature of periarterial space containing the carotid body to an extent and duration sufficient to ablate the carotid body.

Placing the ablation element (e.g., radiofrequency electrode) at a suitable location for carotid body ablation may be facilitated by a structure at a distal region of an ablation device (e.g., endovascular catheter) that comprises two arms configured to couple with a carotid bifurcation. The structure comprising two arms may comprise an ablation element on one arm or an ablation element on each of the two arms, or multiple ablation elements on one or each of the arms. The ablation element(s) may be positioned on the arms such that when the structure is coupled to a carotid bifurcation the ablation elements are placed at a suitable location (e.g., at or between about 0 to 15 mm, 4 to 15 mm, or 4 to 10 mm from a carotid bifurcation on an inner wall of an external carotid artery and internal carotid artery and within a vessel wall arc having an arc length of about 25% of the vessel circumference facing the opposing ablation element) on a target ablation site for effective carotid body ablation. The structure may further facilitate apposition of ablation element(s) with tissue.

Devices have been conceived that couple with a carotid bifurcation to facilitate orientation, positioning and apposition of one or more ablation elements at a target ablation site or sites suitable for carotid body ablation. The devices may be configured to measure tissue impedance across an intercarotid septum.

In another exemplary procedure a location of periarterial space associated with a carotid body is identified, then an ablation element is placed against the interior wall of a carotid artery adjacent to the identified location, then ablation parameters are selected and the ablation element is activated thereby ablating the carotid body, whereby the position of the ablation element and the selection of ablation parameters provides for ablation of the carotid body without substantial collateral damage to adjacent functional structures.

In further example the location of the periarterial space associated with a carotid body is identified, as well as the location of important non-target nerve structures not associated with the carotid body, then an ablation element is placed against the interior wall of a carotid artery adjacent to the identified location, ablation parameters are selected and the ablation element is then activated thereby ablating the carotid body, whereby the position of the ablation element and the selection of ablation parameters provides for ablation of the target carotid body without substantial collateral damage to important non-target nerve structures in the vicinity of the carotid body.

Selectable carotid body ablation parameters may include ablation element temperature, duration of ablation element activation, ablation power, ablation element force of contact with a vessel wall, ablation element size, ablation modality, and ablation element position within a vessel.

The location of the perivascular space associated with a carotid body may be determined by means of a non-fluoroscopic imaging procedure prior to carotid body ablation, where the non-fluoroscopic location information is translated to a coordinate system based on fluoroscopically identifiable anatomical and/or artificial landmarks.

A function of a carotid body may be stimulated (e.g., excited with electric signal or chemical) and at least one physiological parameter is recorded prior to and during the stimulation, then the carotid body is ablated, and the stimulation is repeated, whereby the change in recorded physiological parameter(s) prior to and after ablation is an indication of the effectiveness of the ablation.

A function of a carotid body may be temporarily blocked and at least one physiological parameter(s) is recorded prior to and during the blockade, then the carotid body is ablated, and the blockade is repeated, whereby the change in recorded physiological parameter(s) prior to and after ablation is an indication of the effectiveness of the ablation.

A device configured to prevent embolic debris from entering the brain may be deployed in an internal carotid artery associated with a carotid body, then an ablation element is placed within and against the wall of an external carotid artery or an internal carotid artery associated with the carotid body, the ablation element is activated resulting in carotid body ablation, the ablation element is then withdrawn, then the embolic prevention device is withdrawn, whereby the embolic prevention device in the internal carotid artery prevents debris resulting from the use of the ablation element form entering the brain.

A method has been conceived in which the location of the perivascular space associated with a carotid body is identified, then an ablation element is placed in a predetermined location against the interior wall of vessel adjacent to the identified location, then ablation parameters are selected and the ablation element is activated and then deactivated, the ablation element is then repositioned in at least one additional predetermine location against the same interior wall and the ablation element is then reactivated using the same or different ablation parameters, whereby the positions of the ablation element and the selection of ablation parameters provides for ablation of the carotid body without substantial collateral damage to adjacent functional structures.

A method has been conceived by which a location of perivascular space associated with a carotid body is identified, an ablation element configured for tissue freezing is placed against an interior wall of a vessel adjacent to the identified location, ablation parameters are selected for reversible cryo-ablation and the ablation element is activated, effectiveness of the ablation is then determined by at least one physiological response to the ablation, and if the determination is that the physiological response is favorable, then the ablation element is reactivated using the ablation parameters selected for permanent carotid body ablation.

A system has been conceived comprising a vascular catheter configured with an ablation element in the vicinity of the distal end, and a connection between the ablation element and a source of ablation energy at the proximal end, whereby the distal end of the catheter is constructed to be inserted into a peripheral artery of a patient and then maneuvered into an internal or external carotid artery using standard fluoroscopic guidance techniques and positioned in a predetermined position at a carotid bifurcation.

A system has been conceived comprising a vascular catheter configured with an ablation element in vicinity of a distal end configured for carotid body ablation and further configured for at least one of the following: neural stimulation, neural blockade, carotid body stimulation and carotid body blockade; and a connection between the ablation element and a source of ablation energy, stimulation energy and/or blockade energy.

A system has been conceived comprising a vascular catheter configured with an ablation element and at least one electrode configured for at least one of the following: neural stimulation, neural blockade, carotid body stimulation and carotid body blockade; and a connection between the ablation element to a source of ablation energy, and a connection between the ablation element and/or electrode(s) to a source of stimulation energy and/or blockade energy.

A system has been conceived comprising a vascular catheter with an ablation element mounted in the vicinity of a distal end configured for tissue heating, whereby, the ablation element comprises at least one electrode and at least one temperature sensor, a connection between the ablation element electrode(s) and temperature sensor(s) to an ablation energy source, with the ablation energy source being configured to maintain the ablation element at a temperature in the range of 36 to 100 degrees centigrade, during ablation using signals received from the temperature sensor(s). For example, in an embodiment the at least one ablation element in contact with blood is maintained at a temperature between 36 and 50 degrees centigrade to minimize coagulation while targeted periarterial tissue is heated to a temperature between 50 and 100 degrees centigrade, such as to 50 to 55 degrees centigrade, to ablate tissue but avoid boiling of water and steam and gas expansion in the tissue.

A system has been conceived comprising a vascular catheter with an ablation element mounted in vicinity of a distal end configured for tissue heating, whereby, the ablation element comprises at least one electrode and at least one temperature sensor and at least one irrigation channel, and a connection between the ablation element electrode(s) and temperature sensor(s) and irrigation channel(s) to an ablation energy source, with the ablation energy source being configured to maintain the ablation element at a temperature in the range of 36 to 100 degrees centigrade during ablation using signals received from the temperature sensor(s) and by providing irrigation to the vicinity of the ablation element. For example, in an embodiment the at least one ablation element in contact with blood is maintained at a temperature between 36 and 50 degrees centigrade to minimize coagulation while targeted periarterial tissue is heated to a temperature between 50 and 100 degrees centigrade to ablate tissue but avoid boiling of water and steam and gas expansion in the tissue.

A carotid artery catheter has been conceived with a user-actuated structure on a distal region, where actuation of the structure is facilitated by a pull wire within the catheter in communication between the distal region and a handle containing an actuator at the proximal end, and an ablation element mounted in the vicinity of the distal end, whereby the user-actuated structure is configured to provide the user with a means for placing the ablation element against the wall of a carotid artery and means to place arms of the catheter on both sides of carotid septum.

A carotid artery catheter has been conceived with a structure comprising at least two arms configured for user actuation on a distal region of the catheter, a radiopaque ablation element mounted on at least one arm of the structure and at least one radiopaque element on the opposite arm of the structure, whereby the structure provides a user with a means for creating apposition between the ablation element against a wall of a carotid artery, and the combination of the radiopaque ablation element and the radiopaque element provide the user with a substantially unambiguous fluoroscopic determination of the location of the ablation element within a carotid artery.

A system for endovascular transmural ablation of a carotid body has been conceived comprising a carotid artery catheter with an ablation element mounted on a distal region of the catheter, a means for pressing the ablation element against a wall of a carotid artery at a specific location, a means for connecting the ablation element to a source of ablation energy mounted at a proximal region of the catheter, and a console comprising a source of ablation energy, a means for controlling the ablation energy, a user interface configured to provide the user with a selection of ablation parameters, indications of the status of the console and the status of the ablation activity, a means to activate and deactivate an ablation, and an umbilical to provide a means for connecting the catheter to the console.

A method has been conceived to reduce or inhibit chemoreflex generated by a carotid body in a human patient, to reduce afferent nerve sympathetic activity of carotid body nerves to treat a sympathetically mediated disease, the method comprising: positioning a catheter in a vascular system of the patient such that a distal section of the catheter is in a lumen proximate to a carotid body of the patient; pressing an ablation element against a wall of the lumen adjacent to the carotid body, supplying energy to the ablation element wherein the energy is supplied by an energy supply apparatus outside of the patient; applying the energy from the energy supply to the ablation element to ablate tissue proximate to or included in the carotid body; and removing the ablation device from the patient; wherein a carotid body chemoreflex function is inhibited or sympathetic afferent nerve activity of carotid body nerves is reduced due to the ablation.

A method has been conceived to treat a patient having a sympathetically mediated disease by reducing or inhibiting chemoreflex function generated by a carotid body including steps of inserting a catheter into the patient's vasculature, positioning a portion of the catheter proximate a carotid body (e.g., in a carotid artery), positioning an ablation element toward a target ablation site (e.g., carotid body, intercarotid septum, carotid plexus, carotid body nerves, carotid sinus nerve), holding position of the catheter, applying ablative energy to the target ablation site via the ablation element, and removing the catheter from the patient's vasculature, wherein the ablative energy is sufficient to cool or heat tissue sufficiently to substantially reduce chemoreflex or afferent nerve signals from the carotid body while avoiding ablation of nearby important non-target nerve structures.

The methods and systems disclosed herein may be applied to satisfy clinical needs related to treating cardiac, metabolic, and pulmonary diseases associated, at least in part, with augmented chemoreflex (e.g., high chemosensor sensitivity or high chemosensor activity) and related sympathetic activation. The treatments disclosed herein may be used to restore autonomic balance by reducing sympathetic activity, as opposed to increasing parasympathetic activity. It is understood that parasympathetic activity can increase as a result of the reduction of sympathetic activity (e.g., sympathetic withdrawal) and normalization of autonomic balance. Furthermore, the treatments may be used to reduce sympathetic activity by modulating a peripheral chemoreflex. Furthermore, the treatments may be used to reduce afferent neural stimulus, conducted via afferent carotid body nerves, from a carotid body to the central nervous system. Enhanced peripheral and central chemoreflex is implicated in several pathologies including hypertension, cardiac tachyarrhythmias, sleep apnea, dyspnea, chronic obstructive pulmonary disease (COPD), diabetes and insulin resistance, and CHF. Mechanisms by which these diseases progress may be different, but they may commonly include contribution from increased afferent neural signals from a carotid body. Central sympathetic nervous system activation is common to all these progressive and debilitating diseases. Peripheral chemoreflex may be modulated, for example, by modulating carotid body activity. The carotid body is the sensing element of the afferent limb of the peripheral chemoreflex. Carotid body activity may be modulated, for example, by substantially ablating a carotid body or afferent nerves emerging from the carotid body. Such nerves can be found in a carotid body itself, in a carotid plexus, in an intercarotid septum, in periarterial space of a carotid bifurcation and internal and external carotid arteries. Therefore, a therapeutic method has been conceived that comprises a goal of restoring or partially restoring autonomic balance by reducing or removing carotid body input into the central nervous system.

One aspect of the disclosure is an endovascular carotid septum ablation catheter comprising first and second diverging arms, the first arm comprising an ablation element and configured so that the ablation element is in contact with a carotid septal wall in one of an external carotid artery and an internal carotid artery when the catheter is coupled with a common carotid artery bifurcation, the second arm configured to be disposed in the other of the internal carotid artery and external carotid artery when the catheter is coupled with the bifurcation.

One aspect of the disclosure is an endovascular carotid septum ablation catheter comprising first and second diverging arms, the first arm comprising a first ablation element and configured so that the first ablation element is in contact with an external carotid artery wall when the catheter is coupled with a common carotid artery bifurcation, the second arm comprising a second ablation element and configured so that the second ablation element is in contact with an internal carotid artery when the catheter is coupled with the bifurcation, wherein the first and second ablation elements are positioned on the first and second arms so that when the catheter is coupled with the bifurcation, a straight line passing through the first and second ablation elements passes through a carotid septum.

One aspect of the disclosure is a method of ablating a carotid septum, comprising advancing a first diverging arm of an ablation catheter into an external carotid artery and a second diverging arm of the ablation catheter into an internal carotid artery so that a first ablation element on the first diverging arm is in apposition with a carotid septum wall in the external carotid artery and a second ablation element on the second diverging arm is positioned in the internal carotid artery; and ablating carotid septal tissue by delivering ablation energy between the first and second ablation elements so that the ablation energy passes through a carotid septum.

### BRIEF DESCRIPTION OF THE DRAWINGS

FIG. 1 is a lateral view illustrating a patient's left intercarotid septum.

FIG. 2 is a transverse cross sectional view illustrating a patient's intercarotid septum.

FIG. 3 is a schematic view showing exemplary endovascular access of a catheter to a left common carotid artery of a patient lying in supine position.

FIG. 4A is a schematic view of a steerable sheath.

FIG. 4B is a schematic view of a steerable sheath in a deflected state.

FIGS. 5A and 5B are schematic views showing suitable placement of ablation elements on an intercarotid septum.

FIG. 5C is a schematic illustration of a force test.

FIGS. 6A, 6B, 6C, and 6D are schematic views of an endovascular ablation catheter having arms with ablation elements.

FIG. 7 is a cutaway illustration of a lateral view a patient's right carotid artery system with a schematic view of an endovascular ablation catheter having arms with ablation elements positioned in the patient's internal and external carotid arteries for transmural ablation of a carotid body.

FIG. 8 is a schematic view of an endovascular ablation catheter having arms comprising flex circuits with ablation elements.

FIGS. 9 and 10 are cross sectional views of flex circuits with ablation elements.

FIG. 11 is a schematic view of an endovascular ablation catheter having arms.

FIG. 12 is a cross sectional view of an embodiment of an arm.

FIGS. 13A, 13B, 13C, and 13D are schematic illustrations of ablation elements.

FIG. 14 is a schematic view of a distal region of an Endovascular Transmural Ablation Precision-Grip catheter with normally closed arms.

FIG. 15 is a schematic view of a preformed structural wire for an arm.

FIG. 16A is a cutaway illustration of a lateral view a patient's right carotid artery system with a schematic view of an endovascular ablation catheter having arms with ablation elements positioned in the patient's common carotid artery.

FIG. 16B is a cutaway illustration of a lateral view a patient's right carotid artery system with a schematic view of an endovascular ablation catheter having arms with ablation elements positioned on the patient's intercarotid septum.

FIG. 17 is a schematic view of an elastic structural member having a preformed shape that may be incorporated in to an Endovascular Transmural Ablation Precision-Grip catheter.

FIG. 18 is a schematic diagram of a distal region of an Endovascular Transmural Ablation Precision-Grip catheter.

FIG. 19A is a schematic diagram of a distal region of an Endovascular Transmural Ablation Precision-Grip catheter.

FIG. 19B is a schematic diagram of a distal region of an Endovascular Transmural Ablation Precision-Grip catheter.

FIG. 20 is a schematic diagram of a distal region of an Endovascular Transmural Ablation Precision-Grip catheter.

FIGS. 21A, 21B, 21C, 21D, and 21E are schematic diagrams of a distal region of an Endovascular Transmural Ablation Precision-Grip catheter.

FIG. 22 is a schematic diagram of a distal region of an Endovascular Transmural Ablation Precision-Grip catheter.

FIGS. 23A and 23B are schematic diagrams of a distal region of an Endovascular Transmural Ablation Precision-Grip catheter.

FIGS. 24A, 24B, 24C and 24D are schematic diagrams of a distal region of an Endovascular Transmural Ablation Precision-Grip catheter.

FIGS. 25A and 25B are schematic diagrams of a distal region of an Endovascular Transmural Ablation Precision-Grip catheter.

FIGS. 26A and 26B are schematic diagrams of a distal region of an Endovascular Transmural Ablation Precision-Grip catheter.

FIGS. 27A and 27B are schematic diagrams of a distal region of an Endovascular Transmural Ablation Precision-Grip catheter.

FIGS. 28A and 28B are schematic diagrams of a distal region of an Endovascular Transmural Ablation Precision-Grip catheter with controllable deflection.

FIGS. 29A, 29B, 29C and 29D are schematic views and a distal region of an Endovascular Transmural Ablation Precision-Grip catheter with controllable deflection and open/close actuation

FIGS. 30A and 30B are illustrations of an Endovascular Transmural Ablation Precision-Grip catheter configured for controllable deflection with a slide-on arm configuration.

FIGS. 31A, 31B, and 31C are illustrations of an Endovascular Transmural Ablation Precision-Grip catheter configured for controllable deflection with a slide-on arm configuration in use.

FIG. 32A is an illustration of an Endovascular Transmural Ablation Precision-Grip catheter configured for controllable deflection with a slide-on arm configuration. FIGS. 32B-32H are illustrations of electrodes.

FIG. 32I is an illustration of a structural member.

FIG. 32J is a chart demonstrating how horizontal and vertical radiopaque markers may be oriented to indicate a rotational angle.

FIGS. 33A-33C are illustrations of Endovascular Transmural Ablation Precision-Grip catheters having a larger electrode contact surface area in an internal carotid artery and a smaller electrode in an external carotid artery.

FIGS. 34A, 34B and 34C are schematic diagrams of a distal region of an Endovascular Transmural Ablation Precision-Grip catheter with a guide wire lumen.

FIG. 35 is a schematic diagram of a distal region of an Endovascular Transmural Ablation Precision-Grip catheter with a guide wire lumen. FIGS. 36A, 36B, 36C, 36D, 36E, 36F, 36G and 36H are schematic diagrams of a distal region of an Endovascular Transmural Ablation Precision-Grip catheter with a guide wire lumens.

FIGS. 37A-37E are schematic diagrams of a distal region of an Endovascular Transmural Ablation Precision-Grip catheter with a guide wire lumen in a first arm and actuation of a second arm.

FIGS. 38 and 39 are schematic diagrams of a distal region of an Endovascular Transmural Ablation Precision-Grip catheter having irrigation or guide wire lumens.

FIG. 40 is a schematic illustration of a bipolar RF carotid septum ablation catheter having expandable structures.

FIG. 41 is a schematic illustration of a bipolar RF carotid septum ablation catheter having expandable structures.

FIG. 42A is a schematic illustration of a bipolar RF balloon catheter.

FIGS. 42B and 42C illustrate ablation catheters including an expandable structure with an ablation element mounted thereon.

FIGS. 43-45 are schematic illustrations of bipolar RF balloon catheters.

FIGS. 46-52 are schematic illustrations of catheter configured to key or couple with a carotid bifurcation for carotid body ablation.

FIGS. 53A and 53B are schematic illustrations of a carotid body ablation catheter having an inflatable balloon configured to couple with a carotid bifurcation.

FIG. 54 is a schematic illustration of an Endovascular Transmural Ablation Precision-Grip catheter configured for monopolar ablation and intercarotid septum monitoring.

FIG. 55A is a schematic illustration of a lateral view of a monopolar ablation in a carotid septum.

FIG. 55B is a schematic illustration of a transverse view of a monopolar ablation in a carotid septum.

FIG. 56A is a schematic illustration of a lateral view of a bipolar ablation in a carotid septum.

FIG. 56B is a schematic illustration of a transverse view of a bipolar ablation in a carotid septum.

FIG. 57A is a schematic illustration of a lateral view of an energy-directed ablation in a carotid septum.

FIG. 57B is a schematic illustration of a transverse view of an energy-directed ablation in a carotid septum.

FIG. 58 is a graph of temperature vs. time of an active electrode and reference electrode during an energy-directed ablation experiment.

FIGS. 59A and 59B are schematic views showing suitable placement of an active electrode and an energy-directed reference electrode in relation to an intercarotid septum.

FIG. 60 is a schematic illustration of a lateral view of a catheter comprising diverging arms and configured for an energy-directed ablation in a carotid septum.

FIG. 61 is a cutaway illustration of a lateral view a patient's right carotid artery system with a schematic illustration of an energy-directed carotid body modulation catheter positioned in the patient's internal and external carotid arteries for endovascular ablation of a carotid body.

FIG. 62 is a cutaway illustration of a lateral view a patient's right carotid artery system with a schematic illustration of an energy-directed carotid body modulation catheter positioned in the patient's internal and external carotid arteries for endovascular ablation of a carotid body.

FIG. 63 is a cutaway illustration of a lateral view a patient's right carotid artery system with a schematic illustration of an energy-directed carotid body modulation catheter positioned in the patient's internal and external carotid arteries for endovascular ablation of a carotid body.

FIG. 64 is a cutaway illustration of a lateral view a patient's right carotid artery system with a schematic illustration of an energy-directed carotid body modulation catheter positioned in the patient's internal and external carotid arteries for endovascular ablation of a carotid body.

FIG. 65 illustrates placement of a monopolar RF catheter in an external carotid artery in a porcine model.

FIGS. 66-70 illustrate histological results and assessment of ablations created by monopolar RF catheters in a porcine model.

FIG. 71 illustrates a bipolar RF arrangement for carotid body ablation.

FIG. 72 illustrates placement of bipolar RF electrodes on an arterial septum in a porcine model.

FIGS. 73-75 illustrate histological results and assessment of ablations created by bipolar RF catheters in a porcine model.

FIG. 76 illustrates histological results of a monopolar RF ablation in a narrow septum.

FIGS. 77A and 77B illustrate finite element modeling of a monopolar RF carotid septum ablation.

FIGS. 78A and 78B illustrate finite element modeling of a bipolar RF carotid septum ablation.

FIGS. 79A-79C illustrate finite element modeling of a bipolar RF carotid septum ablation.

FIG. 80 illustrates an exemplary carotid body ablation catheter.

### DETAILED DESCRIPTION

In the following detailed description, reference is made to the accompanying drawings, which form a part hereof, and in which is shown by way of illustration exemplary embodiments in which the disclosure may be practiced. These embodiments are described in sufficient detail to enable those skilled in the art to practice the inventions, and it is to be understood that the embodiments may be combined, or that other embodiments may be utilized and that structural, logical and electrical changes may be made without departing from the spirit and scope of the present disclosure.

References to “an”, “one”, or “various” embodiments in this disclosure are not necessarily to the same embodiment, and such references contemplate more than one embodiment. The following detailed description provides exemplary embodiments.

Systems, devices, and methods have been conceived for carotid body ablation (that is, full or partial ablation of one or both carotid bodies, carotid body nerves, intercarotid septums, or peripheral chemoreceptors) to treat patients having a sympathetically mediated disease (e.g., cardiac, renal, metabolic, or pulmonary disease such as hypertension, congestive heart failure, atrial fibrillation, ventricular tachycardia, dyspnea, sleep apnea, sleep disordered breathing, diabetes, insulin resistance, atrial fibrillation, chronic kidney disease, polycystic ovarian syndrome, post MI mortality) at least partially resulting from augmented peripheral chemoreflex (e.g., peripheral chemoreceptor hypersensitivity, peripheral chemosensor hyperactivity), heightened sympathetic activation, or an unbalanced autonomic tone.

A reduction of peripheral chemoreflex or reduction of afferent nerve signaling from a carotid body (CB) resulting in a reduction of central sympathetic tone is a main therapy pathway of the methods described herein. Higher than normal chronic or intermittent activity of afferent carotid body nerves is considered enhanced chemoreflex. Other therapeutic benefits such as increase of parasympathetic tone, vagal tone and specifically baroreflex and baroreceptor activity, as well as reduction of dyspnea, hyperventilation, hypercapnea, respiratory alkalosis and breathing rate may be expected in some patients. Secondary to reduction of breathing rate additional increase of parasympathetic tone can be expected in some patients. Reduced breathing rate can lead to increased tidal lung volume, reduced dead space and increased efficiency of gas exchange. Reduced dyspnea and reduced dead space can independently lead to improved ability to exercise. Shortness of breath (dyspnea) and exercise limitations are common debilitating symptoms in CHF and COPD. Augmented peripheral chemoreflex (e.g., carotid body activation) leads to increases in sympathetic nervous system activity, which is in turn primarily responsible for the progression of chronic disease as well as debilitating symptoms and adverse events seen in our intended patient populations. Carotid bodies contain cells that are sensitive to partial pressure of oxygen and carbon dioxide in blood plasma. Carotid bodies also may respond to blood flow, pH acidity, glucose level in blood and possibly other variables. Thus carotid body ablation may be a treatment for patients, for example having hypertension, heart disease or diabetes, even if chemosensitive cells are not activated.

The disclosure herein includes methods of endovascular transmural carotid body ablation, which in some embodiments includes inserting a catheter in the patient's vascular system, positioning a distal region of the catheter in a vessel proximate a carotid body (e.g., in a common carotid artery, internal carotid artery, external carotid artery, at a carotid bifurcation, proximate an intercarotid septum), coupling the distal region of the catheter to a carotid bifurcation, positioning an ablation element proximate to a target site (e.g., a carotid body, afferent nerves associated with a carotid body, a peripheral chemosensor, an intercarotid septum), and delivering an ablation agent from the ablation element to ablate the target site. Exemplary methods and devices configured to perform these methods are described herein.

### Targets:

To inhibit or suppress a peripheral chemoreflex, anatomical targets for ablation (also referred to as target tissue, targeted tissue, target ablation sites, or target sites) may include at least a portion of at least one carotid body, an aortic body, nerves associated with a peripheral chemoreceptor (e.g., carotid body nerves, carotid sinus nerve, carotid plexus), small blood vessels feeding a peripheral chemoreceptor, carotid body parenchyma, chemosensitive cells (e.g., glomus cells), tissue in a location where a carotid body is suspected to reside (e.g., a location based on pre-operative imaging or anatomical likelihood), an intercarotid septum, a portion of an intercarotid septum or a combination thereof. As used herein, ablation of a carotid body or carotid body ablation may refer to ablation of any of these target ablation sites.

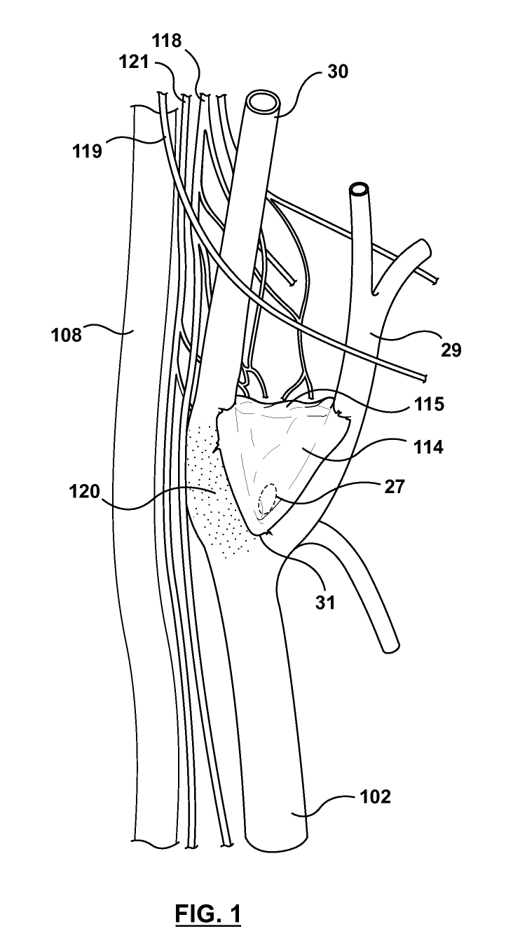

As shown in FIG. 1, a carotid body (“CB”) 27, housing peripheral chemoreceptors, modulates sympathetic tone through direct signaling to the central nervous system. Carotid bodies represent a paired organ system located near a bifurcation 31 of a common carotid artery 102 bilaterally, that is, on both sides of the neck. The common carotid artery 102 bifurcates to an internal carotid artery 30 and an external carotid artery 29. Typically, in humans each carotid body is approximately the size of a 2.5-5 mm ovoid grain of rice and is innervated both by the carotid sinus nerve (CSN, a branch of the glossopharyngeal nerve), and the ganglioglomerular (sympathetic) nerve of the nearby superior cervical ganglion. Infrequently other shapes are encountered. The CB is the most perfused organ per gram weight in the body and receives blood via an arterial branch or branches typically arising from internal or external carotid artery.

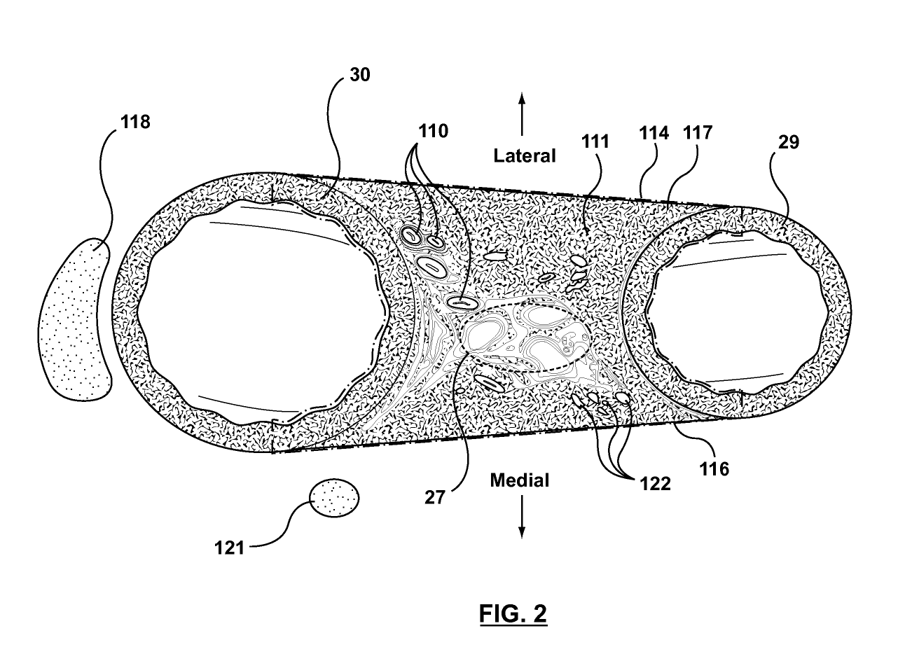

Inventors have conducted extensive human cadaver anatomy studies to understand variability in geometry and relative position of carotid arteries, carotid bodies, carotid nerves, and important non-target nerves. This information is an important part of the inventive step to determine aspects of a procedure and device that could effectively ablate a targeted tissue (e.g., carotid body, carotid body nerves, substantial portion of a carotid body) while safely avoiding iatrogenic injury of important non-target nerves. Inventors have discovered that a volume of tissue, which is referred to herein as an intercarotid septum, carotid septum, or septum, may be a suitable target for ablation in a carotid body ablation (“CBA”) procedure. Endovascular catheter assemblies, such as those described herein, were designed to be configured to ablate at least a significant portion of, and containing an ablation within or substantially within, an intercarotid septum. An exemplary intercarotid septum 114, shown in FIGS. 1 and 2, is herein defined as a wedge or triangular segment of tissue with the following boundaries: a saddle of a carotid bifurcation 31 defines a caudal aspect (an apex) of a carotid septum 114; facing walls of internal 30 and external 29 carotid arteries define two sides of a carotid septum; a cranial boundary 115 of a carotid septum extends between these arteries and may be defined as cranial to a carotid body but caudal to any important non-target nerve structures (e.g., hypoglossal nerve) that might be in the region, for example a cranial boundary may be about 7 mm to 15 mm (e.g., about 10 mm) from the saddle of the carotid bifurcation; medial 116 and lateral 117 walls of the carotid septum 114 are generally defined by planes approximately tangent to the internal and external carotid arteries; one of the planes is tangent to the lateral wall of the internal and external carotid arteries and the other plane is tangent to the medial walls of these arteries. An intercarotid septum is between the medial and lateral walls. The medial plane of an intercarotid septum may alternatively be defined as a carotid sheath on a medial side of a septum or within about 2 mm outside of the medial side of the carotid sheath. An intercarotid septum 114 may include a carotid body 27 and is typically absent of important non-target nerve structures such as a vagus nerve 118, important non-target sympathetic nerves 121, or a hypoglossal nerve 119 (see FIG. 1). Creating an ablation that is maintained or substantially maintained within an intercarotid septum may therefore effectively modulate (e.g., ablate) a carotid body while safely avoiding collateral damage of important non-target nerve structures. Probability of effectiveness may be increased as the percentage of the septum encompassed by an ablation, at the level of a carotid body or cranial to the carotid body, increases. An intercarotid septum may include some baroreceptors 120 or baroreceptor nerves. An intercarotid septum may also include small blood vessels 110, nerves 122 associated with the carotid body, and fat 111.

As used herein, a “wall” of an external or internal carotid artery, or any other vessel, is not limited to the endothelial layer, but includes any other tissue or non-tissue associated with the vessel. For example, a wall includes plaque or any other material deposited thereon. As used herein, a “wall” of a blood vessel is anything that at least partially defines the lumen through which blood flows. For example, when an electrode is in apposition with a wall of a blood vessel, it may be in contact with an endothelial layer, plaque, etc.

Carotid body nerves are anatomically defined herein as carotid plexus nerves 122 (see FIG. 2) and carotid sinus nerves. Carotid body nerves are functionally defined herein as nerves that conduct information from a carotid body to a central nervous system.

An ablation may be focused exclusively on targeted tissue, or be focused on the targeted tissue while safely ablating tissue proximate to the targeted tissue (e.g., to ensure the targeted tissue is ablated or as an approach to gain access to the targeted tissue). An ablation may be as big as a peripheral chemoreceptor (e.g., carotid body or aortic body) itself, somewhat smaller, or bigger and can include tissue surrounding the chemoreceptor such as blood vessels, adventitia, fascia, small blood vessels perfusing the chemoreceptor, or nerves connected to and innervating the glomus cells. An intercarotid plexus or carotid sinus nerve may be a target of ablation with an understanding that some baroreceptor nerves will be ablated together with carotid body nerves. Baroreceptors are distributed in the human arteries and have high degree of redundancy.

Tissue may be ablated to inhibit or suppress a chemoreflex of only one of a patient's two carotid bodies. Alternatively, a carotid body ablation procedure may involve ablating tissue to inhibit or suppress a chemoreflex of both of a patient's carotid bodies. For example a therapeutic method may include ablation of one carotid body, measurement of resulting chemosensitivity, sympathetic activity, respiration or other parameter related to carotid body hyperactivity and ablation of the second carotid body if needed to further reduce chemosensitivity following unilateral ablation. The decision to ablate one or both carotid bodies may be based on pre-procedure testing or on patient's anatomy.

An embodiment of a therapy may substantially reduce chemoreflex without excessively reducing the baroreflex of the patient. The proposed ablation procedure may be targeted to substantially spare the carotid sinus, baroreceptors distributed in the walls of carotid arteries (e.g., internal carotid artery), and at least some of the carotid sinus baroreceptor nerves that conduct signals from said baroreceptors. For example, the baroreflex may be substantially spared by targeting a limited volume of ablated tissue possibly enclosing the carotid body, tissues containing a substantial number of carotid body nerves, tissues located in periadventitial space of a medial segment of a carotid bifurcation, or tissue located at the attachment of a carotid body to an artery. Said targeted ablation is enabled by visualization of the area or carotid body itself, for example by CT, CT angiography, MRI, ultrasound sonography, IVUS, OCT, intracardiac echocardiography (ICE), trans-esophageal echocardiography (TEE), fluoroscopy, blood flow visualization, or injection of contrast, and positioning of an instrument in the carotid body or in close proximity while avoiding excessive damage (e.g., perforation, stenosis, thrombosis) to carotid arteries, baroreceptors, carotid sinus nerves or other important non-target nerves such as vagus nerve or sympathetic nerves located primarily outside of the carotid septum. CT angiography and ultrasound sonography have been demonstrated to locate carotid bodies in most patients. Thus imaging a carotid body before ablation may be instrumental in (a) selecting candidates if a carotid body is present, large enough and identified and (b) guiding therapy by providing a landmark map for an operator to guide an ablation instrument to the carotid septum, center of the carotid septum, carotid body nerves, the area of a blood vessel proximate to a carotid body, or to an area where carotid body itself or carotid body nerves may be anticipated. Note that although a landmark map may be useful, the need for it may be reduced or eliminated by using devices configured to create and contain an ablation within an intercarotid septum, such as the devices disclosed herein, therefor reducing costly pre-procedural planning and operator dependency on following a landmark map. It may also help exclude patients in whom the carotid body is located substantially outside of the carotid septum in a position close to a vagus nerve, hypoglossal nerve, jugular vein or some other structure that can be endangered by ablation. In one embodiment only patients with carotid body substantially located within the intercarotid septum are selected for ablation therapy. Pre-procedure imaging can also be instrumental in choosing the right catheter depending on a patient's anatomy. For example a catheter with more space between arms can be chosen for a patient with a wider septum.

Once a carotid body is ablated, surgically removed, or denervated, the carotid body function (e.g., carotid body chemoreflex) does not substantially return in humans (in humans aortic chemoreceptors are considered undeveloped). To the contrary, once a carotid sinus baroreflex is removed (such as by resection of a carotid sinus nerve) it is generally compensated, after weeks or months, by the aortic or other arterial baroreceptor baroreflex. Thus, if both the carotid chemoreflex and baroreflex are removed or substantially reduced, for example by interruption of the carotid sinus nerve or intercarotid plexus nerves, baroreflex may eventually be restored while the chemoreflex may not. The consequences of temporary removal or reduction of the baroreflex can be in some cases relatively severe and require hospitalization and management with drugs, but they generally are not life threatening, terminal or permanent. Thus, it is understood that while selective removal of carotid body chemoreflex with baroreflex preservation may be desired, it may not be absolutely necessary in some cases.

### Ablation:

The term “ablation” may refer to the act of altering tissue to suppress or inhibit its biological function or ability to respond to stimulation permanently or for an extended period of time (e.g., greater than 3 weeks, greater than 6 months, greater than a year, for several years, or for the remainder of the patient's life). For example, ablation may involve, but is not limited to, thermal necrosis or irreversible electroporation of target tissue cells.

Carotid Body Ablation (“CBA”) herein refers to ablation of a target tissue wherein the desired effect is to reduce or remove the afferent neural signaling from a chemosensor (e.g., carotid body) or reducing a chemoreflex. Chemoreflex or afferent nerve activity cannot be directly measured in a practical way, thus indexes of chemoreflex such as chemosensitivity can sometimes be used instead. Chemoreflex reduction is generally indicated by a reduction of an increase of ventilation and respiratory effort per unit of blood gas concentration, saturation or blood gas partial pressure change or by a reduction of central sympathetic nerve activity in response to stimulus (such as intermittent hypoxia or infusion of a drug) that can be measured directly. Sympathetic nerve activity can be assessed indirectly by measuring activity of peripheral nerves leading to muscles (MSNA), heart rate (HR), heart rate variability (HRV), production of hormones such as renin, epinephrine and angiotensin, and peripheral vascular resistance. All these parameters are measurable and their change can lead directly to the health improvements. In the case of CHF patients blood pH, blood PCO₂, degree of hyperventilation and metabolic exercise test parameters such as peak VO₂, and VE/VCO₂ slope are also important. It is believed that patients with heightened chemoreflex have low VO₂ and high VE/VCO₂ slope measured during cardiopulmonary stress test (indexes of respiratory efficiency) as a result of, for example, tachypnea and low blood CO₂. These parameters are also related to exercise limitations that further speed up patient's status deterioration towards morbidity and death. It is understood that all these indexes are indirect and imperfect and intended to direct therapy to patients that are most likely to benefit or to acquire an indication of technical success of ablation rather than to proved an exact measurement of effect or guarantee a success. It has been observed that some tachyarrhythmias in cardiac patients are sympathetically mediated. Thus, carotid body ablation may be instrumental in treating reversible atrial fibrillation and ventricular tachycardia.

In the context of this disclosure ablation includes denervation, which means destruction of nerves or their functional destruction, meaning termination of their ability to conduct signals. Selective denervation may involve, for example, interruption of afferent nerves from a carotid body while substantially preserving nerves from a carotid sinus, which conduct baroreceptor signals. Another example of selective denervation may involve interruption of nerve endings terminating in chemo sensitive cells of carotid body, a carotid sinus nerve, or intercarotid plexus which is in communication with both a carotid body and some baroreceptors wherein chemoreflex or afferent nerve stimulation from the carotid body is reduced permanently or for an extended period of time (e.g., years) and baroreflex is substantially restored in a short period of time (e.g., days or weeks). As used herein, the term “ablate” refers to interventions that suppress or inhibit natural chemoreceptor or afferent nerve functioning, which is in contrast to electrically neuromodulating or reversibly deactivating and reactivating chemoreceptor functioning (e.g., with an implantable electrical stimulator/blocker).

Carotid body ablation may include methods and systems for the thermal ablation of tissue via thermal heating mechanisms. Thermal ablation may be achieved due to a direct effect on tissues and structures that are induced by the thermal stress. Additionally or alternatively, the thermal disruption may at least in part be due to alteration of vascular or peri-vascular structures (e.g., arteries, arterioles, capillaries or veins), which perfuse the carotid body and neural fibers surrounding and innervating the carotid body (e.g., nerves that transmit afferent information from carotid body chemoreceptors to the brain). Additionally or alternatively thermal disruption may be due to a healing process, fibrosis, or scarring of tissue following thermal injury, particularly when prevention of regrowth and regeneration of active tissue is desired. As used herein, thermal mechanisms for ablation may include both thermal necrosis or thermal injury or damage (e.g., via sustained heating, convective heating or resistive heating or combination). Thermal heating mechanisms may include raising the temperature of target neural fibers above a desired threshold, for example, above a body temperature of about 37° C. e.g., to achieve thermal injury or damage, or above a temperature of about 45° C. (e.g., above about 60° C.) to achieve thermal necrosis. It is understood that both time of heating, rate of heating and sustained hot or cold temperature are factors in the resulting degree of injury.

In addition to raising temperature during thermal ablation, a length of exposure to thermal stimuli may be specified to affect an extent or degree of efficacy of the thermal ablation. For example, the length of exposure to thermal stimuli may be for example, longer than or equal to about 30 seconds, or even longer than or equal to about 2 minutes. Furthermore, the length of exposure can be less than or equal to about 10 minutes, though this should not be construed as the upper limit of the exposure period. A temperature threshold, or thermal dosage, may be determined as a function of the duration of exposure to thermal stimuli. Additionally or alternatively, the length of exposure may be determined as a function of the desired temperature threshold. These and other parameters may be specified or calculated to achieve and control desired thermal ablation.

In some embodiments, ablation of carotid body or carotid body nerves may be achieved via direct application of ablative energy to target tissue. For example, an ablation element may be applied at least proximate to the target, or an ablation element may be placed in a vicinity of a chemosensor (e.g., carotid body). In other embodiments, thermally-induced ablation may be achieved via indirect generation or application of thermal energy to the target neural fibers, such as through application of an electric field (e.g., radiofrequency, alternating current, and direct current) to the target tissue. For example, thermally induced ablation may be achieved via delivery of a pulsed or continuous thermal electric field to the target tissue such as RF and pulsed RF, the electric field being of sufficient magnitude or duration to thermally induce ablation of the target tissue (e.g., to heat or thermally ablate or cause necrosis of the targeted tissue). Additional and alternative methods and apparatuses may be utilized to achieve ablation, as described hereinafter.

### Endovascular Access:

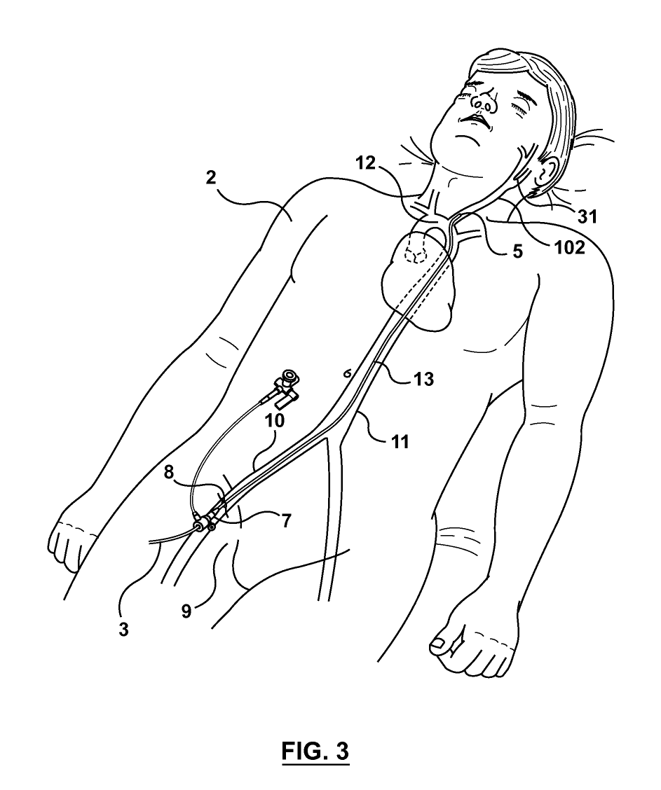

An endovascular catheter for transmural ablation may be delivered into a patient's vasculature via percutaneous introduction into a blood vessel, for example a femoral, radial, brachial artery or vein, or even via a cervical or temporal artery approach into a carotid artery. For example, FIG. 3 depicts in simplified schematic form the placement of a carotid access sheath 13 into a patient 2. The sheath is depicted in position for insertion of an endovascular carotid body ablation catheter 3 into the vicinity of the left carotid artery bifurcation 31 through a central lumen of the carotid access sheath 13. The distal end of the sheath 5 is shown residing in the left common carotid artery 102. The proximal end of the sheath 7 is shown residing outside of the patient 2, with the sheath's entry point 8 into the patient being in the vicinity of the groin 9. From the sheath's entry point 8, the sheath enters a peripheral artery 10, and traverses the abdominal aorta 11, the aortic arch 12, and into the left common carotid artery 102. The carotid access sheath 13 may be commercially available, or may be configured specifically for endovascular transmural ablation of a carotid body. An endovascular procedure may involve the use of a guide wire, delivery sheath, guide catheter, introducer catheter or introducer. Furthermore, these devices may be steerable and torquable (i.e. able to conduct rotation from proximal to distal end). Techniques for placing a carotid access sheath 13 into position as depicted are known to those skilled in the art of endovascular carotid procedures. A carotid access sheath may include lumens for guide wire placement, contrast injection and steerable mechanisms for deflection. Guide wire(s) can be buddy wires placed in the sheath or traverse through the separate limens in sheath or in the catheter itself. Where catheter or sheath lumens are used for contrast injection they also can be used to inject drugs and specifically chemicals that excite or suppress the carotid body. This way carotid body function can be tested during and after a CBM procedure to determine procedure success in stimulating or suppressing carotid body function. Examples of such agents known in medicine and include for example adenosine and dopamine.

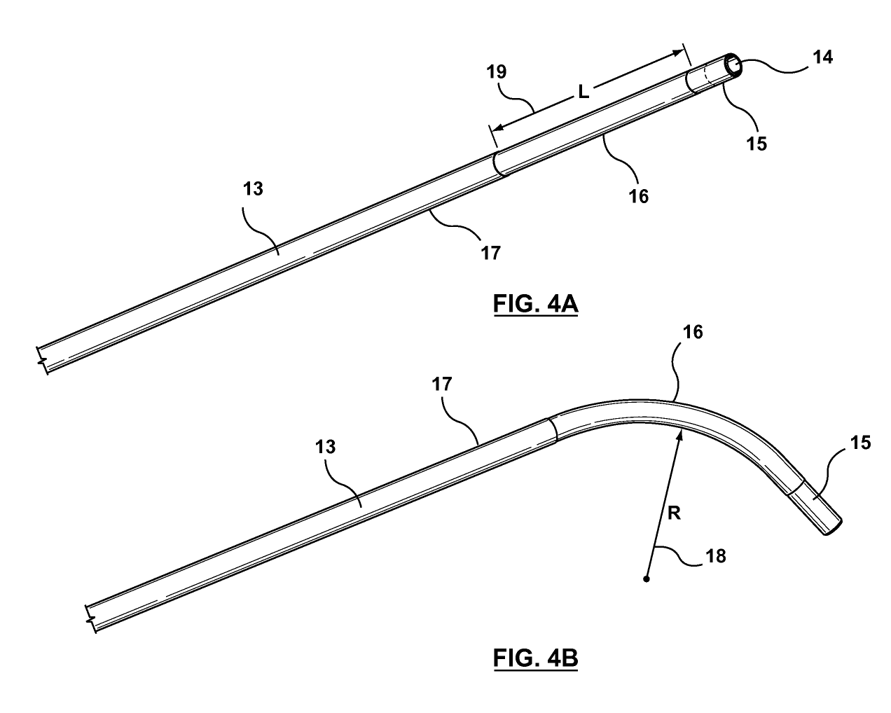

FIG. 4A and FIG. 4B depict a distal end of a carotid access sheath specifically configured for Endovascular Transmural Ablation of a carotid body, which will hereby be referred to as an ETA Carotid Access Sheath 13. The ETA Carotid Access Sheath comprises a central lumen 14 that traverses the length of the sheath from the distal end depicted in FIGS. 4A and 4B to the proximal end (not shown). An ETA Carotid Access Sheath may be sized to accommodate an ablation catheter plus a space sufficient to allow for injection of contrast fluid. The maximum diameter of the sheath is limited by the smallest vessel diameter in which the sheath will be inserted. However, invasiveness of the procedure is minimized as sheath diameter is reduced. For example, the central lumen 14 of the sheath may have a diameter between about 3 French and 12 French (e.g., about 7 French when used with a 6 French ablation catheter). The ETA Carotid Access Sheath 13 may comprise a distal tip 15, a deflectable segment 16 proximal to the distal tip 15, and a non-deflectable segment 17 proximal to the deflectable segment 16. In addition, not shown, is a handle mounted at the proximal end of the catheter with an actuator configured for user-actuated deflection of the deflectable segment 16. A pull wire in communication between the distal tip 15 and the handle mounted actuator at the proximal end is configured to deflect the deflectable segment 16 in response to user actuation. The techniques for constructing a deflectable tipped sheath are known to those skilled in the art, and therefore are not further elaborated. The ETA Carotid Access Sheath is arranged specifically for endovascular transmural ablation of a carotid body in at least one of the following manners: the radius of curvature 18 and length 19 of the deflectable segment are configured for use in the vicinity of the carotid bifurcation with the radius of curvature 18 being between 5 mm and 20 mm, and the length of the deflectable segment 19 being between 10 mm and 25 mm; distal tip 15 may comprise at least one electrode, not shown, configured for at least one of the following: transmural ablation of a carotid body, stimulation of a carotid body, blockade of a carotid body, stimulation of nervous function not associated with a carotid body, and blockade of nervous function not associated with the function of a carotid body, whereby for these specific arrangements the ETA Carotid Access Sheath 13 is used for transmural ablation, and the central lumen 14 is used to place into the region of the carotid bifurcation 31 an additional procedural instrument, the stimulation or blockade is used to locate a preferred position for transmural ablation of a carotid artery, and stimulation or blockade of nervous function not associated with a carotid body is used to avoid damage to important non-target nervous structures such as the vagal nerve.

Alternatively, a guide wire may be delivered through a patient's vasculature to carotid arteries and a sheath may be delivered over the guide wire. The sheath may or may not have steering or deflectable capabilities. For example, if a sheath is delivered over a wire to a common carotid artery and an ablation catheter is delivered through the sheath, deflection may facilitate positioning of the ablation catheter at a target site and reduce unnecessary contact with non-target portions of carotid vasculature, thus reducing risk of dislodging plaque. An ablation catheter may have deflection capabilities to facilitate positioning at a target site, in which case it may not be necessary for a sheath to have deflection capabilities.

### Endovascular Transmural Ablation Precision-Grip Catheters:

Devices have been conceived for endovascular transmural carotid body ablation comprising two arms, herein referred to as Endovascular Transmural Ablation Precision-Grip (ETAP) catheters, which may also be referred to herein as Endovascular Transmural Ablation Forceps (ETAF) catheters. Embodiments of ETAP catheters disclosed herein comprise a distal end and a proximal end, wherein the distal end is inserted into a patient's vasculature and delivered proximate a target site, and the proximal end is maintained outside the patient's body. In some embodiments the distal region of an ETAP catheter comprises ablation element(s) positioned on two arms (which may also be referred to herein as splines, diverging structures, diverging arms, fingers, bifurcated structures, prongs, together as forceps arms, or individually as a forceps arm) in a configuration that positions at least one ablation element in an internal carotid artery and at least one second ablation element in an external carotid artery on an intercarotid septum at a position relative to a target carotid body or nerves associated with a carotid body that is suitable for carotid body ablation. Ablation elements may be, for example, a pair of bipolar radiofrequency electrodes; a pair of bipolar irreversible electroporation electrodes; more than two electrodes; or a single monopolar radiofrequency electrode and second electrode used as current return or to measure properties of target tissue such as electrical impedance, temperature, or blood flow. Apposition of one or both of the ablation elements with an intercarotid septum is achieved by causing a closing force of the arms, for example via resilient forces of the arms or a mechanical actuation means. Structural aspects of catheters may be described herein as bifurcated, but it is not intended that catheter be limited to only two of the structures. For example, when bifurcated is used to describe structural components, at least two are present, and there may be more than two.

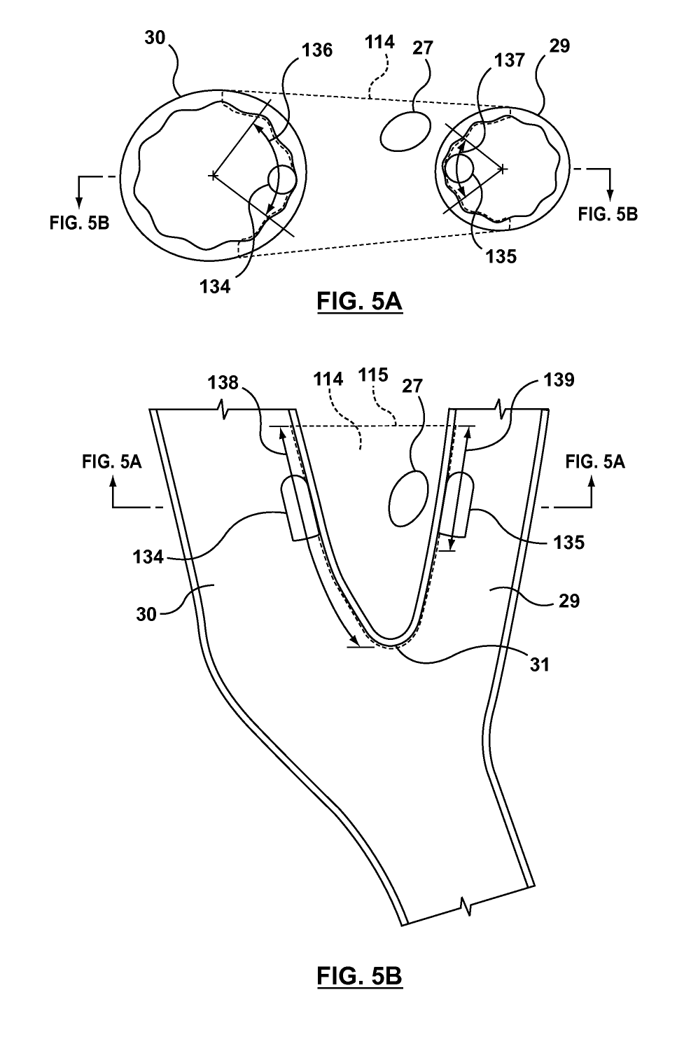

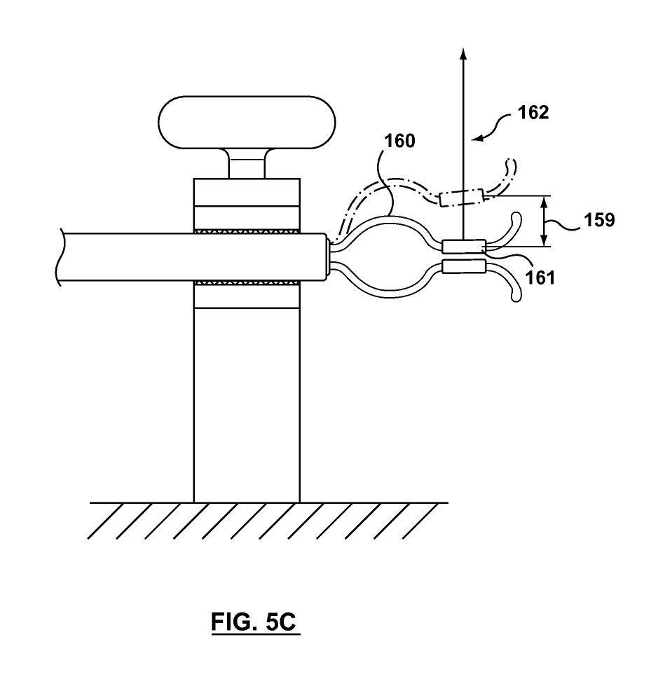

FIGS. 5A and 5B illustrate an example of ablation element positioning that may effectively and safely ablate a carotid body 27. FIG. 5A shows, outlined with a dashed line, a transverse cross-section of an intercarotid septum 114 bordered by an internal carotid artery 30 and an external carotid artery 29. In this embodiment, a first ablation element 134 is placed in the internal carotid artery 30 in contact with the vessel wall within a vessel wall arc 136 directed toward the external carotid artery; a second ablation element 135 is placed in the external carotid artery 29 in contact with the vessel wall within a vessel wall arc 137 directed toward the internal carotid artery. Each vessel wall arc 136 and 137 is contained within limits of the intercarotid septum 114 and comprises an arc length no greater than about 25% (e.g., about 15 to 25%) of the circumference of the respective vessel. In this example, the ablation elements 134 and 135 may be bipolar radiofrequency electrodes or irreversible electroporation electrodes wherein electrical current is passed from one electrode to the other electrode through the intercarotid septum. Placement of ablation elements as described may facilitate targeted deposition of energy and the creation of an ablation lesion that is contained within the intercarotid septum, thus avoiding injury of non-target nerves that reside outside the septum, and an ablation that is sufficiently large (e.g., with respect to a width dimension, extending approximately from the internal carotid artery to the external carotid artery) to effectively modulate a carotid body or its associated nerves. Specifically, this configuration and placement facilitates deposition of energy along a line between the electrodes and inhibits it in the medial direction (towards the spine).

FIG. 5B shows, outlined with a dashed line, a longitudinal cross-section of an intercarotid septum 114 bordered by an internal carotid artery 30, an external carotid artery 29, a saddle of a carotid bifurcation 31 and a cranial (towards the head) boundary 115 that is between about 10 to 15 mm cranial from the saddle 31. In this example, the first ablation element 134 is placed in the internal carotid artery 30 in contact with the vessel wall within a first range 138; a second ablation element 135 is placed in the external carotid artery 29 in contact with the vessel wall within a second range 139. The first range 138 may extend from the inferior apex of the bifurcation saddle 31 to the cranial boundary 115 of the septum (e.g., about 10 to 15 mm from the bifurcation saddle). The second range 139 may extend from a position about 4 mm superior from the bifurcation saddle 31 to the cranial boundary 115 of the septum (e.g., about 10 or 15 mm from the bifurcation saddle). As an example, an ETAP catheter may be configured to place a distal tip of a 4 mm long electrode in an internal carotid artery about 10 mm from a carotid bifurcation and a distal tip of a second 4 mm long electrode in a corresponding external carotid artery at about 10 mm from the carotid bifurcation. The electrodes 134 and 135 may be equidistant from the saddle 31 or they may be unequal distances from the saddle.

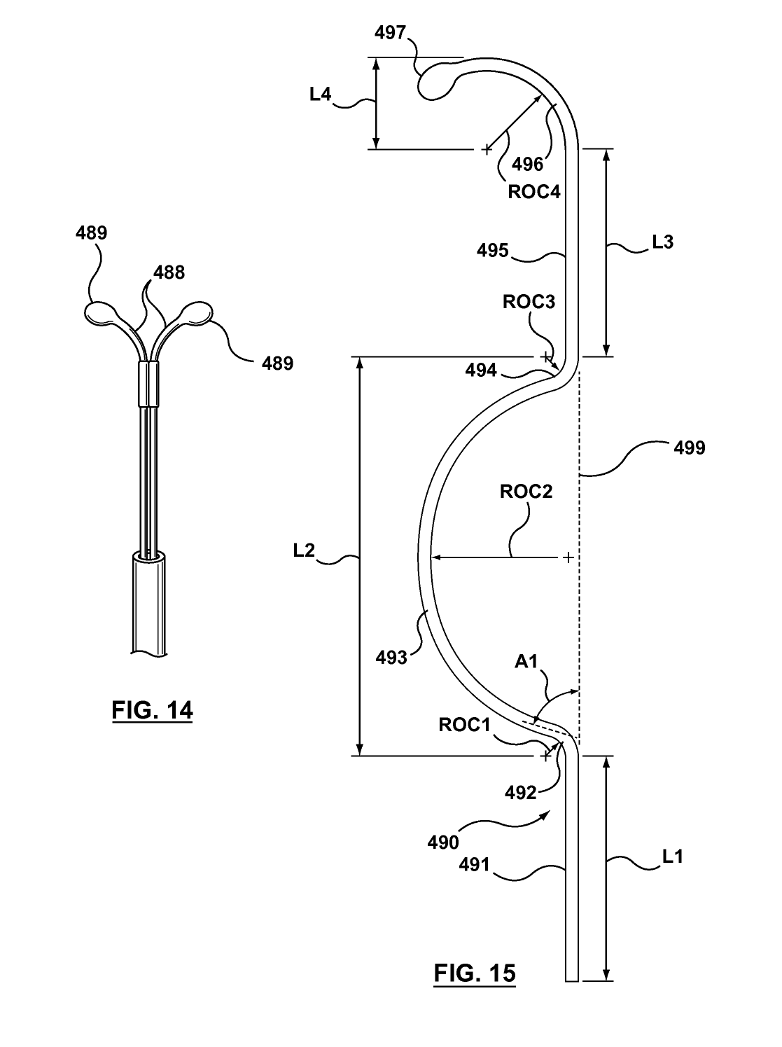

The method and devices herein take advantage of natural anatomy to position ablation elements at a suitable position for carotid body ablation. For example, the diverging arms of an ETAP catheter, or another aspect of the catheter, may be configured to couple with a carotid bifurcation by advancing one finger into an internal carotid artery and the other finger into an external carotid artery until the region where the arms diverge (divergence point) and the saddle, or apex, of the bifurcation contact and further advancement the catheter into a patient's vasculature is physically impeded by the contact. The dimensions of the arms and position of ablation elements on the arms are configured so the ablation elements will be positioned relative to the saddle of the bifurcation as shown in FIG. 5B. For example, ablation elements may be approximately 3 to 10 mm long (e.g., about 4 mm long); a finger placed in an internal carotid artery may have a length, including the length of the ablation elements, of 3 to 15 mm (e.g., about 10 mm); and a finger placed in an external carotid artery may have a length, including the length of the ablation elements, of approximately 7 to 15 mm (e.g., about 10 mm). The arms may have substantially equal length or one may be longer than the other (e.g., the finger placed in the external carotid artery may be longer than the finger placed in the internal carotid artery). The ETAP catheters may be configured to apply a closing force to the arms, in other words, a force in each finger or ablation element that is directed approximately toward the other finger or ablation element. The closing force may be active or passive. Passive closing force may be accomplished, for example, via elastic resiliency of the arms, or transition of shape memory Nitinol wire in arms from a martensitic to austenitic state (with a transition temperature within a few degrees centigrade below blood temperature, e.g., 34-36 degrees centigrade). Active closing force may be accomplished for, example, via mechanical actuation, or transition of shape memory Nitinol wire in arms from a martensitic to austenitic state (with a transition temperature reached by applying electrical current to the wires). When the catheter arms are positioned in an internal and external carotid artery and a closing force is applied to the arms the ablation elements will move toward one another until opposed by the internal and external carotid artery vessel walls. Then the ablation elements will slide along the vessel walls and towards one another until they settle within the vessels approximately at two positions having the shortest distance between them at a desired height from a carotid bifurcation saddle. This action is herein referred to as self-alignment. For example, in some embodiments in which the closing force is passive (for example without limitation, FIGS. 14-17, 30A-32A, 32I, 33A-C, 34A-C, and 80), the self-alignment is due at least in part to resiliency of the arms. This positioning that uses natural anatomy is within the suitable position range shown in FIG. 5A. Since arms are generally flexible and elastic the ablation elements will adapt to pulsations of vessel walls and resettle in the suitable position range even if the patient moves. For example without limitation, the embodiments in FIGS. 14-17, 30A-32A, 32I, 33A-C, 34A-C, and 80 are configured as such.

In some situations, common, internal and external carotid arteries may be aligned in a plane or close to a plane. However, carotid artery geometry is highly variable and in many situations the common, internal and external carotid arteries may be out of plane with one another. An ETAP catheter may comprise arms that are configured to adjust alignment with one another and with the catheter shaft in order to become aligned with carotid arteries that are out of plane. For example, the arms may pivot on a catheter shaft to accommodate out of plane vessel geometry. Alternatively, arms may comprise an elastic flexibility that allows them to bend in any radial direction to conform to vessels that are out of plane. In such an embodiment, the arms may be flexible enough to deform or deflect and adjust to vessel direction while elastic or resilient enough to apply ablation element contact force suitable for applying ablation energy. For example, the arms may comprise a structural segment that provides flexibility and elasticity. A structural segment may be, for example, a Nitinol or stainless steel spring wire with a round cross section and a diameter of about 0.004″ to 0.018″ (e.g., about 0.006″ to 0.012″). In such an embodiment, a first finger may be placed in an internal carotid artery and a second finger in an external carotid artery, closing force may be applied, and if the vessels are not in plane the arms can be configured to flex as the ablation elements contact vessel walls and slide toward two positions having approximately a shortest distance between them at a desired height from a carotid bifurcation saddle, that is to say the ablation elements are self-aligned. In these embodiments the fingers are configured to flex independently of one another with respect to the catheter shaft.

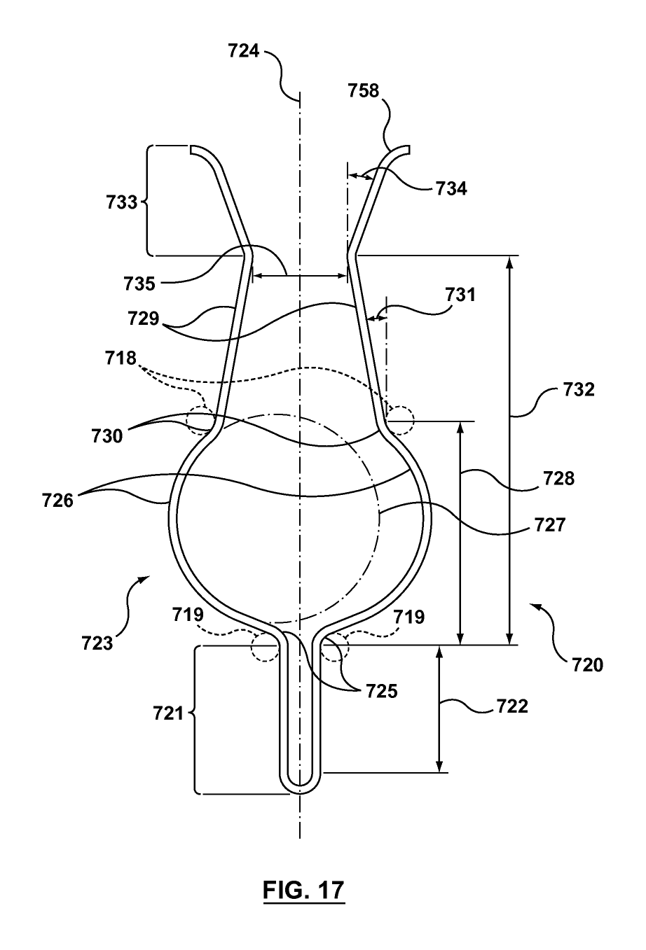

In addition to a self-aligning action, the closing force of the arms, weather passive or active, also provides contact force between ablation elements and target vessel walls of an intercarotid septum. Too little closing force may result in undesired electrode contact such as intermittent contact, contact along only part of the length of an electrode, movement of electrodes during energy delivery, unpredictable temperature measurement, excessively small ablation, or unpredictable ablation formation. Too strong a closing force may result in excessive trauma to vessel walls, plaque dislodgement, excessively large ablation, unpredictable ablation formation, or difficulty retracting the arms into a sheath. Closing force also impacts electrode contact area, as greater force within a range increases the contact area between the ablation element and the wall by pressing an electrode into distensible vessel tissue. For example, electrode contact area may be in a range of about 4 mm² to about 7.5 mm² per electrode. A closing force of a catheter arm may be characterized using force testing. For example, a mechanical test as shown in FIG. 5C comprises applying a pulling force 162 substantially orthogonal to a cantilevered catheter arm to characterize flexure of the arm. Force is applied by a force tester at a known rate of 20 mm/minute to a consistent location on the arm, for example at a proximal, distal or middle point of an electrode 161 mounted to an arm 160. This force characterizes a force needed to deflect an arm with respect to deflected distance 159. This test, performed using a number of prototypes that were found to perform well in animal tests, resulted in a deflection force in a range of 0 to 0.924N over a deflection range of 0 to 10 mm. Prototypes having superelastic structural Nitinol wires having a diameter of 0.010″ to 0.012″ were found to have a suitable balance of flexibility, allowing easy retraction into a sheath and minimal traumatic force on vessels, and resiliency, allowing deployment to a preformed shape when advanced from a sheath and suitable closing force to apply contact force between electrodes and carotid septum walls for septa having a thickness of about 2 to about 8 mm and arms having a moment arm (e.g., 728 of FIG. 17 or L2 of FIG. 15) of about 5 to 7 mm. These results are merely illustrative and are not intended to suggest that catheters must include the illustrative dimensions or properties.

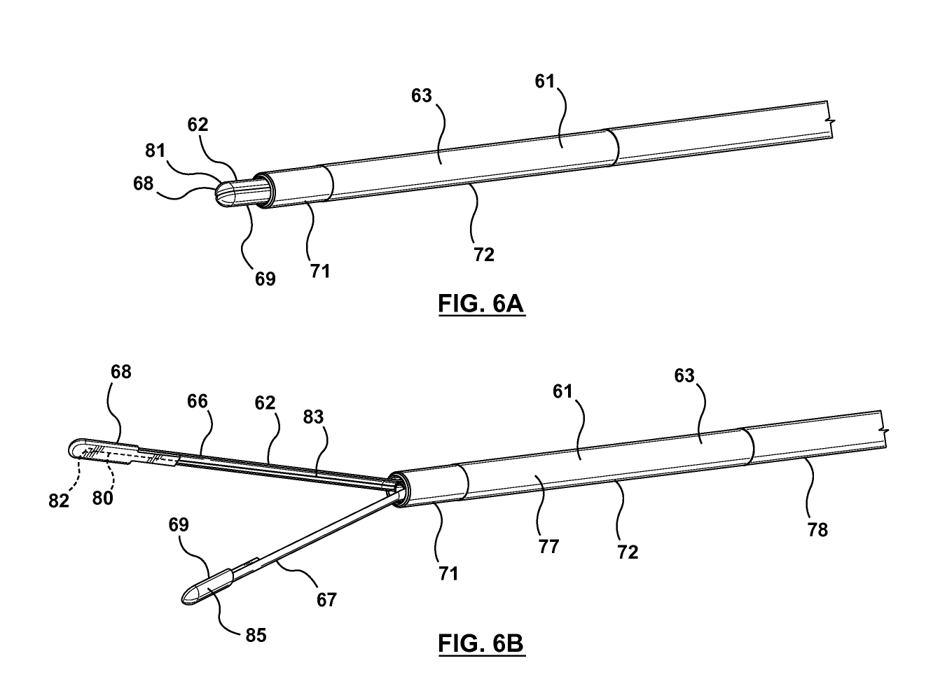

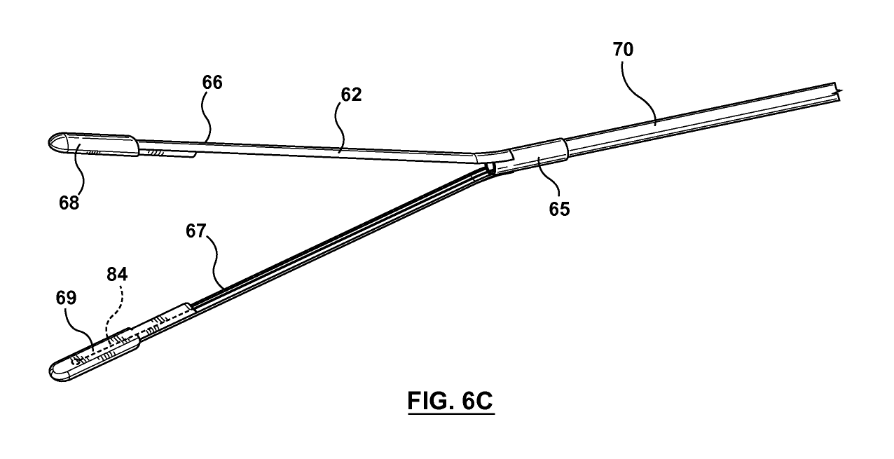

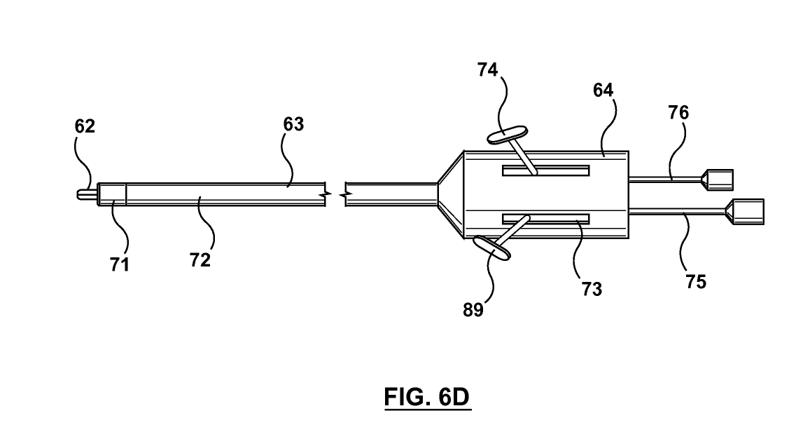

FIG. 6A, FIG. 6B, FIG. 6C, and FIG. 6D depict a distal region of an embodiment of an Endovascular Transmural Ablation Precision-Grip (ETAP) catheter 61 (which may also be referred to herein as an Endovascular Transmural Ablation Forceps “ETAF” catheter). The ETAP catheter 61 comprises an arms, or forceps, assembly 62, an arms, or forceps, sheath 63, and a proximal terminal 64. The arms assembly 62 comprises an arms end piece 65 with two arms 66 and 67, one ablation element, which may be referred to herein as a forceps pad, 68 mounted at the end of finger, or jaw strut, 66, and a second ablation element, or forceps pad, 69 mounted on the end of finger 67 as shown, and a central tube 70 that has the arms end piece 65 mounted at the distal end. The arms sheath 63 comprises a distal tip 71, and a sheath shaft 72. Mounted on the proximal end of the sheath shaft 72 is the proximal terminal 64 comprising a handle 73, with an arms actuator, or forceps actuator, 74, and electrical connector 75, and a hub and tube 76, in communication with central tube 70. Optionally, the arms sheath 63 may be configured with a user deflectable segment 77 proximal to the distal tip 71, and a non-deflectable segment 78 immediately proximal to the deflectable segment 77. Proximal terminal 64 may further comprise a deflectable segment actuator 89 which is in communication with deflectable segment 77 be means of a pull wire, not shown. The arms assembly 62 resides within arms sheath 63 in a slidable relationship. In this embodiment arms 66 and 67 are constructed to be biased to an open configuration as depicted in FIG. 6B. When the arms sheath 63 is slidably advanced forward, arms 66 and 67 are forced towards one another by distal tip 71. When the arms sheath 63 is advanced over arms assembly 62 the ablation elements 68 and 69 are in a closed position as depicted in FIG. 6A and can be fully retracted into the sheath. The advancement and retraction of the arms sheath 63 over the arms assembly 62 may be controlled by actuator 74 mounted in proximal terminal handle 73. Alternatively sheath and catheter can be slidably manipulated by hand or by other ways and mechanisms suitable for advancing one tube inside another. The pinching force of the ablation elements on tissue may also be controlled by actuator 74. Actuator 74, may optionally provide means for the user to select a ablation element contact force, observe by means of a force gage a contact force, or to provide the user with a tactile feedback of the contact force. Alternatively visualization by fluoroscopy can be used to gage the apposition of ablation elements to the walls of the intercarotid septum.

Ablation element 68 may be configured as an electrode whereby inner surface 80 may be bare metal and outer surface 81 may be electrically insulated. Ablation element 68 may be configured as an electrode whereby a portion of outer surface 81 is bare metal and where inner surface 80 is may be insulated. Ablation element 68 may be configured as an electrode with a temperature sensor 82 mounted within the walls of ablation element 68 or attached to a surface of an electrode or proximate an electrode. Temperature sensor lead wire(s) 83 connect temperature sensor 82 to electrical connector 75 of proximal terminal 64 through central tube 70. Ablation element 69 may be configured as an electrode whereby inner surface 84 may be bare metal and outer surface 85 may be insulated. Ablation element 69 may be configured as an electrode whereby a portion of outer surface 85 is bare metal and where inner surface 84 is may be insulated. Ablation element 69 may be configured as an electrode with a temperature sensor 82 mounted within the walls of ablation element 69. Temperature sensor lead wire(s) 83 connect temperature sensor 82 to electrical connector 75 of proximal terminal 64 through central tube 70. Ablation element 68 may be solid metal, or a polymer/metal composite structure or a ceramic/metal composite structure. Ablation element 69 may also be solid metal, or a polymer/metal composite structure or a ceramic/metal composite structure. Arms 66 and 67 may be fabricated from a super-elastic metallic alloy such as Nitinol, but may be fabricated from another metallic alloy, or may be a composite structure. Central tube 70 may be fabricated from a super-elastic alloy, or may be constructed from another metallic alloy, or may be composite structure. Central tube 70 is configured to work in conjunction with arms actuator 74 a to apply a tensile force on the arms assembly 62 for advancement of arms sheath 63 over arms assembly 62 to close arms, and to apply a compressive force on the arms assembly 62 to withdraw arms sheath 63 from over arms assembly 62 to open arms or to apply torque to rotate arms. Central tube 70 can be configured as an electrical conduit between ablation element 68 or ablation element 69 and electrical connector 75. It may include guide wire lumens and irrigation fluid delivery lumens. Alternatively, center tube 70 may be configured with wires to connect ablation element 68 or ablation element 69 to electrical connector 75. Electrical connector 75 is configured to connect an electrode surface on ablation element 68 or an electrode surface of ablation element 69 to one pole of an electrical generator. Electrical connector 75 may be configured to connect an electrode surface of ablation element 68 to one pole of an electrical generator, and to connect an electrode surface of ablation element 69 to the opposite pole of an electrical generator. An electrical generator may be configured for connection to electrical connector 75 and to supply RF ablation current to an electrode surface on ablation element 68 or an electrode surface on ablation element 69. The electrical generator may be further configured to provide an electrode surface on ablation element 68 with neural stimulation current or neural blockade current or to provide an electrode surface on ablation element 69 with neural stimulation current or neural blockade current. The electrical generator may be further configured to provide impedance measurement. Impedance can be measured using the same frequency generator RF at a low current/voltage/power compared to ablation power. Ablation elements 68 and 69 may be constructed in a manner where their fluoroscopic appearance is distinct to provide the user with an ability to distinguish ablation element 68 from ablation element 69. Ablation elements 68 and 69 may be of same size and surface area or different. For example it can be desired to have an electrode 69 in an internal carotid artery with a larger surface area than electrode 68 placed in an external carotid artery to achieve lower current density in the internal carotid artery where risk of embolization, char and clot is more severe. Arm 66 placed in an external carotid artery may be longer than arm 67 placed in an internal carotid artery to allow for better fixation and more distal lesion while taking advantage of lower embolization risk from manipulations in an external carotid artery.

In alternative embodiments arms 66 and 67 are biased, or pre-formed, in more of a closed configuration such that they can be slid over a carotid bifurcation, as is described below with reference to alternative embodiments. In some embodiments they can be biased to a completely closed configuration in which arms 66 and 67 are engaged with each other or very nearly touching each other (e.g., 1 mm or less apart).

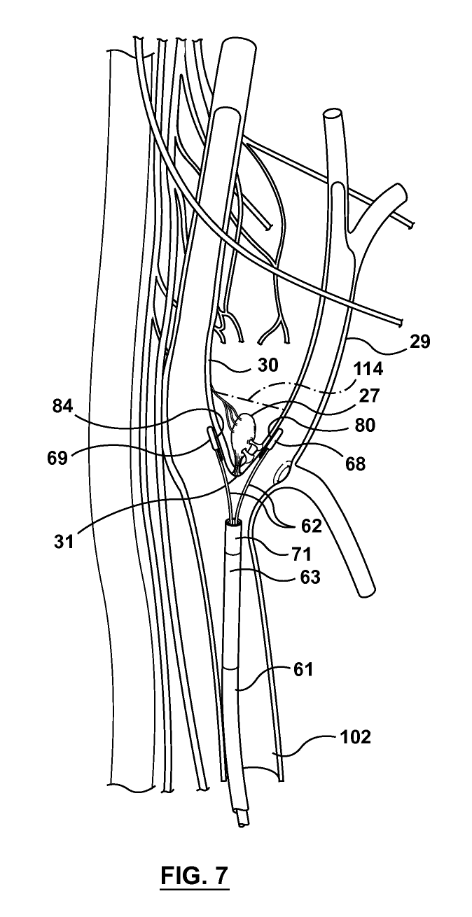

FIG. 7 depicts an ETAP catheter 61 in position for an exemplary carotid body ablation method. The ETAP catheter is positioned in the vicinity of the carotid bifurcation 31 with the distal sheath tip 71 just proximal to the carotid bifurcation 31, with ablation element 68 positioned against the wall of the external carotid artery 29, and ablation element 69 positioned against the wall of the internal carotid artery 30 within the range suitable for carotid body ablation. ETAP catheter sheath 63 has been advanced over arms assembly 62 to apply a gentle squeezing force on the intercarotid septum 114 within which at least partially lies a carotid body 27. In one embodiment depicted here, inner surface 80 of ablation element 68 is configured as an electrode. In an additional embodiment, inner surface 84 of ablation element 69 is configured as an electrode. In another embodiment inner surface 80 of ablation element 68, and inner surface 84 of ablation element 69 are both configured as electrodes, where inner surface 80 and inner surface 84 are connected to the same pole, or opposite poles of an electric generator. The electrical generator may be configured to supply RF ablation current, or neural stimulation current or neural blockade current or impedance measurement current and sensing. During RF ablation the squeezing force of arms 62 may enhance ablation by compressing the intercarotid septum 114 to achieve apposition of electrodes to a target ablation site (e.g., the inner surface of internal and external carotid arteries forming the V surface of an intercarotid septum) or to reduce the distance of the carotid body 27 from the inner surfaces 80 and 84, or to reduce the blood flow within the intercarotid septum, and associated convective cooling normally associated with interstitial blood flow. In addition to the embodiment where the ETAP catheter is configured for electrical neural stimulation, the presence of a carotid body 27 and carotid body nerves within an intercarotid septum may be confirmed by squeezing the septum as depicted. Since the carotid body is a chemoreceptor whose function is to signal hypoxia, squeezing an intercarotid septum may result in ischemic hypoxia of a carotid body, which may cause a user detectable physiological response to ischemia induced by the arms.

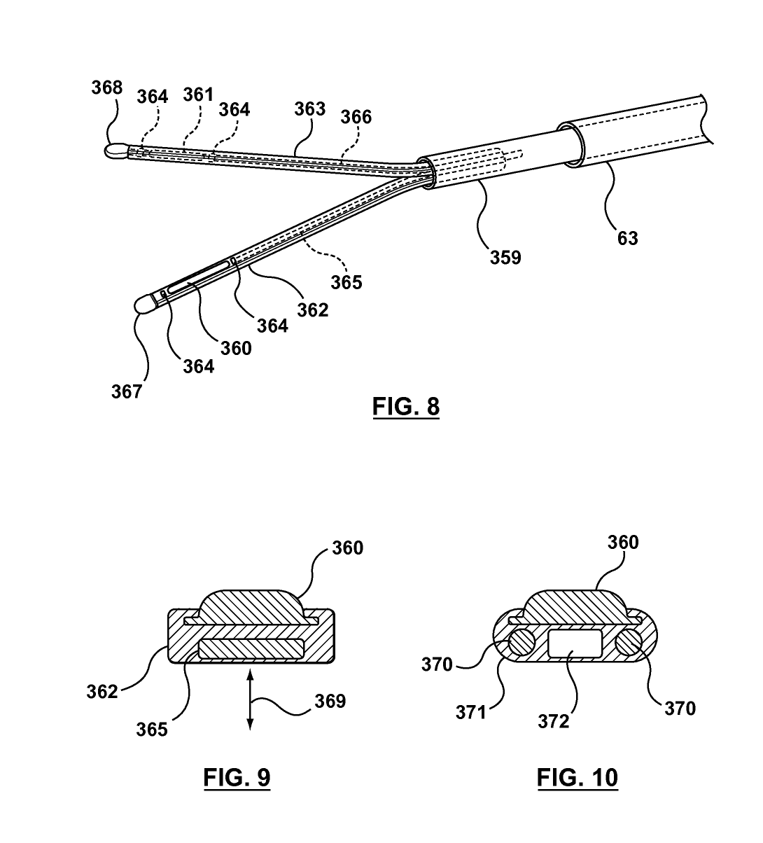

An alternative embodiment of an ETAP catheter 359, as shown in FIG. 8, comprises electrodes 360 and 361 mounted in flex circuits 362 and 363. Electrodes made, for example, from an electrically conductive material such as stainless steel, copper, gold, platinum-iridium, or alloy such as 90% Au 10% Pt may be mounted on a flexible plastic substrate, such as polyimide, PEEK or polyester film. Potential advantages of flex circuit designs include the ability to use relatively thin and flexible electrodes, which may provide better tissue conformation and contact than more rigid electrodes resulting in better electrode apposition; manufacturing may be faster and at a reduced cost; and electrode geometry may be customizable. Electrodes 360 and 361 are mounted to face toward one another such that when the ETAP catheter is placed on a carotid bifurcation the electrodes contact vessel walls of the internal and external carotid arteries only and do not substantially face in to the lumens of the vessels, thus providing maximum contact with the intercarotid septum and minimal electrical contact with blood flow. It is appreciated that thermal conduction to the blood flow may still be desired. This arrangement may allow for more focused ablation energy in the septum, lower and less variable energy losses and more accurate measurements (e.g., tissue impedance and temperature) of the septum since much less current is conducted through the blood stream. Additional sensors 364 (e.g., temperature sensors such as a thermocouple or thermistor) may be mounted in the flex circuit proximate to the electrodes and may be used to monitor or control delivery of ablation energy. Additional energy delivery electrodes and impedance measurement electrodes combined with or separate from ablation electrodes can be added to the design. The flex circuits may be mounted on arms 365 and 366 for mechanical structure and resiliency. Arms 365 and 366 may be made from a superelastic material such as Nitinol and they may be laminated to the flex circuits, embedded in a flex circuit substrate, or the flex circuits may contain a lumen through which the arms are positioned. An arm may be embedded in a flex circuit by placing a Nitinol sheet as one of the layers of the flex circuit. Then, when the layers are laser cut into the individual circuits' shape, the Nitinol sheet layer will be laminated between layers and integral to the flex circuit. Nitinol or another thermally conductive material such as copper may beneficially act as a heat sink to electrodes 360 and 361, which may improve ablation profile and decrease risk of charring due to high surface temperature or high current density. The arms may be substantially straight or preformed into a shape that facilitates electrode contact with vessel walls of an intercarotid septum, examples of which are provided below. As used herein, a preformed configuration refers to an unstressed configuration. Atraumatic tips 367 and 368 may be formed at or connected to the distal ends of the arms to facilitate insertion of the arms over a carotid bifurcation while reducing risk of dissection, endothelial injury or dislodging of plaque. The atraumatic tips may also reduce risk of iatrogenic injury due to sliding or torquing the arms. An atraumatic tip or edge may be formed by attaching a thermoplastic (e.g., Pebax) sheath to the flex circuit. For example, a sheath could be fitted over the flex circuit during the manufacturing process and reflowed into place. The sheath may extend distal to the flex circuit and could be thermally “tipped” to create a dome shape at the distal end. The thermoplastic could mask the edges and tip of a flex circuit and provide an atraumatic surface for tissue contact. The thermoplastic could be removed from the ablation electrode surface, either mechanically or using a laser ablation process. This thermoplastic covering could also serve as a method to embed a structural arm (e.g., Nitinol shape wire) to the back of a flex circuit. In order to achieve good bonding between a flex circuit and a thermoplastic, holes may be placed in the flex circuit material during fabrication. The holes would allow thermoplastic to reflow into them and hold firmly onto the flex circuit to prevent delamination. Flex circuits and ablation electrodes coupled thereto can be incorporated into suitable alternative catheters described herein, which can replace the described ablation electrode or can be added to the embodiments. Additionally, arms 364 and 365 can be modified in any suitable manner as described below in additional exemplary embodiments. For example only, arms 364 and 365 can be asymmetric, such as by having different lengths, or have a curvature that may enhance performance.

Exemplary configurations of the arms 364 and 365 are shown in FIGS. 9 and 10. FIG. 9 shows a cross section of an arm with an electrode 360 having a raised surface with rounded edges to improve tissue contact and force by slightly distending into the vessel wall. The rounded edges may reduce radiofrequency edge effects that can be caused by high current density at sharp corners. Arm 365 is a flat, ribbon shape, or other shape, which may allow the arm to preferentially flex in direction 369 such as an elliptical shape. FIG. 10 shows a cross section of an arm having two superelastic wires 370 spaced apart. Flexible plastic substrate 371 contains a lumen 372 through which a fluid may be irrigated to cool electrode 360.

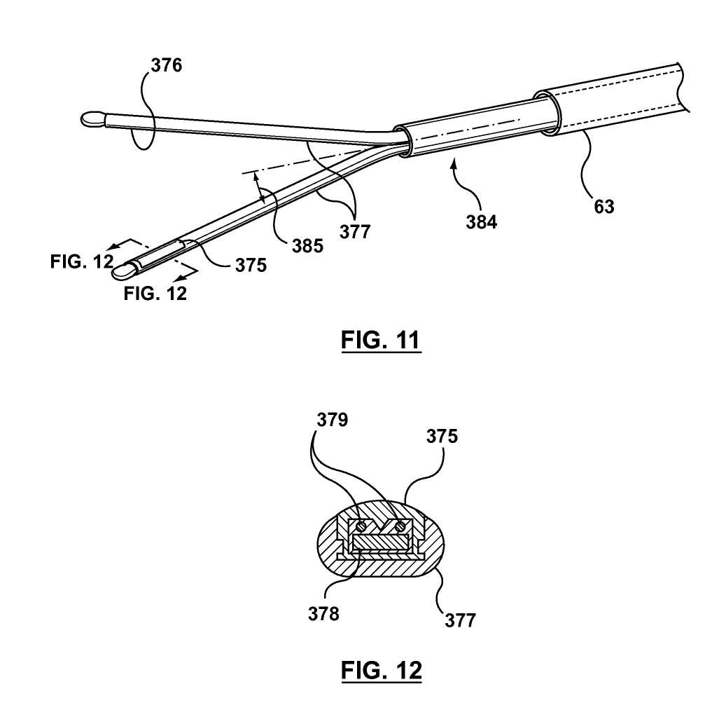

FIG. 11 is a schematic illustration of another embodiment of an ETAP catheter 384. Arms 377 comprise a superelastic Nitinol structural wire coated with dielectric insulation such as Pebax, with a machined electrode 375 and 376 mounted to the Nitinol structural wire. FIG. 12 shows a cross section of arm 377. Electrode 375 may be made (e.g., machined or molded) from an electrically conductive metal such as platinum iridium, stainless steel, Liquidmetal or gold. The electrode shape may have a slight curvature at an exposed section to facilitate tissue contact, such as a general barrel-shape described below. The electrode may comprise a lumen through which the structural wire 378 is positioned. The electrode may be connected to the structural wire, for example by welding, soldering, or adhesive. The lumen in the electrode 375 may be configured to hold electrical conductors 379, for example conductors for a temperature sensor (e.g., thermistor, thermocouple). The electrode may comprise side grooves for adhesion of dielectric material 377 (e.g., Pebax). This embodiment of an ETAP catheter 384 may be configured with arms 377 normally open. For example, structural wires 378 may be preformed with a bend near a junction with a shaft of the catheter to configure the arms at an angle 385 from an axis of the catheter shaft in a range of about 15 degrees to 45 degrees (e.g., about 20 degrees). Alternatively, the ETAP catheter 384 may be configured with arms 377 normally closed with an angle 385 of less than 15 degrees.

### Ablation Elements

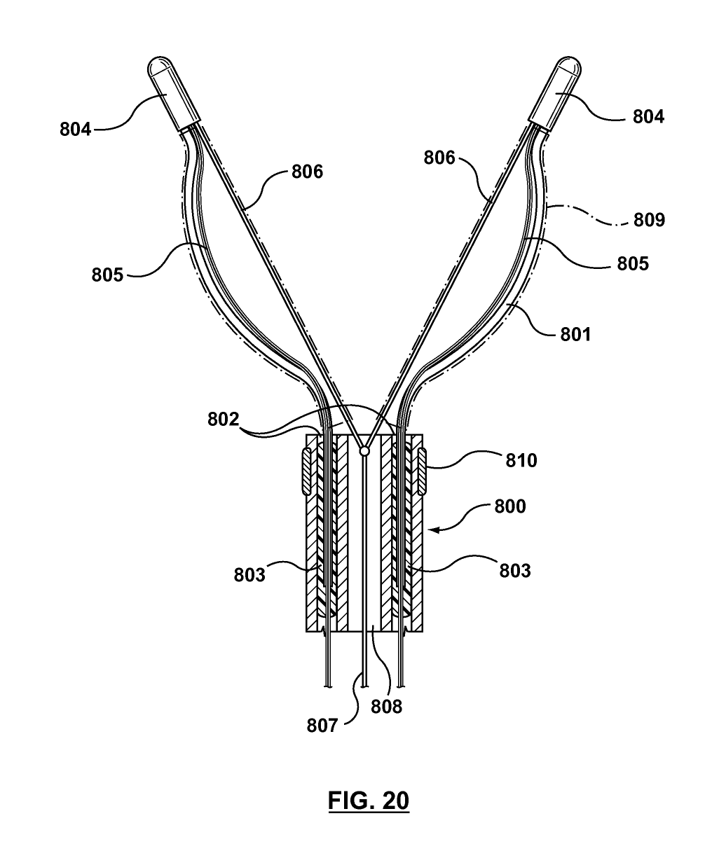

In any of the embodiments herein, one or more of the ablation elements may be electrodes configured for radiofrequency ablation, bipolar radiofrequency ablation, or irreversible electroporation. For example, electrodes configured for bipolar radiofrequency ablation may be of a size that can create an effective thermal ablation contained approximately within an intercarotid septum when the electrodes are placed in an internal and external carotid artery on an intercarotid septum and a radiofrequency signal of predefined characteristics is delivered. Electrodes that are too small may create a lesion that is uncontrolled, too small, or too hot due to high electrical impedance caused by tissue coagulation or charring. Electrodes that are too large may create a lesion that is uncontrolled, too large, or too cool due to unfocused concentration of RF over a large surface area. Additionally, the size of a sheath used to deliver a catheter limits electrode diameter. In any of the embodiments herein, the ablation devices may comprise electrodes, for example, with a surface area in a range of about 8 to about 65 mm² (e.g., about 12 to 17 mm²). For example, as shown in some of the embodiments herein (e.g., FIG. 20) electrodes may be cylindrical with a hemispherical domed end having a circumference of about 0.8 to 2 mm (e.g., about 1.2 mm) and a length of about 3 to 10 mm (e.g., about 4 mm). A radiofrequency signal delivered to such electrodes may have a frequency in a range of about 300 to 500 kHz and a maximum power between about 5 W and about 12 W (e.g., a maximum power of about 5 W, 6 W, 7 W, 8 W, 9 W, 10 W, 11 W, or 12 W) and a duration of about 15 to 120 seconds (e.g., between about 15 and about 60 seconds, between about 15 and about 40 seconds, between about 20 and about 40 seconds, and about 30 seconds). In some embodiments there is an initial ramp up of power of 2 W/s until the power reaches 8 or 10 W. In some embodiments there is a ramping up of 2 W/s to 4 W, then holding for about 10 s to watch for errors, then continuing to ramp up at 2 W/s to a max power (e.g., 8 W or 10 W), and then holding the power for a duration of about 20 to about 40 s (e.g., 30 seconds).

Electrodes may be made (e.g., machined) from an electrically conductive material such as stainless steel, copper, gold, platinum-iridium, or alloy such as 90% Au 10% Pt. For example, electrodes may be machined in a shape of a circular cylinder with hemispherical domed end with a hollow cavity, which may be used to position sensors (e.g., temperature sensor, impedance sensor), connect to structural segments of ETAP catheter arms, or for cooling irrigation. Other shapes may be used for electrodes such as elliptical cylinder, cuboids, ribbon or complex shapes.

Ablation elements may be positioned on ETAP catheter arms so they are aligned with a force vector applied by the arms. For example, a structural segment of an arm that applies a closing force toward the opposite arm may be positioned in the center of a cylindrical electrode. In this example a force vector applied by the arm is approximately equal to a force vector applied by the electrode. When these electrodes are positioned in an internal and external carotid artery and closing force is applied by the arms the electrodes may settle within the vessels approximately at two positions having the shortest distance between them (e.g., the center of the intercarotid septum). Alternatively, an ablation element may be positioned on an ETAP catheter arm so it is offset from a force vector applied by the arm. For example, an ablation element may be positioned at a distance (e.g., about 1 to 3 mm, 2 mm) perpendicular to the force vector applied by the arm so that when positioned the ablation element settles at a distance from the center of the intercarotid septum toward the medial or lateral side. A structural segment of an arm may have a preformed shape comprising a shaft that applies a force vector approximately toward the opposite arm and an extension that holds the ablation element at a distance perpendicular to the force vector. This embodiment may allow the creation of an ablation that is offset from the center of the septum toward the medial or lateral side of the septum. This may be advantageous if the position of a target (e.g., carotid body or carotid body nerves) or non-target nerves is known and an offset ablation would be more effective or safe.

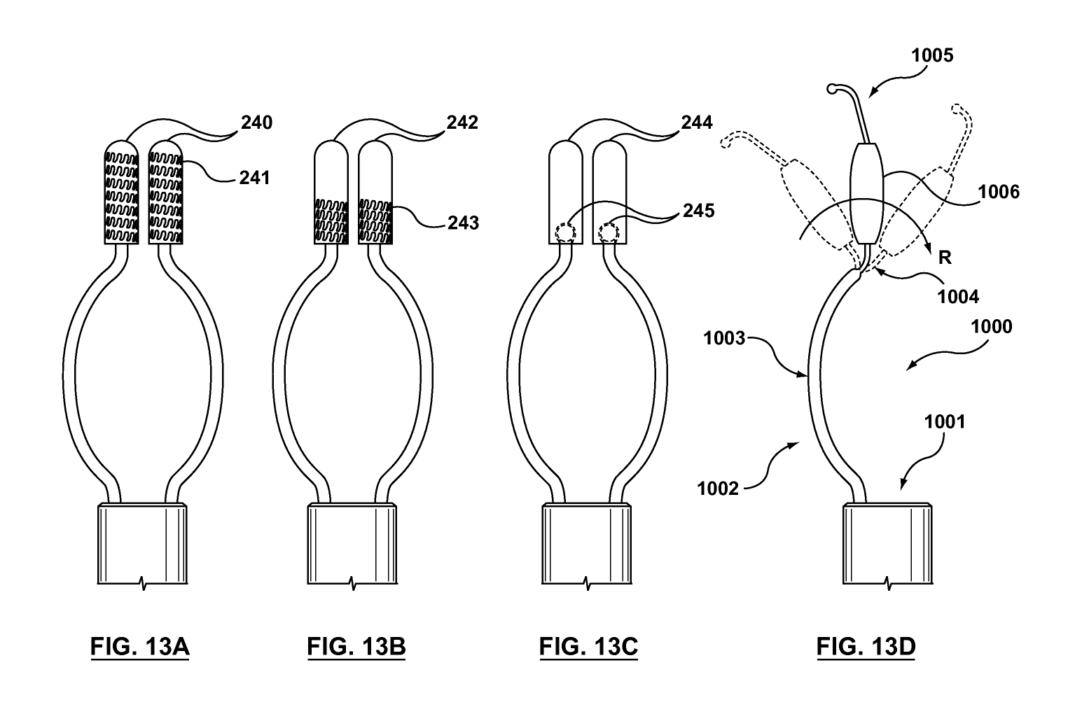

Electrodes may be configured for improved consistency of alignment and surface contact with vessel walls. Consistent electrode alignment and surface contact with internal and external carotid arteries may produce more repeatable and predictable lesions contained substantially in an intercarotid septum and thus greater efficacy and safety. FIGS. 13A, 13B, 13C, and 13D show exemplary embodiments of electrodes of an ETAP catheter that are designed and configured to flexibly move with respect to exemplary arms. Flexibility may be imparted along the full length of the electrode, a portion of the electrode, or at the connection of the electrode to the arm. FIG. 13A illustrates electrodes that are configured to have flexibility along their full length or most of their length. Electrodes 240 can be fabricated from a rigid metal such as a metal tube and comprise laser cut channels 241 to impart flexibility of the electrodes along the full length or most of the length of the electrode. The laser cut channels may be in a continuous spiral pattern or a non-continuous pattern. In some embodiments the electrodes have sections that are flexible separated by solid sections of material that are relatively inflexible (or at least have less flexibility). The channels can have varying patterns along the length of the channel, such as varying pitch or varying distance between channels. Alternatively, flexible electrodes may be made from a coiled spring. FIG. 13B illustrates an embodiment in which the electrodes are flexible in a proximal region of the electrodes next to where they are connected to arms, and the distal regions of the electrodes are rigid. For example, electrodes 242 may be fabricated from metal tubes with laser cut channels 243 in the proximal regions to impart electrode flexibility. The distal, rigid portions of the electrodes may flexibly move in relation to the arms. FIG. 13C illustrates an additional embodiment of electrodes that are configured for improved consistency of alignment and surface contact that comprises a rigid electrode connected to arms with a flexible joint. As shown in FIG. 13C, rigid electrodes 244 are connected to the arms via a ball and socket joint 245. Alternative flexible joints (not shown) may be used such as a dowel hinge, or an elastically flexible member, such as a spring, joining an electrode to an arm.

FIG. 13D illustrates an exemplary embodiment in which an arm is configured for electrode pivoting, which may improve the electrode surface contact with vessel walls and self-alignment. In this embodiment an arm is configured to provide electrode pivoting with a change in flexibility and resiliency along its length. In FIG. 13D only one arm is shown for clarity but it is understood that the ablation catheter can include a second arm that is or is not symmetrical with the arm shown. In FIG. 13D, catheter 1000 includes shaft 1001, which supports arm 1002 extending distally therefrom. Arm 1002 includes first section 1004 adjacent and proximal to the electrode mounting region that is more flexible and less resilient than second arm section 1003, which is proximal to first section 1004. A thickness or diameter of first section 1004 provides the greater flexibility, wherein the thickness is less than the thickness or diameter of second section 1003. The flexibility of first section 1004 allows electrode 1006 to pivot, or preferentially bend, about the thinner section in the directions of arrow R as shown. Two configurations of arm 1002 with first section 1004 bending and electrode 1006 pivoting are shown in phantom, including atraumatic tip 1005.

In a mere example, arm 1002 is a round superelastic Nitinol wire having a diameter in second section 1003 that is about 0.012 inches, and a diameter in first section 1004 of about 0.006 inches to about 0.008 inches. In this example, first section 1004 starts about 1 to about 2 mm proximal to the electrode. First section 1004 with a thickness or diameter need not extend completely to the proximal end of electrode 1006. For example, there can be a small section of arm 1002 immediately proximal to electrode 1006 with a thickness or diameter slightly greater than the thickness or diameter of 1004.

Both electrodes in each of the embodiments in FIGS. 13A-D need not have the same flexibility or ability to pivot. For example, in FIG. 13A only one electrode may have a channel formed therein to impart flexibility, while the other electrode is a length of solid material. Additionally for example, in FIG. 13D the arms, in sections proximal to the electrode, can have slightly different thicknesses and thus slightly different flexibility. FIGS. 13A-D illustrate exemplary embodiments endovascular carotid septum ablation catheters comprising first and second diverging arms with free distal ends, the arms extending generally distally from the catheters, the first arm comprising an ablation element secured to and flexibly movable relative to the first arm. The second arm can include a second ablation element secured to and flexibly movable relative to the second arm.

In some embodiments the ablation catheter includes one or more coiled electrodes. For example, the electrodes can be made from a tightly wound, coiled conductive wire. Coiled electrodes can be configured with sufficient flexibility such that they may improve the electrode surface contact with vessel walls, such as by conforming to the geometry of the vessel's surface, and self-alignment. A coiled electrode may also distribute current density in proximate tissue, thus potentially avoiding hot spots in the tissue. Well distributed current density may also result in predictable lesion formation in target tissue and may reduce risk of thrombus forming on a vessel surface. In an exemplary embodiment a coiled electrode wire (e.g., round wire made from Nitinol, stainless steel, gold-platinum alloy, platinum-iridium alloy) has a diameter of about 0.008″, and the coil has a pitch of about 0.008″ to 0.012″. The coil may be wrapped around mandrel (e.g., with a diameter of about 0.030″) and held in place with epoxy. The mandrel may have a lumen along its axis and a structural arm wire may be positioned in the lumen.

The electrodes described in any of the embodiments herein (e.g., in FIGS. 13A-D, coiled electrodes, etc.) can be incorporated into any other suitable embodiment described herein, and are not intended to be limited to use with arm configurations shown.

### Slide on Design

An ETAP catheter may be configured to slide over a carotid bifurcation to place ablation elements in position in an internal and external carotid artery. In some embodiments arms of an ETAP catheter are configured as normally, in un-stressed configurations, open, in which elastically flexible arms are pre-formed to hold ablation elements apart when unconstrained by a sheath or vessel anatomy. The embodiment shown in FIGS. 6A-6D is an example of a catheter configured in this manner. In some embodiments in which the arms are pre-formed in unstressed configurations to be open, the arms hold ablation elements greater than about 6 mm apart, such as, for example, between about 10 and about 20 mm apart. Once the device is advanced over a carotid septum the arms may be closed to bring the ablation elements into contact with the carotid septum, such as is shown in FIG. 7. Alternatively, arms or splines of an ETAP catheter may be configured as normally closed, in which elastically flexible arms are preformed in unstressed configurations to hold ablation elements close together (e.g., less than about 6 mm apart, less than about 4 mm apart, or less than about 2 mm apart) when unconstrained by a sheath or vessel anatomy. The arms are configured to elastically spread apart as they are advanced over a carotid bifurcation while the ablation elements slide into place. For example, shown in FIG. 14 is a distal region of an ETAP catheter having elastically flexible arms configured in a normally closed configuration and having distal outward bends 488 and atraumatic tips 489. The arms of this embodiment are opened by sliding the outward bends 488 over a carotid bifurcation, which separates or opens the arms. An elastic force in the arms applies a passive closing force that presses ablation elements into contact with vessel walls of an intercarotid septum. Thus apposition of electrodes is achieved. The passive closing force can also urge portions of the external and internal carotid arteries towards each other.

In some embodiments herein in which at least one diverging arm (with or without an ablation element thereon) is configured to make passive apposition with a carotid septum wall in a desired or known location in an external or internal carotid artery, the arm is configured so that when some aspect of the catheter is coupled, or engaged with, a common carotid artery bifurcation, a portion of the arm will be in contact with the septal wall in the desired or known location. That is, the arm is configured so that the act of engaging some aspect of the catheter with the bifurcation causes a portion of the arm (e.g., an electrode thereon) to be in contact with the septal wall in the desired or known location. The arm can still be configured to be in contact with the septal wall when some aspect of the catheter has not yet engaged the bifurcation, but a portion of the arm may not yet be in the known or desired location until the engagement occurs.

Geometrical characteristics of carotid bifurcation or intercarotid septums may vary, for example, septum width, bifurcation angle, and vessel or septum shape. Regardless of whether the catheter is configured for active or passing closing forces, the geometrical characteristics of carotid bifurcation or intercarotid septums can interfere with the contact between an electrode and target tissue. For example, a U-shaped surface, convex surface or irregular surface may cause substantially straight arms to contact the surface, which may reduce or impede electrode contact with the surface. In some embodiments an ETAP catheter may therefore include a distal region comprising one or more arms having pre-formed, or unstressed, shapes or configurations that facilitate consistency of electrode contact when used on various carotid bifurcation and septum geometries. FIGS. 14-17, 30A-32A, 32I, 33A-C, 34A-C, and 80, for example without limitation, illustrate catheters or components thereof configured in this manner. Consistency of electrode contact area or pressure may improve consistency or predictability of a lesion formed in a carotid septum while substantially avoiding important non-target tissue. Arms having a preformed (i.e., unstressed) shape may be configured to resiliently conform to an undeployed state inside a sheath, so the distal region may be slidably delivered through a sheath to a carotid artery, and to elastically deploy to the preformed shape when no longer constrained by the sheath, such as after being advanced out of the sheath.

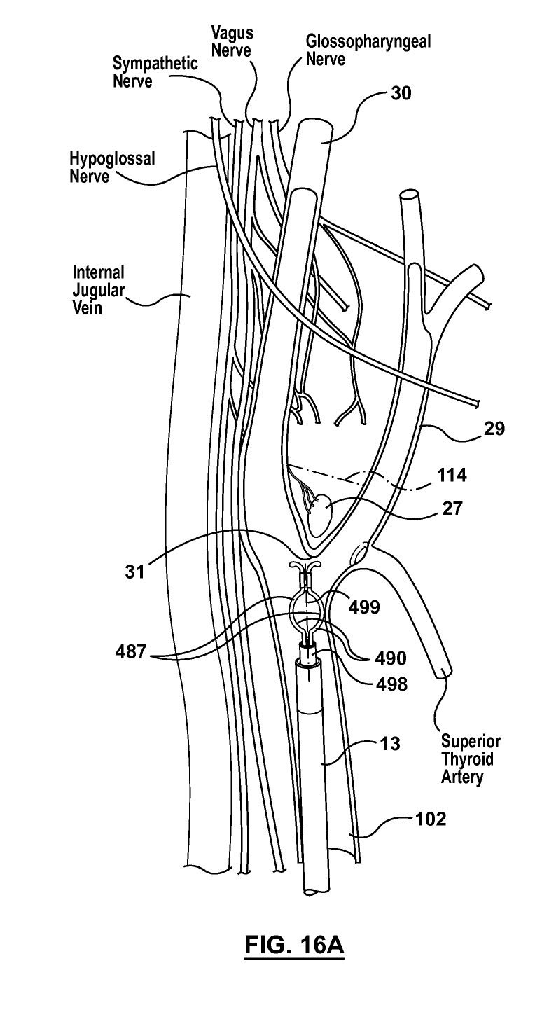

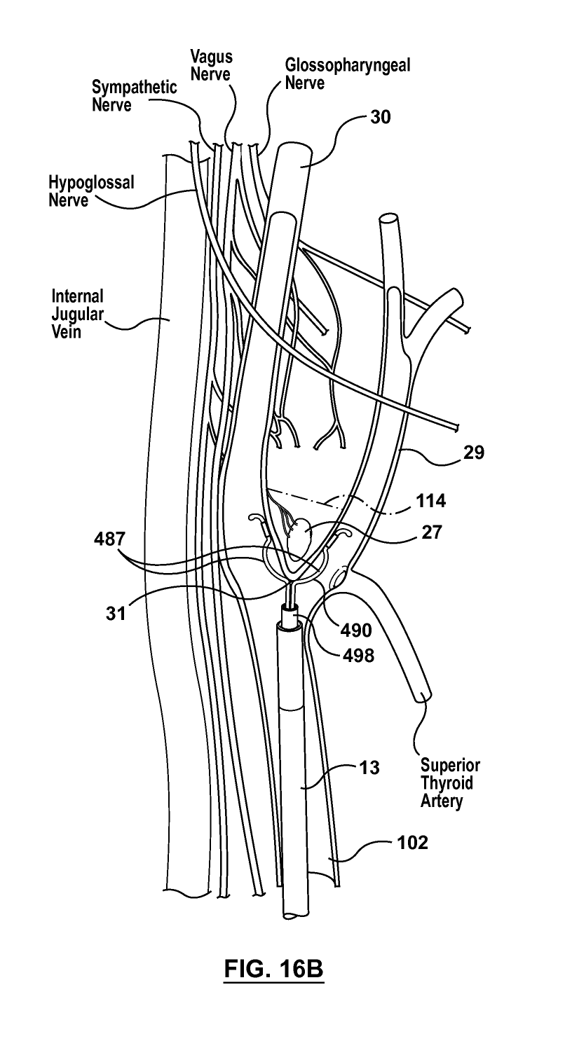

The preformed, or unstressed, shape of the distal region may comprise a predetermined aperture between the arms that allows capture of a carotid septum and advancement over the septum in sliding apposition to walls of the septum. The predetermined aperture may also be configured to prevent the arms from opening excessively, which may cause undesirable contact with non-target regions of the carotid vessel walls. For example, arms may comprise superelastic or elastic structural members 490 having a preformed shape having an outward arch that may avoid or reduce contact between the arms and the vessel surface. The arms may be constrained to undeployed configuration when contained within a delivery sheath. The arms may elastically deform to the preformed shape when deployed from the delivery sheath. FIG. 15 illustrates an embodiment in which a structural member 490 is configured to achieve apposition and facilitate electrode contact while accommodating varying carotid bifurcation geometries. Each elastic structural member 490 (only one is shown in FIG. 15 for clarity) comprises a proximal substantially straight portion 491 that is partially or fully positioned within ETAP catheter shaft 498, which is shown in FIG. 16A. Straight portion 491 length L1 may be greater than about 10 mm, measured along the shaft axis 499 as shown, for secure placement within the shaft 498. Structural member 490 also includes a first outward bend 492 that bends the arm away from the catheter shaft axis 499. In exemplary embodiments outward bend 492 has a radius of curvature “ROC1” of about 0.01 to 1 mm and may bend the arm away from the axis at an angle A1 of about 45 to 90 degrees. Member 490 also includes an inward curve 493 that bends the arm toward the axis 499. In exemplary embodiments inward curve 493 has a radius of curvature “ROC2” of about 2 to 10 mm, an arc length that brings the arm substantially back to the shaft axis, and an axial length L2 of about 4 to 10 mm measured along the shaft axis 499 as shown. Structural member 490 includes a second outward bend 494 that bends the arm so it extends substantially along the axis. In exemplary embodiments outward bend 494 has a radius of curvature “ROC3” of about 0.01 to 1 mm. Structural member 490 includes a distal substantially straight portion 495 comprising an ablation element such as a radiofrequency electrode with a temperature sensor. In exemplary embodiments straight portion 495 has a length L3 of about 4 to 6 mm). Alternatively, ablation elements may be angled such that the distal tips are angled toward one another. In an angled electrode embodiment a preformed arm directs the distal end of electrodes at an angle of about 10-30 degrees toward the axis. In other embodiments the electrode is angled toward the axis at an angle of more than 0 and up to and including 30 degrees. Angling electrodes in such a manner may facilitate even contact with tissue along the length of the electrodes. For example, when angled arms are advanced over an intercarotid septum and opened, the electrodes may be more parallel to the vessel walls in the target region. Optionally, the elastic structural member 490 may continue past the ablation element and may comprise a third outward bend 496 that bends the arm away from the axis (e.g., bend 496 may have a radius of curvature of about 1 to 3 mm, an arc length of about 2 to 4 mm, and an axial length L4 of about 2 to 4 mm); and an atraumatic distal tip 497. Lengths L2, L3, and L4 may sum to about 10 to 20 mm. The ablation element mounted to the distal straight portion 495 may be at or between about 4 mm to 10 mm away from a junction where the arms are joined to the shaft. Terminology used with respect to the embodiment in FIGS. 14 and 15 can similarly be used with respect to structural members herein. For example, the curves and bends described with respect to the embodiment in FIGS. 14 and 15 similarly describe other structural members herein even if not expressly stated. In the embodiment in FIG. 15, any of the electrodes disclosed herein can be mounted to the mounting regions.

FIG. 16A shows an ETAP catheter, with arms 487 comprising elastic structural members 490 configured as in FIG. 15, to facilitate electrode contact, positioned in a common carotid artery with a delivery sheath 13 retracted to deploy the arms 487 to their preformed shape. The outward bend 496 and atraumatic tip 497 of each arm extend away from the axis 499 of the shaft. When the catheter is advanced to contact carotid bifurcation 31 the outward bend and atraumatic tip of each arm slides over the corresponding vessel wall opening the elastic arms 487. FIG. 16B shows the ETAP catheter in a suitable position for carotid body ablation with ablation elements contacting the intercarotid septum from an internal carotid artery and an external carotid artery, respectively. In addition, after the deployment and advancement upon the septum, catheter shaft can be torqued in order to twist the arms 487 in order to tighten the grip of the electrodes on the septum, squeeze the septum and improve apposition of electrodes.

The catheter shown in FIGS. 14-16 includes first and second arms that are configured such that substantially all contact that occurs between the first and second arms and the walls of the internal carotid artery and the external carotid artery is contact between the ablation elements and the walls. In this context substantially all contact includes at least 60%, at least 70%, at least 80%, at least 90%, and more than 90%. In this embodiment the arms include a clearance portion that includes curved portion 493, the clearance portion being configured to substantially avoid contact with a wall of the external carotid artery or internal carotid artery when the catheter is coupled with a common carotid artery bifurcation, as shown in FIG. 16B. In this embodiment the clearance portions are also configured to make less surface area contact with the walls of the carotid arteries than the ablation elements. Additionally, the arms are configured so that the ablation elements apply a greater force on the wall of the carotid arteries than the clearance portions.

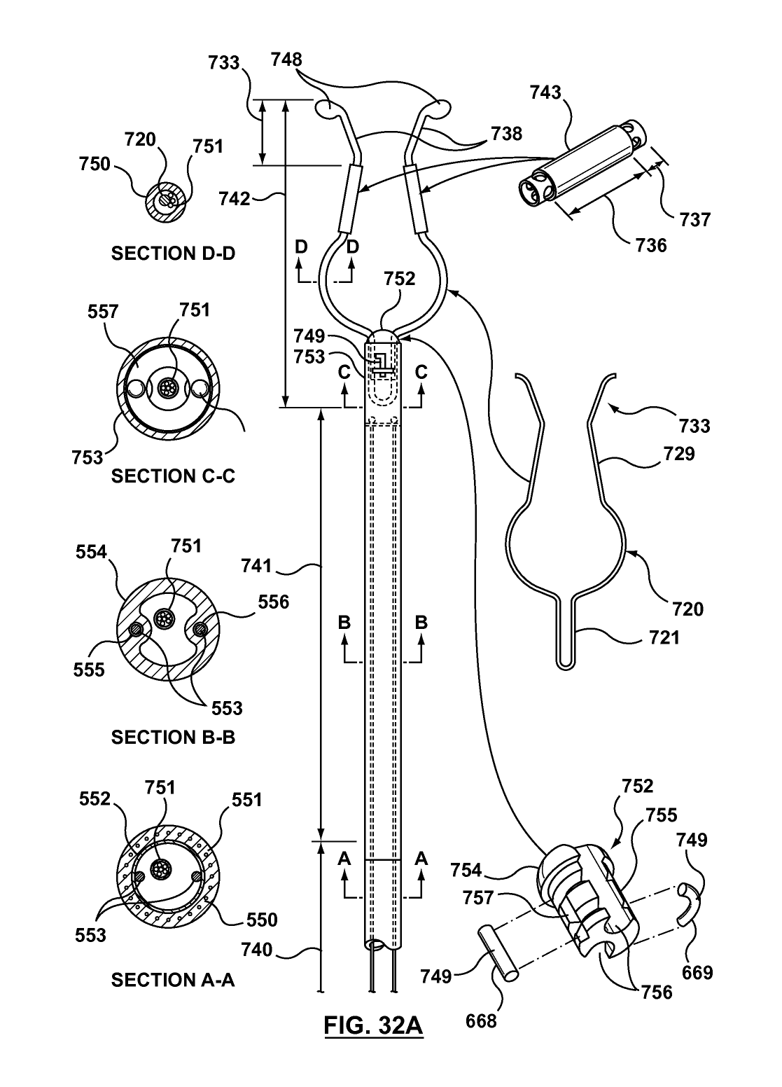

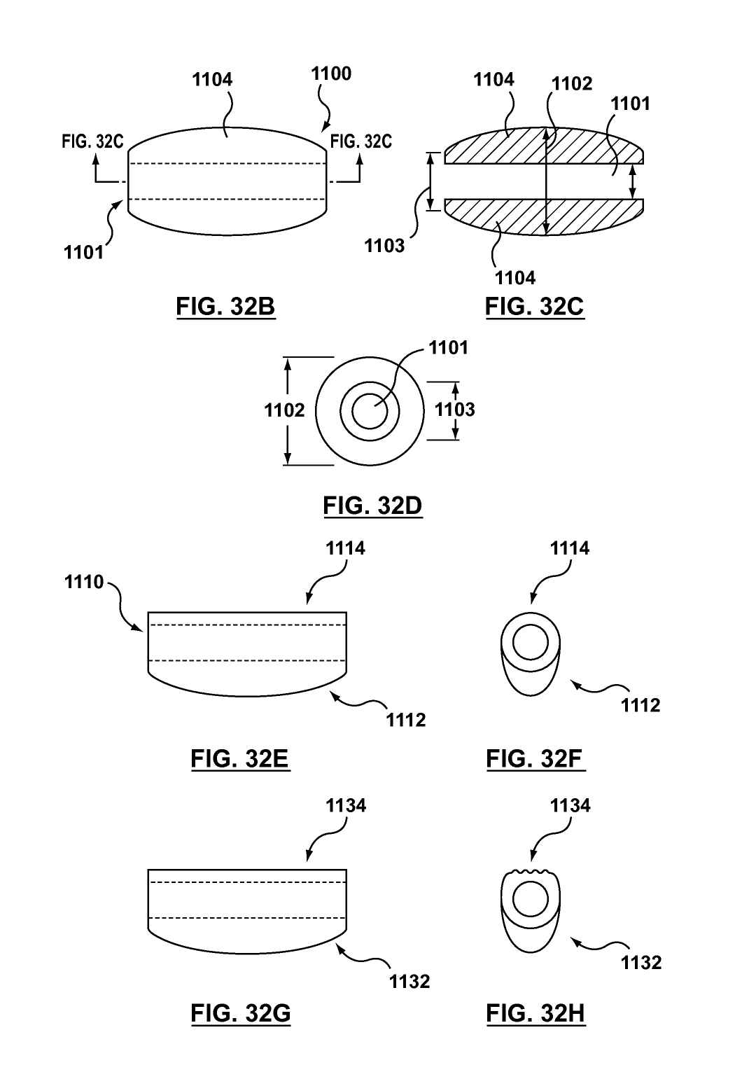

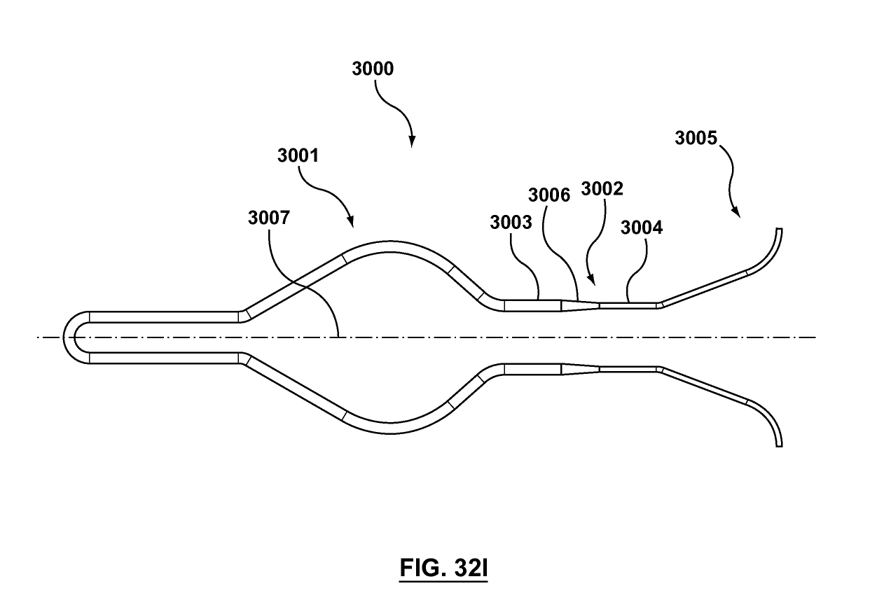

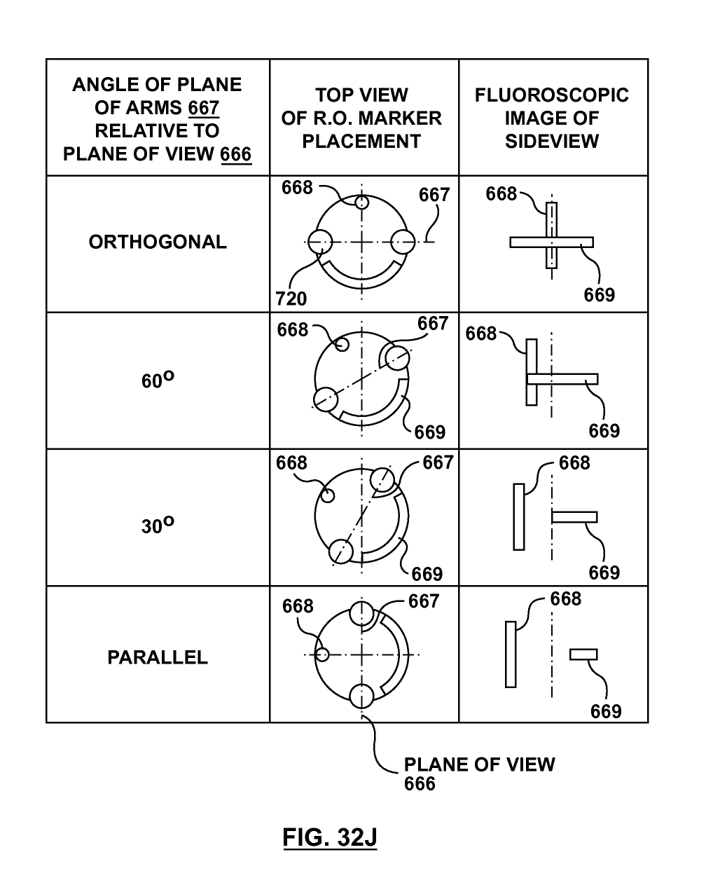

Another embodiment of an elastic structural member 720 with a preformed, or unstressed, shape or configuration configured to facilitate consistency of electrode contact when used on various carotid bifurcation geometries is shown in FIG. 17. Elastic structural members for both arms are made from a single wire 723, or monolithic, with a preformed, or unstressed, shape configured to hold ablation elements (not shown for clarity) in a substantially closed configuration such that a distance 735, measured along a line perpendicular to the shaft axis, between portions of the wire 723 in electrode-mounting region 729 is less than or equal to about 4 mm. In some embodiments distance 735 is less than or equal to about 2 mm. In some embodiments distance 735 is less than or equal to about 1 mm. In some embodiments distance 735 is about 0 mm. In alternative embodiments structural member 720 is made from more than one element and not formed from a single element. The wire may be a material with super elastic or elastic properties, such as spring stainless steel or superelastic Nitinol (e.g., Nitinol having a transformation temperature below body temperature). In some embodiments the wire is a round wire form having a diameter of about 0.004″ to 0.018″ (e.g., about 0.006″ to 0.012″ or about 0.0100″+/−0.0005″). The wire may have a substantially constant diameter along its full length. Alternatively, the wire may have a narrower diameter on sections that may have less elasticity or more flexibility, such as in the embodiment in FIGS. 13D and 32I. For example, a wire may be ground to have a narrower diameter (such as less than about 0.0100″, less than about 0.0080″, less than about 0.0060″, or less than about 0.0040″) in regions, allowing more flexibility, such as in an electrode-mounting region 729 (electrode not shown) or funnel, or atraumatic, section 733. Alternatively, a combination of wire diameters may be applied to a wire along the spline length 732 that effectively creates desired closing force or contact force. Using one wire for both elastic structural members may facilitate manufacturing and help to maintain alignment and position of each arm with respect to one another. As shown in FIG. 17 the wire forms a shape that is symmetrical along an axis of symmetry 724, which can be considered to be substantially the same axis as the axis of the catheter shaft, for an embodiment having symmetrical arms. In such an embodiment it may not matter which arm is placed in an internal carotid artery and which is placed in an external carotid artery. However, in an alternative embodiment, an elastic structural member in one arm may be asymmetrical to an elastic structural member in a second arm. For example, one arm may be longer than the other.

In the embodiment shown in FIG. 17 elastic structural member 720 comprises a proximal section 721 that may be positioned in a catheter shaft to cantilever both arms. Proximal section 721 may have a length 722 sufficient to cantilever arms in a catheter shaft yet short enough to remain in a section of a catheter shaft distal to a deflectable region so as to not interfere with deflection. For example, length 722 may be about 0.13″ to 0.20″ (e.g., about 0.16″). Proximal section 721 may also comprise a 180° bend, as shown, that connects an elastic structural member of a first side to that of a second side. For example, the bend may have a diameter of curvature of about 0.03″. The diameter of curvature of the bend may form a gap between the sides of the proximal section 721, which may facilitate anchoring of the arms in a catheter shaft.

On the elastic structural member in FIG. 17, distal to the proximal section 721, the wire 723 may bend away from the axis of symmetry 724 as shown, for example the bend 725 may have a diameter of curvature 719 of about 0.03″ and an angle of about 45° to 80° (e.g., about 70°). Distal to the bend 725, wire 723 may form an arch 726 that bends the wire toward the axis of symmetry 724 as shown. For example, arch 726 may have a diameter of curvature 727 of about 0.25″ and an axial length 728 of about 0.27″. Electrodes may be mounted to a region 729 of the elastic structural member 720 distal to the arch 726. The electrode-mounting region 729 may have a length sufficient to hold an electrode. For example, about an electrode-mounting region 729 about 0.2″ long, may be suitable to hold an electrode that is about 0.2″ long having and exposed length of about 4 mm (0.157″). There may be an outward bend 730 in the wire 723 between the arch 726 and the electrode-mounting region 729. For example, bend 730 may have a diameter of curvature 718 of about 0.06″ and an angle of about 0° to 50° (e.g., about 40°) or an angle such that the electrode-mounting region is angled parallel or slightly toward the axis of symmetry 724, for example the electrode-mounting region may be at an angle 731 of about 10° from the axis of symmetry 724 with the distal end angled toward the axis of symmetry. Angling electrodes in such a manner may facilitate even contact along the length of the electrodes, for example, when the arms are advanced over an intercarotid septum and opened, the electrodes may be more parallel to the vessel walls in the target region. Even contact along the length of the electrodes with the target vessel wall is important to create a predictable ablation temperature, size and geometry. Even electrode contact also facilitates self-alignment of the electrodes in the desired target region of the internal and external carotid arteries. In any suitable embodiment herein one or both electrodes are substantially parallel to an axis of the structural member. In any suitable embodiment herein one or both electrodes are angled between 0 and about 30° relative to an axis of the structural member. In some embodiments the angle is less than or equal to about 15°. In some embodiments the angle is less than or equal to about 10°. In some embodiments the angle is less than or equal to about 5°. The distal ends of the electrodes can be angled inward or outward relative to the axis of the structural member. In FIG. 17 any of the electrodes herein can be mounted to one or more of the electrode mounting regions.

An arch or other clearance portion, in any of the embodiments herein, may provide multiple functions. For example, when the arms are advanced over an intercarotid septum the flexibility of the bend 725, arch 726 and optional bend 730 allows the arms to open; when the arms are advanced over an intercarotid septum the elasticity of the bend 725, arch 726 and optional bend 730 applies a closing force that provides a contact force between electrodes and vessel walls, and also facilitates self-alignment of electrodes within a desired target region 136 and 137 as shown in FIG. 5A; the axial length between the electrodes and cantilevered proximal section of the elastic structural member 720 provides a moment arm that also contributes to closing force; the curvature 727 of the arch also contributes to the closing force; the structural features of all components of the arms contribute to the closing force, including the elastic structural member material, diameter, cross sectional profile and preformed shape, as well as arm electrical insulation material and dimensions; when placed on an intercarotid septum the arch may allow the arms to place electrodes in contact with vessel walls with minimal contact of the arch with vessel walls, which may be particularly important with carotid bifurcations having a U-shaped saddle as opposed to a more V-shaped saddle, so electrode contact and self-alignment is not impeded; the outside surface of the arches may also provide an atraumatic surface that may reduce traumatic impact to vessel walls; the combined length of the axial length of the arch 728 and the electrode length, herein referred to as spline length 732, ensures that the electrodes are placed in a desired target region 138 and 139 (see FIG. 5B) on an intercarotid septum, for example, spline length 732 may be about 0.276″ to 0.591″ (7 to 15 mm) (e.g., about 0.433″ or 11 mm).

On the elastic structural member in FIG. 17, distal to the electrode-mounting region 729 may be a funnel region 733, or atraumatic tip region. The function of the funnel region 733 of the curved elastic structural member 720 is to provide an opening in the arms in to which an intercarotid septum may be guided with minimal traumatic contact. As a funnel region 733 is advanced over a septum the arms are flexibly opened while elastically applying a contact force with the septum to allow electrode contact and self-alignment. The space between the arms in the funnel region and the outward angle of wire 723 provide a gap and increase surface area into which a saddle of a carotid bifurcation to be directed. The wire 723 may be angled away from the axis of symmetry at an angle 734 of about 15° to 25° (e.g., about 20°). The distal end 758 of the funnel region 733 may optionally be further angled away from the axis of symmetry 724 to ensure the distal tip does not catch a vessel wall. Optionally, the wire 723 of the funnel region 733 may have a ground down diameter (e.g., a tapered diameter) to provide increased flexibility toward the distal end, which may reduce traumatic contact, with gradually increasing elasticity toward the electrode-mounting region, which may facilitate an arm opening force. A decreased diameter toward the distal end 758 may also facilitate pulling the arms back into a sheath due to increased flexibility that prevents distal ends 758 from catching on a sheath opening. Other optional features may be added to the funnel region 733 to improve functionality, such as an atraumatic rounded tip or a coiled wire, as described later. The distal region 733 of the arm may be flexible to deform when very little force is applied to them by a vessel wall so the arm has a reduced risk of causing trauma to a vessel from scraping the vessel or a reduced risk of brain embolism from scraping off plaque. Flexibility of the distal arm regions 733 may be balanced with elastic resiliency, which may transmit a force of contact with a carotid bifurcation to the proximal portion of the arms to cause the proximal portions to bend, thus opening the arms so they can slide over a bifurcation.

The distal regions 733 of the arms may be configured to be more flexible or less elastically resilient that the proximal portion of the arms disposed proximal to regions 733. For example, the elastic structural member may be made for example from a Nitinol wire, and may have a thinner diameter in region 733 distal of the electrode than the diameter of the region proximal to the electrode. The relative thickness of the distal region provides it with more flexible or less elastically resilience than the proximal regions. In some embodiments the structural member is a round superelastic Nitinol wire with a diameter in region proximal to the electrode between about 0.010″ and about 0.014″, such as about 0.012.″ In the distal region 733 the wire can be, for example, ground down to about 0.003″ to about 0.009,″ such as about 0.006″. In alternative embodiments, separate wires are used for the regions of the structural member distal and proximal to the electrode, respectively, and connected or secured to or relative to one another in the electrode lumen.

The structural member in FIG. 17 provides another example of diverging arms that are configured such that substantially all contact that occurs between the first and second arms and the walls of the internal carotid artery and the external carotid artery occurs between the ablation elements and the walls. In this context substantially all contact includes at least 60%, at least 70%, at least 80%, at least 90%, and more than 90%. In this embodiment the arms include a clearance portion configured to substantially avoid contact with a wall of the external carotid artery or internal carotid artery when the catheter is coupled with a common carotid artery bifurcation. In this embodiment the clearance portions are also configured to make less surface area contact with the walls of the carotid arteries than the ablation elements. Additionally, the arms are configured so that the ablation elements apply a greater force on the wall of the carotid arteries than the clearance portions.

FIG. 17 also illustrates first and second arms that are substantially the same length. The lengths of the arms of the structural member in FIG. 17 can be the same as the illustrative lengths provided in the embodiment in FIGS. 14-16B.

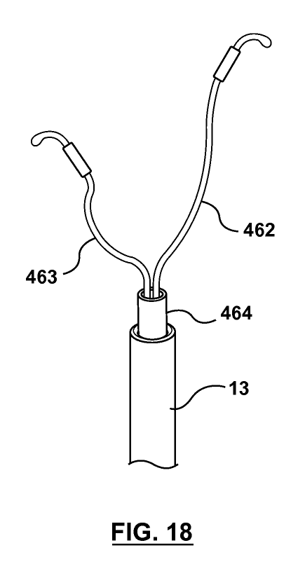

FIG. 18 is a schematic illustration of an ETAP catheter having asymmetrical arm lengths extending from catheter shaft 464. In general, FIG. 18 illustrates an ablation catheter with asymmetrical arms. A first arm 462 is longer than a second arm 463, measured along the catheter axis, by approximately 4 to 10 mm. As the catheter is advanced toward a carotid bifurcation the longer first arm 462 may engage the bifurcation 31 and slide in to an external carotid artery 29 then, after catheter position is established in relation to the intercarotid septum, the second arm 463 may engage the bifurcation 31 and slide into an internal carotid artery 30. The arm 462 placed in the external carotid artery has an ablation element (e.g., radiofrequency electrode) while the second arm 463 may have a second ablation element (e.g., configured for bipolar radiofrequency) or may not have an ablation electrode or have an impedance measurement electrode. Either way the second arm provides alignment, contact pressure, and retention force of the ablation element against a target ablation site. Arms of an ETAP catheter may also have asymmetric flexibility. For example, arm 463 may be more flexible than arm 462, which may apply less force to an internal carotid artery and reduce risk of dislodging plaque and causing a brain embolism. One aspect of this disclosure is an endovascular carotid septum ablation catheter comprising first and second diverging arms with free distal ends, the arms extending generally distally from the catheters, at least one of the first and second arms comprising an ablation element, the first and second arms having asymmetric flexibility. FIG. 18 illustrates an example of an endovascular carotid septum ablation catheter comprising first and second diverging arms with free distal ends, the arms extending generally distally from the catheters, at least one of the first and second arms comprising an ablation element, the first and second arms being asymmetrical along a catheter axis in unstressed configurations.

### Open/Close Actuation

An ETAP catheter may comprise a means to actively control arms configuration, that is, to open, close, adjust a degree of openness, or tighten the arms. For example, arms may be elastically predisposed to a substantially closed configuration (e.g., such that ablation elements mounted on the arms are held less than about 4 mm apart, less than about 2 mm apart, about 0 mm apart, or less than 0 mm apart) and opened by user actuation; or the arms may be elastically predisposed to an open configuration (e.g., such that ablation elements mounted on the arms are held greater than about 6 mm apart, such as between about 10 to 20 mm apart) and closed by user actuation; or the arms may be both opened and closed by user actuation. Such user control of an open or closed configuration of an ETAP catheter may allow ablation elements mounted to arms to be placed on a target site (e.g., both sides of an intercarotid septum at an appropriate height from a carotid bifurcation for effective and safe CBM) with minimal intrusion of non-target regions of the vessel wall. For example, the arms may be placed without sliding over a vessel wall. This may be particularly important to reduce a risk of dislodging atheromatous plaque, if it exists in the area, which could potentially flow up the internal carotid artery to the brain. Embodiments of ETAP catheters having open/close actuation as disclosed herein may comprise elastically flexible arms that are substantially straight (for example as shown in FIGS. 6, 7, 8, 11, 14), or have a preformed shape, for example, such as those shown in FIG. 15.

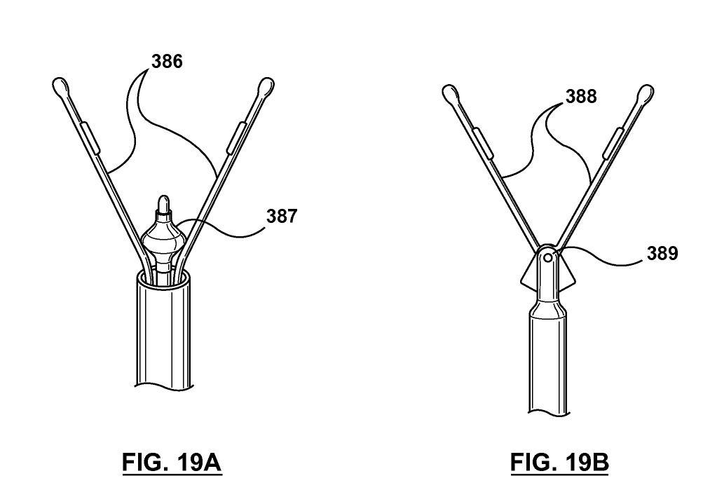

An example embodiment of an ETAP catheter having a means for actively controlling an open or closed configuration is shown in FIG. 19A. Arms 386 are elastically predisposed in a closed position and comprise elastic structural wires (e.g., superelastic Nitinol or spring steel) that are substantially straight. It is understood that superelastic structural wires may alternatively be shaped to facilitate use of elastic forces and accommodate varying geometry of intercarotid septums. For example, arms may comprise structural wires formed in a shape as shown in FIG. 15, 17, or 32I. In this embodiment, the arms may be forced opened by an actuator that is an inflatable balloon. A balloon 387 positioned between the arms 386 is inflated causing the arms 386 to open. The greater the balloon is inflated, the wider the arms are opened. A balloon is an example of an actuator and other mechanical urging devices can be envisioned. After the arms are positioned on the carotid septum in an opened configuration they may be closed to squeeze the septum or to bring electrodes into contact with the septum by deflating the balloon.

In another example embodiment shown in FIG. 19B arms 388 are connected to the shaft of the catheter with a hinge joint 389 and are opened or closed with a pull wire that is actuated by a lever at a proximal end of the ETAP catheter. A spring (not shown) may be used to cause an opening force on the arms 388 when tension on a pull wire is released. When the pull wire is pulled a torque may be applied to the arms to oppose the spring causing the arms to close. Conversely, a spring and pull wire may be configured so the spring causes the arms to close and the pull wire causes the arms to open.

As shown in FIG. 20 an ETAP catheter may be configured to close via actuation of a pull wire. The arms in FIG. 20 comprise elastic structural wires 801 such as pre-formed superelastic Nitinol wires or spring steel wires shaped in a normally open configuration. The superelastic structural wires may be connected to a catheter shaft 800 by inserting them into lumens 802 and securing with adhesive 803. Ablation elements 804 (e.g., radiofrequency electrodes) may be attached to distal ends of the superelastic structural wires 801. Electrical conductors 805 may be placed in the lumens 802 along the length of the catheter shaft 800 and extend to the ablation elements 804 to communicate electrical signals (e.g., temperature sensor electrical signals, impedance) or deliver electrical energy (e.g., electrical energy configured for radiofrequency ablation or irreversible electroporation). A sensor such as a temperature sensor 817 may be positioned in or on ablation elements 804. Tension wires 806 may be connected to the arms, such as on the distal ends of the superelastic structural wires 801, or to the ablation elements 804. Tension wires may be made, for example, from stainless steel wire or Kevlar thread. Both of the tension wires may be connected to a pull wire 807 that is slidably positioned in a pull wire lumen 808 along the length of the catheter shaft 800. When tension is applied by pulling on the pull wire from the proximal end of the catheter, the tension wires pull on the distal ends of the arms causing them to close. Alternatively, tension wires may both pass through the pull wire lumen along the full length of the catheter, instead of being coupled to a single pull wire, and they may be pulled independently for independent control of each arm (not shown). Arms may be electrically insulated. For example, electrical installation 809 may comprise heat shrink insulation, Parylene, PTFE, polyimide, or an extruded polymer that contains the tension wires, superelastic structural wires and electrical conductors. To facilitate fluoroscopic visualization a radiopaque marker 810 may be connected to a distal end of the catheter shaft 800. The ablation elements may also be radiopaque.

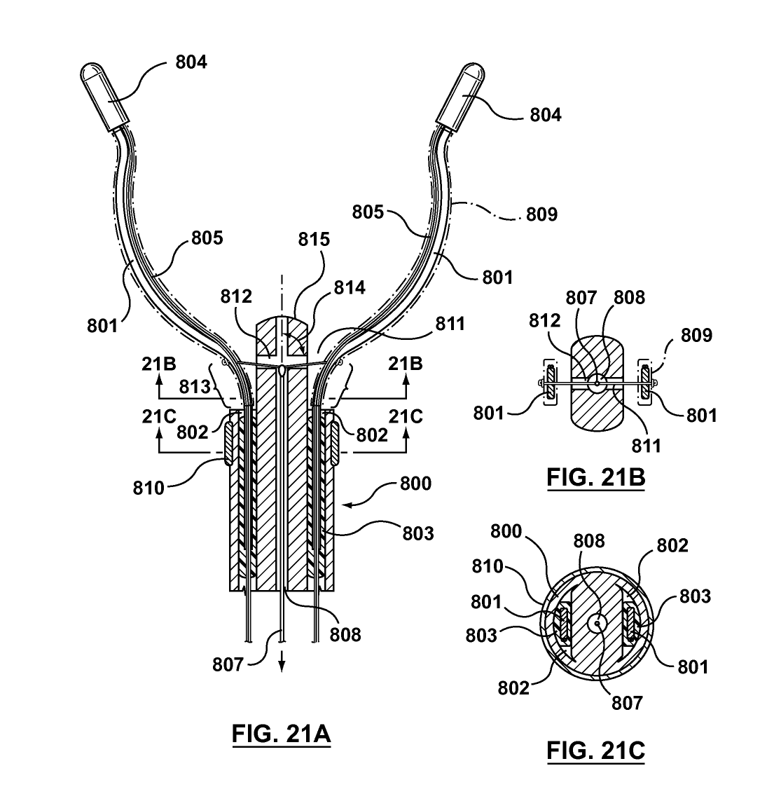

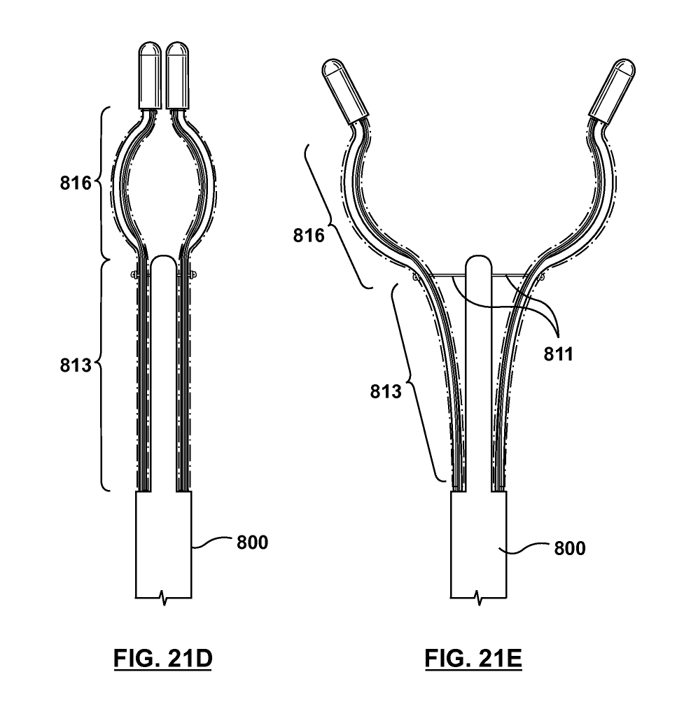

Another embodiment having arms preformed to be normally open that may be closed via application of tension in a pull wire is shown in FIGS. 21A, 21B and 21C. The pull wire 807 is connected to tension wires 811 that are connected to proximal regions 813 of the arms. The tension wires 811 (e.g., Kevlar thread) connect to the arms at a proximal region 813 and pass through tension wire lumens 812 to a central pull wire lumen 808 where they are connected to a pull wire 807 (e.g., stainless steel pull wire). Alternatively, a single tension wire may be connected to both arms and pass through the tension wire lumens 812, where it may be connected to the pull wire 807. The tension wire lumens 812 may be at an angle 814 (e.g., between about 20 to 90 degrees) to the axis of the catheter shaft 800 and connect with the central pull wire lumen 808 that is positioned approximately along the axis of the catheter shaft. The tension wire lumens 812 may be positioned short distance (e.g., between about 1 to 5 mm) from the distal tip. The distal tip 815 may be rounded. Arms may comprise superelastic structural arms 801 (e.g., Nitinol wires), electrical conductors 805 and electrical installation 809. Ablation elements 804 may be connected to the distal tips of the arms. Sensors 817 such as temperature sensors may be positioned in or on ablation elements 804. The arms may be placed in the catheter shaft 800 within lumens 802. In this embodiment the catheter shaft 800 is recessed on both sides where revealing lumens 802 at a position proximal to the distal tip 815. For example, lumens 802 may be recessed between about 5 mm and 30 mm from the distal tip 815. Electrical conductors 805 may be placed in the lumens 802 along the length of the catheter shaft 800 and extend to the ablation elements 804 to communicate electrical signals (e.g., temperature sensor electrical signals, impedance) or deliver electrical energy (e.g., electrical energy configured for radiofrequency ablation or irreversible electroporation). As shown in FIG. 21B superelastic structural wires may comprise a rectangular or oblong shape at the proximal region 813. Structural wires 801 may continue as rectangular ribbons the full-length to the ablation elements, or they may transition to a round profile. The rectangular profile at the proximal region may provide increased elastic strength to move the arms into an open position. Furthermore, as shown in the cross-section of FIG. 21C, the rectangular or oblong profile of structural wires 801 may help to secure the structural wires in lumens 802 extruded in the shaft 800. Lumens 802 may have a rectangular, oblong or other non-circular profile to hold the structural arms in a defined orientation, for example an orientation in which the curvature of the splines and open/close motion is aligned in plane as shown. A rounded profile of the distal region 816 of the elastic members may allow the ablation elements to flex in any cross-sectional direction, which may allow them to self-align to a configuration in the internal and external carotid arteries where the ablation elements are at the center of the intercarotid septum. FIG. 21D shows the device of FIG. 21A in a closed configuration and FIG. 21E in an open configuration. In the closed configuration the pull wire 807 is pulled at a proximal end of the catheter shaft 800 (e.g., by an actuator on a handle) and tension is applied to the tension wire(s) 811, which pull the arms toward the axis of the catheter shaft 800, that is, toward a closed position. The proximal region 813 of the superelastic structural wires comprises a preformed outward bend that opposes the tension of the tension wires 811 moving the arms into an open position when the pull wire 807 is released. When the arms are closed on an intercarotid septum the elastic force of the arms creates electrode apposition with the vessel walls.

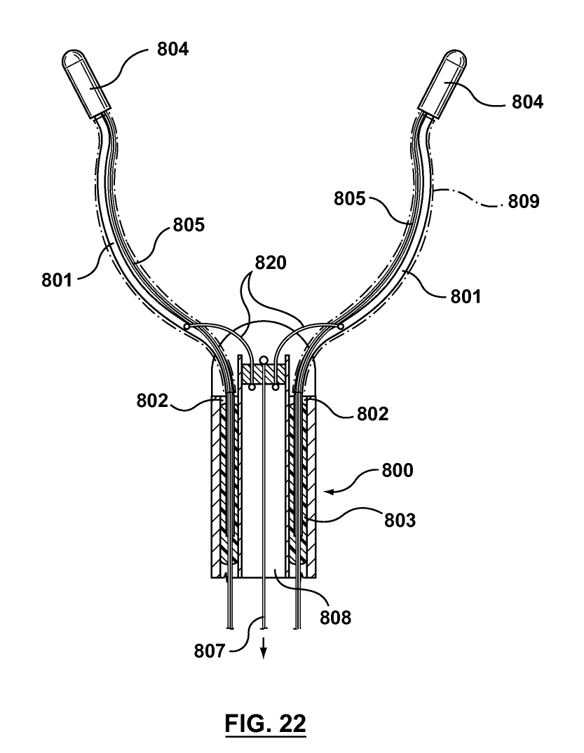

FIG. 22 shows another embodiment of an ETAP configured to close via actuation of a pull wire 807. This embodiment is similar to those in FIGS. 20 and 21 however tension wires are replaced with superplastic preformed wires 820. Super elastic structural wires 801 in the arms are pre-formed to a naturally opened configuration. Application of tension to pull wire 807 creates tension in wires 820 causing arms to close.

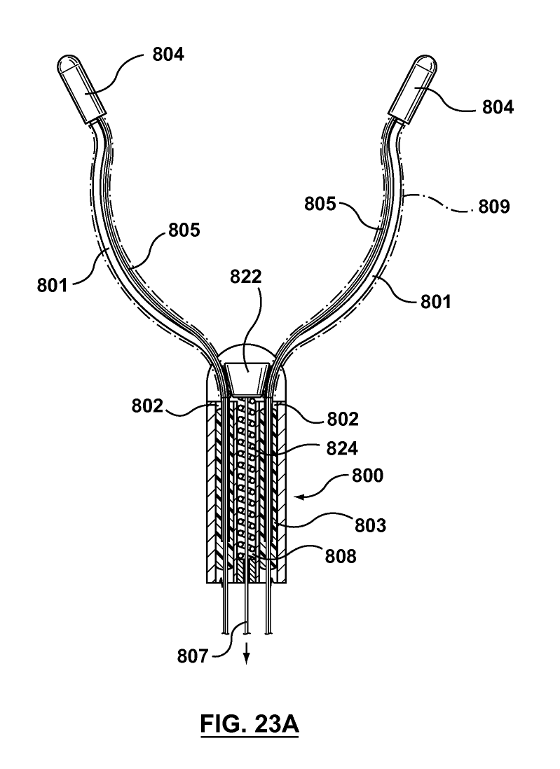

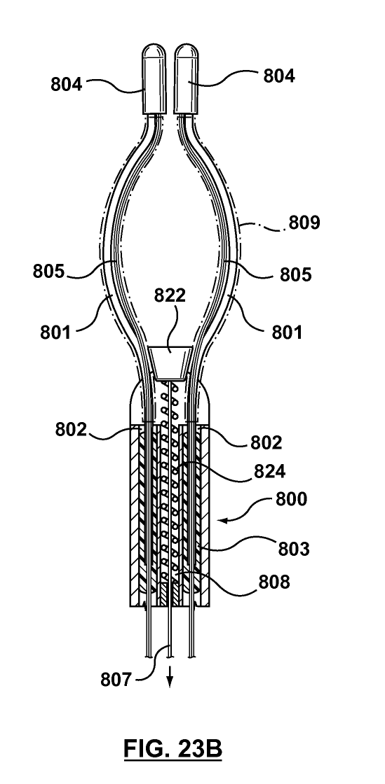

An embodiment of an ETAP catheter configured to open via actuation of a pull wire 807 is shown in FIGS. 23A and 23B. In this embodiment actuation of forearms is created by movement of a wedge 822. The wedge 822 is connected to a pull wire that runs the length of the catheter shaft 800. A compression spring 821 is compressed when tension is applied to the wire. When tension on the pull wire 807 is released the spring 821 causes the wedge 822 to move distally. Superelastic structural members 801 may be pre-formed in a normally closed configuration and comply with the wedge 822 to open as shown. A wedge may be advanced or retracted using other means. For example, in an alternative embodiment a wedge may be advanced and retracted via the rotating motion on a central threaded wire mating with a threaded lumen (not shown).

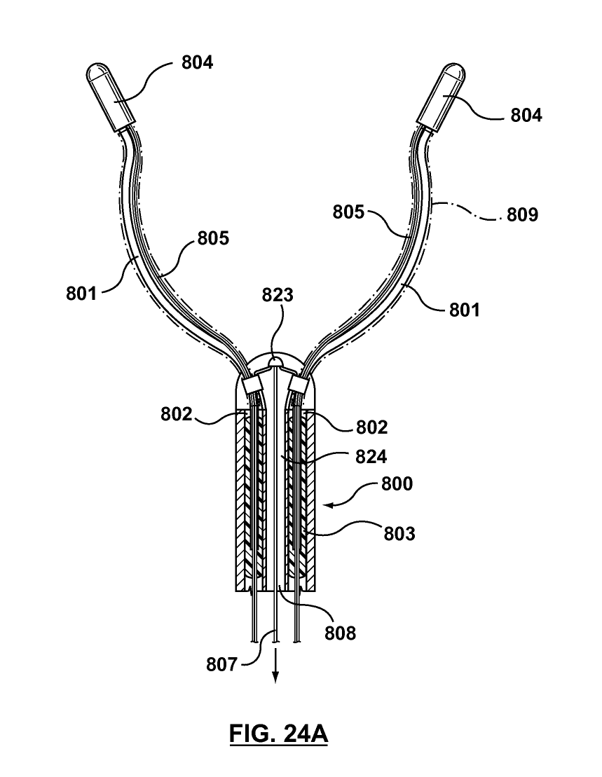

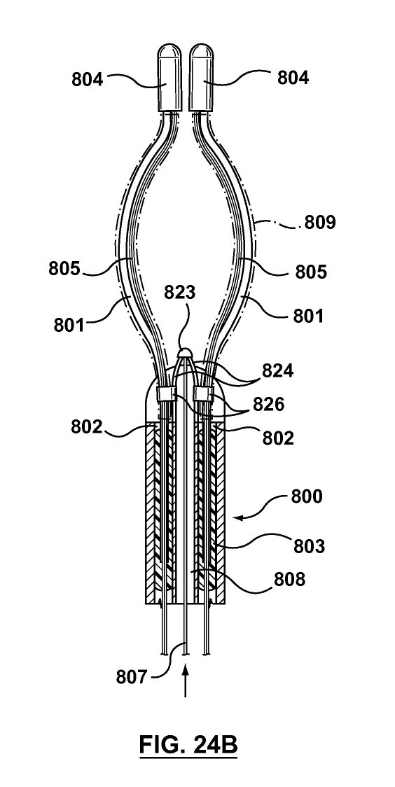

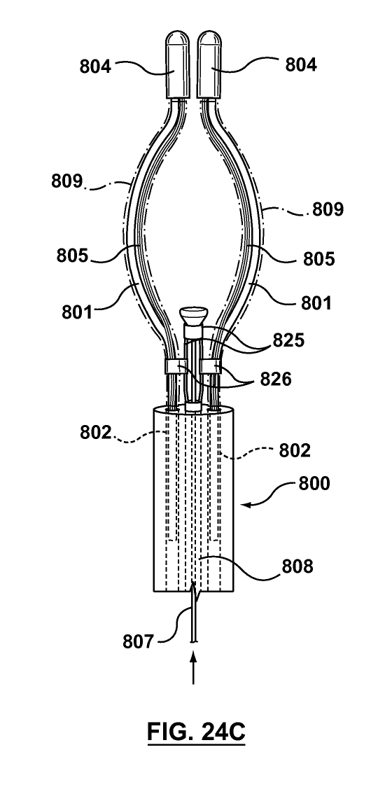

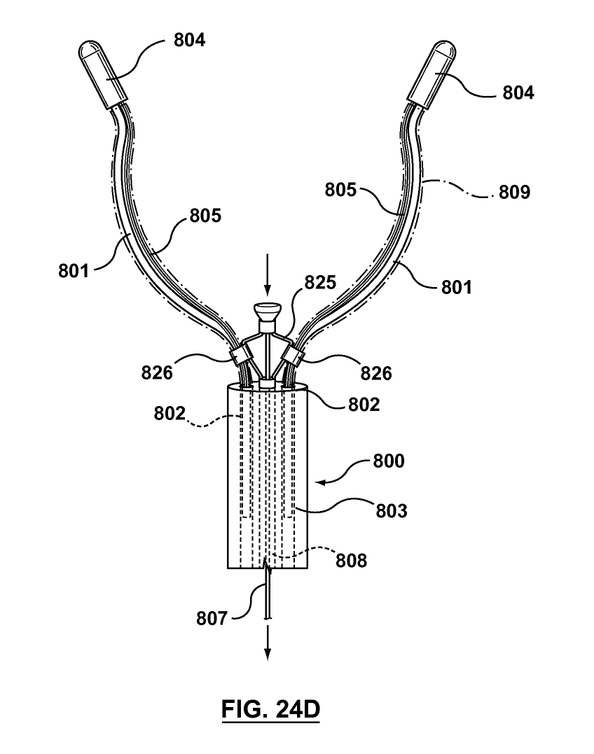

FIG. 24A shows another embodiment of an ETAP catheter configured to open via actuation of a pull wire 807. The arms may comprise superelastic structural wires (e.g., Nitinol, or spring steel) pre-formed with a normally closed configuration as shown in FIG. 24B. The pull wire 807 is connected to a distal cap 823. The distal cap 823 is connected to two elastic spreaders 824. The spreaders 824 may be made from superelastic material such as Nitinol. Tension applied to the pull wire 807 causes distal end cap to move proximally, which causes spreaders 824 to spread radially applying an opening force to the arms. When tension on the pull wire is released the spreaders elastically return to straight configuration causing arms to return to the normally closed configuration. In an alternative embodiment spreaders may be constructed from the laser-cut Nitinol thin wall hypotube 825 as shown in FIGS. 24C and 24D. In these embodiments the spreaders, whether they be wires 824 or laser cut hypotube may be connected to the arms, for example with a collar 826, to maintain contact.

The catheter in FIGS. 24A-D is an example of an endovascular carotid septum ablation catheter comprising first and second diverging arms with free distal ends, the arms extending generally distally from the catheters, the first and arm comprising a first ablation element and the second arm comprising a second ablation element, wherein the catheter has an actuation mechanism therein configured to actuate at least of the first and second arms to change the position of the first and second arms relative to one another. The first and second arms can have unstressed configurations such that the ablation elements are more than about 4 mm apart measured along a line perpendicular to the longitudinal axis of the catheter.

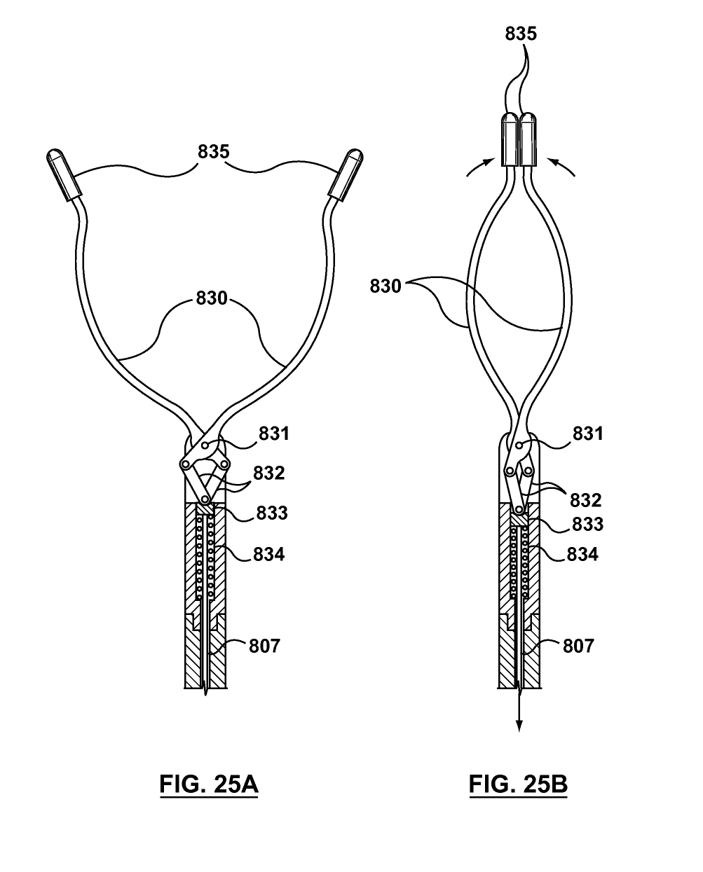

FIGS. 25A and 25B show an embodiment of an ETAP catheter configured to close via actuation of a pull wire 807. The arms may comprise elastic structural wires (e.g., Nitinol or spring steel) that pivot about a pin joint 831 and are connected to mechanical linkages 832 which are connected to a plunger 833. The plunger 833 is joined to a pull wire 807. When tension is applied to the pull wire 807 the mechanical linkages cause the arms 830 to close (FIG. 25B). When tension is released from the pull wire a compression spring 834 pushes the plunger to cause the arms 830 to open (FIG. 25A). The elastic structural wires may be covered in an electrical insulation (not shown). Ablation elements 835, such as electrodes, may be connected to the arms 830 and electrical conductors (not shown) may extend along the length of the catheter and connect the ablation elements 835 or sensors (not shown) to an electrical connector on the proximal region of the catheter.

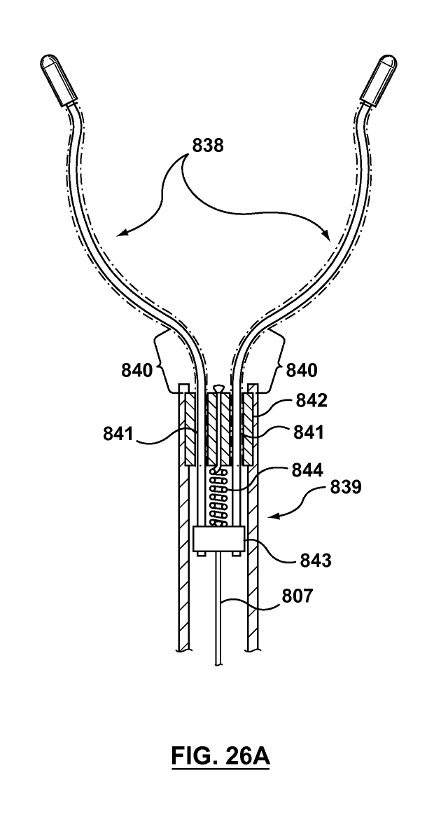

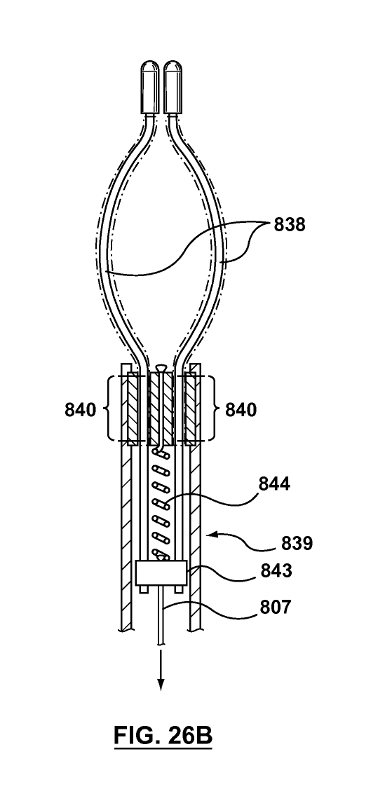

FIGS. 26A and 26B show an embodiment of an ETAP configured to open and close by advancing or retracting elastic arms 838 from a shaft 839. The arms 839 comprise an outwardly curved portion 840. When the outwardly curved portions 840 are unconstrained the arms 838 are in an open configuration. The arms pass through lumens 841 in an end piece 842 and are connected to a plunger 843. The plunger is connected to a pull wire 807 that extends approximately the length of the catheter and is connected to an actuator, for example on a handle. The end piece 842 is connected to the shaft 839 and a tension spring 844 joins the end piece to the plunger. When tension is applied to the pull wire 807 via the actuator, the plunger pulls the outwardly curved portion 840 of the arms 838 through the lumens 841 straightening the curves and closing the arms 838 toward one another (FIG. 26B). When tension is released from the pull wire 807 the tension spring 844 pulls the plunger toward the end piece 842 pushing the arms through the lumens so the outwardly curved portions 840 are unconstrained and the arms open (FIG. 26A).

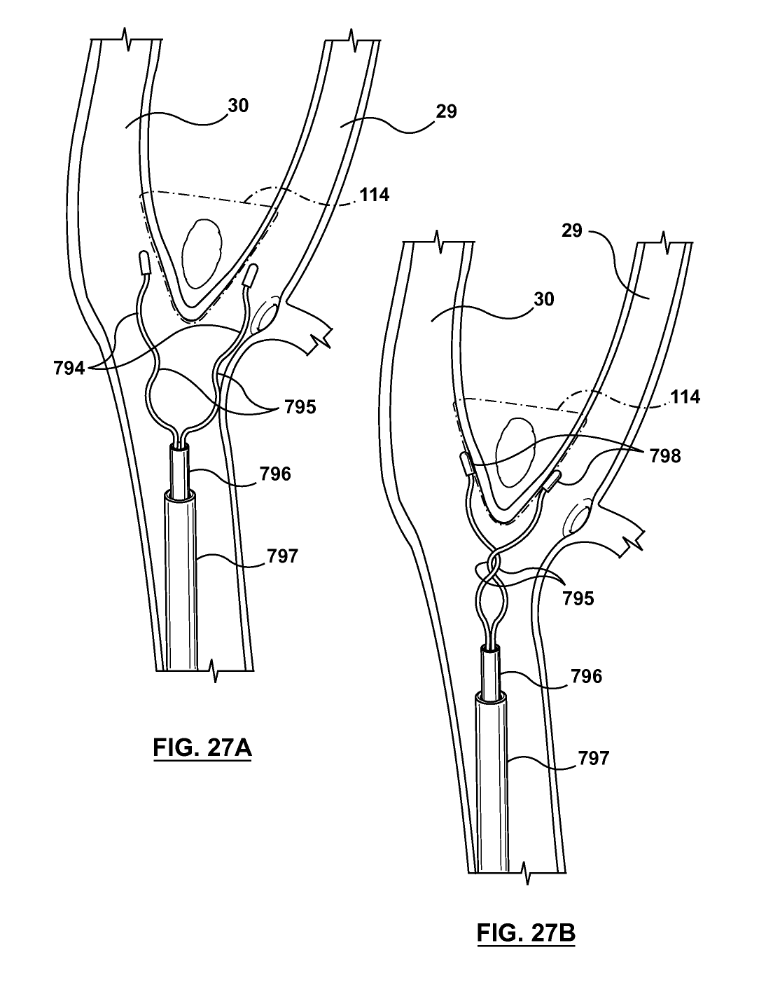

An alternative embodiment of an ETAP catheter configured to be opened and closed by a user is shown in FIGS. 27A and 27B. Aims 794 are elastically flexible and have a shape similar to that shown with curvature having a waist 795. The arms are connected to a shaft 796. The catheter may be delivered through a sheath 797 to a carotid artery. When the catheter is in the sheath the arms flexibly conform to be contained within the sheath. When the sheath is retracted the arms deploy to their preformed shape. The catheter may be advanced so one arm is in an internal carotid artery 30 and the other is in an external carotid artery 29. The user may rotate the proximal end of the catheter wherein torque is transmitted along the shaft 796 rotating the arms causing them to twist around one another and the waists 795 may interlock with one another as shown in FIG. 27B. The waists may be approximately 5 to 20 mm from the distal ends of the ablation elements 798.

Controllable Deflection with Open/Close Actuation

An ETAP catheter may be configured to have controllable deflection, that is, user actuated bending of a portion of the catheter in a distal region. As described earlier, an ETAP catheter may be delivered through a sheath to a common carotid artery 102 where it may be deployed from the sheath. Carotid artery anatomy is quite variable from patient to patient or side to side and alignment of a common carotid artery with internal and external carotid arteries may involve a range of angles or planarity. Controllable deflection may allow a user to account for variable anatomy by aiming the distal end of the catheter at a carotid bifurcation prior to advancing it on to the intercarotid septum. Controllable deflection may allow a user to place ablation elements on target sites while minimizing contact with vessel walls, which may be especially important in the presence of atheromatous plaque to reduce risk of dislodging plaque. Once ablation elements are generally placed on target sites, controllable deflection may allow a user to adjust an angle of the distal section of a catheter to improve electrode wall contact. Controllable deflection may be configured to deflect in more than one plane (multi-planar) or in one plane (uni-planar), and deflection may be toward one side (unilateral) or two sides (bilateral) of a plane. Multi-planar deflection may be achieved, for example, with multiple pull wires. For example, with four pull wires, pulling any one of the wires will deflect the catheter in that direction. Pulling two adjacent wires will deflect the catheter in the 45 degree direction between the two wires.

In an example embodiment, an ETAP catheter may be configured to deflect toward both sides of a single plane and said plane may be coplanar with open and close movement of catheter arms. Such an embodiment may be delivered through a sheath to a common carotid artery, rotated so the deflection and open/close plane is approximately in plane with a plane created by internal and external carotid arteries, deflected so the distal end is aimed approximately at the carotid bifurcation, opened, advanced over the carotid septum, and closed to place ablation elements in contact with the septum one in the internal carotid artery and one in the external carotid artery. Alternatively, multi-planar deflection may reduce the need for, or amount of, rotating a catheter to align an open/close or arm plane with a bifurcation.

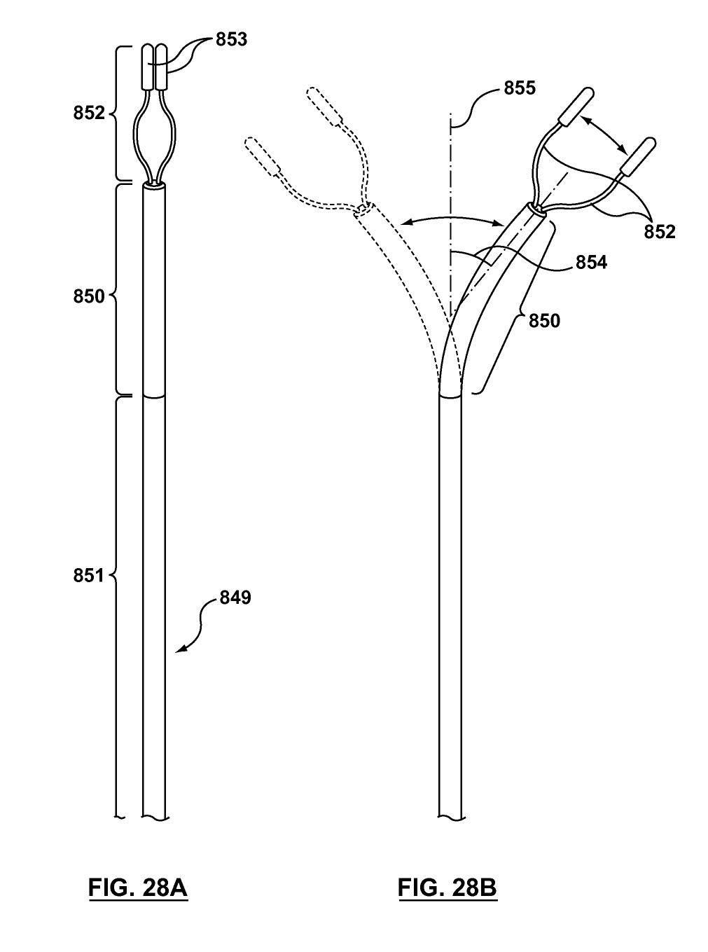

Referring to FIG. 28A an ETAP catheter may comprise a catheter shaft 849 having an elongate region, configured to deliver the distal region of the catheter to a target site in the area of a carotid bifurcation, a controllably deflectable region 850 distal to the elongate region configured to be deflected via user actuation, and arms 852 distal to the controllably deflectable region configured to place ablation elements 853 on an intercarotid septum at positions suitable for carotid body ablation (as shown in FIGS. 5A and 5B). The catheter shaft may have a length in a range of about 90 to 135 cm (e.g., about 120 cm), in which the elongate region 851 spans approximately the length of the shaft up to the controllably deflectable region (e.g., about 85 to 134 cm), the controllable deflectable region 850 spans approximately 10 to 50 mm of the distal end of the shaft, and the arms 852 are approximately 5 to 15 mm in length (e.g., about 10 mm). As shown in FIG. 28B a controllably deflectable region 850 may deflect the arms 852 to both sides of the shaft axis 855, and deflection may be limited to a predetermined maximum angle 854 of about 20 to 60 degrees (e.g., about 30 degrees). The arms 852 may open and close in a plane that is coplanar with the plane of deflection.

The catheter shaft may be made similar to catheter fabrication methods know in the art. For example, the controllably deflectable section may comprise two pull wires positioned on opposite sided of the shaft such that tension in one wire caused by user actuation causes the shaft to deflect toward the side containing the pull wire in tension. The pull wires may be contained in lumens extruded in the catheter shaft and span approximately the full length of the catheter from the distal end to a handle. The handle may comprise a deflection actuator, such as a lever, knob, or dial that pulls one of the two pull wires at a time. The catheter shaft 849 may be made from different durometer materials to provide functionality. For example, the elongate region 851 may comprise a Pebax extrusion with a higher durometer (e.g., about 55 D to 75 D, about 63 D) than the controllably deflectable region 850, which may comprise a Pebax extrusion with a softer durometer (e.g., about 35 D to 55 D, about 40 D) so deflection is limited to the softer controllably deflectable region. In the case of a uni-directional deflection catheter embodiment, a controllably deflectable region may comprise a lumen off-axis to contain a pull wire. Tension in the pull wire would compress the controllably deflectable region causing it to deflect in the direction of the lumen from the axis. In the case of a bi-directional deflection catheter embodiment, at controllably deflectable region may comprise 2 lumens off-access on opposing sides to contain to pull wires. The pull wire lumens in the controllably deflectable region may connect to a single coaxial lumen in the elongate region. The controllable deflection described with respect to FIGS. 28A and 28B can be incorporated into any catheter herein.

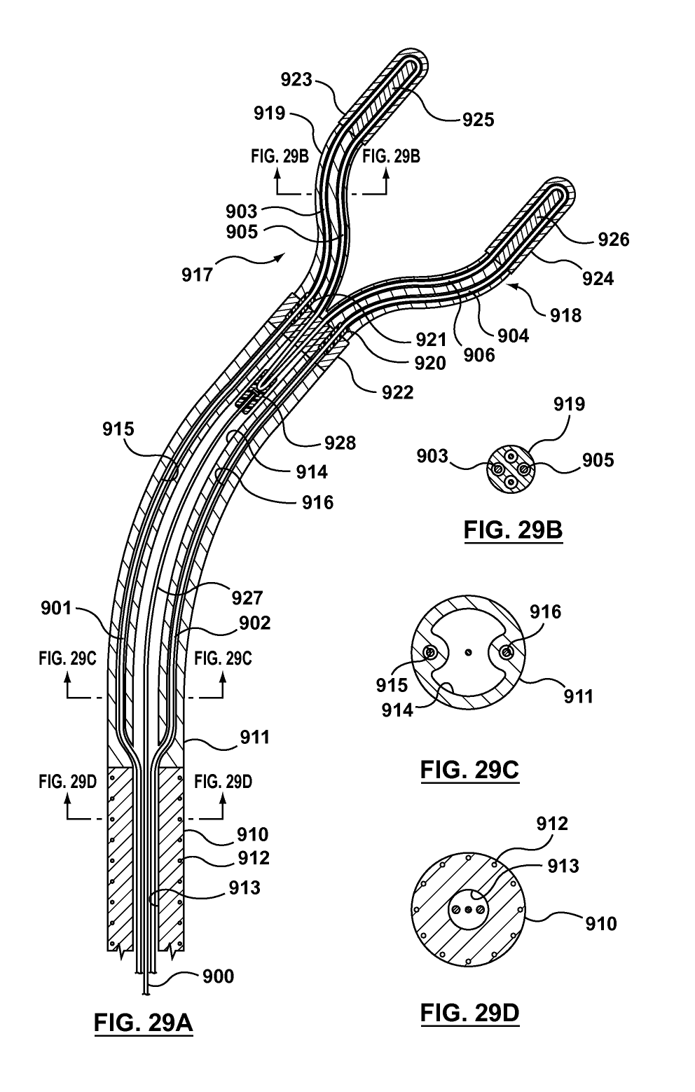

An embodiment of an ETAP catheter, as shown in FIGS. 29A, 29B, and 29C, is configured for bi-directional controllable deflection in a plane that is coplanar with open/close actuation of two splines. The catheter is configured to place electrodes, mounted to each of the two splines, on an intercarotid septum in a region 136, 137, 138, and 139 suitable for carotid body ablation (as shown in FIGS. 5A and 5B). In this embodiment, a shaft comprises an elongated section 910 and a controllably deflectable section 911. The elongated section 910 may be made from extruded Pebax with a durometer of about 55 D to 75 D (e.g., about 63 D) and a wire braid 912 to enhance transmission of torque and translation from a handle (not shown) on a proximal end of the catheter. The elongated section 910 comprises a coaxial lumen 913 (shown in FIG. 29D) and may be approximately 120 cm long and have a diameter of about 6 French (e.g., about 2 mm). The controllably deflectable section 911, positioned distal to the elongated section, may be approximately 1 to 5 cm long (e.g., about 2.54 cm long) with a diameter of about 2 mm and made from extruded Pebax with a durometer that is softer than the elongated section, (e.g., about 25 to 55 D). The controllably deflectable section 911 may comprise a coaxial lumen 914, a first off-axis lumen 916 and a second off-axis lumen 917 (shown in FIG. 29C). Distal to the controllably deflectable section 911 the catheter diverges into a first spline 917 and a second spline 918, which may be opened apart from one another and closed toward one another via an actuator on a handle (not shown). The first and second splines comprise electrical insulation such as an extruded tube 919, for example made from soft Pebax (e.g., about 40 D) or silicone. The extruded tubes 919 may have a length of about 5 to 10 mm (e.g., about 6 mm) and a diameter of about 0.8 mm.

A preformed superelastic Nitinol wire 900 is used to function as a first deflection pull wire 901, a second deflection pull wire 902, a first spline structural segment 903, a second spline structural segment 904, a first spline actuation segment 905, and a second spline actuation segment 906. The Nitinol wire 900 may have a diameter of approximately 0.006″ to 0.012″. The Nitinol wire may optionally have a varying diameter to provide desired flexibility or stiffness that varies along its length. As shown the Nitinol wire 900 is slidably positioned in the coaxial lumen 913 of the elongate section 910 then passes in to the first off axis lumen 915 of the controllably deflectable section 911 where it acts as the first deflection pull wire 901. The first deflection pull wire 901 is anchored with a first crimp 921 to a distal end piece 922 at the distal end of the controllably deflectable section. The distal end piece 922 may be made from a rigid radiopaque material such as radiopaque thermoplastic and functions as a radiopaque marker, an anchor for the first and second pull wires, an anchor for the first and second spline structural segments, and provides a protected opening to the coaxial lumen 914. The proximal ends of the deflection pull wires 901 and 902 are connected to an actuator in a handle (not shown). When tension is applied to one of the deflection pull wires the controllably deflectable section 911 compresses on the side of the tensioned wire and deflects toward said side.

The first and second structural segments 903 and 904 are made from the Nitinol wire 900 and may comprise a preformed shape as shown that elastically holds the splines in an open configuration, for example such that the electrodes 923 and 924 are approximately 10 to 20 mm apart, when unconstrained by a sheath and when tension in an open/close pull wire is released. The Nitinol wire 900 forms a 180-degree bend at the distal end of the spine where it is inserted in an electrode 923 and held in place by a friction fitted core 925. The Nitinol wire 900 returns along the spline as a first spline actuation segment 905 and enters through a central opening in the distal end piece 922 to the coaxial lumen 914. In the coaxial lumen the Nitinol wire forms another 180-degree bend to form a second spline actuation segment 906, second spline structural segment 904, and second deflection pull wire 902. In the coaxial lumen 914 the Nitinol wire 900 is connected to an open/close pull wire 927, for example with a crimp 928. The open/close pull wire is slidably contained in the coaxial lumen 914 and 913 and passes to an actuator on a handle (not shown). When tension is applied to the open/close pull wire 927 via the actuator, the first and second spline actuation segments 905 and 906 are pulled into the coaxial lumen 914 while the length of the first and second spline structural segments 903 and 904 remains consistent due to anchoring at the distal end piece 922 and the electrodes 923 and 924, thus causing the splines to move toward a closed configuration. The splines 917 and 918 may be approximately the same length or may be offset so one is longer than the other. For example, a first spline 917 may be about 6 mm long while the second spline 918 is about 11 mm long. Electrical conductors (not shown) may pass from an electrical connector on a proximal region of the catheter, through the catheter shaft and diverging arms to the electrodes.

The embodiment in FIGS. 29A-C is an exemplary embodiment in which first and second arms have unstressed configurations that are in substantially the same plane, and wherein the catheter is configured for bi-directional controllable deflection in the plane of the first and second arms.

Controllable Deflection with Slide on Arms

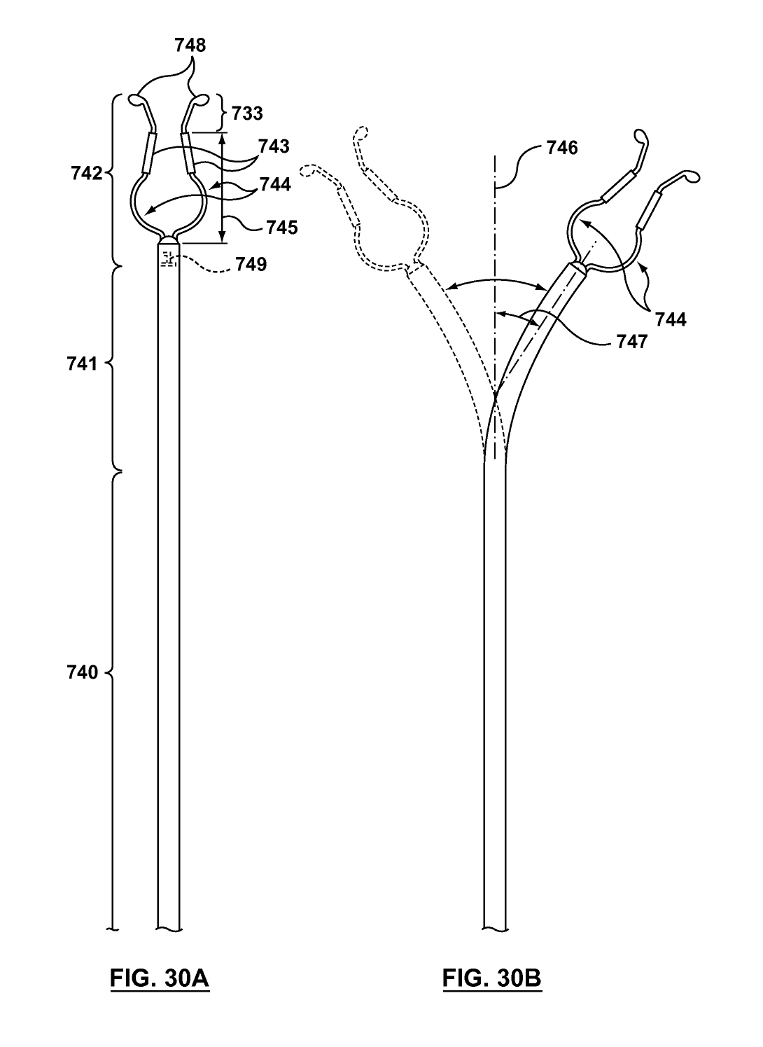

An example embodiment of an ETAP catheter configured for controllable deflection with a slide-on arm configuration is shown in FIG. 30A in an undeflected state and FIG. 30B in a deflected state. The catheter comprises a catheter shaft having an elongate region 740, configured to deliver a distal region 742 of the catheter to a common carotid artery in the area of a carotid bifurcation via endovascular access (e.g., through a 7 French sheath), and a controllably deflectable region 741 distal to the elongate region 740 configured to be deflected via user actuation. Distal region 742 is distal to the controllably deflectable region 741 and includes structural member 720 including first and second arms described above with respect to FIG. 17. All of the features of the arms described above with respect to FIG. 17 are reiterated with respect to FIGS. 30A and 30B. Distal region 742 includes elastically flexible, preformed, or unstressed, diverging arms 744, ablation elements 743 mounted to the arms, and a distal funnel region 733. Each of the arms includes a clearance portion as described herein proximal to ablation elements 743. The distal region may further comprise rounded atraumatic tips 748. The distal region 742 is configured to slide on to an intercarotid septum and place ablation elements 743 on an intercarotid septum within desired target regions 136, 137, 138, and 139 suitable for carotid body ablation (as shown in FIGS. 5A and 5B). To aid fluoroscopic visualization, the distal region 742 may comprise radiopaque markers 749 or various components of the distal region may be radiopaque such as the ablation elements 743 or arms 744. A user may control deflection of a distal region of the catheter, for example, by manipulating an actuator on a handle connected to a pull wire that passes through the catheter shaft to a deflectable section 741. The deflectable section may deflect the distal region toward both sides of a single plane and said plane may be coplanar with alignment of the catheter arms 744.

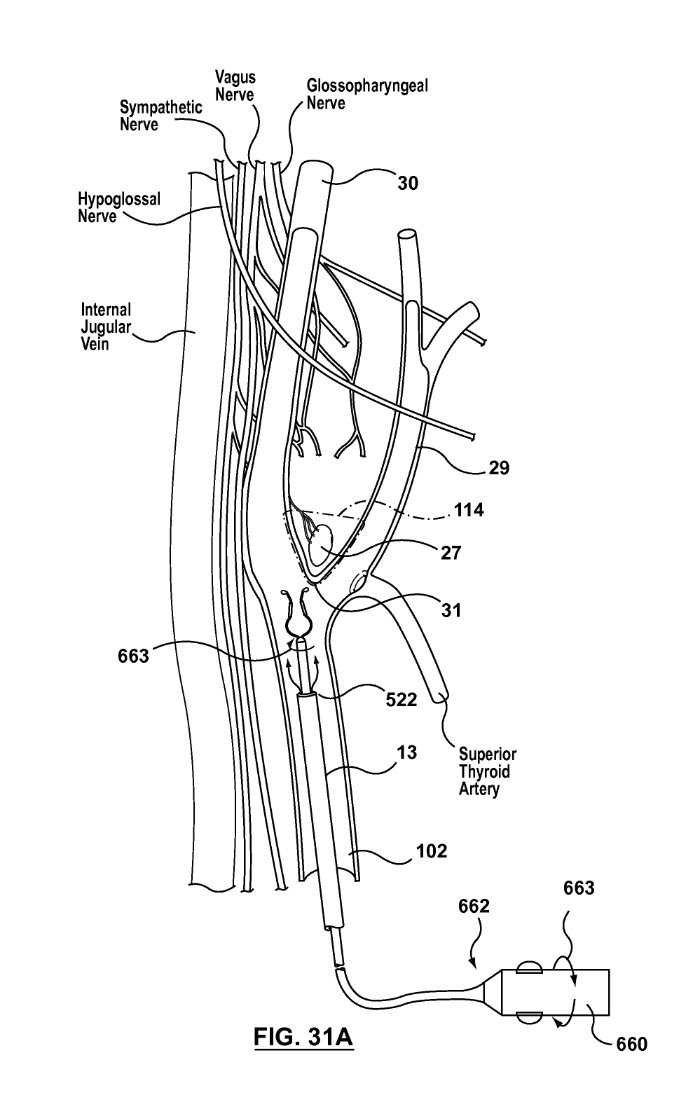

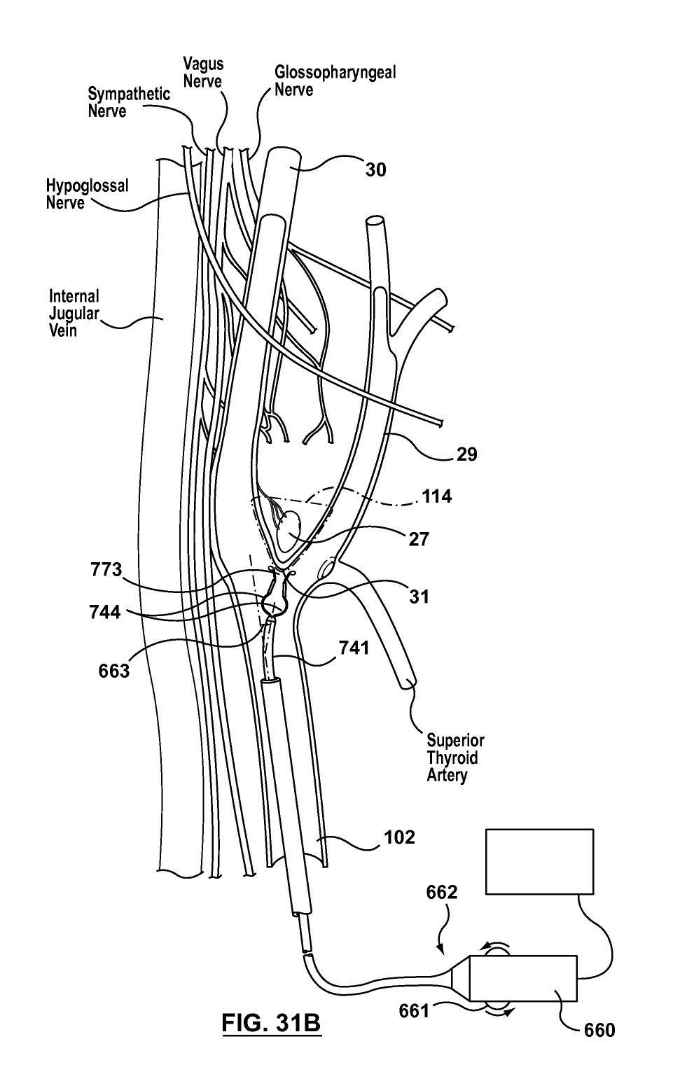

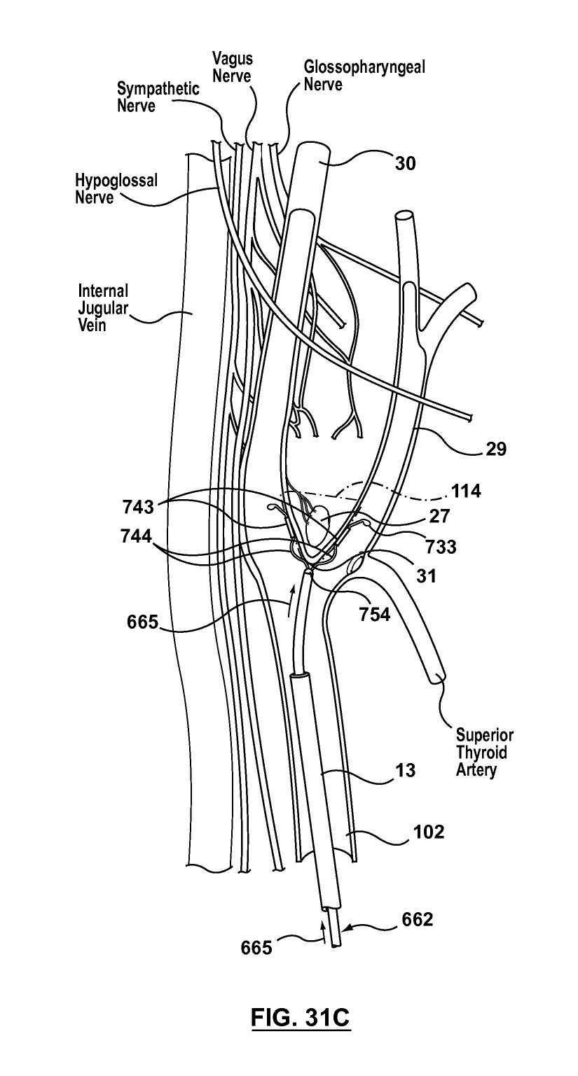

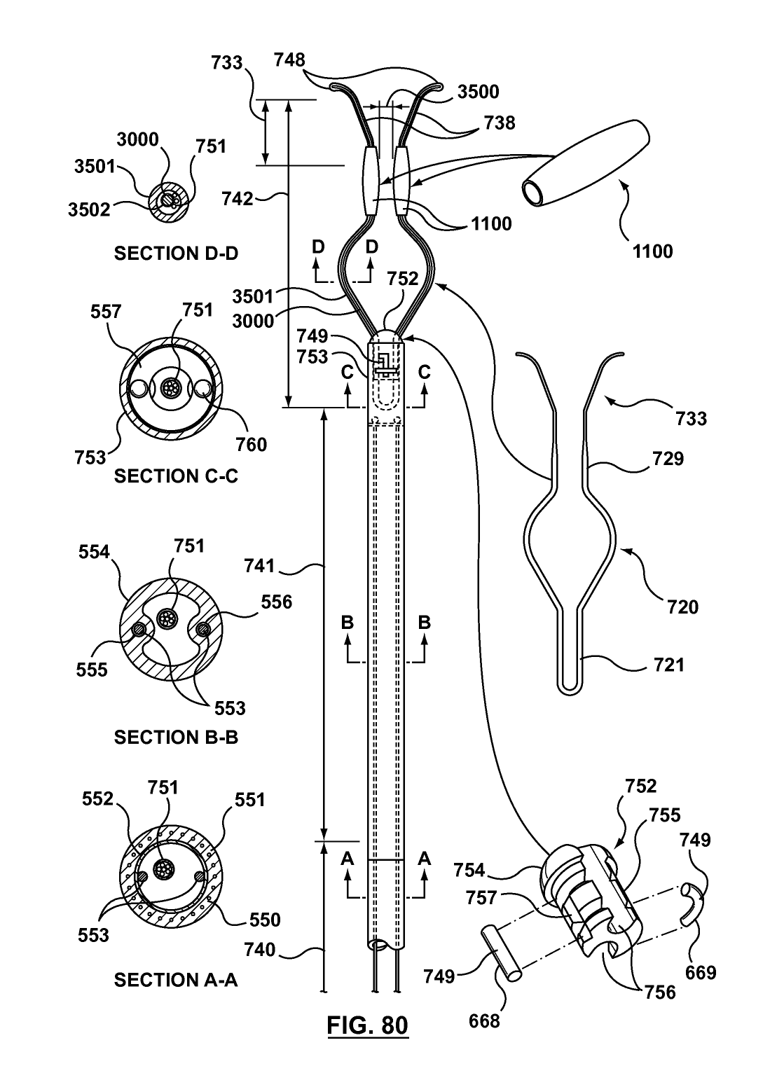

FIG. 31A illustrates a catheter such as the catheter shown in FIGS. 30A and 30B (or FIG. 80, for example) being delivered through a sheath 13 to a common carotid artery 102 and rotated 663, for example by rotating a proximal region of the catheter 662 such as a handle 660, so the deflection and open/close plane is approximately in plane with a plane created by internal and external carotid arteries, referred to as the carotid plane. Radiopaque contrast 522 may be injected, for example through the sheath 13, to common carotid artery 13 to allow a user to visualize radiopaque aspects of the distal region 742 with respect to common carotid artery 102, internal carotid artery 30 and external carotid artery 29. A carotid plane may be ascertained by rotating a C-arm until the carotid arteries appear the widest distance apart on a fluoroscopic monitor. This indicates that the C-arm is substantially orthogonal to the carotid plane. As shown in FIG. 31B deflectable section 741 may be deflected 664 by manipulating a deflection actuator 661 located at a proximal region of the catheter 662, for example on a handle 660, so the funnel section 733 is aimed approximately at the carotid bifurcation 31. As shown in FIG. 31C funnel section 733 may be advanced 665 over the carotid bifurcation 31 and on to an intercarotid carotid septum 114, for example by advancing a proximal region of the catheter 662 in to sheath 13, such that contact force on the funnel section created by the advancement of the catheter elastically spreads the arms 744 apart as ablation elements 743 are advanced and self-aligned on to a desired target region on the intercarotid septum. If required, further small adjustments in deflection may improve consistency of contact with both ablation elements 743 (e.g., electrodes). Alternatively, multi-planar deflection may reduce the need for, or amount of, rotating a catheter to align an open/close or arm plane with a bifurcation.

The endovascular carotid septum ablation catheter shown in FIGS. 30A and 30B, shown in use in FIGS. 31A-C, includes first and second diverging arms, the first arm comprising an ablation element and configured so that the ablation element is in contact with a carotid septal wall in an external carotid artery when the catheter is coupled with a common carotid artery bifurcation, the second arm comprising a second ablation element and configured so that the second ablation element is in contact with a carotid septal wall in an internal carotid artery when the catheter is coupled with the bifurcation, as shown in FIG. 31C. The ablation elements are disposed on the arms so that the ablation elements are in contact with the carotid septal walls between the bifurcation and about 4-15 mm cranial to the bifurcation when the catheter is coupled with the bifurcation, as shown in FIG. 31C. In this embodiment each of the ablation elements is disposed on the arms about 4 mm to about 15 mm distal to a distal end of a catheter shaft, the distance being measure along the longitudinal axis of the shaft. This allows the ablation elements to be positioned at desired regions along the septal wall when the catheter is engaging the bifurcation.

In the embodiment shown in FIGS. 30A and 30B, the arms are each configured such that substantially all contact that occurs between the arms and the walls of the internal and external carotid arteries occurs between the ablation elements and the walls, as is described herein with respect to other embodiments. The arms each have a clearance portion, in this embodiment with a general arch configuration, proximal to the electrode mounting region, as can be seen in FIGS. 30A and 30B, the clearance portion being configured to substantially avoid contact with the walls of the external and internal carotid arteries when the catheter is coupled with a common carotid artery bifurcation such that substantially all contact that occurs between the arms and the walls of the internal and external carotid arteries occurs between the ablation elements and the walls, as shown in FIG. 31C. Each of the clearance portions can be electrically insulated from the ablation element. Each of the clearance portions has an arch configuration. Each of the clearance portions is flexible and resilient such that the clearance portion can be deformed to a straighter configuration for delivery, and is adapted to assume the arch configuration when unconstrained. Each of the clearance portions is configured to make less surface area contact with the wall of the carotid artery than the ablation element, as shown in FIG. 31C. As described herein, the first and second arms are configured to self-align within the internal and external carotid arteries, such as to the positions shown in FIG. 5A. The first and second arms are in substantially the same plane in unstressed configurations, and each arm is flexible so that they are configured to be deflectable out of plane, as is described in more detail herein.

In the catheter shown in FIGS. 30A and 30B, the first and second arms have unstressed configurations in which the first and second ablation elements are 6 mm or less apart measured along a line perpendicular to a longitudinal axis of a catheter axis. The ablation elements can be 4 mm or less apart measured along a line perpendicular to a longitudinal axis of a catheter axis. The ablation elements can be 2 mm or less apart measured along a line perpendicular to a longitudinal axis of a catheter axis.

Each of the arms in the catheter shown in FIGS. 30A and 30B comprises a distal region 733 distal to the ablation element that extends away from a longitudinal axis of the catheter relative to the ablation element. This is labeled as funnel region 733 and is described in more detail herein. The distal regions 733 are more flexible than the regions of the diverging arm region proximal to the first and second ablation elements, an example of which is described in FIG. 32I. The distal regions 733 are each in plane with the respective diverging arm, and are each electrically insulated from the respective ablation element. In some instances the distal regions 733 have a diameter dimension less than a diameter dimension of the arm proximal to the electrode region, an example of which is described below with respect to the embodiment in FIG. 32I.

The catheter shown in FIGS. 30A and 30B includes diverging arms that are in substantially the same plane in unstressed configurations. In FIGS. 30A and 30B the plane is the plane of the page. The catheter is also configured for controllable deflection in the plane in which the arms are in, first plane, as shown in FIG. 30B, that is approximately coplanar with a plane in which the first and second diverging arms are disposed. The catheter in FIGS. 30A and 30B is also an example of diverging arms that have free ends. In general, diverging arms with free distal ends generally refers to distal ends of arms that are not physically connected to another structure. Ablation elements 743 shown in FIGS. 30A and 30B are each angled inward with respect to a longitudinal axis of a catheter shaft.

One or both of the arms can have a coating layer around the arm as is disclosed herein. In some embodiments the coating layer is an insulative material.

As shown in FIG. 31C, the first and second arms are configured to urge portions of the internal carotid artery and the external carotid artery towards each other when positioned therein. The catheter in FIGS. 30A and 30B are also an example of first and second arms that are symmetrical about a longitudinal axis of the catheter.

While not shown, the ablation elements in FIGS. 30A and 30B are in electrical communication with a generator configured to deliver RF energy to the ablation element. The generator can be configured to deliver any of the delivery parameters described herein, such as operating the ablation elements in bipolar RF mode.

In the embodiment in FIGS. 30A and 30B, one or both of the arms can have an unstressed length measured along a longitudinal axis of a catheter shaft between about 3 mm and about 20 mm. A distance between a distal end of the catheter shaft and a distal end of one or both of the ablation elements can between about 4 mm and about 15 mm. The ablation elements can have lengths of between about 3 and about 10 mm. As shown in FIGS. 30A and 30B but more easily seen in FIG. 32, the inner portion of the ablation elements are not flush with the arms. This is partly because the ablation elements are mounted on the arms. The ablation element is therefore in position to make tissue contact while distancing the arm from the tissue. Any of the ablation elements herein, including the barrel configurations, can be used in place of the ablation elements shown in FIGS. 30A and 30B.

The catheter in FIGS. 30A and 30B also illustrates an example of first and second ablation elements that are disposed on the arms at substantially the same distance from the a distal end of the catheter shaft. The catheter can also include a temperature sensor coupled to the ablation elements configured to sense temperature proximate the ablation elements.

Any other structure or feature described herein in any other embodiment of an ablation catheter can be incorporated into the catheter shown in FIGS. 30A and 30B either in combination or as a replacement to a particular component.

An illustration of an ETAP catheter configured for controllable deflection with a slide-on arm configuration is shown in FIG. 32A. The ablation catheter in FIG. 32A is considered the same as the catheter in FIGS. 30A and 30B unless it is indicated herein to the contrary, and it can be used in the same manner shown in FIGS. 31A-C. The relevant description of the catheter in FIGS. 30A and 30B will therefore not be duplicated here. The catheter includes an elongate section 740, a deflectable section 741, a distal region 742, and a handle on a proximal end (not shown). The catheter shown in FIG. 32A can be used in the same manner shown in FIGS. 31A-C. The catheter shaft may have a length in a range of about 90 to 135 cm (e.g., about 120 cm), in which the elongate region 740 spans approximately the length of the shaft up to the controllably deflectable region (e.g., about 85 to 134 cm), the controllable deflectable region 741 spans approximately 10 to 50 mm, and the distal region 742 comprises arms 744 having a spline length 745 of approximately 5 to 15 mm in length (e.g., about 11 mm). As shown in FIG. 30B a controllably deflectable region 741 may deflect the distal region 742 to both sides of the shaft axis 746, and deflection may be limited to a predetermined maximum angle 747 of about 20 to 60 degrees (e.g., about 30 degrees). The arms 744 may be aligned in a plane that is coplanar with the plane of deflection. The distal region 742 includes the structural member 720 shown and described above in FIGS. 17 and 30A-31C, including diverging arms with unstressed configurations as shown. The distal region, including the diverging arms, are configured to resiliently conform to an undeployed state when contained within a sheath and elastically adopt the preformed, or unstressed, shape of a deployed state when not contained within a sheath. The expandable distal region may be mounted on a distal end of a catheter shaft adopted for advancement through a sheath (e.g., a 7 French sheath), for example from a femoral artery puncture in a patient's groin, advancement to a common carotid artery under fluoroscopic guidance and placement on an intercarotid septum.

In the embodiment shown in FIG. 32A the distal region 742 may comprise an elastic structural member 720 as described above with respect to FIGS. 17 and 30A-31C, a wire spacer 752, energy ablation elements (e.g., RF electrodes, irreversible electroporation electrodes) 743 mounted on the two arms, a funnel region 733, atraumatic tips 748, electrical insulation 750, electrical conductors 751, temperature sensors, radiopaque markers 749, and a distal region shaft tubing 753. The elastic structural member 720, for example as shown in FIGS. 17 and 30A-C provides an elastic skeleton on which to mount the other components, a preformed or unstressed shape or configuration configured to slide on to an intercarotid septum and apply contact force between ablation elements and the septum, self-align the ablation elements within target regions 136, 137, 138, and 139 (see FIGS. 5A and 5B), and an ability to collapse to an undeployed state when contained in a sheath. Elastic structural member 720 may be held in to the distal region shaft tubing 753 with adhesive.

A wire spacer 752 having a cap 754, a column 755, wire grooves 756, and radiopaque marker grooves 757 may be placed in proximal section 721 between both sides of the elastic structural member 720 with the column 755 glued in to the distal region shaft tubing 753, the elastic structural member 720 held in wire grooves 756, and the cap 754 covering the distal opening in the tubing 753. The wire spacer 752 functions to maintain a consistent distance between the two sides of the wire 720 in the proximal section 721, hold radiopaque markers 749, and its cap may provide a rounded, atraumatic surface that may come in to contact with a carotid bifurcation 31 as shown in FIG. 31C. Radiopaque markers 749, such as bands or wires made from radiopaque material (e.g., platinum, platinum-iridium) may be held in radiopaque marker grooves 757. The wire spacer may be made from a molded polymer such as polycarbonate.

Electrically insulative sleeves 750 may cover the elastic structural member 720 and function to provide dielectric strength as well as contain electrical conductors 751. Sleeves 750 may be made from a soft material (e.g., Pebax with a durometer of about 25 D). Electrical conductors 751 may comprise an ablation energy delivery (e.g., radiofrequency or irreversible electroporation) conductor and temperature sensor (e.g., T-type thermocouple) conductors. Electrical conductors 751 may pass through the catheter shaft to the proximal end terminating at an electrical connector, for example on a handle 660.

Ablation elements 743 (e.g., radiofrequency electrodes, irreversible electroporation electrodes) may be placed on the elastic structural member 720 on the electrode-mounting region 729, or on any other arm described herein. Ablation elements 743 may be, for example, electrically conductive (e.g., gold, platinum, stainless steel, or an alloy such as 90% gold 10% platinum) cylinders with a lumen passing through. Ablation elements 743 may have an exposed length 736 of about 0.157″+/−0.002″ (4 mm+/−0.5 mm) and an exposed diameter of about 0.048″+/−0.005″, and an additional mounting length 737 of about 0.030″ to which insulation 750 and 738 may be connected. Ablation elements 743 may comprise an axial lumen of about 0.032″+/−0.002″. Electrode-mounting region 729 of the elastic structural member 720 may be placed in the lumen along with electrical conductors 751. Ablation energy conductors may be electrically connected (e.g., soldered, welded) to an inner surface of the ablation elements 743. For example, a first pole of an electrical circuit connected to a first ablation energy conductor may be connected to a first electrode 737 and an opposing pole of the electrical circuit connected to a second ablation energy conductor may be connected to a second electrode such that the first and second electrodes are in a bipolar configuration. Other conductors 751 may be used for one or more temperature sensors. For example, a copper and constantan conductor may be joined to make a T-type thermocouple positioned in thermal communication with the electrode 743. Once the components are placed in the cavity of the ablation elements 743 empty space in the cavity may be filled, for example with solder, epoxy, thermally conductive epoxy, or radiopaque solder.

Any of the ablation elements can be mounted to any of the arm structures described herein even if it is not specifically stated herein.

FIGS. 32B, 32C, and 32D illustrate an alternative ablation electrode that can be used in place of any of the ablation elements herein, such as electrodes 743 in FIG. 32A. While the flexing and pivoting electrodes in the embodiments in FIGS. 13A-D are described as being configured to increase the consistency of electrode contact and self-aligning, the electrode shown in FIGS. 32B-32D are also configured to increase the consistency of electrode contact. Ablation electrode 1100 has a width, or diameter, that is not constant over its length. As can be seen in the side views in FIGS. 32B and 32C, and in the end view of FIG. 32D, ablation electrode 1100 has a central width 1102 greater than end width 1103, with a gently curving profile as shown. The central width dimension in this specific embodiment is measured at the axial midline of the electrode. This is in contrast to a cylindrical shape, which in the same cross section as shown has linear outer surfaces. In general, electrode 1100 has a barrel configuration, with a central region that has a width greater than a width of a region disposed axially to the central region. The curved profile of the sides of the electrode may facilitate electrode contact with tissue. For example, a greater width in a central region may facilitate distension of the electrode into the elastic wall of a carotid artery. In some embodiments the radius of curvature at the midline can be about 9.5 mm to about 10.5 mm. In some embodiments the radius of curvature varies along the length of the curved surface.

In other embodiments the curved profile need not extend the entire length of the electrode. For example, in some embodiments the curved profile does not extend completely to the end of the electrode. In other embodiments the central region can include any length of electrode that has a linear surface in cross-section (i.e., looks like a cylinder in cross section) rather than being curved.

In a mere example, the length of electrode 1100 is about 4 mm, central width 1102 is about 0.048″+/−0.004″, and end width 1103 is about 0.008″+/−0.002″ less than center width 1102. Inner lumen 1101 can be, for example, about 0.016″. While these dimensions are not intended to be limiting, a maximum outer diameter of 0.048″+/−0.004″ may in some instances be preferred in the configuration of the embodiments described by FIGS. 30-33 to allow the catheter to be inserted through a 7F sheath.

Electrode 1100 can be secured to any arm described or not described herein in any suitable manner. Electrode 1100 is shown with lumen 1101 along its axis, which can be, for example, about 0.016″, through which the structural members may be mounted along with conductors, electrical insulation, and adhesive (e.g., epoxy). For example, electrode can be mounted onto electrode mounting region 3002 of structural member 3000 in FIG. 32I with epoxy.

FIGS. 32E-32H illustrate exemplary electrodes in which portions of the electrode that are configured to make contact with carotid artery contact have different surface configurations that other portions that are not configured to make tissue contact (i.e., portions configured to make contact with blood flow). FIGS. 32E-32H illustrate two exemplary electrodes, 1110 and 1134, wherein the tissue contacting region 1112 and 1132 has the same general configuration as the tissue contacting region of electrode 1100. In the side view and end views of FIGS. 32E and 32F, blood contact region 1114 is substantially shaped like a cylinder and does not have a radius of curvature as does region 1112. FIGS. 32G and 32H illustrate exemplary electrode 1134 in which blood contacting region includes striations configured to increase conduction of heat to flowing blood.

Electrode 1100, or any other electrode herein, can be made from a biocompatible, electrically conductive material to conduct RF to tissue, and optionally a material of high thermal conductivity to conduct heat from the tissue or electrode to blood flow, and optionally a material that is radiopaque so it can be discerned in a fluoroscopic image. An example material is 90% gold, 10% platinum.

Additionally, electrodes with a curved surface, may facilitate electrode contact that is more consistent when the arm is configured to allow the electrode to be applied to the carotid artery wall over a range of angles, such as parallel to the carotid vessel wall +/− about 10°. In the case of a slide-on embodiment such as those shown in FIGS. 30-33, consistency of electrode contact area or pressure may be further facilitated by flexibility of the arms. This is important particularly when the arm assembly (i.e., two arms with electrodes) is not perfectly centered on a carotid septum or when a carotid septum is not symmetrically shaped. As set forth here, consistency of electrode contact area or pressure may improve consistency or predictability of a lesion formed in a carotid septum. Electrical insulation 738 may be placed distal to ablation elements 743 on a funnel region 733 of the elastic structural member 720. Insulation 738 may provide dielectric strength and a lubricious surface to slide easily over an intercarotid septum as the distal region 742 is advanced into position. Insulation 738 may be a soft polymer such as Pebax with a durometer of about 25 D and it may comprise a lubricious outer coating. A rounded, atraumatic tip 748 may be applied on the distal tip, for example by applying a bead of UV adhesive. Alternative embodiments of distal tips that provide a reduced risk of trauma to vessels or of plaque dislodgement may include a tapered wire 723 for the funnel region 733 to provide greater flexibility toward the distal tip.

As shown in FIG. 32A in this embodiment, a shaft comprises an elongated section 740 and a controllably deflectable section 741. The elongated section 740 may comprise a tube 551 made from extruded Pebax with a durometer of about 55 D to 75 D (e.g., about 63 D) and a wire braid 550 to enhance transmission of torque and translation from a proximal region of the catheter, for example a handle 660 (see FIGS. 31A and 31B), and optionally an inner coating or inner tube 552 (e.g., polyimide) to reduce a coefficient of friction of the inner surface of tube. Pull wires 553 may be contained in an inner lumen of the elongate section 740 and reduced friction may allow the pull wires to slide more easily within the lumen. Electrical conductors 751 may also be contained within a lumen in elongate region 740. Elongate region 740 may be approximately 90 to 135 cm long (e.g., about 120 cm) and have a diameter between about 3 to 8 F (e.g., 6 F).

The controllably deflectable section 741, positioned distal to the elongated region, may be approximately 1 cm to 5 cm long (e.g., about 2.54 cm long) with a diameter of about 2 mm and made from extruded Pebax 554 with a durometer that is softer than the elongated region 740, (e.g., about 25 D to 55 D, about 40 D). The controllably deflectable section 741 may comprise a coaxial lumen that contains electrical conductors 751, a first off-axis lumen 555 and a second off-axis lumen 556. Pull wires 553 may be slidably contained in the first and second off-axis lumens. At a distal end of the deflectable region 741 the extrusion 554 may terminate and pull wires 553 may be anchored to the distal end of the deflectable region 741. For example, pull wires 553 may pass through holes in an anchor plate 557 and terminate in a ball 760 or bend that will not pass through the holes in the anchor plate 557. The anchor plate may be for example a relatively rigid material such as a polyimide, polycarbonate or metallic disc. The distal region 742 of the catheter may be connected to the catheter shaft for example by thermally welding distal region shaft tubing 753 to deflectable region tubing 554. When tension is applied to one of the pull wires 553 by pulling a proximal end of the pull wire, for example by manipulating an actuator 661 on a handle 660 as shown in FIG. 31B, the side of the extrusion 554 containing the pulled wire compresses and the deflectable region 741 deflects toward the compressed side.

As shown in FIG. 32A radiopaque markers may be added to the distal region 742 of the catheter. In this embodiment, radiopaque wires (e.g., gold, silver, platinum, platinum iridium) are positioned in radiopaque marker grooves in the wire spacer 752, which allows a user to visualize position of the end cap 754 on fluoroscopy. For example, an end cap 754 seen on fluoroscopy to be touching or within a few millimeters of a carotid bifurcation 31 with ablation elements 743 positioned on each side of an intercarotid septum may indicate that the ablation elements 743 are within a desired region 138 and 139 (see FIG. 5B) due to spline length 732 (see FIG. 17). Furthermore, radiopaque markers may be configured to provide an indication of rotational orientation of distal region 742 with respect to a carotid plane or a C-arm. For example, as shown in FIG. 32A radiopaque markers 749 may comprise a horizontal wire 669 and a vertical wire 668 placed on opposing sides of the wire spacer 752. As the catheter shaft is rotated with respect to a plane of view the apparent position of the horizontal and vertical radiopaque wires relative to one another may appear to align differently due to parallax. A chart shown in FIG. 32J demonstrates how horizontal 669 and vertical 668 radiopaque markers may be oriented to indicate a rotational angle of a plane of arms 668 relative to a plane of view 666. A plane of view 666 may be a plane of a C-arm. A plane of arms 668 may be a plane dissecting the two sides of the elastic structural member 720. In this embodiment, a fluoroscopic image of a side of the catheter shows vertical radiopaque marker 668 to be centered on horizontal radiopaque marker 669 when the plane of view 666 of a C-arm is orthogonal to a plane of the arms 667. When the plane of view 666 and plane of the arms 667 is at any angle other than orthogonal, such as 60, 30, or parallel the vertical radiopaque marker 668 will not appear centered on the horizontal radiopaque marker 669 as shown in FIG. 32J.

FIG. 32I illustrates an exemplary structural member 3000 with monolithic diverging arms that can be used as a structural member for any of the catheters herein. For example, structural member 3000 can replace structural member 720 in any of the embodiments in FIG. 17, 30A-31C, or 32A. Structural member 3000 is, in this embodiment, a wire of superelastic material such as Nitinol. Structural member 3000 includes clearance portions 3001 in each of the first and second aims, electrode mounting regions 3002 in each of the first and second arms to which any of the electrodes described herein can be mounted, including proximal sections 3003 and distal sections 3004, and atraumatic tips 3005 in each of the first and second arms. Electrode mounting regions 3002 include proximal sections 3003 that have a diameter of about 0.012 inches, wherein the diameter in sections 3004 is about 0.006 inches, which in this embodiment is the same as the diameter of atraumatic sections 3005. Sections 3003 and 3004 are separated by transition sections 3006, which have a tapering diameter extending from section 3003 to sections 3004. In other respects structural member 3000 is the same as the structural member shown in the catheter of FIGS. 30A and 30B and 32A and can be used in the same manner.

FIG. 80, like FIG. 32A, illustrates a distal region of an exemplary endovascular carotid septum ablation catheter that includes first and second diverging arms, the first arm comprising an ablation element and configured so that the ablation element is in contact with a carotid septal wall in an external carotid artery when the catheter is coupled with a common carotid artery bifurcation, the second arm comprising a second ablation element and configured so that the ablation element is in contact with a carotid septal wall in an internal carotid artery when the catheter is coupled with the bifurcation. The catheter in FIG. 80 can be positioned for use as is described in the embodiments in FIGS. 31A-C.

The ablation catheter in FIG. 80 is the same as the catheter in FIG. 32A in structure and in use unless indicated in the description of FIG. 80. One difference between the catheters in FIGS. 80 and 32A is that in FIG. 80 the structural member is the structural member 3000 from FIG. 32I. Mounted on the first and second arms of structural member 3000 in the electrode mounting regions are two electrodes 1100 having a barrel shape or curving profile as shown in and described with respect to FIGS. 32B-32D, which facilitates electrode-tissue contact, which is described in more detail herein. The electrodes 1100 can be approximately 90% gold and 10% platinum, which can be chosen for its electrical, thermal, radiopaque, and machinability properties. The electrodes 1100 are about 4 mm long and have a maximum diameter of about 0.048″, which are able to pass through a 7F sheath next to one another. An electrical conductor (e.g., insulated copper) used to deliver RF energy is electrically connected (e.g., soldered or welded) to each of the first and second electrode 1100 (e.g., in the wall of the electrode or on the inner surface of the lumen 1101 in the electrode). A thermocouple (e.g., T-type) is placed in the lumen 1101 (see FIG. 32C) of each electrode 1100 and its conductors are threaded along the proximal part of the structural member and through the shaft of the catheter. Collectively the RF conductor and thermocouple conductors are 751 in section D-D of FIG. 80. The electrodes 1100 are adhered to the electrode mounting region 3002 on the structural member 3000 with epoxy and insulated from the structural member by a heat shrink insulation such as PET 3502. The structural member, which includes first and second arms, is made from superelastic shape-set Nitinol that has a diameter of about 0.012″ in regions 3001 proximal of the electrodes 1100, which provides sufficient resiliency to apply an electrode apposition force to a carotid septum and self-align when the arms are advanced over a carotid bifurcation to couple with a carotid septum, and yet sufficient flexibility to deform when pulled into a sheath, additional details of which are described in more detail herein. Each of the arms in the structural member is ground down to have a diameter of about 0.006″ in regions 3004 distal to the electrodes 1100, which provides sufficient flexibility for atraumatic contact with vessel walls yet enough resiliency to capture a bifurcation and open the arms as they are passed over a septum, additional details of which are described herein. An electrical insulation 3501 (e.g., thin wall Pebax of about 40 D) is applied to each of the arms distal to and proximal to the electrodes 1100 encompassing the electrical conductors 751, the structural member 3000 and the PET insulation and adhered using UV-curable adhesive. The insulation 3501 may be clear to allow UV light to pass through it when curing the adhesive. UV-curable adhesives may also be used to close the distal end of the electrical insulation 3501 and form a dome or ball on the end, which may smoothly glide over a vessel wall with reduced risk of trauma. When the distal structure is assembled as shown there is a space or gap 3500 between the electrodes of about 1 mm+/−0.5 mm measured along a line perpendicular to the longitudinal axis of the catheter shaft, which facilitates advancement of the arms over a septum and allows the arms to deploy smoothly and without getting twisted when advanced from a sheath. As stated above, the embodiment shown in FIG. 80 comprises other features in common with and described in reference to FIG. 32A including a wire spacer 752 holding the structural member 3000 into tube of the catheter shaft, radiopaque markers 749, a deflectable section near or at the distal end of the shaft controlled by pull wires 553, and a non-deflectable section proximal to the deflectable section. As an example, the elongate catheter shaft may have a braid embedded in its wall to improve transmission of torque and may be approximately 90 to 135 cm long (e.g., about 120 cm) and about 6F diameter. A handle (not shown) may be connected on a proximal end of the elongate shaft.

As shown in use in FIGS. 31A-C, the catheter in FIG. 80 includes ablation elements disposed on the first and second arms so that the ablation elements are in contact with the carotid septal wall in the external and internal arteries between the common carotid bifurcation and about 10-15 mm cranial to the bifurcation when the catheter is coupled with the bifurcation. The ablation elements are in contact with the tissue based on passive contact force. Each of the ablation elements is disposed on the arms about 4 mm to about 15 mm distal to a distal end of a catheter shaft. As in any other embodiment herein, more than two diverging arms may be included in the catheter.

As is described in more detail herein, the first and second arms are configured such that substantially all contact that occurs between the arms and the walls of the carotid arteries occurs between the ablation elements and the wall. Substantially all contact includes contact that is at least 60% between the ablation elements and the walls, at least 70% between the ablation elements and the walls, at least 80% between the ablation elements and the walls, at least 90% between the ablation elements and the wall, or more. The first and second arm in the catheter in FIG. 80 include clearance portions proximal to the ablation element, the clearance portions configured to substantially avoid contact with the carotid artery wall when the catheter is coupled with a common carotid artery bifurcation such that substantially all contact that occurs between the arms and the walls of the carotid arteries occurs between the ablation elements and the walls.

In the catheter in FIG. 80, the clearance portions are electrically insulated from the ablation element, and they are shown with arch configuration with a first region that extends away from the catheter shaft axis and a second region that extends back towards the catheter shaft axis. As described in more detail herein, the clearance portions in each arm in the catheter in FIG. 80 is flexible and resilient such that the clearance portion can be deformed to a straighter configuration for delivery, and is adapted to assume the arch configuration when unconstrained. The clearance portions in this embodiment are also configured to make less surface area contact with the walls of the carotid arteries than the ablation element when the catheter is coupled to the bifurcation.

As is described in more detail herein, the first and second arms in the embodiment in FIG. 80 are configured to self-align within the internal and external carotid arteries against the septum. As examples, the first and second arms can comprise a round superelastic wire of between about 0.008″ and about 0.016″ in diameter, such as between about 0.010″ and about 0.014″.

The arms in the embodiment in FIG. 80 are in substantially the same plane in unstressed configurations, and can flexible so that they are configured to be deflectable out of plane, and yet are resilient to allow them to return to the plane. The first and second arms have sufficient resiliency to allow them to move from one stress state to a lower stress state when positioned in contact with the walls of the internal and external carotid arteries. The first and second arms are configured to urge portions of the external carotid arterial wall and the internal carotid artery wall towards each other when positioned in the external and internal carotid arteries and when the catheter is coupled to the bifurcation.

In the embodiment in FIG. 80, the first and second arms have unstressed configurations in which the first and second ablation elements are less than about 6 mm apart measured along a line perpendicular to a longitudinal axis of a catheter axis, and can be less than about 4 mm apart measured along a line perpendicular to a longitudinal axis of a catheter axis, and can be less than about 2 mm apart measured along a line perpendicular to a longitudinal axis of a catheter axis.

In FIG. 80 the first and second arms each comprise a distal region distal to the ablation element that extends away from a longitudinal axis of the catheter relative to the ablation element. The distal region is more flexible than a diverging arm region proximal to the first and second ablation elements. The increased flexibility can be due to a smaller diameter. Additional details of atraumatic tip regions are described herein. The distal regions are each in plane with the respective diverging arm, and are each electrically insulated from the respective ablation element.

The first and second arms of the catheter in FIG. 80 are in substantially the same plane in unstressed configuration, and each of the arms has a free end.

In the embodiment in FIG. 80 the first and second ablation elements are substantially parallel with each other when the first and second arms are in unstressed configurations, but can be angled inward or outward with respect to a longitudinal axis of a catheter shaft. The catheter is also configured for controllable deflection in a first plane that is approximately the plane in which the first and second diverging arms are disposed.

The catheter in FIG. 80 is an example of first and second diverging arms that are symmetrical about a longitudinal axis of the catheter, but they can also be asymmetrical about a longitudinal axis of the catheter. The length of the ablation elements measured along a longitudinal axis of the catheter shaft are the same in this embodiment, but they can be different or have different surfaces areas as described herein. The surface areas of the first and second electrodes are the same but they can be different. The second arm can include a third ablation element different than the second ablation element as is described in more detail herein. The first and second ablation elements are in electrical communication with a generator configured to deliver RF energy to the ablation elements.

The catheter in FIG. 80 includes first and second arms that have substantially the same length, and the lengths in unstressed configurations measured along a longitudinal axis of a catheter shaft are between about 3 mm and about 20 mm, but the arms can have different lengths.

In FIG. 80 a distance between a distal end of the catheter shaft and a distal end of the ablation elements is between about 4 mm and about 15 mm.

In FIG. 80 the ablation elements can have lengths between about 3 and about 10 mm, such as between about 3 mm and about 6 mm, such as about 4 mm.

FIG. 80 shows barrel shaped ablation elements, wherein a central portion of the ablation element is disposed further radially inward than portions of the arm immediately proximal and distal to the ablation element when the arms are in unstressed configurations. The ablation elements also have a greater width dimension along their centers than at the proximal and distal ends. In the embodiment in FIG. 80 the first and second electrodes are disposed at substantially the same distance from a distal end of the catheter shaft measured along a longitudinal axis of the shaft. Each of the ablation elements is also coupled to a temperature sensor configured to sense temperature proximate the ablation element. In alternative embodiments one or both of the arm in the embodiment is configured to be delivered over a guidewire, examples of which are described herein.

The catheter in FIG. 80 is an example of an endovascular carotid septum ablation catheter in comprising first and second diverging arms, the first arm comprising an ablation element and configured so that the ablation element is in contact with a carotid septal wall in one of an external carotid artery and an internal carotid artery when the catheter is coupled with a common carotid artery bifurcation, the second arm comprising a second ablation element and configured so that the second ablation element is in contact with a carotid septal wall in the other of the external carotid artery and an internal carotid artery when the catheter is coupled with a common carotid artery bifurcation, wherein the first and second arms are configured to self-align within the internal and external carotid arteries against the septum.

The catheter in FIG. 80 is an example of an endovascular carotid septum ablation catheter comprising first and second diverging arms with free distal ends, the arms extending generally distally from the catheter; the first arm comprising a first ablation element, the second arm comprising a second ablation element, wherein the first and second arms are, in unstressed configurations, flexible so that they are configured to be deflectable out of plane, and are resilient to allow them to return to the plane.

The catheter in FIG. 80 is an example of an endovascular carotid septum ablation catheter comprising first and second diverging arms, the first arm comprising an ablation element and configured so that the ablation element is in contact with a carotid septal wall in one of an external carotid artery and an internal carotid artery when the catheter is coupled with a common carotid artery bifurcation, the second arm configured to be disposed in the other of the internal carotid artery and external carotid artery when the catheter is coupled with the bifurcation, wherein the first arm includes a clearance portion configured to substantially avoid contact with the wall in the one of the external carotid artery and internal carotid artery when the catheter is coupled with a common carotid artery bifurcation such that substantially all contact that occurs between the first arm and the wall of the one of the internal carotid artery or the external carotid artery is made by the ablation element.

The catheter in FIG. 80 is an example of an endovascular carotid septum ablation catheter comprising first and second diverging arms, the first arm comprising an ablation element and configured so that the ablation element is in contact with a carotid septal wall in one of an external carotid artery and an internal carotid artery when the catheter is coupled with a common carotid artery bifurcation, the second arm configured to be disposed in the other of the internal carotid artery and external carotid artery when the catheter is coupled with the bifurcation, the first arm comprising a distal region distal to the ablation element that extends away from a longitudinal axis of the catheter relative to the ablation element.

The catheter in FIG. 80 is an example of an endovascular carotid septum ablation catheter comprising first and second diverging arms, the first arm comprising an ablation element and configured so that the ablation element is in contact with a carotid septal wall in one of an external carotid artery and an internal carotid artery when the catheter is coupled with a common carotid artery bifurcation, the second arm configured to be disposed in the other of the internal carotid artery and external carotid artery when the catheter is coupled with the bifurcation, the first arm comprising a distal region distal to the ablation element that is more flexible than a diverging arm region proximal to the ablation element.

The catheter in FIG. 80 is an example of an endovascular carotid septum ablation catheter comprising first and second diverging arms, the first arm comprising no more than a first ablation element and configured so that the ablation element is in contact with a carotid septal wall in one of an external carotid artery and an internal carotid artery when the catheter is coupled with a common carotid artery bifurcation, the second arm comprising no more than a second ablation element and configured to be disposed in the other of the internal carotid artery and external carotid artery when the catheter is coupled with the bifurcation.

The catheter in FIG. 80 is an example of an endovascular carotid septum ablation catheter comprising first and second diverging arms with free distal ends, the arms extending generally distally from the catheter; the first arm comprising a first ablation element, the second arm comprising a second ablation element, and wherein the first and second arms have unstressed configurations in which the first and second ablation elements are less than about 6 mm apart, such as less than about 4 mm apart, and such as less than about 2 mm apart, measured along a line perpendicular to a catheter axis.

The catheter in FIG. 80 is an example of an endovascular carotid septum ablation catheter comprising first and second diverging arms with free distal ends, the arms extending generally distally from the catheter; the first arm comprising a first ablation element, the second arm comprising a second ablation element, wherein the first and second ablation elements are substantially parallel when the arms are in unstressed configurations.

The catheter in FIG. 80 can be modified to be an example of an endovascular carotid septum ablation catheter comprising first and second diverging arms with free distal ends, the arms extending generally distally from the catheter; the first arm comprising a first ablation element, the second arm comprising a second ablation element, at least one of the first and second ablation elements having a distal end angled towards a catheter axis when the first and second arms are unstressed configurations, such as between about 10 and about 30 degrees relative to the catheter axis.

The catheter in FIG. 80 is an example of an endovascular carotid septum ablation catheter comprising first and second diverging arms with free distal ends, the arms extending generally distally from the catheter; the first arm comprising a first ablation element, the second arm comprising a second ablation element, the first and second arms comprising a monolithic structural member.

The catheter in FIG. 80 is an example of an endovascular carotid septum ablation catheter comprising: first and second diverging arms with free distal ends, the arms extending generally distally from the catheter and being in a first plane in unstressed configurations, at least one of the first and second arms comprising an ablation element, wherein the catheter is configured for controllable deflection in the plane.

The catheter in FIG. 80 is an example of an endovascular carotid septum ablation catheter comprising: first and second diverging arms with free distal ends, the arms extending generally distally from the catheter; the first arm comprising a first ablation element, the second arm comprising a second ablation element; and a coating layer, such as an electric insulator, around at least a portion of one of the first and second arms.

The catheter in FIG. 80 is an example of an endovascular carotid body ablation catheter, comprising a structural member comprising a first arm and a second arm, the first arm configured to engage with a wall of the internal carotid artery and the second arm configured to be engaged with a wall of the external carotid artery, a first ablation electrode mounted on the first aim in an electrode-mounting region, and a second ablation electrode mounted on the second arm in a second electrode-mounting region, the first arm, in a region proximal to the electrode-mounting region, has a configuration that extends away from the axis of the structural member and extends toward the axis of the structural member, and the second arm, in a region proximal to the electrode-mounting region, has a configuration that extends away from the axis of the structural member and extends toward the axis of the structural member.

The arm lengths of the catheter in FIG. 80 can be modified such that the catheter is an endovascular carotid septum ablation catheter comprising first and second diverging arms with free distal ends, the arms extending generally distally from the catheters, at least one of the first and second arms comprising an ablation element, wherein a length of the first arm measured along a catheter axis is different than a length of the second arm measured along a catheter axis.

The ablation element(s) on the catheter in FIG. 80 can be modified as described herein such that the catheter is an endovascular carotid septum ablation catheter comprising first and second diverging arms with free distal ends, the arms extending generally distally from the catheters, the first arm comprising at least one energy delivery region, the second arm comprising at least one second energy delivery energy region, wherein that at least one energy delivery region has a tissue contact surface area greater than a tissue contact surface area of the at least one second delivery region.

The arms of the ablation element(s) on the catheter in FIG. 80 can be modified as described herein so that the catheter is an endovascular carotid septum ablation catheter comprising first and second diverging arms with free distal ends, the arms extending generally distally from the catheters, the first arm comprising an ablation element, the first aim comprising a flex circuit including the first ablation element. The second arm can comprise a flex circuit including a second ablation element.

The arms in the catheter in FIG. 80 can be modified as described herein so that the catheter is an endovascular carotid septum ablation catheter comprising first and second diverging arms with free distal ends, the arms extending generally distally from the catheters, at least one of the first and second arms comprising an ablation element, wherein at least one of the first and second arms comprises a guidewire lumen. Both of the arms can also comprise a guidewire lumen.

The catheter in FIG. 80 can be modified as described herein to be an endovascular carotid septum ablation catheter comprising first and second diverging arms with free distal ends, the arms extending generally distally from the catheters, at least one of the first and second arms comprising an ablation element, wherein the first and second arms are secured together distal to a distal end of a catheter shaft.

The catheter in FIG. 80 can be modified as described herein so that it is an endovascular carotid septum ablation catheter comprising first and second diverging arms with free distal ends, the arms extending generally distally from the catheters, at least one of the first and second arms comprising an ablation element, wherein at least one of the arms comprises a pressure or force sensor thereon.

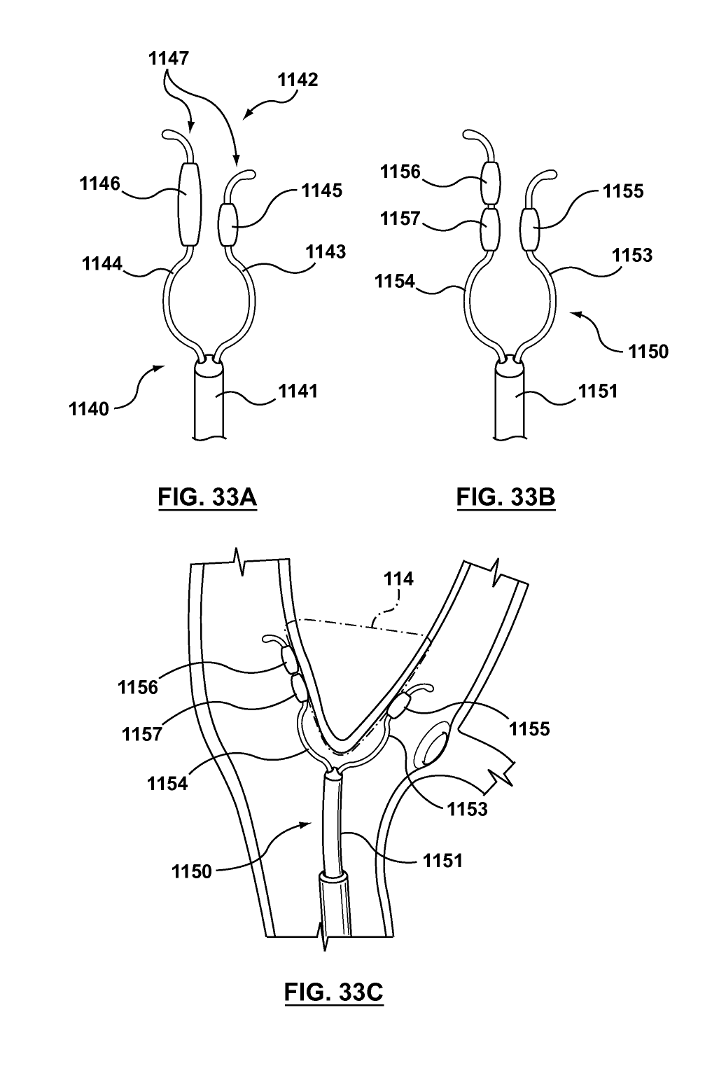

In any of the embodiments herein in which an ablation electrode is configured to be positioned in an external carotid artery to facilitate the ablation method, one or more electrodes can be configured to be positioned within the internal carotid artery. Placement of electrodes in an internal carotid artery can present a risk that if a thrombosis forms on the internal carotid artery wall from the ablation and the thrombus is released from the vessel wall to the blood stream, it creates a risk of brain embolism. FIGS. 33A to 33C illustrate devices and methods configured to reduce a risk of a thrombosis formation in an internal carotid artery wall. The one or more electrodes configured to be positioned in the internal carotid artery can have a size or surface area that is greater than the size of the electrode positioned in the external carotid artery. The increased size or surface area reduces the current density localized around the electrode in internal carotid artery tissue. This can also be referred to herein as dispersing current. Localized current density around an electrode is reversely proportional to the electrode's size. The same RF current delivered from two electrodes will produce a greater localized current density in tissue around the smaller of the two electrodes. By increasing the size or surface area of the internal carotid artery electrode(s), the localized current density applied to the internal carotid artery vessel wall can be reduced while still delivering enough RF energy and current density in septal tissue to create an appropriate ablation in a carotid septum.

FIG. 33A illustrates an exemplary catheter with first and second diverging arms in which a first electrode 1146 has a different length, measured along catheter axis, than the length of a second electrode 1145. First electrode 1146 has a greater surface area than second electrode 1145, and is adapted to disperse current more than first electrode 1145, reducing the current density in tissue adjacent electrode 1146. Catheter 1140 includes first and second arms 1143 and 1144, wherein the length of the electrode mounting region of arm 1144 is greater than the length of the electrode mounting region of arm 1143.

In some embodiments the length of electrode 1146 is about 1.25 to about 2.5 times the length of electrode 1145, although it may be any length greater. In some embodiments it is about 1.5 to about 2 times longer. In this embodiment electrodes 1145 and 1146 have the same or similar diameters, but they need not have. The two electrodes also both have a barrel configuration as described herein, but the electrodes can have any other suitable configuration and any other type of attachment with the arms (e.g., they can be flex circuits). Any other aspect of the catheters herein can be incorporated into this embodiment. For example, any arm configuration can be used for either of arms 1143 and 1144.

FIG. 33B illustrates a distal region of an alternative ablation catheter including first and second diverging arms wherein one arm has more electrodes disposed on it than the other arm, and the total size and surface area of the plurality of electrodes is greater than the size and surface area of the electrode on the other arm. First arm 1154 of catheter 1150 has electrodes 1156 and 11157 disposed thereon that electrically connected, while arm 1153 has electrode 1155 disposed thereon. Electrodes 1157 and 1156 can have the same size or they can be different sizes, and they can be the same size as electrode 1155 or not. Electrodes 1157 and 1156 can have the same general configuration as one another or not. Electrodes 1157 and 1156 can have the same general configuration as electrode 1155 or not. In some embodiments total length of electrodes 1157 and 1156 measured along their lengths is between about 1.25 and about 2.5 the length of electrode 1155. In some embodiments the total length is between about 1.5 and about 2 times longer.

In some embodiments electrodes 1157 and 1156 are between about 0.005″ and 0.060″ apart. A small gap may exist between the two electrodes, which can allow them to flex relative to one another. The relative flexing may facilitate passage through a tortuous sheath, such as around tight bends.

FIG. 33C illustrates catheter 1150 disposed near the carotid artery bifurcation, with electrode 1155 engaging the septal wall in the external carotid artery, and with electrodes 1144 and 1146 in contact with the septal wall in the internal carotid artery. As energy passes from electrode 1145 to electrodes 1144 and 1146, the current density is reduced, thus reducing the risk of thrombosis formation in the wall of internal carotid artery.

In alternative embodiments electrodes differ in a dimension other than length to provide them with different surface areas and hence different abilities to disperse current. For example, one electrode on one arm can have the same length as a second electrode on a second arm, but can have a configuration that gives it greater surface area. For example, one electrode could have a general cylinder shape, while one has a barrel shape, perhaps with a greater central width than embodiments herein. The barrel shaped electrode would have a greater surface area, and thus would be configured to reduce current density more than the generally cylindrically shaped electrode. In another example, one electrode could have an increased surface area by being an expandable electrode mounted to an inflatable balloon. The inflatable balloon may be positioned in an internal carotid artery and occlude blood flow. The expandable electrode may be a metallic foil or flex circuit mounted to the balloon. The second electrode positioned in an external carotid artery may be a barrel electrode such as 1155 having a surface area less than the first electrode. Any aspect of the electrode(s) can be varied to impart the desired dispersion properties. Additionally, any arm described herein can be incorporated into dispersive electrode designs.

### Over a Guide Wire Designs

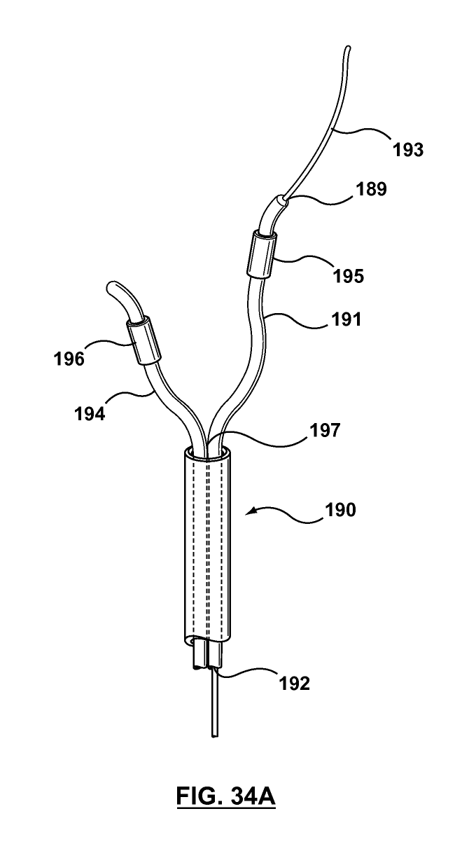

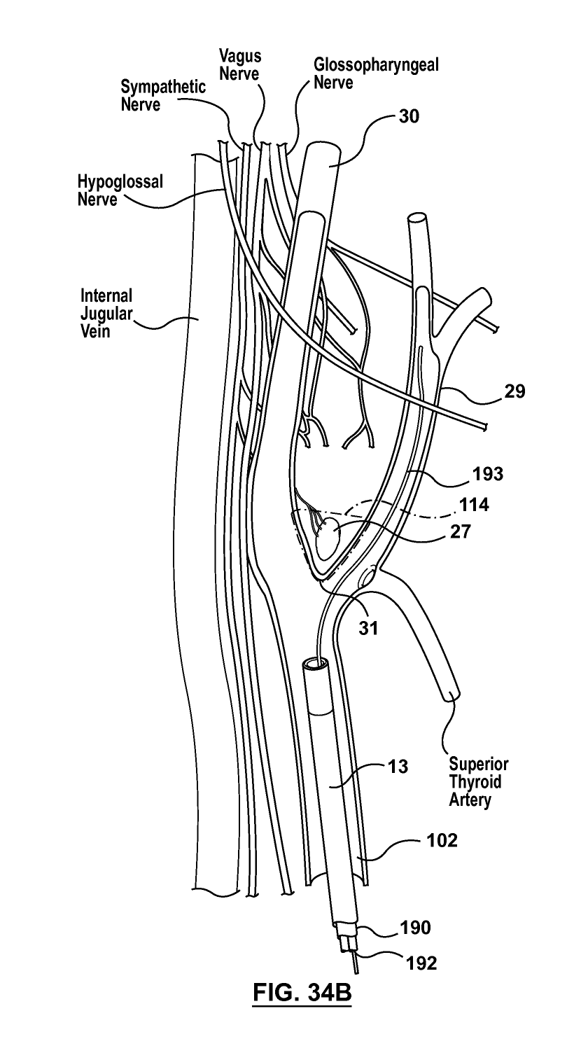

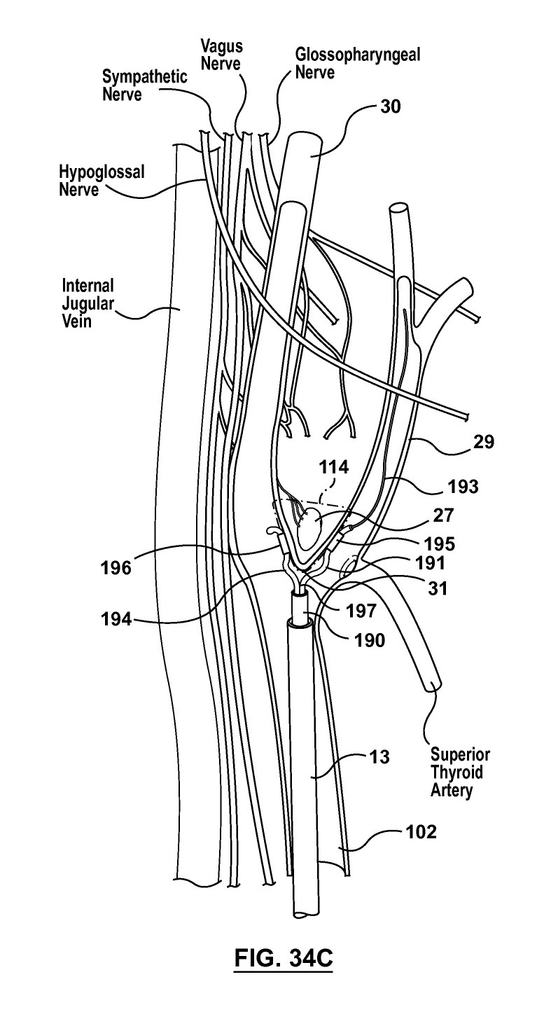

Other embodiments of ETAP catheters that may be delivered over a guide wire may comprise guide wire lumens that pass through one or both arms of a catheter. For example, as shown in FIG. 34A an arm 191 of an ETAP catheter 190 may comprise a guide wire lumen 192 with an exit port 189 at a distal end of the arm 191. As shown in FIG. 34B a guide wire 192 may be delivered to an external carotid artery 29. Then the ETAP catheter 190 may be delivered in an undeployed state within a delivery sheath 13 to a common carotid artery 102 in a vicinity of a carotid bifurcation 31, over the guide wire 192, which is passed through the lumen 192. The delivery sheath 13 may be retracted or the ETAP catheter 190 may be advanced out of the delivery sheath exposing arms 191 and 194. As shown in FIG. 34C ETAP catheter 190 is advanced over the guide wire 192 and arm 191 follows the guide wire 192 into the external carotid artery 29. Fine torquing of the ETAP catheter with the arms in the common carotid artery, and preferably with minimal contact to artery walls, can align the second arm 194 with an internal carotid artery 30. The arms 191 and 194 may be advanced over the carotid bifurcation 31 and intercarotid septum until ablation elements 195 and 196 (e.g., radiofrequency electrodes or electroporation electrodes) are placed at a target ablation site suitable for carotid body ablation. Alternatively electrode 195 may be an ablation electrode configured for monopolar radiofrequency ablation and electrode 196 may be absent or may be used for measuring electrical characteristics across an intercarotid septum (e.g., electric impedance). Measurement of impedance across a septum may enable fine resolution of the impedance signal change and monitoring of tissue properties. Components of impedance such as phase shift and resistance can be measured separately. Subtle changes in these signals can assist guiding an ablation process by the operator or software embedded in the RF generator. For example the arms may be advanced until a junction 197 of the arms contacts the carotid bifurcation 31, wherein length of the arms is appropriate for placing the ablation elements at a desired position on the intercarotid septum suitable for carotid body ablation (as shown in FIG. 5). FIG. 34 shows an endovascular carotid septum ablation catheter comprising first and second diverging arms with free distal ends, the arms extending generally distally from the catheters, at least one of the first and second arms comprising an ablation element, wherein at least one of the first and second arms comprises a guidewire lumen. Both of the arms can also comprise a guidewire lumen.

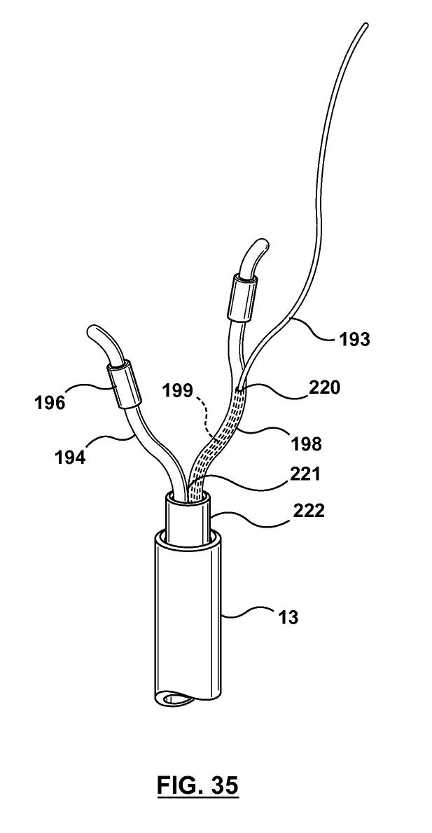

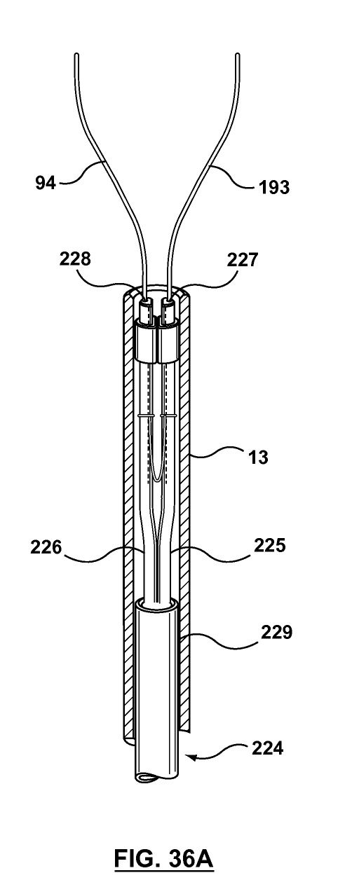

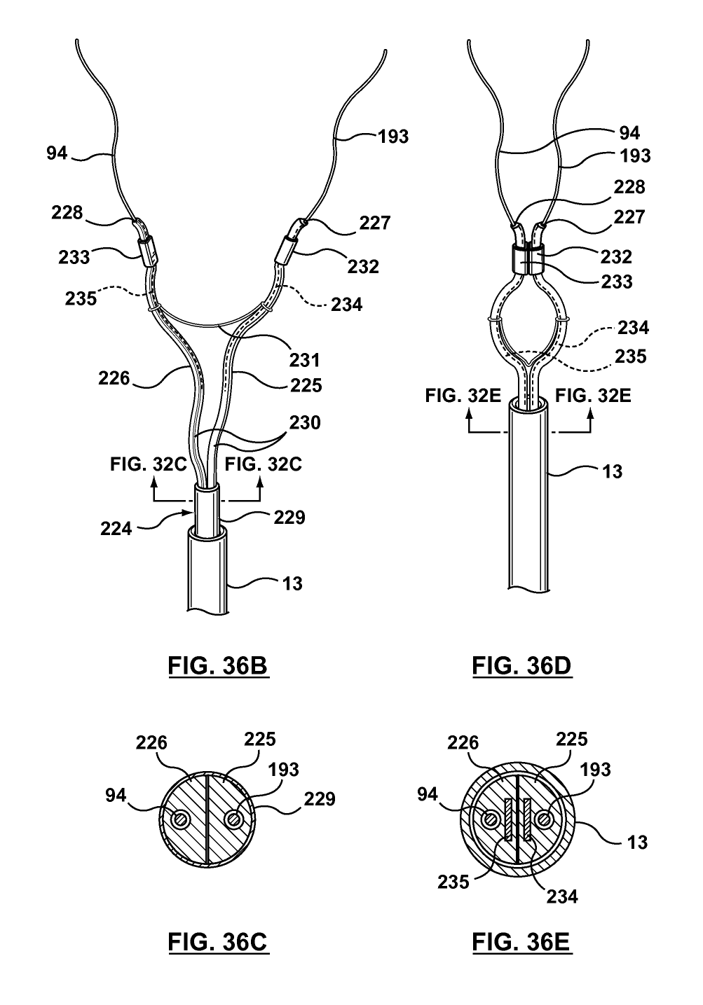

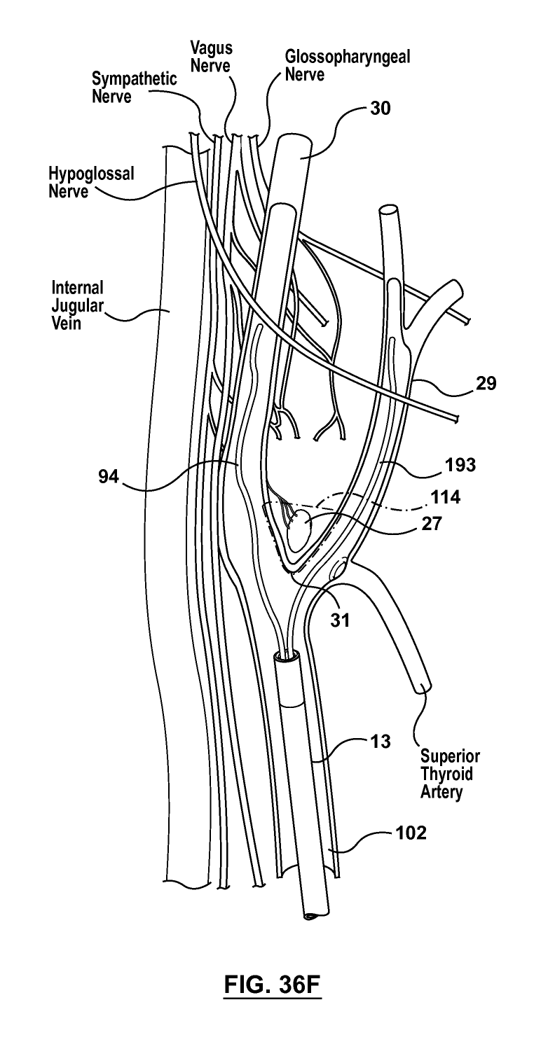

FIG. 35 is an alternative embodiment of an ETAP catheter 222 configured to be delivered over a guide wire. Arm 198 comprises a guide wire lumen 199 with an exit port 220 proximal to the distal end of the arm 198 and distal to arms junction 221. A method of using ETAP catheter 222 may be similar to the method described above for the embodiment shown in FIG. 34A. A groove in the distal part of the arm 198 (not shown) can be made to facilitate the exit of the wire 193 from the lumen (e.g., catheter monorail design) in order to further facilitate positioning of the system in the correct apposition to the desired walls of the septum.

An ETAP catheter may be configured for use with two guide wires, in which a first guide wire may be placed in an external carotid artery 29 and a second guide wire is placed in an internal carotid artery 30. Two guide wires may facilitate positioning a distal region of an ETAP catheter at a carotid bifurcation by minimizing or reducing a need to manipulate the catheter thus reducing a risk of trauma to vessels or dislodging of plaque. An example of a two-guide wire ETAP catheter is shown in FIGS. 36A, 36B, 36C, 36D, 36E, 36F, 36G, and 36H. FIG. 36A shows the two-guide wire ETAP catheter 224 contained within a delivery sheath 13 in an undeployed delivery state. The ETAP catheter 224 comprises two arms 225 and 226, each having a guide wire lumen with an exit port at the distal ends. Each arm may be made from a polymer tube (e.g., Pebax, PEEK) extending approximately the length of the catheter 224. The arms may be of different length. The arms may be held together in a shaft tube 229, which may have a lubricious or hydrophilic coating to facilitate motion within a delivery sheath 13. FIGS. 36B and 36C show the ETAP catheter 224 with the delivery sheath 13 retracted to expose a distal region of the catheter. Arms 225 and 226 each may comprise a proximal floppy section 230 (e.g., with a length of about 10 to 40 mm), and a distal resilient section (e.g., with a length of about 10 to 40 mm) as shown comprising resilient structural wires 234 and 235 such as Nitinol wire with a preformed shape such as the shape shown in FIG. 15. The structural wires may have a flat, rectangular, ribbon, or elliptical cross sectional profile, which may control bending in a preferential manner, that is, preferential bending in a plane that allows the arms to open and close. Arms 225 and 226 are tethered together with tether 231. The purpose of tether 231 is to limit distance between electrodes 232 and 233 (e.g., about 15 to 40 mm) so when advanced over a carotid bifurcation the electrodes are positioned appropriately on an intercarotid septum for carotid body ablation. The tether can also be a thin septum made of polymer like duck foot webbing. Tether 231 may be made from a thin, floppy, strong material such as Kevlar. FIGS. 36D and 36E show the catheter 224 with delivery sheath 13 advanced distal to floppy section 230 and over part of the resilient section, which creates a gentle closing force of the arms. Arms 225 and 226 may have a cross sectional profile such as an oval or half-circle as shown in FIGS. 36C and 36E, which may facilitate alignment of the arms with one another as the sheath is advanced over them. FIG. 36F shows the catheter 224 in use in a patient's carotid arteries in a delivery state contained within delivery sheath 13. Guide wires 193 and 94 are delivered into the patient's external 29 and internal 30 carotid arteries. The catheter 224, contained within delivery sheath 13, is delivered over the guide wires into common carotid artery 102 in vicinity of carotid bifurcation 31. Next, the distal region of the catheter is advanced from the delivery sheath, or the delivery sheath is retracted to expose the distal region. Floppy section 230 provides sufficient flexibility of arms 225 and 226 to follow the guide wires with minimal restriction. As shown in FIG. 36G as the catheter 224 is advanced over guide wires 193 and 94, arms 225 and 226 follow the guide wires with little or no contact or contact force against vessel walls of the carotid arteries or carotid bifurcation 31. The catheter 224 may be advanced until tether 231 contacts carotid bifurcation 31 which may be indicated to the user by tactile feedback or visualization (e.g., fluoroscopy). As shown in FIG. 36H the delivery sheath 13 may then be advanced over the floppy section 230 and a proximal portion of the resilient section causing arms 225 and 226 to close until electrodes 232 and 233 come into apposition with the vessel walls of the intercarotid septum. Depth markers or radiopaque markers on the sheath and catheter may provide indication of suitable alignment of the sheath and catheter to cause the arms to close. This embodiment may allow delivery of arms into internal and external carotid arteries with minimal contact or contact force against vessel walls or plaque layers as well as appropriate orientation and placement of ablation element(s) for carotid body ablation. Ablation energy may be delivered while ablation elements are positioned at the target ablation site. Following ablation, energy may be ceased and the catheter 224 may be removed in an opposite fashion: by pulling the delivery sheath back to release the closing force of the arms, retracting the catheter 224 into the common carotid artery 102, retracting the catheter 224 into the delivery sheath 13, and removing the guide wires. The catheter in FIGS. 36A-H is an example of an endovascular carotid septum ablation catheter comprising first and second diverging arms with free distal ends, the arms extending generally distally from the catheters, at least one of the first and second arms comprising an ablation element, wherein the first and second arms are secured together distal to a distal end of a catheter shaft.

Over a Guide Wire with Open/Close Actuation

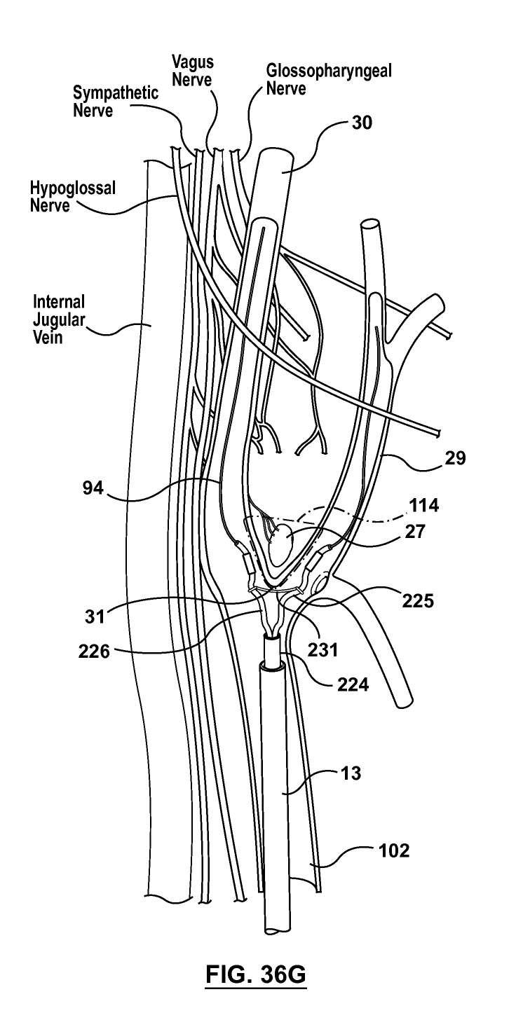

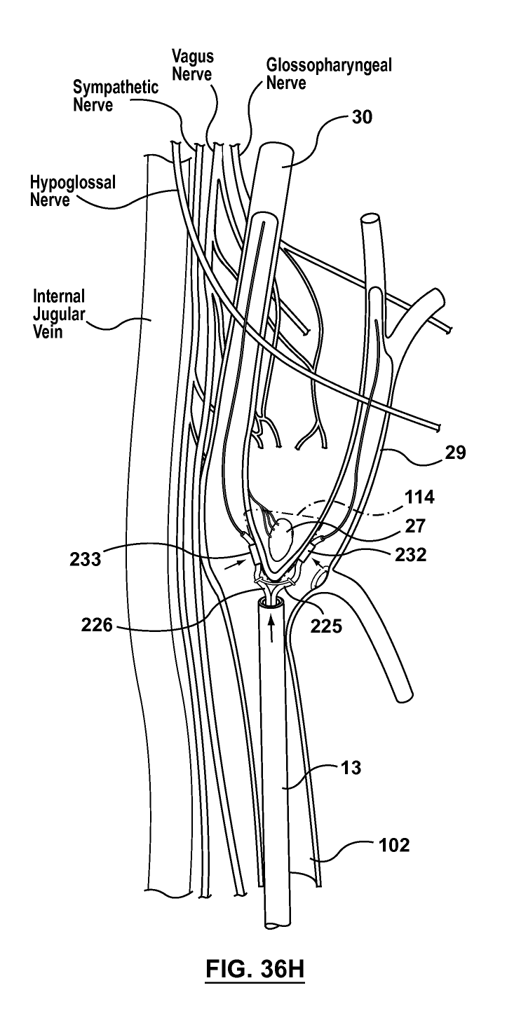

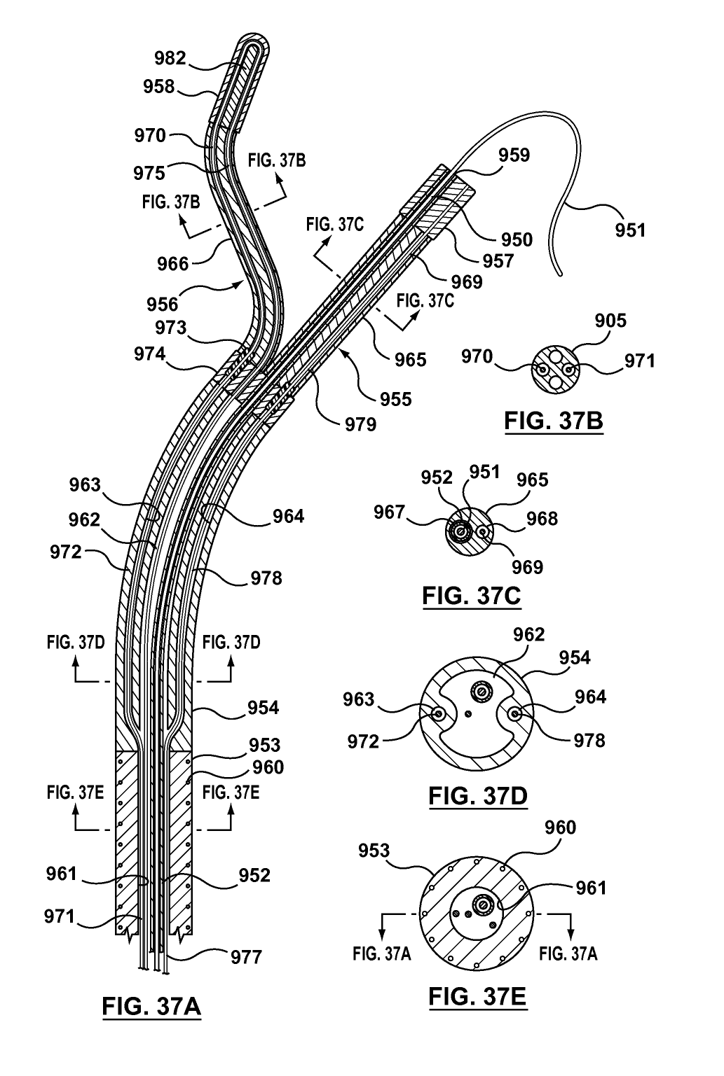

FIG. 37A shows an embodiment of an ETAP catheter configured to be delivered over a guide wire 951, to have bi-directional controllable deflection in a plane that is coplanar with open/close actuation of one spline or arm with respect to a second spline. The catheter is configured to place electrodes, mounted to each of the two arms, on an intercarotid septum in a location suitable for carotid body ablation (as shown in FIGS. 5A and 5B). The catheter comprises a guide wire lumen 950, which may be formed with a tube such as a polyimide tube 952 with an inner diameter of about 0.018″ and wall thickness of about 0.004″ and a lubricious inner coating to facilitate sliding over a guide wire. The guide wire lumen 950 may pass from a port on a proximal region of the catheter (not shown) through an elongated section 953 and a controllably deflectable section 954 of a catheter shaft, through a first arm 955, and finally through a first electrode 957 to a distal guide wire port 959 on a distal end of the first electrode 957. The guide wire may be, for example between 200 and 250 cm long and have a diameter of about 0.014″. The guide wire may be first delivered through a patient's vasculature from a femoral artery to an external carotid artery, and then facilitate delivery of the ETAP catheter through the vasculature to the patient's carotid artery, where the first diverging arm 955 may be advanced into the patient's external carotid artery.

The shaft comprises an elongated section 953 and a controllably deflectable section 954. The elongated section 953 may be made from extruded Pebax with a durometer of about 63 D and a wire braid 960 to enhance transmission of torque and translation from a handle (not shown) on a proximal end of the catheter. The elongated section 953 comprises a coaxial lumen 961 (shown in FIG. 37E) and may be approximately 100 cm long and have a diameter of about 2 mm. The controllably deflectable section 954, positioned distal to the elongated section, may be approximately 1 to 5 cm long (e.g., about 2.54 cm long) with a diameter of about 2 mm and made from extruded Pebax with a durometer that is softer than the elongated section, (e.g., about 40 D). The controllably deflectable section 954 may comprise a coaxial lumen 962, a first off-axis lumen 964 and a second off-axis lumen 963 (shown in FIG. 37D). Distal to the controllably deflectable section 954 the catheter diverges into a first arm 955 and a second arm 956, the first arm comprising a guide wire lumen and the second arm configured for open/close actuation. The first and second arms comprise electrical insulation such as extruded tubes, for example made from soft Pebax (e.g., about 25 D) or silicone. The extruded tubes may have a length of about 5 to 10 mm (e.g., about 6 mm) and a diameter of about 0.8 mm. The first extruded tube 965 covering the first arm 955 (shown in FIG. 37C) comprises a lumen 967 for the polyimide tube 952 and a lumen 968 for a first Nitinol structural segment 969. The second extruded tube 966 covering the second arm 956 (shown in FIG. 37B) comprises lumens for a second Nitinol structural segment 970 and an actuation segment 971 and optionally other lumens for electrical conductors.

A first superelastic Nitinol wire 977 is used to function as a first deflection pull wire 978 and a first arm structural segment 979. The Nitinol wire 977 may have a diameter of approximately 0.006″ to 0.012″. As shown the Nitinol wire 977 is slidably positioned in the coaxial lumen 961 then passes in to the first off axis lumen 964 of the controllably deflectable section 954 where it acts as a first deflection pull wire 978. The first deflection pull wire 978 is anchored with a crimp 980 to a distal end piece 974 at the distal end of the controllably deflectable section. The distal end piece 974 may be made from a rigid radiopaque material (e.g., radiopaque thermoplastic) and functions as a radiopaque marker, an anchor for the first and second deflection pull wires 978 and 972, an anchor for the first and second arm structural segments, and provides a protected opening to the coaxial lumen 962. When tension is applied to the first deflection pull wire 978 the controllably deflectable section may bend toward the side containing the first off-axis lumen 964.

A second preformed superelastic Nitinol wire 971 is used to function as a second deflection pull wire 972, a second arm structural segment 970, and an arm actuation pull wire 975. The Nitinol wire 971 may have a diameter of approximately 0.006″ to 0.012″. As shown the Nitinol wire 971 is slidably positioned in the coaxial lumen 961 then passes in to the second off-axis lumen 963 of the controllably deflectable section 954 where it acts as a second deflection pull wire 972. The second deflection pull wire 972 is anchored with a crimp 973 to the distal end piece 974 at the distal end of the controllably deflectable section. When tension is applied to the second deflection pull wire 972 the controllably deflectable section may bend toward the side containing the second off-axis lumen 963. The second structural segment 970 may be made from the Nitinol wire 971 and may comprise a preformed shape as shown that elastically holds the second diverging arm 956 in an open configuration, for example such that the electrodes are approximately 10 to 20 mm apart, when unconstrained by a sheath and when tension in an open/close pull wire is released. The Nitinol wire 971 forms a 180-degree bend at the distal end of the arm where it is inserted in an electrode 958 and held in place by a friction fitted core 982. The Nitinol wire 971 returns along the arm as an actuation segment 975 and enters through a central opening in the distal end piece 974 to the coaxial lumen 962 where it passes along the length of the shaft to an actuator on a handle (not shown). When tension is applied to the actuation segment 975 the second arm 956 is moved toward a closed configuration, bringing electrodes 958 and 957 closer together. The arms 955 and 956 may be approximately the same length or may be offset so one is longer than the other. For example, a first arm 955 may be about 11 mm long while the second arm 956 is about 6 mm long. Electrical conductors (not shown) may pass from an electrical connector on a proximal region of the catheter, through the catheter shaft and diverging arms to the electrodes.

### Contrast Lumen

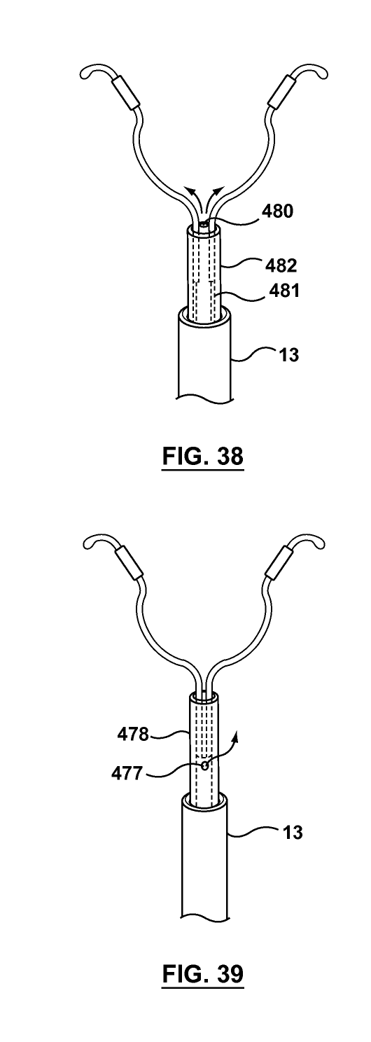

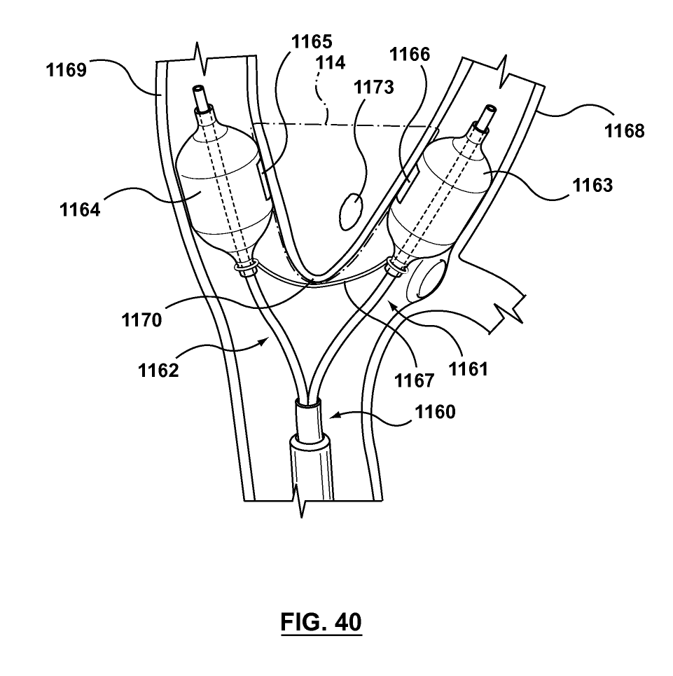

Any of the embodiments disclosed herein may further comprise an irrigation lumen 480 as shown in FIG. 38, which shows an ablation catheter with first and second diverging arms. The irrigation lumen 480 may be a lumen in a tube 481 extending approximately the length of catheter shaft 482 and may be positioned between arms or have an exit port within about 10 cm proximal from the arms. Irrigation with saline serves to improve electrode and vessel wall cooling and prevent damage to vessel walls, char formation, blood stagnation, and clot formation. The irrigation lumen may be used to deliver contrast agent to facilitate CTA or fluoroscopic visualization while positioning the catheter at a target ablation site. A lumen 480 such as that shown in FIG. 38 may also be used as a guide wire lumen. A user may deliver a guide wire to a common carotid artery then deliver the ETAP catheter over the guide wire. Alternatively, as shown in the ablation catheter with first and second diverging arms of FIG. 39, an irrigation lumen may be formed by a lumen in the catheter shaft 478 and may have an exit port 477 in catheter shaft 478 proximal to the arms. Alternatively, contrast agent may be injected through space between a delivery sheath and a catheter shaft.] Any of the arms described herein can be incorporated into such as design, as can any of the ablation elements described herein.

In some embodiments the ablation catheter may include one or more expandable or deployable structures that are configured to be positioned in the external or internal carotid artery and configured to, when in a deployed or expanded configuration, substantially stabilize the electrode with respect to the carotid artery wall and to urge or press the electrode into contact with the arterial wall. In some embodiments the deployable structures can be adapted to occlude the external or internal carotid arteries, and in some embodiments have diameters between about 4 mm and about 6 mm.

Some embodiments include a catheter configured to ablate a carotid body or its associated nerves, comprising a first diverging member comprising a first expandable structure and a first energy delivery element disposed on the first expandable structure, the first diverging member configured to be positioned in an external carotid artery; and a second diverging member comprising a second expandable structure and a second energy delivery element disposed on the second expandable structure, the second diverging member configured to be positioned in an internal carotid artery, wherein at least one of the first and second energy delivery elements is an ablation element configured to delivery ablation energy to tissue disposed between the first and second expandable structures. The first and second energy delivery elements can be disposed about the expandable structures such that they are oriented towards each other when the expandable structures are in expanded configurations, such as facing the center of the other vessel +/−about 45 degrees, such as +/−25 degrees. At least of the first and second expandable structures can be an inflatable structure with the energy delivery element mounted thereon. The first and second energy delivery elements can be RF ablation energy delivery elements configured to operate in bipolar mode to delivery RF energy to tissue disposed between the first and second ablation energy delivery elements. The catheter can further comprise a stabilizing element extending between the first and second diverging members, and configured to engage carotid bifurcation tissue provide a determination of the position of the first and second expandable structures.

FIG. 40 illustrates an exemplary embodiment of a carotid septum ablation catheter including a first expandable structure 1163 configured to be expanded and stabilized in external carotid artery 1168 and second expandable structure 1164 configured to be expanded and stabilized in internal carotid artery 1169. Catheter 1160 also includes first and second elongate structures 1161 and 1162 configured to be advanced into external and internal carotid arteries, respectively. Catheter 1160 includes first ablation element 1166 disposed on first expandable structure 163 and second ablation element 1165 disposed on second expandable structure 1164 in positions on the expandable structures such that when the expandable structures are expanded to their expanded configurations as shown, the electrodes are facing towards one another and are positioned into contact with the respective carotid arterial walls. In this embodiment the expandable structures are inflatable balloons mounted on elongate structures, which can be considered arms as used herein. The inflatable balloons are in separate or joined fluid communication with a fluid delivery lumen through which an inflation fluid can be advanced. The expandable structures may have outer dimensions and internal pressures when expanded to occlude either one or both of the external and internal carotid arteries. In some embodiments one or both of the expandable structures can have an outer diameter of about 4 mm to about 6 mm. For example, the balloons can be made of non-elastic material and have substantially cylindrical configurations.

Catheter 1160 includes bifurcation stabilizer 1167, extending between the diverging elongate structures 1161 and 1162 of the catheter 1160. The stabilizer is configured so that as the catheter is advanced towards bifurcation 1170, stabilizer 1167 will engage with bifurcation 1170 such that electrodes 1165 and 1166 are positioned between about 4 mm and about 15 mm cranial to bifurcation 1170. The stabilizer limits how far the catheter may be advanced by coupling with a bifurcation and positioning the electrodes at an appropriate distance cranial from the bifurcation.

Any of the embodiments of the ablation catheters herein can include a bifurcation stabilizer, which can also be referred to herein as a bifurcation pad or cushion. Tether 231 in FIG. 36B is another example of a bifurcation pad or cushion. The bifurcation pad can both position one or more ablation elements at desired locations along the septum, and can also be configured to contact the common carotid artery bifurcation and distribute force on the bifurcation along the pad, reducing pressure on the bifurcation. In some embodiments the bifurcation pad can have a rounded dome configuration, while in other embodiments it is a deployable device such as a deployable mesh or balloon, etc. A bifurcation pad may reduce risk of injuring the bifurcation or dislodging plaque that may be deposit on the bifurcation, especially if the user pushes too hard. A bifurcation pad can allow the user to push firmly to be sure the catheter is coupling with a bifurcation without worrying that pushing will cause injury. A bifurcation pad can be incorporated into any other catheter herein, such as the catheter shown in FIG. 32A. In FIG. 32A, the pad can be coupled to arms in the clearance portion, for example.

In alternative embodiments the device does not include stabilizer 1167, and rather the length of elongate structures between the location at which they diverge to the electrodes is between about 4 to about 15 mm so when the diverging region of the elongate structures engage the bifurcation the arms are positioned in the carotids arteries, respectively, so that the electrodes are positioned about 4 mm to about 15 mm from the bifurcation.

In use, the balloons are inflated after the electrodes are in the proper position, either when the stabilizer engages the bifurcation or when the divergence engages the bifurcation. The balloons can be in communication with a cooling fluid such as saline or chilled saline to cool the electrodes allowing them to deliver ablative energy without over heating tissue in contact with the electrodes. A cooling medium, if used, may also be used to inflate the balloons to expand the balloons. The cooling fluid may flow into the balloon through a lumen in the catheter. Optionally, the cooling medium may exit the balloon through a separate lumen in the catheter or through small holes in the balloon into the blood stream. The electrodes mounted to a balloon can be part of a flex circuit or electrically conductive film bound to the balloon material.

While an embodiment with an inflatable balloon has been provided in FIG. 40, one or more of the deployable structures can be a wire cage, expandable mesh, or other expandable structure adapted to radially expand. In a collapsed state, the deployable structures along with electrodes may be retracted into a delivery sheath having an inner diameter, for example, of about 7F (or less than 11F).

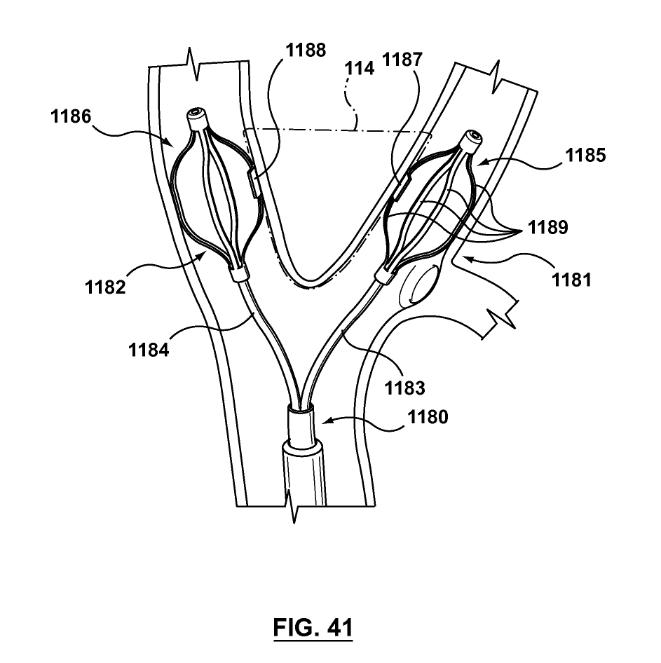

A deployable structure that allows blood flow through the structure or past the electrodes during energy delivery may be beneficial since the blood flow may help to cool the electrodes. In some embodiments one or more of the inflatable balloons can be configured as perfusion balloons to allow blood to flow past the balloon when they are inflated. FIG. 41 illustrates an alternative embodiment in which the expandable structure allows blood to flow in the carotid arteries during use. Catheter 1180 includes diverging structures 1181 and 1182, each of which include an arm 1183, 1184, and expandable structure 1185, 1186 in the form of an expandable cage. Each cage includes a plurality of splines 1189 (only labeled for cage 1185). In some embodiments splines 1189 may be made from an electrically non-conductive material, such as a polymer or insulated Nitinol. In some embodiments the splines are configured to be expanded upon user actuation so that they are only expanded after the electrodes have been properly positioned within the respective carotid arteries. For example, the splines can be coupled to central actuatable hub extending centrally within the splines such that, upon retraction of the hub, the splines deflect outward, thus expanding the cage. The expansion, like the balloon above, can both stabilize the electrodes in the arteries, as well as urge them into contact with the vessel wall.

Like the balloon embodiment above, an electrode is positioned on at least one spline in such as position that once the cages are expanded, the electrodes are facing one another, in the positions shown in FIGS. 5A and 5B.

While four splines are shown in this embodiment, more or fewer can be used. For example, three splines about 120 degrees apart can be used.

In alternative embodiments one expandable structure is an inflatable balloon, wherein the other expandable structure is not a balloon. For example, the second expandable structure could be an expandable cage like those shown in FIG. 41.

In alternative embodiments the catheter includes a first arm with an expandable structure and the second arm does not have an expandable structure. For example, a catheter could include a first arm with an inflatable balloon configured for expansion in the external carotid artery, and a second arm configured to apply a passive closing force form within the internal carotid artery. One use for such a catheter would be to avoid occluding the internal carotid artery during use, while there may be less concern for occluding the external carotid artery.

Any of the arm structures described herein can be a first arm of a catheter and any arm structure herein can be the second arm of the catheter. That is, any suitable combination of first and second arm structures can be combined into a single ablation catheter.

In some embodiments the first arm comprises first and second electrodes configured to be used in bipolar configuration when disposed in an external carotid artery to ablate septal tissue, wherein the catheter also supports a second arm configured to be positioned in an internal carotid artery. The second arm can be thought of as a keying element, that when deployed within the internal carotid artery, both positions the electrodes at desired axial locations within the external carotid artery as well as orients the electrodes towards the carotid septum so that the electrodes can effectively ablate septal tissue.

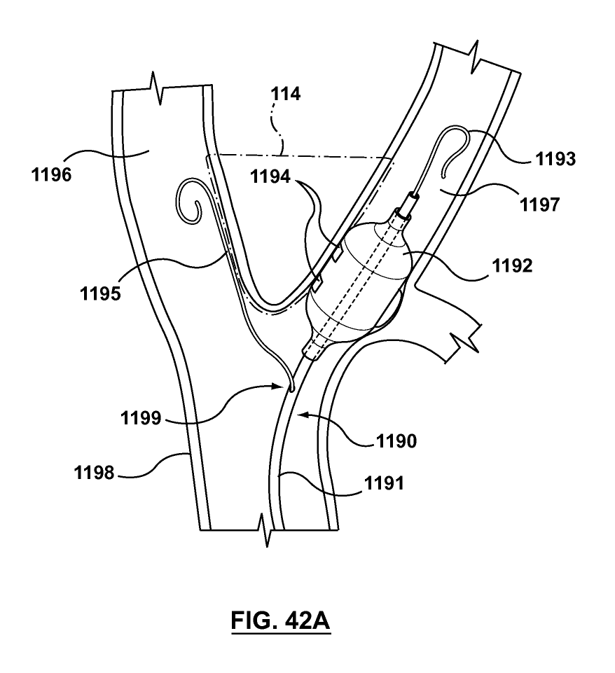

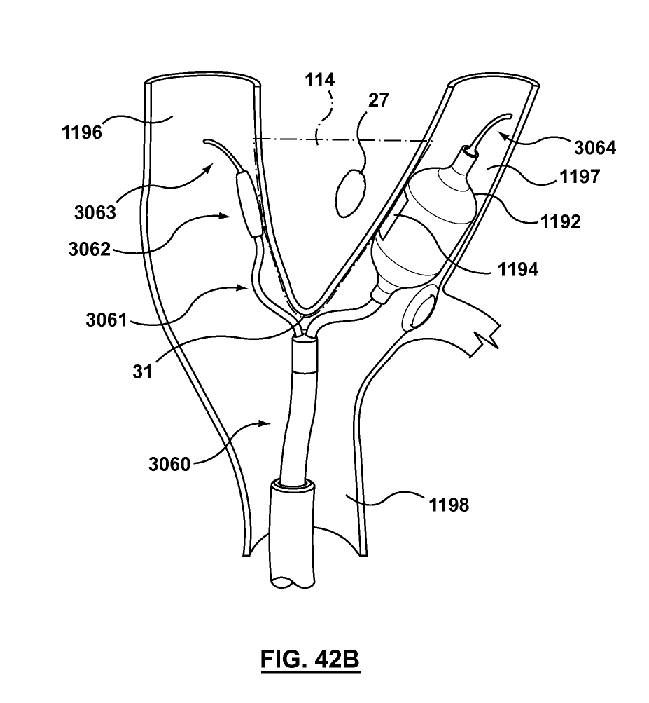

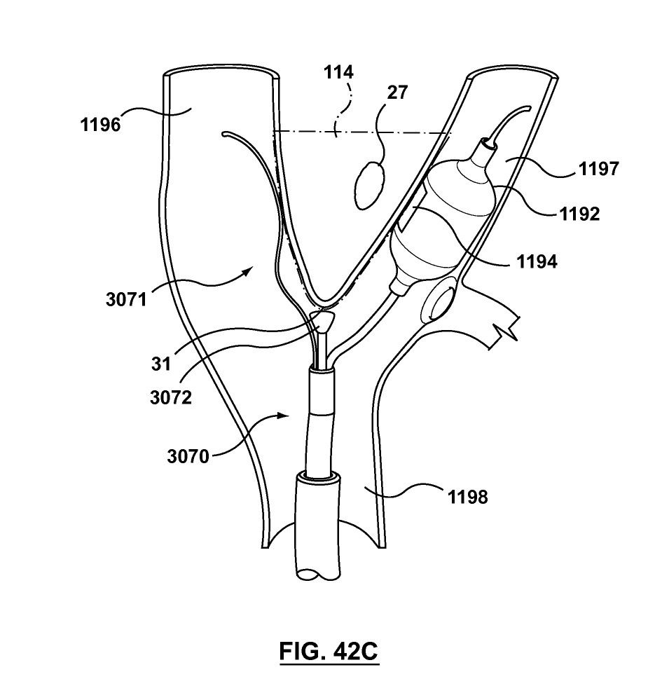

FIG. 42 illustrates an exemplary carotid septum ablation catheter in use that supports a keying element configured to be positioned in an internal carotid artery. Catheter 1190 includes shaft 1191 with port 1199 from which keying element 1195 extends radially from shaft 1191. Keying element 1195 is shown in internal carotid artery 1198. In some embodiments the keying element is a guidewire or guidewire like structure, deployable as described herein. Shaft 1191 also supports radially expandable device 1192, in this embodiment in the form of an inflatable balloon (but which can be any suitable expandable structure such as a caged structure), configured to be expanded and engage external carotid artery 1197. Balloon 1192 has electrodes 1194 deposited thereon, which are configured to be used in bipolar mode to ablate septal tissue. The bipolar electrodes may be independently connected to an energy delivery console via electrical conductors that run through the shaft of the catheter to an electrical connector at a proximal end of the catheter. The energy delivery console can deliver RF energy to the two electrodes in a bipolar configuration (i.e., so that RF current passes from one electrode through septal tissue to the other electrode).

Optionally, the balloon may comprise more than two electrodes and when the balloon is deployed a pair from the more than two electrodes that is aligned with a carotid septum may be chosen for energy delivery.

In this embodiment electrodes 1194 are mounted on a section of the balloon facing the keying element, or oriented in the same direction as the keying element. For example, the electrodes are mounted to be in substantial alignment with the port 1199 and/or keying element 1195 when deployed. The alignment of the keying element and electrodes may facilitate alignment of the bipolar electrodes with a carotid septum, ensuring effective ablation.

In some embodiments the bipolar electrodes are made from flex circuits or a thin conductive film. Electrodes may be, for example without limitation, between about 3 mm to 5 mm long, about 0.5 mm to about 4 mm wide, and separated by a linear distance of about 3 mm to about 5 mm. In particular embodiments the electrodes are about 4 mm long and about 2 mm wide and separated by a distance of about 4 mm.

In some embodiments the balloon is configured to occlude the external carotid artery. For example, it can be a compliant balloon with an inflated diameter of about 4 mm to about 6 mm. Occluding blood flow through the external carotid artery, at least immediately around the electrodes may force RF current to flow through the tissue of the carotid septum, thus creating a lesion in the septum, instead of taking a path of least resistance through blood, which may form a shallow lesion. The balloon may also help to press the electrodes in to contact with the septum wall.

The balloon, like other balloons described herein, may be cooled to pull heat from the electrodes and vessel wall, which may allow greater power to be delivered or which may cause a lesion to be formed deeper into the septum. The balloon may be cooled by circulating a cooling fluid such as saline or chilled saline. The cooling fluid may be delivered to the balloon through a port in the catheter shaft, which also may inflate the balloon. The cooling fluid may exit through an exit lumen in the shaft of the catheter or it may weep into the blood stream. Optionally, the cooling fluid may weep from perforations in the balloon.

FIG. 42B illustrates an alternative to the embodiment shown in FIG. 42A. FIG. 42B illustrates an ablation catheter comprising first and second diverging arms, wherein first and second ablation elements are in contact with carotid septal tissue in the internal and external carotid arteries between the common carotid artery bifurcation and about 15 mm away from the bifurcation. Inflatable bipolar RF balloon catheter 3060 is disposed on a first arm, wherein catheter 3060 also includes a second arm in the form of keying element 3061, which is configured to apply apposition force to the wall of a vessel (e.g., internal carotid artery, carotid septum), which may improve stabilization of the balloon 1192 and orient the electrode 1194 on the septum wall of the carotid artery (e.g., external carotid artery). Keying element 3061 may comprise a structural member that is similar in shape to an arm 490 shown in FIG. 15, an arm 720 shown in FIG. 17, or an arm 3000 shown in FIG. 32I and may comprise an outward bend or arch, a tissue contact region 3062, and a distal region 3063 having an outward bend, examples of which are described herein. The structural member may be, for example superelastic, round, shape-set Nitinol wire with diameter of about 0.012″. The structural arm may be coated with an electrically insulating coating that may be lubricious. Optionally, the keying element 3061 may comprise an ablation element such as a bipolar RF electrode as shown positioned on the tissue contact region 3062 of the arm. Alternatively, the keying element need not have an ablation element thereon. A distal region 3064 having an outward bend may also be positioned distal to balloon 1192 on the first arm. While advancing the structure into position, distal regions 3063 and 3064 may be positioned to aim a gap between the distal regions at a bifurcation by deflecting the shaft of the catheter 3060, as described herein in other embodiments. As the catheter is advanced the keying element 3061 may be advanced into an internal carotid artery 1196 and the first arm comprising balloon may be advanced into an external carotid artery 1197. The keying element and balloon arm may have a gap between them in an unconstrained or unstressed state that is between about 3 mm to 8 mm (e.g., about 4 mm). When the structure is advanced until the diverging arms and/or distal end of the catheter shaft couple with the carotid bifurcation the balloon may be inflated (for example with air, saline, chilled fluid), which may cause the electrode 1194 to make contact with the carotid septum wall of the external carotid artery and also cause the keying element to press into the carotid septum wall of the internal carotid artery.

FIG. 42C illustrates an alternative to FIG. 42B wherein the catheter includes first and second diverging arms. The second arm in this embodiment is not shown to include an ablation element thereon, and provides stabilization for an ablation element on the first arm (i.e., on the inflatable balloon). Catheter 3070 is similar to catheter 3060 shown in FIG. 42B, further illustrates an exemplary carotid bifurcation pad 3072. Pad 3072 may provide a soft cushion or increased area to distribute force and reduce pressure when pressing the structure into coupling position with a carotid bifurcation 31, which may reduce risk of injury to the bifurcation or reduce risk of dislodging plaque that could be on the bifurcation. The pad 3072 may be a deployable structure such as a fine wire mesh or balloon, and may be made from an electrically non-conducting material. Alternatively, the pad may be used as an ablation element such as an RF electrode that may be configured as a bipolar electrode along with electrode 1194. Aspects of catheter 3070 that are the same as those in FIG. 42B or can be replaced with other components described herein are not described.

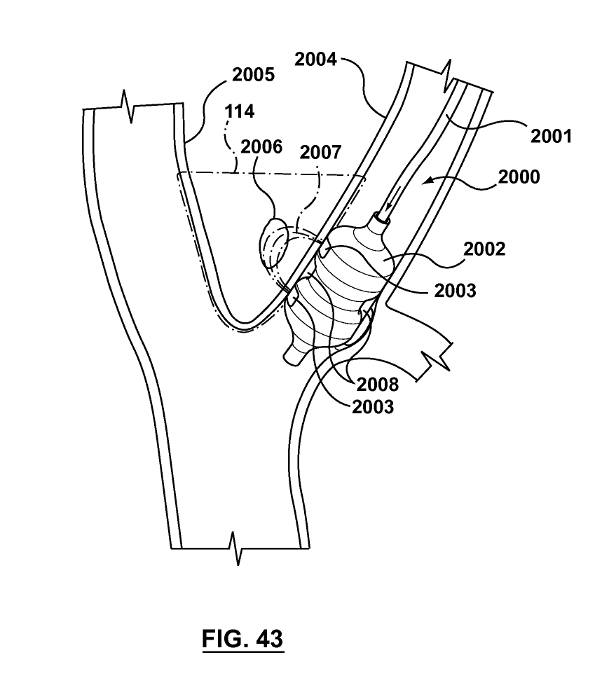

In alternative embodiments the balloon has a configuration that does not occlude the entire volume of the vessel between the distal and proximal ends of the balloon. For example, FIG. 43 illustrates a balloon with a general hourglass configuration when inflated. The general hourglass shape occludes the external carotid artery in two locations near the distal and proximal ends, leaving a volume of non-occluded vessel between the occlusions, as shown in FIG. 43. Chilled coolant such as saline may be circulated or injected into the non-occluded volume to cool tissue adjacent to the volume. The chilled coolant may be delivered to the volume through a lumen in the catheter shaft exiting a port in the shaft next to the volume.

In alternatives to the embodiment shown in FIG. 42, a bipolar RF balloon catheter does not include a keying element. That is, the catheter does not include a second arm or diverging structure positioned within the external carotid artery. Positioning of the electrodes on a septum in this or similar embodiments can be achieved by rotating the balloon under fluoroscopy. The electrodes can be radiopaque allowing their visualization, or radiopaque markers may be positioned on the balloon or shaft to help orient the electrodes in the direction of the septum. Similarly, a catheter without a keying structure can include any type of expandable structure such as a caged expandable structure wherein more than one electrode is positioned on a single spline.

FIG. 43 illustrates an alternative carotid septal ablation catheter adapted to be used in bipolar mode to ablate a carotid body or its associated nerves. As shown in FIG. 43 catheter 2000 is configured to be delivered to a target carotid septum from a retrograde approach. For example, the catheter may be delivered into a patient's vasculature through a superficial temporal artery and down to external carotid artery 2000 to the target carotid septum, which includes carotid body 2006.

Catheter 2000 includes shaft 2001 to which expandable structure 2002, in the general shape of an hourglass, is secured. Expandable structure 2002 is in this embodiment an inflatable balloon, on which electrodes 2003 are mounted and which are engaging external carotid artery tissue adjacent a carotid septum. Balloon can include any of the structure or function of any other balloon described herein (e.g., irrigation).

Catheter 2000 may also have radiopaque markers to facilitate orientation of electrodes 2003 with a carotid septum. The markers may be positioned on the catheter shaft. For example, any of the radiopaque markers and their use described herein can be incorporated onto shaft 2001 and its use. For example, the catheter can be rotated to align the markers with the plane of the bifurcation, which positions the electrodes toward the septum and in position to ablate.

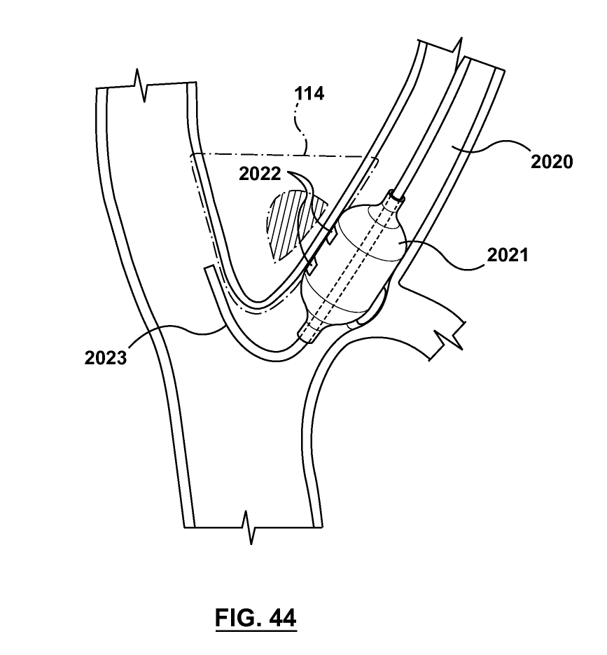

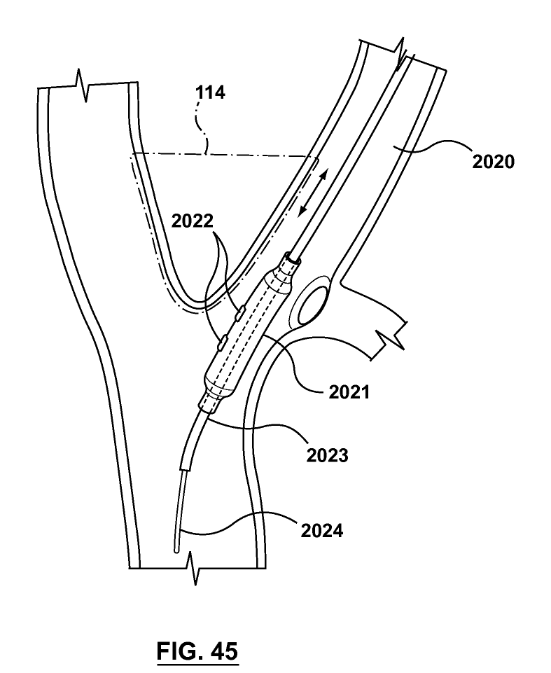

In the alternative embodiment shown in FIGS. 44 and 45, catheter 2020 includes keying element 2023. In this embodiment keying element 2023 comprises a hook at a distal end of the catheter, configured to couple with the carotid bifurcation. Electrodes on the balloon can be positioned on the side of the balloon facing the direction of the keying element. In the embodiment shown in FIGS. 44 and 45, catheter shaft comprises a preformed hook 2023 and a lumen along its axis. A stiff wire may be positioned in the lumen that straightens the hook. When the stiff wire is removed the preformed hook deploys and assumes its pre-formed configuration. In alternative embodiments the catheter includes a deflectable section at its distal end that forms a hook that acts as a keying element.

As set forth herein some catheters are adapted to be advanced to a common carotid artery through a sheath, following by sheath retraction to expose the catheter, and in some instances allowing it to deploy to a pre-formed configuration or shape. The catheter can then be aligned with and advanced over a carotid septum.

In some embodiments the distance between the distal end of the sheath and the distal end of the ablation catheter may be important, for example, to expose a deflectable section of the catheter, to expose the arms fully, and/or to expose enough shaft of a catheter to allow bipolar electrodes to self-align on a carotid septum (i.e., so the stiffness of the sheath doesn't impede the arms from naturally self-aligning). In some embodiments the catheter shaft includes a radiopaque marker and the sheath includes a second radiopaque marker. The markers are positioned on the respective devices such that axial alignment of the markers following sheath retraction indicates a reasonable desired pull back distance. For example, it may be desired to pull a sheath back between about 2 cm to about 5 cm, such as about 3 cm.

System have been conceived comprising a catheter having a means for coupling with a carotid bifurcation or intercarotid septum (e.g., forceps or keyed element) for transmural carotid body ablation and an ablation energy console. The system may additionally comprise a connector cable or several cables for connecting the ablation energy console with the catheter, a delivery sheath, or a guide wire. The console may comprise a user interface that provides the user with a means to select ablation parameters, activate and deactivate an ablation, or to monitor progress of an ablation. The console may have a second user interface that allows the user to select electrical stimulation or blockade used to investigate proximity of an ablation element on the catheter to neural structures. The console may comprise a computer algorithm that controls ablation energy delivery. The algorithm may control energy delivery (e.g., controlled power delivery) based on inputs for example, user selected variables, pre-programmed variables, physiologic signals (e.g., impedance, temperature), or sensor feedback.

### Keyed Bifurcation Coupling

Other devices have been conceived for endovascular transmural carotid body ablation with a distal region of a catheter that couples with a carotid bifurcation using a keyed bifurcation structure, herein referred to as Endovascular Transmural Ablation Keyed (ETAK) catheters. An ETAK catheter may comprise an ablation element on a distal region of the catheter and, proximal to the ablation element, a keyed bifurcation structure that diverges from a central axis of the catheter. A keyed bifurcation structure may comprise, for example a guide wire passed through a side-exiting guide wire port, multiple guide wires passed through multiple guide wire ports, or a deployable side arm. Alternatively, a keyed bifurcation structure may be coaxial with a central axis of an ETAK catheter and an ablation element may be on an arm that diverges from the central axis of the catheter. A user may advance the catheter, placing the keyed bifurcation structure in an internal carotid artery and the ablation element on the distal region of the catheter in an external carotid artery, until the keyed structure is coupled with a carotid bifurcation. The keyed bifurcation structure may diverge from the central axis of the catheter proximal to an ablation element at a distance that places the ablation element at a substantially suitable position on an intercarotid septum for effective carotid body ablation. For example the ablation element may be at or between about 4 mm to 15 mm from the divergence. This distance may be fixed or may be adjustable. Apposition of an ablation element with tissue may be achieved via resilient forces of a structural member in the catheter, deployment of an expandable structure, or deflection of a deflectable section of the catheter. An ablation element may be, for example, a radiofrequency electrode, bipolar radiofrequency electrodes, a cooled radiofrequency electrode, a cryogenic applicator, an ultrasound transducer, or a microwave antenna. ETAK catheter designs may facilitate positioning and orientation, improve apposition of electrodes and protect walls of carotid arteries from injury and plaque disturbance. Contrary to some other common ablation catheters, ETAK catheter design leaves walls of internal 30 and external 29 carotid arteries, opposite to a carotid bifurcation (known as the Y sides of the carotid arteries), practically free from mechanical forces that can dislodge plaque. It is known that plaque is often found on those walls where blood flow velocity is slower.

In some embodiments the ETAK catheter includes an ablation element disposed relative to a catheter shaft such that it is configured to be positioned in an external carotid artery; and a diverging structure that diverges from a central axis of the catheter, wherein the ablation element is about 4 mm to about 15 mm distally from the divergence of the diverging structure. The ablation element can be mounted about a catheter shaft, the shaft configured to be positioned in the external carotid artery. The catheter can include a plurality of ablation elements configured to be positioned in the external carotid artery, such as mounted about a catheter shaft, the catheter shaft configured to be positioned in an external carotid artery (e.g., annular or partially annular electrodes). The ablation element can be configured to be oriented in the direction of the bifurcation structure. The catheter can include an expandable structure, such as an inflatable device or other expanding device, to which the ablation element is secured. For example the ablation element can be mounted about the inflatable structure. An inflatable balloon can include more than one ablation elements, which can be first and second RF electrodes and configured to function in bipolar mode. The ablation element can be an RF electrode configured to be operated in monopolar mode. The catheter can comprise an exit port therein configured to allow the diverging structure to be advanced therethrough. The diverging structure can be configured to rotationally orient the ablation element towards a carotid septum when the diverging structure is positioned in an internal carotid artery. The expandable structure can be configured to be expanded and create apposition between the ablation element and a carotid septal wall. The diverging structure can be configured so that when the expandable structure is in an expanded configuration the ablation element is oriented in the direction of the diverging structure. The diverging structure can diverge at an angle between 0 and about 90 degrees relative to the axis of the catheter, such as between about 30 and 70 degrees. The diverging structure can have a free end.

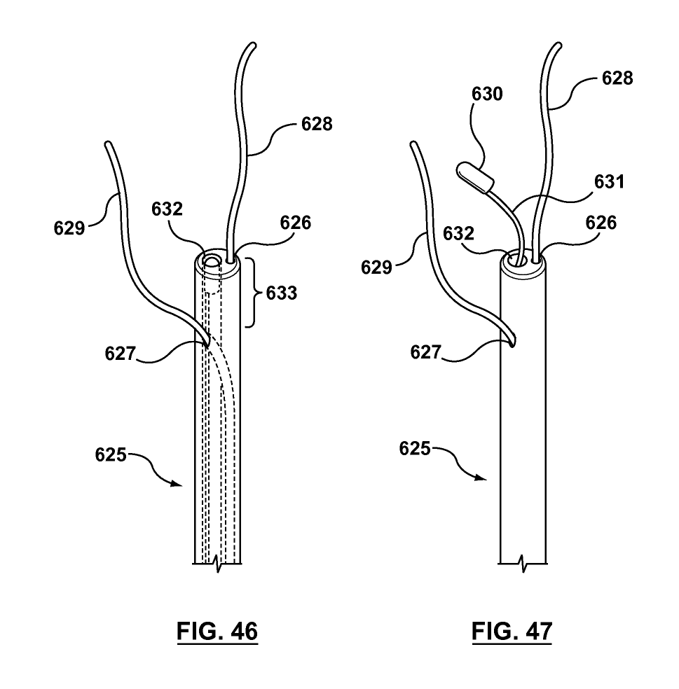

An embodiment shown in FIGS. 46, 47 and 48 comprises an elongate sheath 625 having a first lumen with a distal exit port 626 and a second lumen with a side-exiting port 627. FIG. 46 shows a first guide wire 628 passed through the first lumen and exiting distal exit port 626, and a second guide 629 wire passed through the second lumen exiting side-exit port 627. An ablation catheter may be positioned in a third lumen 632 such that an ablation element (e.g., radiofrequency electrode) is contained within the lumen. FIG. 47 shows an ablation element 630 advanced from the third lumen 632. The ablation element 630 is mounted to a resilient wire 631 (e.g., Nitinol) with a preformed curve, which is mounted to the ablation catheter. In this embodiment the ablation catheter may have a shaft that is rotationally aligned with side-exit port 627 and slidable within the lumen 632 of the catheter 625. For example, the shaft may have a non-circular cross sectional profile, such as a triangle, rectangle, square, or oval and lumen 632 may have a mating profile so that the shaft may slide within the lumen but may not rotate with respect to the lumen. In this manner, the resilient wire 631 mounted to the shaft may resiliently deflect in a predictable direction, such as toward the side-exiting port 627. Distal region 633 of the catheter 625 extends distal to the side exiting port 627 and may be at or between about 4 mm to 10 mm long. Depth markers or radiopaque markers on the ablation catheter and sheath 625 may align when the ablation element 630 extends a predetermined distance from the sheath 625 (e.g., at or between about 2 mm to 10 mm). The predetermined distance may be based on an imaging study of the patient's carotid body (e.g., CTA). FIG. 48 shows the device positioned in a patient's carotid arteries. A method of use may comprise advancing a first guide wire 628 through a patient's vasculature into an external carotid artery 29; advancing sheath 625 over the guide wire until it is in the patient's common carotid artery 102 or external carotid artery 29; advancing a second guide wire 629 through the sheath 625 and out of side-exiting port 627 and into the patient's internal carotid artery; adjusting the sheath 625 such that a bifurcation formed by the side-exiting guide wire 629 and the distal region of the sheath 633 is coupled with a carotid bifurcation formed by the diverging internal and external carotid arteries; advancing ablation element 630 from the sheath such that resilient wire 631 presses the ablation element into apposition with a target ablation site such as an inner wall of external carotid artery 29 (e.g., the ablation element 630 may be placed at or between about 4 mm to 15 mm from the side-exit port 627); delivering ablation energy (e.g., radiofrequency electrical current) from the ablation element 630 to the target ablation site for carotid body ablation; stopping delivery of ablation energy; retracting the ablation element into the catheter 625; retracting the guide wires; and removing the catheter from the patient. Alternatively, an ablation element may be mounted to a user-deflectable catheter that is deflected toward a predefined direction such as toward the side-exiting port 627 using mechanism such as pull wire or thermal electric Nitinol actuator.

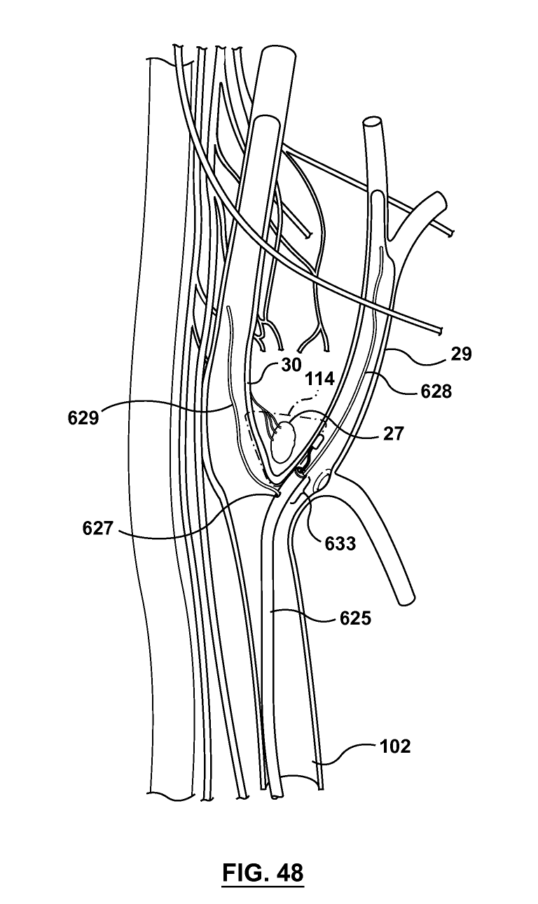

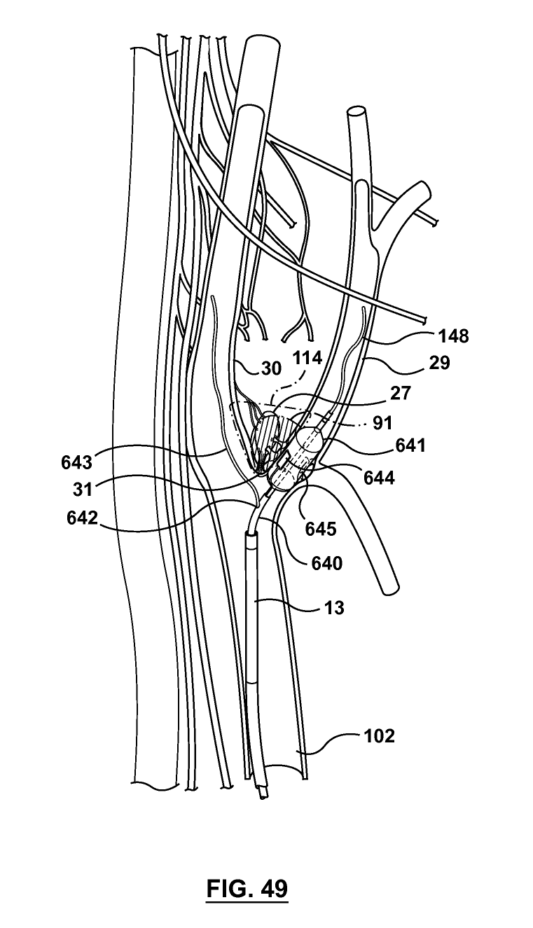

FIG. 49 shows an ETAK catheter 640 having an expandable structure, such as an inflatable balloon 641 with an ablation element 644 (e.g., radiofrequency electrode) mounted to one side of the balloon and a side-exiting guide wire port 642. The balloon 641 may be delivered into a patient's external carotid artery 29 through a delivery sheath or over a guide wire 148 placed in the external carotid artery as shown. Prior to inflating the balloon 641, a guide wire 643 may be passed through a separate lumen and out of side-exiting port 642. The catheter 640 may be torqued to direct the guide wire 643 toward the patient's internal carotid artery 30, and the guide wire may be advanced into the internal carotid artery. With the side-exiting guide wire 643 placed in the internal carotid artery 30 the balloon 641 is rotationally oriented. The catheter 640 may be advanced until the divergence of the side exiting guide wire 643 and balloon-carrying arm is coupled with carotid bifurcation 31. Alternatively, a user may decide to not to advance the catheter to complete bifurcation coupling but may advance the catheter a short distance before completing coupling (e.g., up to about 10 mm), for example if there is a high risk of dislodging plaque located at the bifurcation. The balloon 644 may be inflated with fluid such as saline and with appropriate rotational orientation imposed by the side-exiting guide wire and appropriate distance relative to the carotid bifurcation, the ablation element 644 may be placed at a suitable location for carotid body ablation (e.g., inner wall of the external carotid artery facing a carotid body approximately 4 to 15 mm superior to the carotid bifurcation) and inflation of the balloon may provide suitable apposition (e.g., contact force, contact surface area, contact stability during energy delivery) between the ablation element and tissue. Furthermore, the balloon may require little to no positional manipulation during inflation or once inflated. The ablation element may contain a radiopaque material (e.g., platinum iridium, gold, stainless steel) and the balloon may optionally comprise a second radiopaque marker 645 on an opposite side of the balloon that is visually distinct from the ablation element. Two radiopaque markers may facilitate confirmation of suitable rotational alignment of the balloon in the external carotid artery.

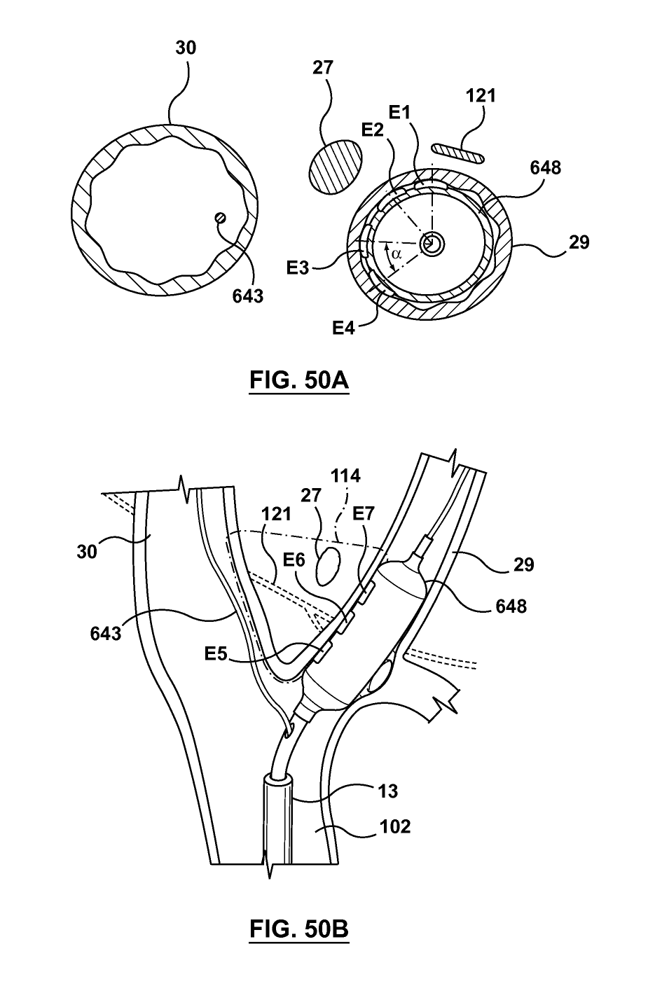

Alternatively, an ETAK catheter may have an expandable structure such as balloon with multiple ablation elements mounted to the balloon. The multiple ablation elements may be rotationally oriented, as described before, by placing a side exiting guide wire in an internal carotid artery. More than one ablation element may be used to deliver ablation energy to create a larger ablation that only one element. Or, a user may choose which ablation element to activate based on a location of a target ablation site. FIG. 50A is a transverse cross sectional view of a patient's internal 30 and external 29 carotid arteries with a multi-electrode ETAK balloon 648 placed in the external carotid artery 29 and oriented by placing a side-exiting guide wire 643 in the internal carotid artery. The balloon 648 comprises multiple ablation electrodes E1, E2, E3, and E4 spaced apart around the diameter of the balloon, for example the electrodes may be spaced apart at an angle α of or between about 20 to 45 degrees. A user may choose which electrode to activate based on an imaging study that determines a location of a patient's carotid body relative to the internal and external carotid arteries and carotid bifurcation. Alternatively electrodes can be used in bipolar or monopolar configuration. Alternatively, electrodes E1, E2, E3, and E4 may be used to deliver a stimulation or blockade signal to identify which electrode has optimal proximity to the carotid body or carotid body nerves, and distance from non-target nerves, and the electrode in the optimal position may be used to deliver ablation energy. In FIG. 50A electrode E1 may be determined to be too close to sympathetic nerve 121 while electrode E2 may be determined to be in a suitable position to ablate carotid body 27. FIG. 50B shows an ETAK catheter balloon having ablation elements E5, E6, and E7 spaced apart along a length of the balloon 648. Electrode E5 and E6 may be determined to be too close to sympathetic nerve 121 while electrode E7 may be at an optimal position for ablating carotid body 27. A multiple electrode balloon may comprise electrodes positioned at various locations along a length and diameter of a balloon. The balloon 641 or 648 may further comprise a sensor used to monitor ablation characteristics such as temperature and impedance. Impedance may be measured between an electrode on the balloon 641 or 648 and a dispersive electrode placed on the patient's skin. Alternatively, impedance may be measured between an electrode on the balloon and a guide wire 643 placed in the patient's internal carotid artery.

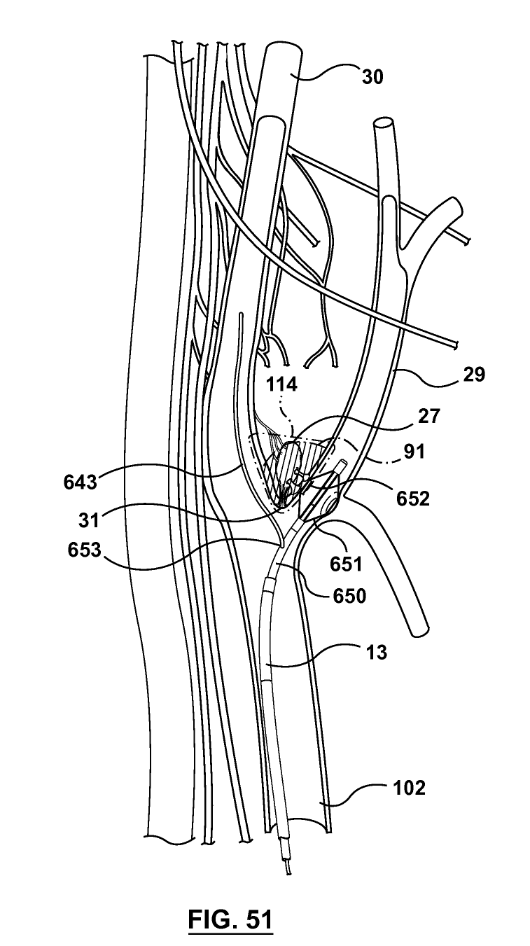

FIG. 51 shows an ETAK catheter 650 having an expandable structure in the form of an expandable wire cage 651 with an electrode 652 mounted to an arm of the cage. The catheter 650 has a side exiting guide wire port 653 through which a guide wire 643 is advanced into a patient's internal carotid artery 30. Placement of the guide wire 643 facilitates rotational orientation of the expandable cage 651 as well as distance in an external artery relative to a carotid bifurcation 31. The electrode 652 is mounted on an arm of the cage 651 so that when the oriented and positioned cage is expanded the electrode 652 is placed in apposition with an internal wall of the vessel at a suitable location for carotid body ablation. As with the balloon designs of FIGS. 49, 50A and 50B, an ETAK catheter having an expandable cage or other expandable structure, may comprise multiple ablation elements and an optimal ablation element may be used to deliver ablation energy.

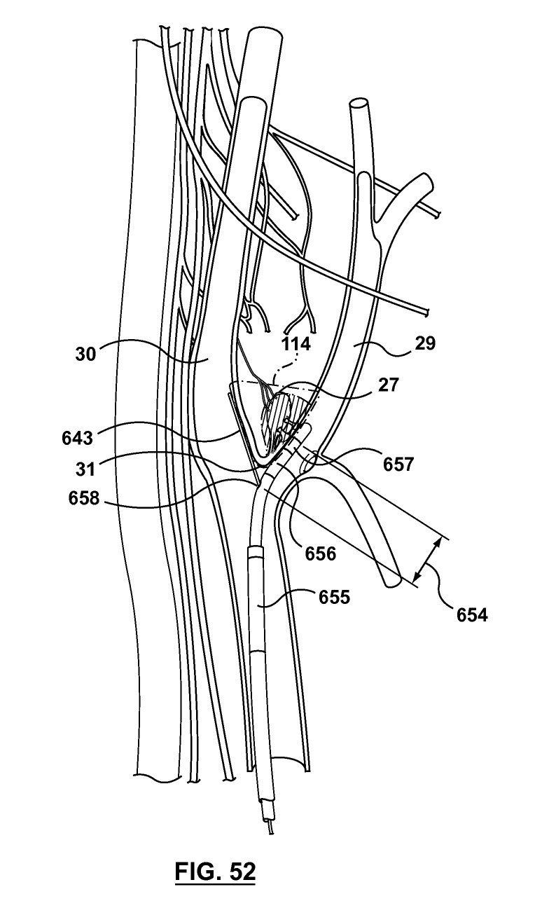

FIG. 52 shows an ETAK catheter 655 having a deflectable distal region 656, an ablation element 657 (e.g., radiofrequency electrode, bipolar radiofrequency electrodes, cryogenic applicator) mounted to the distal region, and a side exiting guide wire port 658 through which a guide wire 643 is advanced into the patient's internal carotid artery 30. The distal region of the catheter 655 is placed in the patient's external carotid artery 29 and the divergence of the side exiting guide wire 643 and the distal region may couple with a carotid bifurcation 31. The ablation element 657 may be positioned on the catheter at a predetermined distance 654 (e.g., between about 4 and 15 mm) from the exit port 658. The deflectable region 656 is configured to deflect, for example in the direction of the side exiting guide wire port, so that when the catheter 655 is rotationally oriented and coupled with a carotid bifurcation 31 deflection of the deflectable region 656 will place the ablation element 657 in apposition with the inner wall of the external carotid artery at a suitable location for carotid body ablation. An additional wire lumen may be incorporated into the catheter shaft to facilitate catheter placement in the external carotid artery 29. This wire can be advanced far up into the external carotid to secure the catheter from accidental dislodgement. Additional lumens can be incorporated to inject radiocontrast and drugs into the blood stream.

A carotid body ablation catheter may comprise a radially expandable structure, such as an inflatable balloon, a perfusion balloon, or a deployable wire cage, configured to position an ablation element (e.g., RF electrode, bipolar RF electrodes, ultrasound transducer, cryogenic element) at a suitable height (e.g., about 4 to 15 mm, 5 to 10 mm, 8 to 10 mm) from a carotid bifurcation for an effective and safe carotid body ablation procedure. The radially expandable structure may engage with carotid vasculature geometry such as a common carotid artery caudal to its bifurcation, a carotid bifurcation, an ostium of an external carotid artery, or an ostium of an internal carotid artery. The ablation element may be disposed on the catheter with respect to the radially expandable structure so that when the radially expandable structure is engaged with the carotid vasculature geometry the ablation element is positioned for carotid body ablation. The radially expandable structure may furthermore facilitate stabilization of the distal portion of the catheter during delivery of ablation energy. The radially expandable structure may furthermore facilitate placement of the ablation element within an external carotid artery at a suitable radial position, for example on the carotid septum or in contact with the wall of the external carotid artery facing the internal carotid artery. The ablation element may optionally be maneuvered with a means such as controllable deflection or a deployable structure such as a balloon.

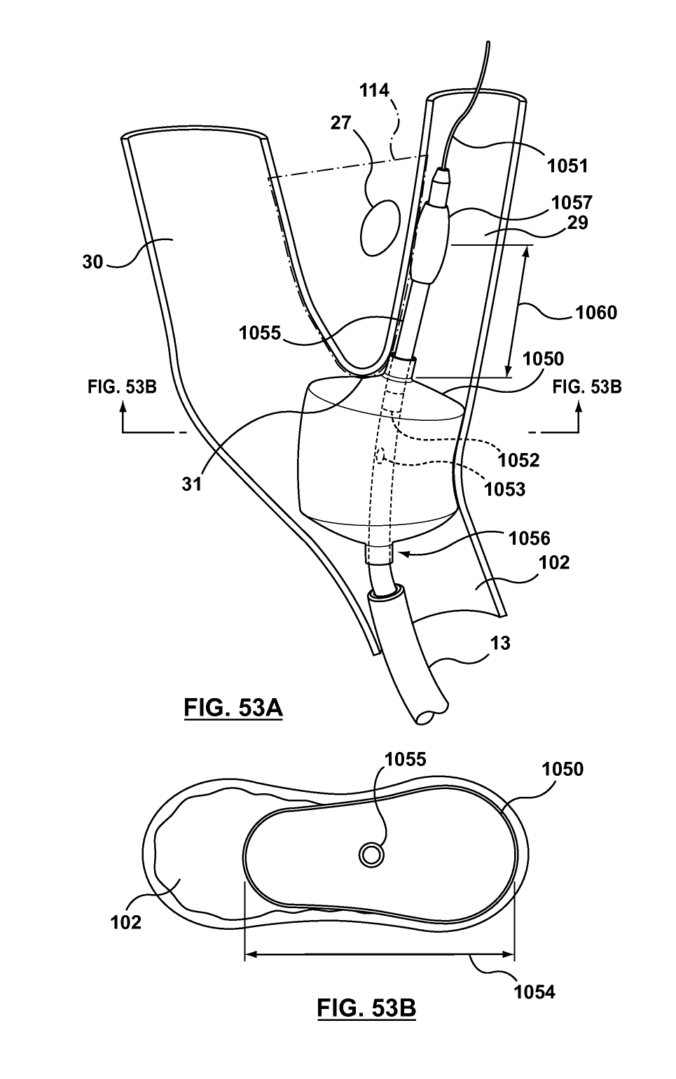

An exemplary embodiment as shown in FIGS. 53A and 53B comprises an inflatable balloon 1050, such as a compliant or semi-compliant balloon, configured to engage with a common carotid artery 102 just caudal to its bifurcation 31. The common carotid artery just caudal to its bifurcation may have a geometry that is different that the common carotid artery further caudal, e.g., about 3 cm caudal from its bifurcation. The balloon may be inflated to a larger diameter than an external carotid artery 29 so it prevents further advancement of the catheter, and optionally to a larger diameter than the common carotid artery 102 further caudal so it prevents retraction of the catheter. The common carotid artery just caudal to its bifurcation may have an oval or bilobular shape as shown in FIG. 53B. The catheter may be delivered over a guide wire 1051 that is delivered in to an external carotid artery 29 and through a delivery sheath 13. Contrast may be injected through the sheath 13 to image the carotid vasculature. The distal portion of the catheter may be advanced into the external carotid artery 29 until a radiopaque marker 1052 identifying the position of the balloon is aligned approximately with the ostium of the external carotid artery or the carotid bifurcation 102. The balloon 1050 may be inflated by injecting a fluid through an inflation lumen 1053 such that it expands beyond the diameter of the external carotid artery. For example, as shown in FIG. 53B the balloon may be inflated to a maximum width 1054 of about 6 mm, 7 mm, 8 mm, 9 mm, or 10 mm. The shaft 1055 of the catheter may be approximately centered in the balloon 1050. When the balloon is inflated in the common carotid artery just caudal to the bifurcation it may prevent the catheter 1056 from advancing further in to the external carotid artery. Furthermore the inflated balloon may position the shaft 1055 of the catheter close to or in contact with the carotid bifurcation 31, which may in turn position the ablation element 1057 in contact with the carotid septum 114. The ablation element may be disposed on the catheter shaft distal to the balloon at a distance 1060 between about 4 to 15 mm (e.g., about 5 to 10 mm, 8 to 10 mm) from the balloon 1050. When the balloon is inflated and its position is confirmed via radiographic imaging to be in the common carotid artery just caudal to its bifurcation or at the ostium of the external carotid artery, it can be expected that the ablation element is appropriately positioned for carotid body ablation. The position of the ablation element 1057 may be confirmed radiographically or vessel wall contact may be confirmed by impedance measurement. Ablation energy may be delivered from the ablation element to the target ablation site. For example, RF energy may be delivered to the carotid septum. If the ablation energy is RF a dispersive electrode may be placed on the skin of the patient, in an internal jugular vein, in an internal carotid artery, or in interstitial space.

### System

A system has been conceived comprising a catheter, having a means for coupling with an intercarotid septum for carotid body ablation, and an ablation energy console. The system may additionally comprise a connector cable or several cables for connecting the ablation energy console with the catheter, a delivery sheath, or a guide wire. The console may be configured to deliver ablation energy to the catheter. For example, the console may be an electrical signal generator such as a radiofrequency generator or an irreversible electroporation generator. The console may further comprise a user interface that provides the user with a means to select ablation parameters, activate and deactivate an ablation, or to monitor progress of an ablation. The console may further allow a user to select electrical stimulation or blockade used to investigate proximity of an ablation element on the catheter to neural structures. The console may comprise a computer algorithm that controls ablation energy delivery. The algorithm may control energy delivery (e.g., controlled power delivery, ramp time, duration) based on inputs for example, user selected variables, pre-programmed variables, physiologic signals (e.g., impedance, temperature), anatomical features (e.g., intercarotid septum width, presence of plaque, bifurcation angle), or sensor feedback. Selectable carotid body ablation parameters may include ablation element temperature, duration of ablation element activation, ablation power, ablation element force of contact with a vessel wall, ablation element size, ablation modality, ablation element position within a vessel, or intercarotid septum width.

Pressure or force sensors may be incorporated into any of the catheter embodiments herein, for example they could be mounted to a flex circuit proximate an ablation element, and could be used to verify contact or indicate contact force. Diverging arms with open/close actuation could be actuated to a position that corresponds to a particular contact pressure range. Alternatively, a catheter could be “pushed” against the wall until contact pressure reaches a desired level. Alternatively, a baseline pressure may be chosen when a desirable contact force is visually confirmed, for example vessel distension caused by ablation element contact force may visually appear using an imaging modality such as angiography. A change of pressure or force, within an acceptable range from the baseline, measured by the sensors may indicate appropriate contact force and deviation from this range could indicate an inappropriate contact force. A computer algorithm that controls delivery of ablation energy may discontinue energy delivery if contact force deviates from the appropriate range. Furthermore, a pressure sensor may be used to indicate absolute or relative blood flow and power delivery could be augmented by feedback from the pressure sensor. Alternatively, a temperature sensor, cooled by blood flow, can be used to determine blood flow velocity. Blood flow cooling can be factored into the control algorithms as correction of energy delivery. Also sudden drop of blood flow can indicate spasm of the carotid vessel. Such an abrupt temperature rise will indicate a need to stop or reduce energy delivery instantly. For example, low flow may equal less power and/or power delivery duration, while greater flow may result in more power and/or longer duration. Power of ablation energy delivery may be decreased or duration of energy delivery may be reduced if the flow decreases. Conversely, should the flow increase power or duration may be increased. Alternatively, a pressure sensor may be used to track potential damage to nerves that are to be preserved. Heart rate may be inferred from a pressure sensor through pulsatile flow. The right vagus nerve primarily innervates the sinoatrial node while the left vagus nerve primarily innervates the atrioventricular node. Should either vagus nerve become stimulated, blocked or damaged the patient's heart rate may fluctuate or decline, which may be indicated by the pressure or flow sensor an energy delivery algorithm may stop power delivery or provide a warning accordingly. Similarly, heart function and some gauge of instantaneous heart rate variability may be measured in other ways (e.g., ECG, plethysmography, pulse oximetry) and used by an energy delivery algorithm for safety.

Contact between electrodes and tissue throughout delivery of energy, contact along a full length of an electrode, contact pressure, or stable contact may be important to create a predictable, well controlled ablation. Temperature sensors in each ablation element may be used to indicate characteristics of tissue contact. For example, as energy is applied (e.g., radiofrequency) and tissue is heated, temperature sensors in the ablation elements may be expected to increase as a function of energy delivered and tissue contact. If there is no tissue contact or contact is partial, intermittent, instable or with soft pressure, measured temperature increase may not be as expected (e.g., a lower temperature rise than expected). Temperature measured from multiple sensors may be compared to indicate characteristics of contact. For example if one sensor measures an expected temperature, increase in temperature, or temperature response to energy delivery while a different sensor does not measure an expected result then inconsistent contact may be detected. An algorithm may detect inconsistent ablation element contact and provide a warning and suggest which ablation element requires repositioning.

Tissue impedance, phase or capacitance may be measured between electrodes on each arm of an ETAP catheter in a bipolar arrangement, or between an electrode on one arm and a dispersive electrode on a second arm. Impedance measurement across an intercarotid septum may be used to indicate distance between electrodes, intercarotid septum width, carotid bifurcation angle, position on a bifurcation, tissue characteristics, ablation characteristics, electrode contact with tissue, catheter integrity, presence of plaque (e.g., calcified or atheromatous plaque). An energy delivery algorithm may incorporate impedance feedback, phase changes, or temperature to control delivery of ablation energy. For example, these feedback variables may be used to modulate energy delivery or as a safety cut-off. Ablation energy may be delivered for a predetermined duration of time (e.g., between about 20 and 90 s, or in a range of about 20-30 s) and energy delivery may be reduced or stopped if there is indication that a traumatic event or a poor ablation is about to happen, such as high temperature or temperature above set point, which may lead to events such as charring or coagulation, or significant movement or poor contact of the electrodes with respect to tissue, which may lead to unpredictable ablation or ablation at a non-target region. Calcified plaque may be detected by high impedance for a given septum width. For example, septum width may be measured using fluoroscopic visualization and if impedance is higher than a predetermined range of normal impedance for the measured septum width then calcified plaque may be present. A computer algorithm may compute presence of plaque based on input septum width and a lookup table of impedance measurements. A bipolar arrangement may be more sensitive to impedance changes and be able to prepare the generator to shut off more quickly than a monopolar arrangement. For example, a bipolar radiofrequency configuration may provide an improved signal to noise ration compared to a monopolar configuration and may provide a clear indication that electrodes are moving. However, an energy delivery control algorithm for either a bipolar or monopolar configuration may incorporate feedback variables for ablation and safety control as discussed herein. For example, prior to charring, which may be indicated by a sharp spike in impedance, several cycles of impedance fluctuation may be measured; if electrode contact with tissue is compromised or electrode position has moved an acute impedance change and simultaneous temperature change at one or both electrodes may be measured; if a catheter is compromised a feedback signal from a temperature sensor may be severed or out of a reasonable range; if a vessel is undergoing spasm impedance and temperature fluctuations as well as power phase changes may be detected simultaneously and in a sinusoidal pattern or may be determined based on hysteresis. Any of these indications may result in a reduction of energy delivery power, power shut off, or a safety warning. Variables such as impedance and temperature may be an indication of a successful ablation. For example, changes in impedance (e.g., value and phase) may be measured when carotid body perfusion is coagulated. This may be an indication that target temperature is exceeding 50-60 C, which may be an indication of technical success. Energy delivery may be stopped or continued for a short amount of time after this occurs to limit a chance that a lesion grows into that hazards medial zone. Another way an energy delivery algorithm may incorporate impedance feedback, phase changes, or temperature to control delivery of ablation energy is to adjust power delivery to meet a set point temperature, impedance, phase or capacitance.

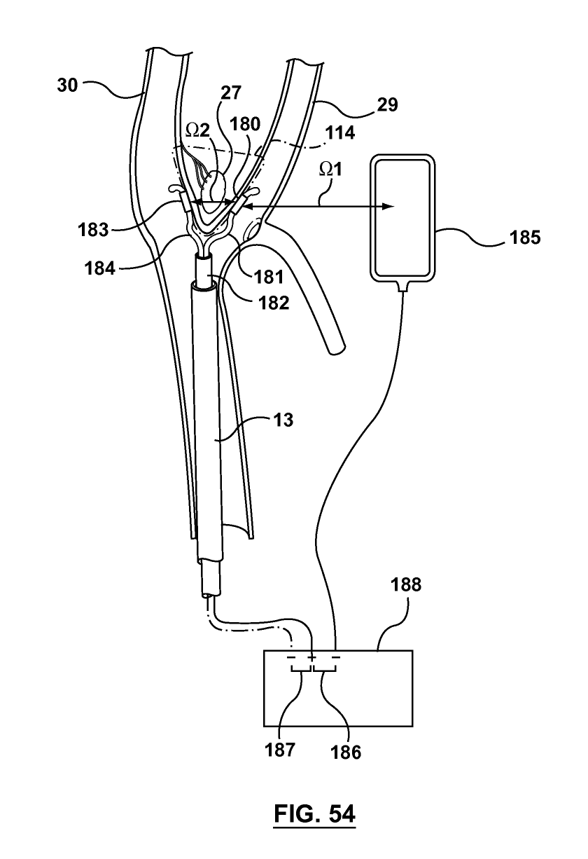

An ETAP or ETAK catheter may be configured for monopolar radiofrequency energy delivery and may comprise only one ablation electrode on an arm and the other arm may not have an electrode but be used for positioning the arms at a carotid bifurcation and in apposition with a target ablation site such as an external carotid artery wall of an intercarotid septum. In this monopolar configuration a dispersive electrode positioned on a patient's skin may compete the radiofrequency circuit. Another embodiment of an ETAP catheter configured for monopolar radiofrequency energy delivery may be constructed the same as embodiments shown in FIGS. 6 through 41, however an additional dispersive electrode connected to an energy source may be placed on an external surface of a patient and an electrical circuit for ablation may be provided by connecting an energy source to one of the electrodes intended for ablation an the dispersive electrode. As shown in FIG. 54 an active electrode 180 on an arm 181 of an ETAP catheter 182 may be placed, for example, in an external carotid artery 29 in contact with a target ablation site (e.g., vessel wall, intercarotid septum) and a second electrode 183 on a second arm of the ETAP catheter may be placed in the other carotid artery (e.g., internal carotid artery 30), which may facilitate positioning and apposition of the active electrode 180 at a target ablation site. However, the second electrode may be inactive for ablation and, optionally, active for electrical measurements such as tissue impedance, phase, or capacitance. During ablation, a circuit 186 may be made between active electrode 180 and dispersive electrode 185 placed on the patient's skin. The active electrode 180 may deliver radiofrequency current through tissue to dispersive electrode 185. Tissue impedance Ω1 may be measured during ablation between the active electrode 180 and the dispersive electrode 185 and may be used as a variable to control ablation energy delivery. A circuit 187 between electrodes 180 and 183 may allow a different tissue impedance Ωn to be measured between these electrodes, which may provide information more specific to the intercarotid septum such as ablation characteristics and electrode contact or motion. Tissue impedance Ω2 may be measured before or after ablation energy is being delivered by transmitting a low power/voltage/current signal between electrodes 180 and 183. Tissue impedance Ω2 may also be measured during ablation, for example, by cycling the ablation energy off periodically (e.g., once every second) for a short duration (e.g., for 1/30 of a second) during which time an impedance measuring signal is delivered between electrode 180 and 183 to obtain tissue impedance Ω2. A control algorithm in an energy console may switch between circuits 186 and 187. Alternatively, two separate radiofrequency energy sources may be used to run circuit 186 and 187. In addition to lower power, voltage, or current for measuring impedance, phase change or capacitance without creating a lesion, circuit 187 may apply a lower frequency, which may capture changes in the underlying tissue (e.g., intercarotid septum) more accurately.

### Bipolar Carotid Septum Ablation

The inventors determined that an intercarotid septum may be an ideal ablation target for a carotid body ablation procedure. With this understanding they conducted studies to establish a safe range and technique of energy delivery to create well-controlled and consistent ablations in intercarotid septa with a goal of a high probability of CB destruction with mitigated risk to the artery walls and important adjacent non-target nerves or organs. A further goal was to assess usability (e.g., ease of delivery, positioning and targeting) of a catheter for a CBA procedure. The studies included ablation studies in animals, histological analysis, finite element modeling, and in bench testing.

A porcine model was developed having an ablation target of a bi-carotid bifurcation, which has a similar arterial bifurcation (vessel diameter of 4.2-6.2 mm, bifurcation angle of 20-45° to a human's carotid bifurcation (vessel diameter of 4-6 mm, bifurcation angle of 48.5+/−) 6.5°. The arteries also have a similar blood flow and cellular makeup.

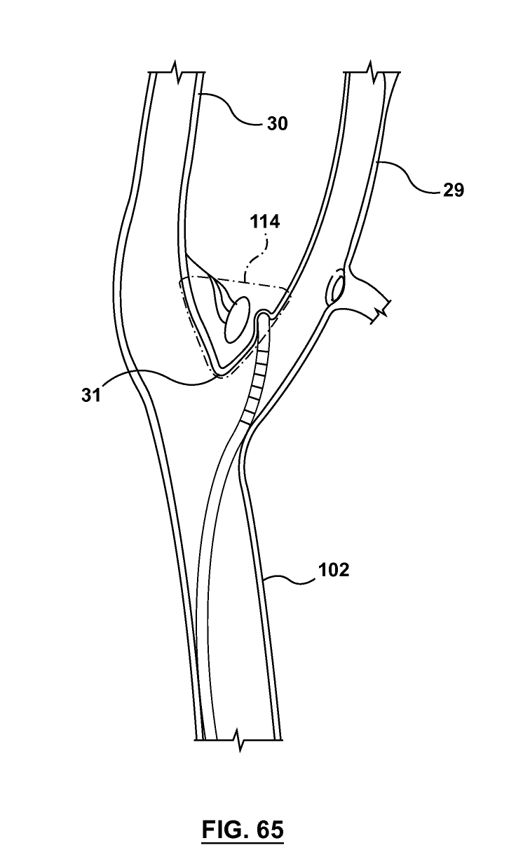

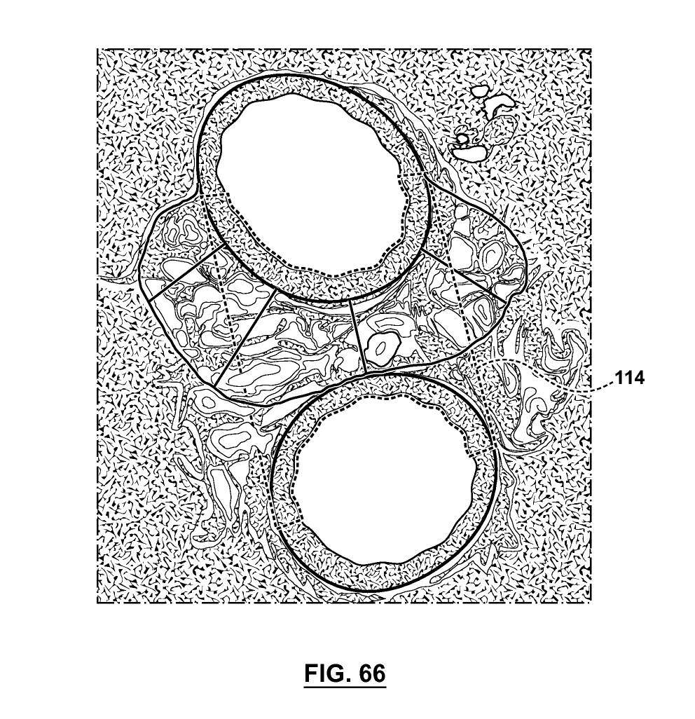

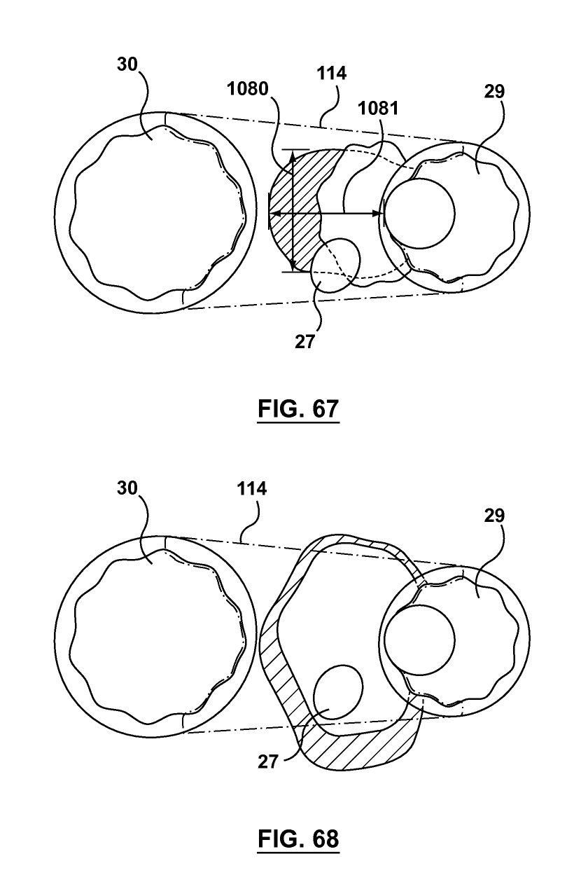

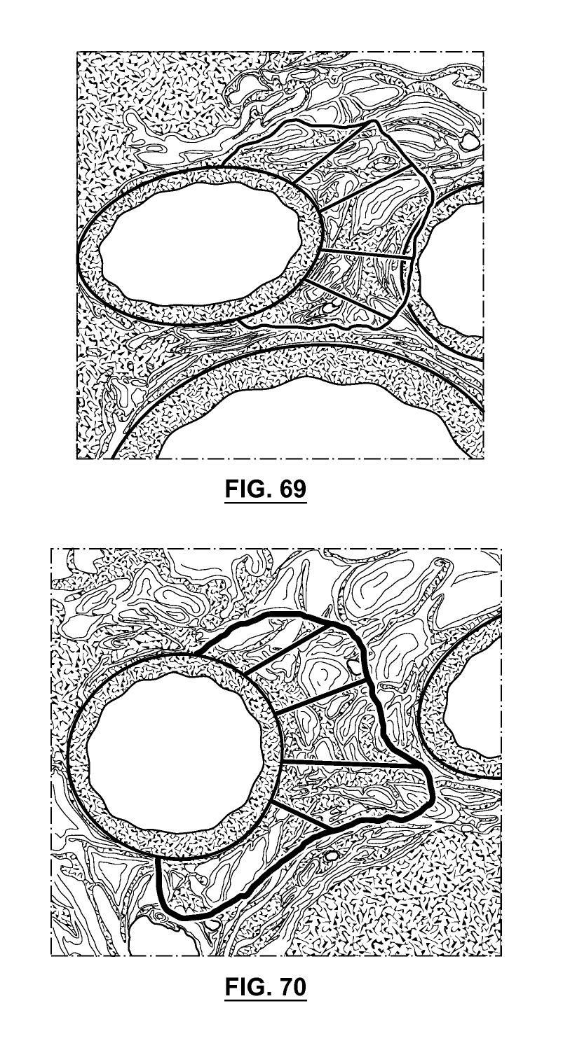

Monopolar RF ablation was assessed in the porcine model. 14 animals were studied with a total of 63 ablations using a RF power between 10 to 40 W and energy delivery of 30 s. A monopolar RF catheter having controllable deflection and a 7 French, 4 mm long electrode was delivered to the bi-carotid septum as shown in FIG. 65. Investigators, who were experts in using endovascular catheters, found it very difficult to accurately position the electrode in a desired target site. Histological assessment (as shown in FIGS. 66-70) found monopolar ablation to be safe in regards to its effect on vessel walls resulting in no incidence of char formation, coagulum, thrombus at the ablation site, or aneurism. Histology further found that ablation zones using 10 W (see FIG. 67 showing a range of ablation size from minimum to maximum) varied in width 1080 (4-5 mm) and vessel-to-vessel depth 1081 (2.4-4.8 mm), which may be sufficient to ablate a portion of a carotid body 27 and remain contained in the carotid septum space 114. The ablations, however, were less consistent compared to bipolar studies. Ablation zones using 15 W (FIG. 68 showing a range of ablation size from minimum to maximum) were larger in width (6.0-8.6 mm) and vessel-to-vessel depth (4-5 mm), which is a greater volume of ablated septum than the 10 W studies, but which is also wider than the septum space 114 or uncontained by the medial and lateral boundaries of the septum creating a potential safety risk. Furthermore, consistency of monopolar ablations was assessed and found to be variable, which could result in unpredictable results. For example, as shown in histology slides illustrated in FIGS. 69 and 70, multiple 15 W monopolar ablations in a porcine bi-carotid septum resulted in lesions that varied in containment within the septum and direction of spread.

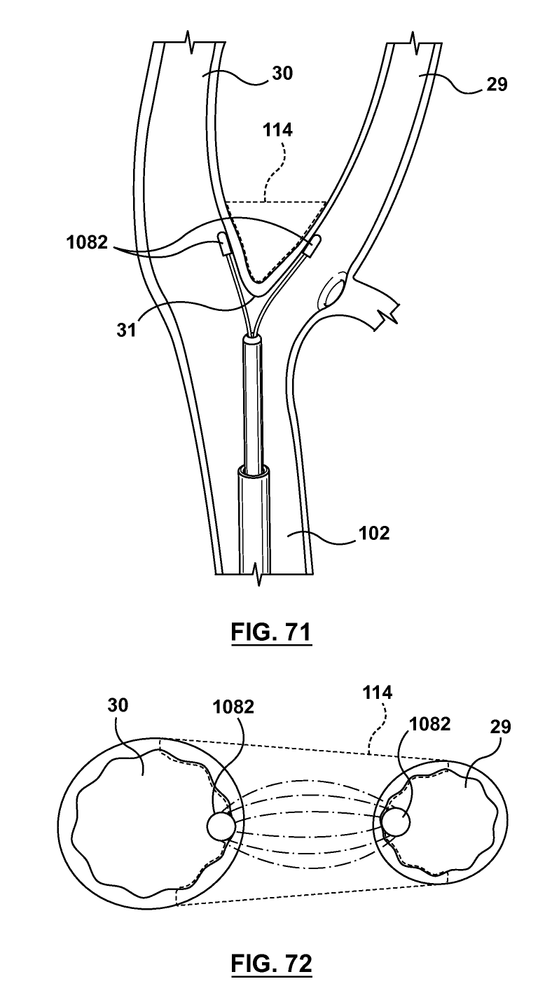

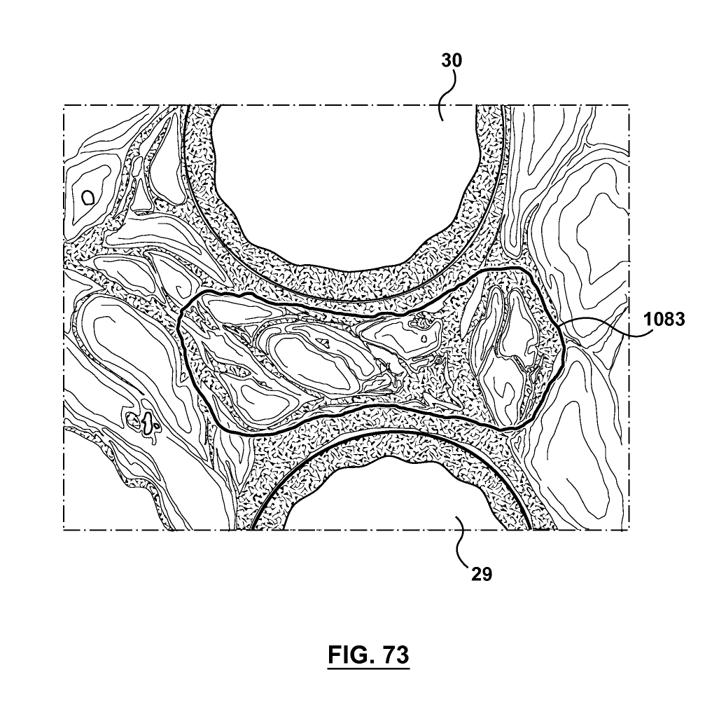

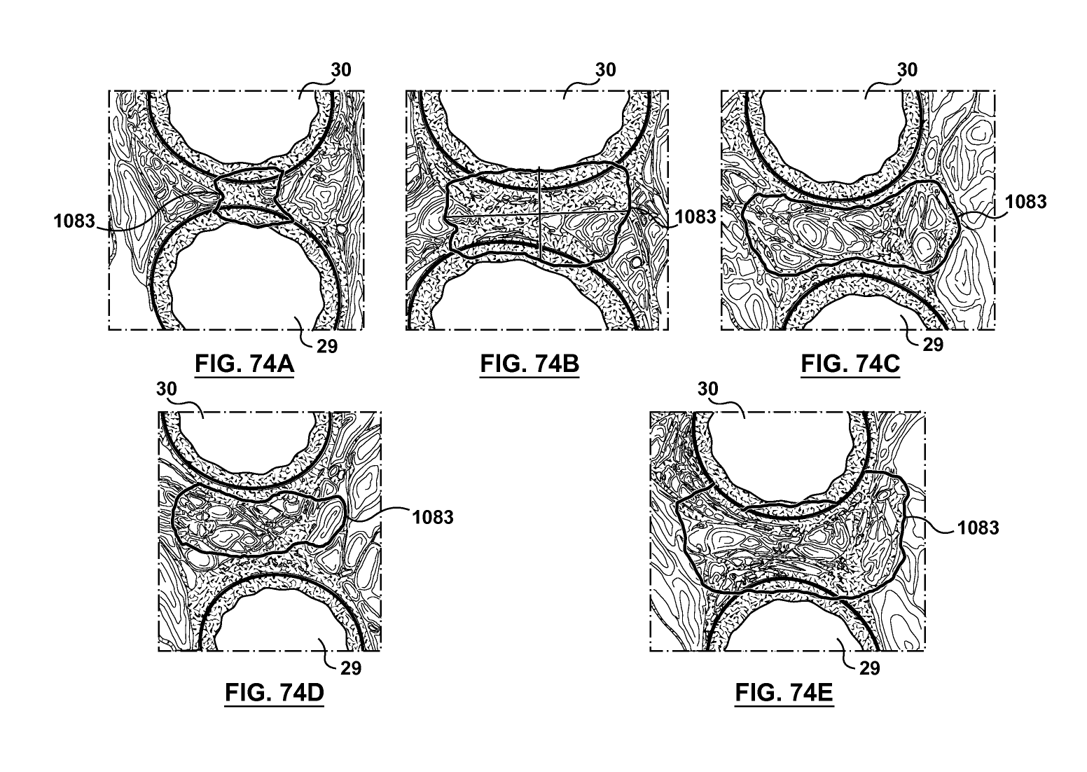

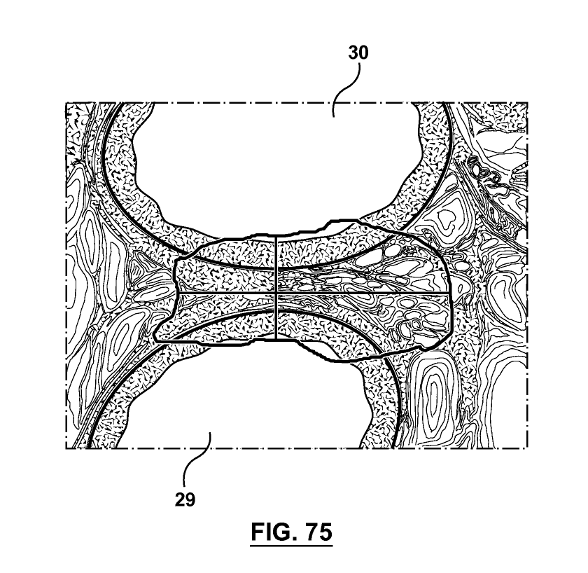

Bipolar RF ablation was assessed in the porcine model and compared to the monopolar results. The hypothesis was that bipolar RF energy may create an ablation that is safely contained within an intercarotid septum and also significantly large enough to ensure a high probability of effectiveness. The bipolar electrode arrangement, as shown in FIGS. 71 and 72, comprised placing electrodes 1082 of similar size (3.5 French diameter) and 4 mm long on both sides of a porcine bicarotid arterial bifurcation to mimic a human scenario of one electrode in an internal carotid artery 30 and one in an external carotid artery 29 on an intercarotid septum 114 (e.g., between 5 and 10 mm cranial to a carotid bifurcation saddle 31). Power delivery ranged between 4 and 10 W for 30 s and 6 W was found to be an ideal power. Histology slides for 6 W bipolar ablations, as shown in FIGS. 73 and 74A to 74E, were found to have appropriately large and contained lesions 1083. Examination of 28 ablation sited in 16 animals confirmed vessel safety with zero incidences of severe hemorrhage, clot formation, platelet aggregation, char formation, coagulum, thrombus, aneurysm, or vessel constriction. As shown in FIG. 75, bipolar ablations using 6 W for 30 s consistently created ablations that were effective in size (i.e. lesions always spread from the internal carotid artery 30 to the external carotid artery 29 and ranged in width across the septum from 4 to 6 mm), safely contained in a septum, safe for the vessel and consistent. The results of the bipolar RF ablation studies performed indicated significant advantages compared to the monopolar RF ablation studies, although monopolar RF ablation can be used to reduce afferent signaling from the carotid body.

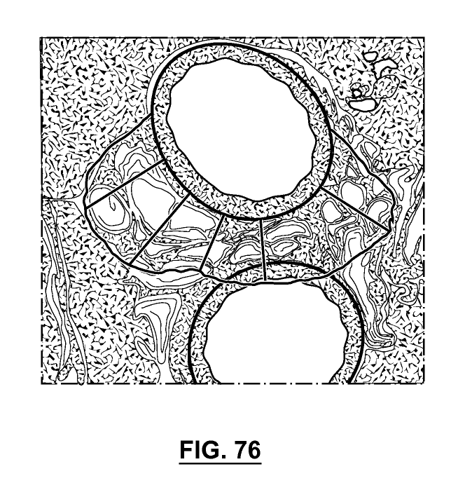

Furthermore, compared to 15 W monopolar ablations (see FIG. 76), 6 W bipolar ablations (see FIG. 75) consistently created a safely contained ablation in narrow bifurcations. Bipolar RF ablation was found to use less energy to yield safer, more contained and effective ablations.

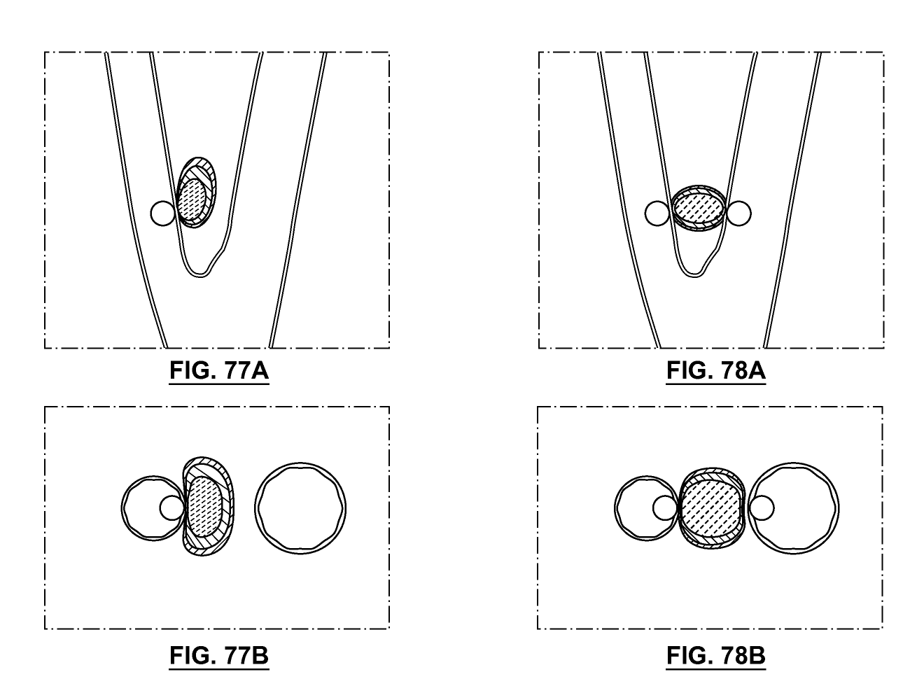

Finite element modeling was done to compare bipolar carotid septum ablation (shown in FIGS. 78A and 78B to monopolar carotid septum ablation (shown in FIGS. 77A and 77B). FIG. 77A is a sagittal cross sectional view of the finite element model illustrating isotherms of a monopolar RF ablation. FIG. 77B is a transverse cross sectional view of the finite element model illustrating isotherms of a monopolar RF ablation. FIG. 78A is a sagittal cross sectional view of the finite element model illustrating isotherms of a bipolar RF ablation. FIG. 78B is a transverse cross sectional view of the finite element model illustrating isotherms of a bipolar RF ablation. The model utilized geometry and properties of average human carotid bifurcation anatomy, with cooling by blood flow in common, internal and external carotid arteries. Differential electrode sizes and locations as well as power levels were studied. The model calculated tissue temperature and estimated ablation size based on a FDA-recommended relationship of tissue temperature and thermal necrosis. The finite element modeling confirmed porcine experiment results.

A challenge of heating a large volume of tissue with conventional monopolar application of radiofrequency or other frequency alternating electric current is that current density is typically greatest in tissue nearest an active electrode. In a relatively homogeneous medium heat is generally proportional to current density. Over time, temperature will begin to increase in tissue nearest the electrode forming a lesion that grows outward by conduction of heat. Overheating tissue nearest the electrode may cause it to char which can have undesired effects such as an increase in electrical impedance of the charred tissue resulting in uncontrolled delivery of energy, unpredictable lesion formation, gas formation, or iatrogenic injury. Lesion size is a function of electrode surface area in contact with tissue, cooling conditions such as perfusion by blood, and energy delivery parameters such as power. Creating a lesion with RF in relatively non-homogeneous tissue is a function of additional factors such as the different electrical and thermal properties of the varying tissues, which may be altered by varying rates of perfusion, blood flow, or tissue composition.

Heating tissue at a distance from an electrode may be limited by overheating of tissue near an active electrode. This may be overcome by cooling the electrode, pulsing energy delivery, increasing electrode size, or adding electrodes.

Bipolar RF is another way to increase the size of a lesion by concentrating current between two active electrodes, thus maintaining a fairly high current density in the tissue between the electrodes, not only in tissue nearest an active electrode. Bipolar RF can also control the size and shape of a lesion. The ability to effectively contain concentrated current between two bipolar electrodes is a function of distance between the electrodes. In a relatively homogeneous medium, even with bipolar RF, current density will be greatest in tissue nearest the electrodes and lesions will begin to form around the electrodes and grow toward one another in the tissue between the electrodes. The greatest thermal injury may be in tissue next to the electrodes. Tissue in between the electrodes, particularly in the center, may reach an ablative deposited thermal energy dose, however the thermal exposure (temperature rise multiplied by time) will be less than that applied to tissue nearer the electrodes.

The application of trans-septal bipolar RF to a carotid septum as described herein has several beneficial mechanisms. The environment is not homogeneous so the thermal profile behaves differently that in a homogeneous medium, particularly due to the cooling action of blood flow. The distance between electrodes placed in an internal and external carotid artery on a carotid septum is variable with anatomy between about 2 to 10 mm, which is within a range sufficient to concentrate current density between electrodes enough to create a substantially trans-septal bipolar ablation. High blood flow in the internal and external carotid arteries, as well as in the common carotid artery and over the carotid bifurcation helps to remove heat from the vessel walls and tissue near the vessel walls. As bipolar RF energy is delivered across a carotid septum tissue temperature between the electrodes and along the current path will rise. Blood flow will temper the thermal increase in the vessel wall and tissue near the vessel walls, and temperature of tissue closer to and at the center will rise. The electric current has a general tendency to follow the path of least resistance. In the case of bilateral trans-septal ablation the simplified current path can be presented as two resistance elements connected in parallel: one through septum tissue and a second through a blood path around the carotid bifurcation. Blood has lower resistivity compared to septum tissue but the distance that current needs to travel is longer since the shortest path between two electrode lies through the septum path (i.e., trans-septal). This bipolar arrangement of electrodes concentrates RF resistive heating in the septum. As the tissue of the septum gets heated by the RF current its impedance drops, because ionic conduction in tissue is a function of temperature, and larger share of current is directed into the septum and lesser into the blood. The thermal dose applied to tissue across the septum will be more even, or the thermal dose of the center tissue may be greater than central tissue in an environment without blood flow. This is beneficial because the target ablation site is across the septum and it is desired to avoid iatrogenic thermal injury to the vessel walls. Additionally as described herein, bipolar RF applied to a carotid septum has been shown to contain an ablation within a thickness suitable for effective ablation of a carotid body or its associated nerves and for safe avoidance of non-target nerves or tissue near the septum.

The total impedance during bipolar carotid septum ablation is a function of resistivity (i.e. resistance per unit of volume) of the septal tissue that decreases with increasing temperature, resistivity of blood and the length of the current paths through tissue and blood. Resistance of blood that is in parallel stays constant. During ablations in animal studies that produced robust lesions total impedance was observed to drop 15-25% after a period of initial heating of septal tissue. Because of high blood flow temperature of blood in the blood path and thus resistivity of the blood does not change.

An ablation is created by resistive heating of tissue that is proportionate to the current density created by field strength in the trans-septal current path. Electric current that travels through blood may not contribute to the ablation. Current density is current that crosses through an area unit of the cross-section of the path. In septal tissue cross-section of a current path may be roughly approximated by the area of the electrode footprint.

A goal of a carotid body ablation system may be to achieve current density along the trans-septal RF current path that is high enough to achieve a robust lesion as a result of resistive heating of tissue along the septal path. Since current density in the septum cannot be measured this was achieved by FEM modeling, bench top tests in phantoms that approximated tissue properties and surrounding conditions and finally by with animal studies.

A finite element model predicted that a thermal profile formed in a carotid septum by bipolar RF energy applied to the septum from the internal carotid artery and external carotid artery would heat sufficiently across the septum while maintaining safe temperatures proximate the electrodes (FIGS. 78A and 78B). The finite element model also showed that as bipolar RF energy is delivered to a carotid septum, heat evolves in tissue nearby electrodes first then eventually from the center out.

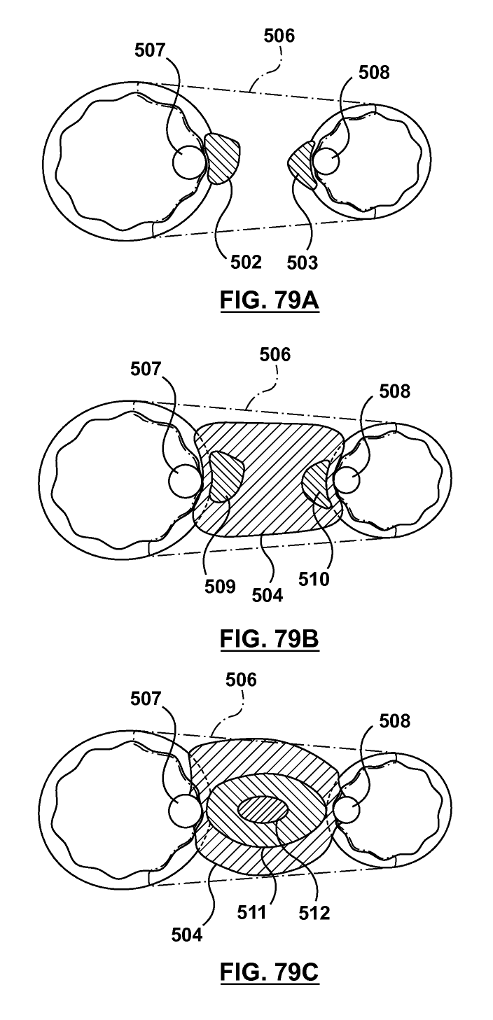

FIG. 79A shows a finite element model of a thermal profile across a carotid septum 506 at 11 s wherein two isotherms 502 and 503 representing temperature between about 40° C. and 50° C., are forming near the bipolar electrodes 507 and 508. A small layer of lower temperature tissue is between the isotherm 502 and electrode 507 and likewise between isotherm 503 and electrode 508 due to cooling by the blood flow.

FIG. 79B shows a finite element model of a thermal profile across a carotid septum 506 at 15 s wherein the 40° C. to 50° C. isotherms 502 and 503 have grown to connect across the septum shown by isotherm 504. Temperature continues to increase in tissue near the electrodes as shown by 50° C. to 60° C. isotherms 509 and 510.

FIG. 79C shows a finite element model of a thermal profile across a carotid septum 506 at 20 s wherein the 40° C. to 50° C. isotherm 504 has grown to fill more of the septum 506; the 50° C. to 60° C. isotherms 509 and 510 have grown to connect across the septum shown by isotherm 511; and tissue in the center has increased in temperature as shown by 60° C. to 70° C. isotherm 512, which is growing from the center out.

### Exemplary Experimental Results

The energy delivery parameters (power, duration, ramp up slope) were studied by inventors using the embodiment described by FIGS. 30-33. These studies may apply to any embodiment placing RF electrodes within desired target regions 136, 137, 138, and 139 (as shown in FIGS. 5A and 5B) having electrodes of similar geometry (e.g., about 4 mm long, about 0.048″ diameter, barrel shaped, elasticity of arms). The objective of the study was to determine a range of RF energy (e.g., power or current) delivery that will create a suitable lesion volume in a carotid septum having a given width that determines the distance between electrodes and the current path, or impedance measured between bipolar electrodes. An objective of carotid septum ablation may be to create a lesion that substantially spans the septum from the internal carotid artery to an external carotid artery and about 50 to 100% of the thickness of the septum from the medial to lateral boundary in order to optimize the probability of ablating or denervating a carotid body. It may be desired to obtain this coverage in a narrow septum as well as a wide septum.

In the study a variety of power levels (6, 8, 10, 12 W) were applied to porcine carotid septa of different widths, but with the aim of achieving an average inter-electrode distance of 5.5 mm, which is a 3rd quartile of inter-septal distance found by a retrospective and prospective computed tomography angiography analysis. Actual inter-electrode distance was determined to range from 3.8 mm to 8.0 mm using angiography. Samples included 14 different bifurcations from 8 different animals performed at 2 different test facilities. The baseline total impedance measured between electrodes, which is a function of impedance through a blood path in carotid vasculature and impedance through septal tissue, for these samples before delivering ablative energy was 240-300 ohms. All trials in samples of varying thicknesses using power of 6, 8, or 10 W resulted in acceptable ablations with sufficient septal coverage and safe containment. Trials using 12 W resulted in electrode temperature over 60° which may be less desirable because it could indicate a high temperature of a vessel wall, which could increase risk of thrombus formation or vessel injury.

In one embodiment, power may be adjusted based on carotid septal width. To make a comparable lesion coverage for a wider septum one may need to apply more energy, for example more power for a given duration, similar power for a longer duration, or more power for longer duration. Conversely, power may be titrated down for narrower septa to ensure the produced lesion is contained in the carotid septum. A RF console may comprise a computer-controlled algorithm that adjusts energy delivery parameters such as power amplitude or duration according to septum width, which may be measured and input as a variable by a user. Septum width may be measured on an angiogram or on fluoroscopy by measuring the distance between radiopaque electrodes placed on the sides of the septum. For example, a septum measured on angiography to be between about 2 to 5 mm may correspond to a chosen power of about 6 W, a septum measured to be between 4 to 8 mm may correspond to a chosen power of about 8 W, and a septum measured to be between 7 to 10 mm may correspond to a chosen power of about 10 W.

In another embodiment power may be adjusted based on impedance measured between the two electrodes. Septum width and impedance measured across the septum between electrodes may generally be correlated. However, impedance is also a function of tissue composition. More power may need to be applied to achieve comparable lesions for a carotid septum having higher impedance, regardless of septal width. Conversely, power may be titrated down for septa measuring lower impedance to ensure the produced lesion is contained in the carotid septum. A RF console may comprise a computer-controlled algorithm that automatically adjusts energy delivery parameters such as power amplitude or duration according to measured impedance.

### Energy Directed RF

As set forth above, the disclosure provides devices, systems and methods for positioning a distal region of a catheter in a vessel proximate a carotid body (e.g., in a common carotid artery, internal carotid artery, external carotid artery, at a carotid bifurcation, proximate an intercarotid septum), positioning an active electrode proximate to a target ablation site (e.g., a carotid body, afferent nerves associated with a carotid body, a peripheral chemosensor, an intercarotid septum), positioning a reference electrode proximate the target ablation site, and delivering ablation energy from the active electrode through the target ablation site to the reference electrode to ablate the target site. Several methods and devices for carotid body modulation are described. As set forth above, in some embodiments a catheter includes a first electrode and a second electrode, wherein one or more aspects of the catheter is configured so that in use the first electrode is in contact with the external carotid artery proximate the carotid septum, and the second electrode is in contact with the internal carotid artery proximate the carotid septum. In use, energy is then delivered between the electrodes to ablate septal tissue to achieve a therapeutic effect.

In some embodiments, however, one or more aspects of the catheter are configured so that one or both of the electrodes are not in contact with the external and internal carotid arteries, respectively, when energy is delivered between the electrodes. These embodiments are examples of “energy-directed” carotid body ablation as used herein. Some embodiments of endovascular energy-directed ablation of a carotid body include delivering a device through a patient's vasculature to a blood vessel proximate to a target ablation site (e.g., carotid body, intercarotid plexus, carotid body nerves) of the patient, placing an active electrode associated with the device against the internal wall of the vessel adjacent to the target ablation site, placing a reference electrode in a vessel adjacent to the target ablation site but not in contact with the vessel wall, such that the target ablation site is between the active electrode and reference electrode and within a distance such that current density is concentrated or directed toward the reference electrode, and delivering ablation energy to ablate the target ablation site. These embodiments of energy-directed ablation of a carotid body differ from monopolar ablation or bipolar ablation as described below. In alternative embodiments of endovascular energy-directed carotid body ablation, neither of the electrodes are in contact with the vessels wall. In energy-directed ablation, the ablation energy may be, for example, electrical energy, irreversible electroporation, radiofrequency energy, cooled radiofrequency energy, or a pulsed electrical signal.

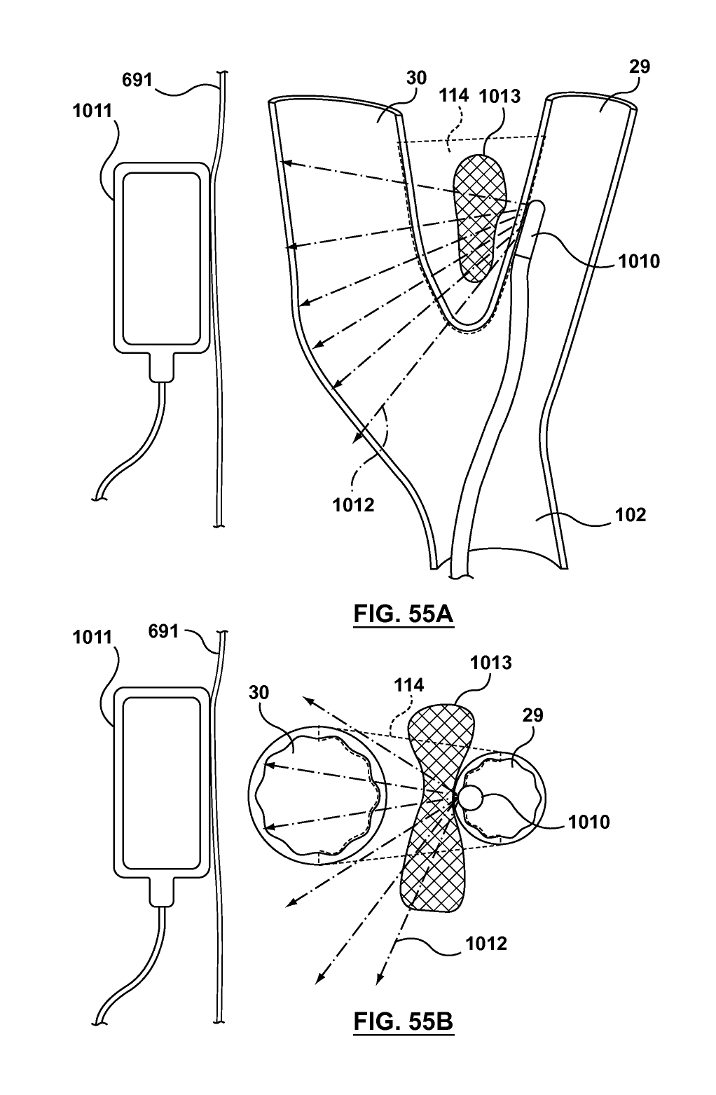

Monopolar radiofrequency (RF) ablation is referred to as a mode of tissue ablation wherein RF current is passed from an active electrode, typically positioned proximate a target ablation site, to a reference electrode, typically positioned on a patient's skin. The active electrode is significantly smaller than the reference electrode so that current density in tissue around the active electrode is high enough to raise tissue temperature and to thermally ablate tissue, while the current density in tissue around the reference electrode (which can also be referred to as an indifferent electrode or a return electrode) is low enough to not thermally ablate the tissue. The reference electrode is typically positioned at a distance from the active electrode such that current path in tissue proximate the active electrode is significantly diffused and a resulting tissue ablation is not directed toward the reference electrode. For example a monopolar RF ablation may comprise placing an active electrode near a nerve in a patient's back and placing a reference electrode on a surface of the patients thigh resulting in a sufficiently omnidirectional thermal ablation around the active electrode. A schematic illustration shown in FIG. 55A and FIG. 55B depicts how monopolar RF ablation of a carotid septum may occur. For example, an active electrode 1010 may be placed in an external carotid artery 29 as shown, where risks of thrombosis and embolization are significantly lower than in the common carotid artery 102 or internal carotid artery 30 that feed the brain. A reference electrode 1011 is typically a conductive patch placed on the skin 691 of the patient 2 (e.g., on a shoulder or thigh). As RF current is passed between the active and reference electrodes, an electric field 1012 emanates from the active electrode 1010 and disperses sufficiently in all directions (shown by the dispersing arrows 1012), or at least unaffected by the position of the reference electrode 1011. The dispersion of the field can be attributed to the distance between the electrodes, high impedance of skin and large surface area of the reference electrode 1011. Resistive heating occurs in a thin layer of tissue just below the surface of the vessel where the ablation electrode firmly contacts (i.e., is in apposition of) the wall of the artery. Beyond this thin layer of tissue (e.g., less than about 1 mm thick), the electric field 1012 quickly dissipates, current density becomes too low for significant resistive heating, and further tissue heating and thermal lesion formation may be caused by convective heat. The expansion of the zone heated by convection and resulting thermal necrosis zone 1013 is governed by: (a) cooling effect of adjacent blood vessels and (b) tissue properties such as electrical impedance or thermal conductance. Specifically in this example an ablation tends to grow cranially (towards the head) and laterally (towards the skin and towards the spine) since convective heating of the septum itself is opposed by the cooling effect from common and internal carotid arteries. A monopolar arrangement may be suitable in some situations for carotid body modulation, particularly if the patient's carotid body is in within an expected monopolar ablation zone and if the patient's important non-target nerves are not within an expected monopolar ablation zone. However, precautions may be warranted to ensure patients are selected appropriately.

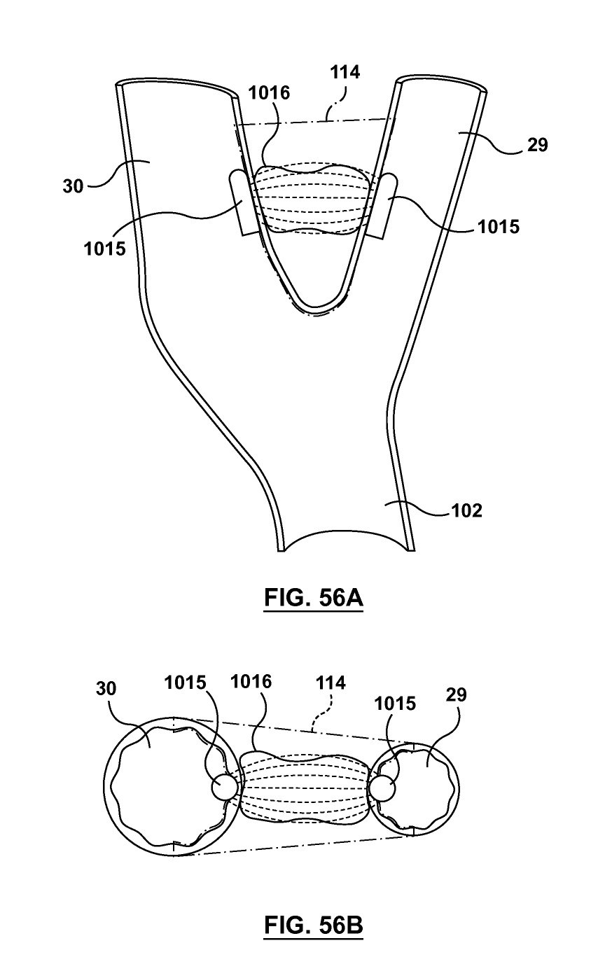

Bipolar RF ablation is referred to as a mode of tissue ablation wherein RF current is passed from a first active electrode to a second active electrode, wherein both active electrodes are typically positioned near one another (e.g., within about 30 mm, within about 15 mm, or within about 5 mm) or within a distance in which current density has a tendency to concentrate between the electrodes, which may create a continuous ablation between the electrodes, or a less omnidirectional ablation having greater concentration between the electrodes, or an ablation that is contained to a narrower path between the electrodes as compared to electrodes placed at a distance from one another that does not concentrate current density between the electrodes. Both active electrodes are similar in size, or at least similar enough that current density in tissue around both active electrodes is high enough to thermally ablate the tissue. The disclosure above includes embodiments for carotid body modulation using bipolar RF ablation with two electrodes applied across a carotid septum. FIGS. 56A and 56B are schematic diagrams of bipolar RF carotid body modulation, which are described in detail herein. Some of these embodiments describe a form of bipolar ablation where both electrodes 1015 are substantially similar in size and create a similar localized current density and thermal ablation zone and are positioned a distance relative to one another sufficient to concentrate current density in the tissue (e.g., carotid septum 114) between the electrodes 1015. Both electrodes are generally required to be in apposition to tissue in order to create resistive heating below the blood vessel surface. In these bipolar RF embodiments there is no reference electrode with low localized current density as in typical monopolar RF ablation. Compared to a monopolar embodiment as shown in FIGS. 55A and 55B, the bipolar embodiment schematically shown in FIGS. 56A and 56B creates an ablation zone 1016 that stretches, or extends, across the septum from electrode to electrode and is contained within the septum spreading less cranially or laterally.

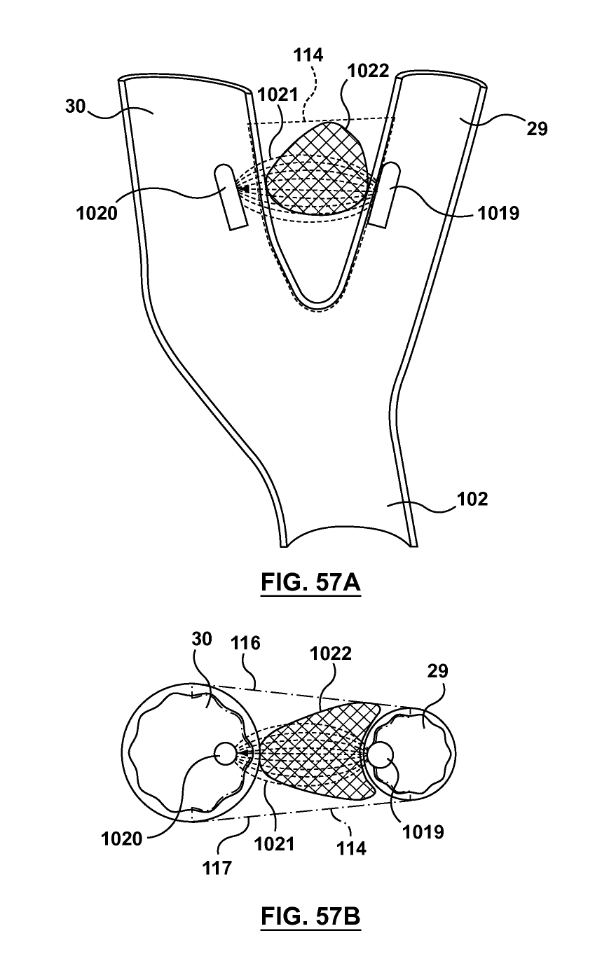

FIGS. 57A and 57B illustrate schematically an exemplary embodiment of energy-directed ablation of a carotid body. As shown in FIGS. 57A and 57B, energy-directed carotid body ablation comprises an ablation electrode 1019 placed in an external carotid artery 29 in contact with the vessel wall, in a similar way to the monopolar ablation example. However, placement, function and design of the reference electrode are different than in monopolar ablation. By placing a reference electrode 1020 in the internal carotid artery 30, a direct current path is created between two electrodes that crosses, or passes through, the carotid septum 114. The electric field 1021 is less dispersed than in monopolar ablation, and resistive heating occurs substantially along the electric current or energy deposition path connecting the two electrodes in a substantially straight line. In addition, as tissue along the current path starts to heat up, its impedance drops. Since current follows the path of lowest impedance, higher current density is maintained inside the carotid septum 114 and more energy is deposited at the target. The reference electrode 1020 may not need to be in full apposition, or electric or thermal contact, with the internal wall of the internal carotid artery 30 to complete the directed current return path across the septum. This configuration can have advantages. By positioning a reference electrode 1020 in the internal carotid artery 30, rather than on the patient's skin as in monopolar ablation, a resulting ablation lesion 1022 may be more contained inside the carotid septum 114 and its shape and volume are more influenced by the relative position of electrodes, amount of applied energy, and less by the steering influences of blood vessels that oppose the convective heating by cooling effects of blood flow. In experiments using animals, conducted by the authors, energy-directed ablation produced lesions that were much more repeatable in size and volume and generally contained within the carotid septum, having a larger volume biased towards the external carotid artery and ablation electrode and with less lateral spread beyond the lateral 117 and medial 116 boundaries of the carotid septum 114. The reduction in lateral spread beyond the lateral and medial boundaries of the carotid septum can help reduce the risk of damaging non-target tissue in those regions.

In a similar fashion to monopolar ablation, some embodiments of energy-directed ablation require only one active electrode to be in direct apposition with the wall of the vessel and associated with a high current density region in proximate tissue and resistive heating, and a reference electrode that serves to close the current return path. Unlike monopolar ablation, however, the energy-directed reference electrode is placed in a blood vessel (for example, in an internal carotid artery) and serves the additional function of directing or steering current in the desired direction, through the carotid septum. Furthermore, the energy-directed reference electrode need not have an extremely large surface area as in a skin patch to avoid a temperature increase. Heating of blood volume around an energy-directed reference electrode 1020 may be prevented or at least minimized by continuous strong blood flow that surrounds it. Compared to air, skin or bone, the impedance of blood and tissue that forms the carotid septum parenchyma is substantially similar (e.g., about 100 to about 300 ohms). This observation is important to understand the benefits on this approach. The impedance of the current path is therefore composed of a thin layer of blood and the volume of tissue in sequence. The total length of the path from an active electrode positioned in an external carotid artery to an energy-directed reference electrode placed in an internal carotid artery may be between about 3-10 mm. The presence of blood in the current path is counterintuitive and goes against tradition in the teaching of endovascular ablation.

Energy-directed RF carotid body ablation may comprise placement of an active electrode and an energy-directed reference electrode such that a target ablation tissue is between the two electrodes and that they are sufficiently close to one another such that the field and resulting ablation zone is influenced to be preferentially contained in the space between them. For example, an active electrode may be placed in an external carotid artery and a corresponding energy-directed reference electrode may be placed in an internal carotid artery. A potential benefit of this arrangement may be to reduce mechanical impact in the internal carotid artery to reduce a potential risk of dislodging plaque and causing a brain embolism. In some embodiments an active electrode is placed in an internal carotid artery and an energy-directed reference electrode is placed in an external carotid artery. Another example comprises placing an active electrode in an internal jugular vein and an energy-directed reference electrode in an external carotid artery. The arrangement may beneficially reduce embolic risk by avoiding the internal carotid artery all together. Furthermore, this arrangement may beneficially allow a catheter of a smaller diameter to be used for the reference electrode, which could be particularly important for a radial artery access catheter or temporal artery access catheter since the radial and temporal arteries are narrow (e.g., 3-5 mm diameter).

### Unexpected Discoveries

In the traditional teachings of endovascular, and especially cardiac, RF ablation, trapping a layer of blood between the electrode and the wall of the vessel is considered a safety risk. It is considered a risk because in traditional ablation delivered power is generally maximized until it is close to a safe limit for the electrode size in order to create a bigger, deeper lesion. Blood flow and velocity near the wall and the electrode is typically relatively low, and the temperature of the electrode is generally brought close to the safe limit. Thus, a thin conductive layer of blood between the electrode and wall can heat up beyond the safe level, which can lead to clot formation. In an effort to prevent heating of blood and clot formation, RF ablation with saline irrigated catheters became popular. Irrigated catheters are, however, more complex, having larger sizes and requiring an external saline pump. Additionally, irrigated catheters cannot take advantage of electrode temperature measurement to control or monitor tissue ablation.

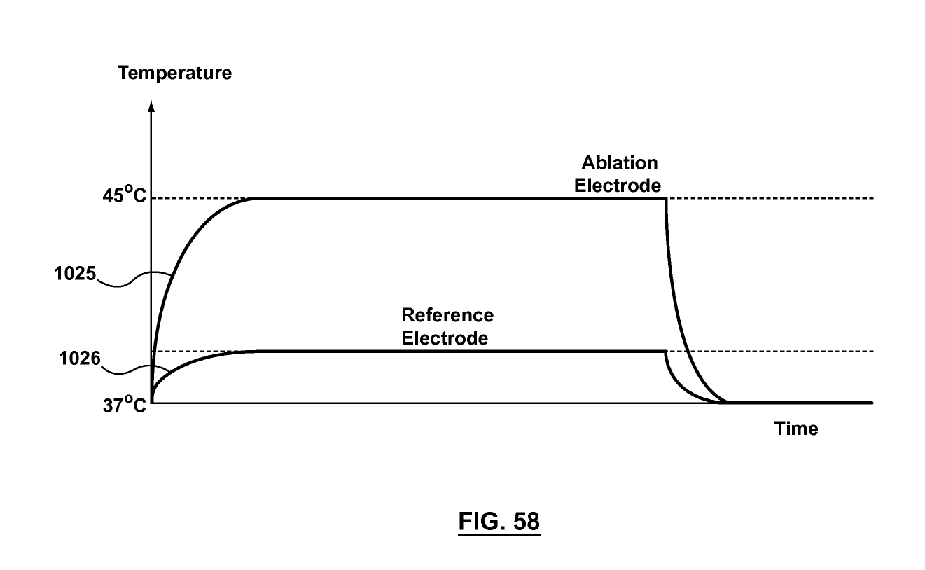

One or more inventors have conducted animal studies to understand the extent of the risk of clotting using bipolar ablation with a custom catheter. During these studies one electrode was placed in good apposition on one side of a carotid septum, and a second electrode was placed on the other side of the carotid septum and was intentionally not contacting the wall of the vessel in which it was placed. There were some directed and consistent lesions contained in the carotid septum without clotting of blood. FIG. 58 shows a graph that illustrates important observations made during studies. It is also applicable to methods to control and monitor ablation in clinical practice. Two traces represent temperature rise inside two electrodes during the unexpected energy-directed ablation studies described above. The ablation, or active, electrode, when positioned in an external carotid artery, exhibited a temperature rise 1025 during an application of 6 watts of RF power that is consistent with bipolar ablation under the same conditions. Since during RF ablation there is no resistive heating of the electrode itself, its impedance being negligible, the electrode temperature rise above the blood ambient temperature of 37° C. to 42-48° C. can be attributed solely to the conduction of heat transferred back from the resistive heating of carotid septum tissue. In contrast, the reference electrode was not in substantive contact with tissue. This is confirmed by the barely noticeable temperature rise 1026 of 2-3° C. This experiment also confirms that there is no dangerous heating of the thin layer of blood separating the reference electrode from the vessel wall. At the same time, post experiment histology of extracted tissue confirmed that the lesion was spanning the space between internal and external carotids, traversing the septum, consistent with the theory of directed current and contained RF field explained above. It was shown that energy-directed ablation as described herein may be used to achieve the therapeutic effects as described herein. While it is understood that embodiments above in which contact is made by electrodes with the septal wall in the external and internal carotid arteries may be able to more consistently create ablations contained within the carotid septum (and avoid ablating important non-target tissue) and may thus be in general more desirable approaches, there may be some instances, such as those described herein, in which an energy-directed approach could be beneficially used.

The described energy-directed ablation has potential advantages over monopolar ablation in that: (a) it can direct and contain heating and ablation of a carotid septum in the desired volume and (b) it does not require an external reference electrode, and that the same or similar size and volume lesion can be achieved at lower power and electrode temperature. Furthermore, energy-directed ablation has a potential advantage over bipolar ablation in that it minimizes, and can even eliminate, contact with the surface of an internal carotid artery. Generally good apposition with the arterial wall is achieved by mechanical pressure, which can potentially lead to disruption of plaque that may be present in an internal carotid artery, and damage to the vessel. Additionally, it may be difficult to achieve good simultaneous apposition in both internal and external carotids in some individuals with complex anatomy.

### Embodiments of Energy-Directed Carotid Body Ablation Catheters:

Devices have been conceived for endovascular carotid body modulation comprising energy-directed ablation catheters. Embodiments of catheters disclosed herein comprise a distal end and a proximal end, wherein the distal end is inserted into a patient's vasculature and delivered proximate a target site, and the proximal end is maintained outside the patient's body.

The distal region of an energy-directed ablation catheter comprises an active electrode positioned on a first spline and an energy-directed reference electrode on a second spline in a configuration that positions the active electrode in an external carotid artery on an intercarotid septum at a position relative to a target ablation site (e.g., carotid body or nerves associated with a carotid body) that is suitable for carotid body modulation, and the energy-directed reference electrode in an internal carotid artery at a position not necessarily in contact with the carotid septum but in a position relative to the active electrode sufficient to direct and concentrate an applied current path through the septum.

In some embodiments the catheter is configured so that the reference electrode is not in contact with the internal carotid artery. In some embodiments neither electrode is in contact with a wall of the artery in which it is positioned. Splines, as used herein, can also be referred to as arms, fingers, prongs, together as forceps arms, or individually as a forceps arm.

Any of the catheters in any of the embodiments described above in which both electrodes are configured to be in contact with a carotid artery wall in use can be modified to be configured such that one or both of the electrodes are not in contact with the vessel wall when in use (i.e., is configured for energy-directed ablation).

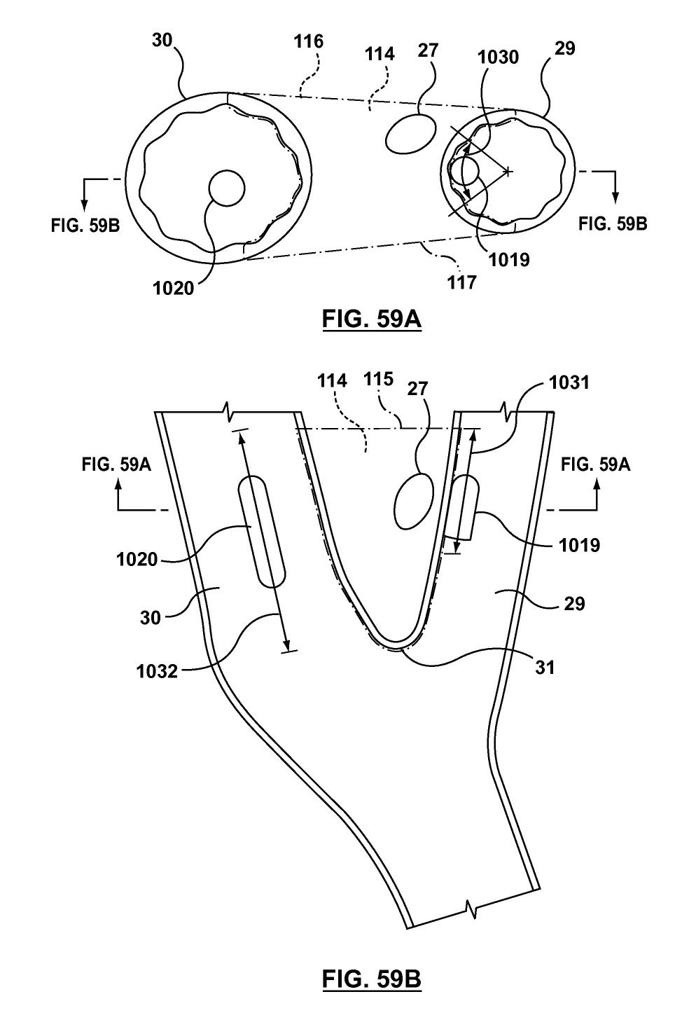

FIGS. 59A and 59B illustrate an example of active electrode 1019 and energy-directed reference electrode 1020 positioning relative to one another and to a carotid septum 114 that may effectively and safely ablate a carotid body 27. FIG. 59A shows, outlined with a dashed line, a transverse cross-section of an intercarotid septum 114 bordered by an internal carotid artery 30 and an external carotid artery 29. In this embodiment, an energy-directed reference electrode is placed in the internal carotid artery; an active electrode is placed in the external carotid artery in contact with the vessel wall within a vessel wall arc 1030 directed toward the internal carotid artery. The vessel wall arc 1030 is contained within limits of the intercarotid septum and comprises an arc length no greater than about 25% (e.g., about 15 to 25%) of the circumference of the vessel. Placement of ablation elements as described may facilitate targeted deposition of energy and the creation of an ablation lesion that is contained within the intercarotid septum 114, thus avoiding injury of non-target nerves that reside outside the septum, and an ablation that is sufficiently large (e.g., extending approximately from the internal carotid artery to the external carotid artery) to effectively ablate a carotid body or its associated nerves. Specifically this configuration facilitates deposition of energy substantially along a direct path between the electrodes. This controlled and selective ablation of septal tissue is described above with respect to the embodiments in which both electrodes are disposed in contact with the lumen walls while energy is delivered.

FIG. 59B shows, outlined with a dashed line, a longitudinal cross-section of an intercarotid septum 114 bordered by an internal carotid artery 30, an external carotid artery 29, a saddle of a carotid bifurcation 31 and a cranial (towards the head) boundary 115 that is between about 10 to 15 mm cranial from the saddle 31. In this example, the energy-directed reference electrode 1020 is placed in the internal carotid artery 30 within a first range 1032; active electrode 1019 is placed in the external carotid artery 29 in contact with the vessel wall within a second range 1031. The first range 1032 may extend from the inferior apex of the bifurcation saddle 31 to the cranial boundary 115 of the septum (e.g., about 10 to 15 mm from the bifurcation saddle). The second range 1031 may extend from a position about 4 mm superior from the bifurcation saddle 31 to the cranial boundary 115 of the septum (e.g., about 10 or 15 mm from the bifurcation saddle). As an example, a catheter may be configured to place a distal tip of an energy-directed reference electrode in an internal carotid artery about 10 mm from a carotid bifurcation and a distal tip of a 4 mm long active electrode in a corresponding external carotid artery at about 10 mm from the carotid bifurcation. The electrodes may be equidistant from the saddle 31 or they may be unequal distances from the saddle.

### Example Embodiments

FIG. 60 shows a distal region of an embodiment of a two-armed carotid body ablation catheter comprising a bipolar electrode on each of the two arms. A first arm 1041 is configured to place a first electrode 1042 in contact with a vessel wall (e.g., external carotid artery 29) on a carotid septum 114 in the suitable range 1031 and 1030 as shown on FIGS. 59A and 59B. A second arm 1043 is configured to place a second electrode 1044 in a vessel (e.g., internal carotid artery 30) but not in contact with the vessel wall. The two arms may be connected to a shaft of the catheter on or near the distal end 1045 so that when the distal end is abutted against the carotid bifurcation 31 the electrodes are placed at an appropriate height from the bifurcation. The shaft of the catheter may comprise a controllably deflectable section 1046 near the distal region, which may be used to press the first electrode 1042 into contact with the vessel wall. First arm 1041 may be configured as described above, such as an arm in the embodiment in FIG. 32A, and electrodes 1042 and 1044 can be any suitable electrode described herein.

FIG. 60 illustrates an endovascular carotid septum ablation catheter comprising first and second diverging arms with free distal ends, the first arm comprising an active ablation element configured to be in apposition with a septal wall of an external carotid artery, the second arm comprising a reference ablation element, the second arm configured to be simultaneously positioned within an internal carotid artery so that the reference ablation element is not in apposition with a wall of the internal carotid artery when the active ablation element is in contact with the septal wall, wherein the reference ablation element is configured to direct ablation energy from the active ablation element through the carotid septum to the reference ablation element

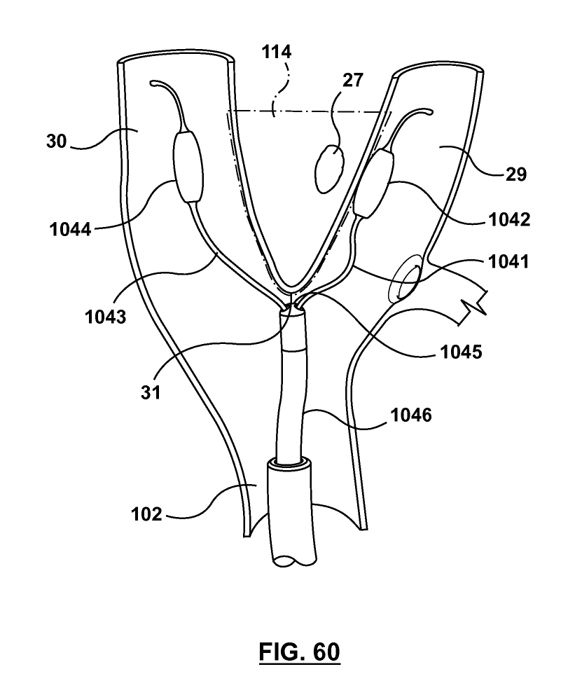

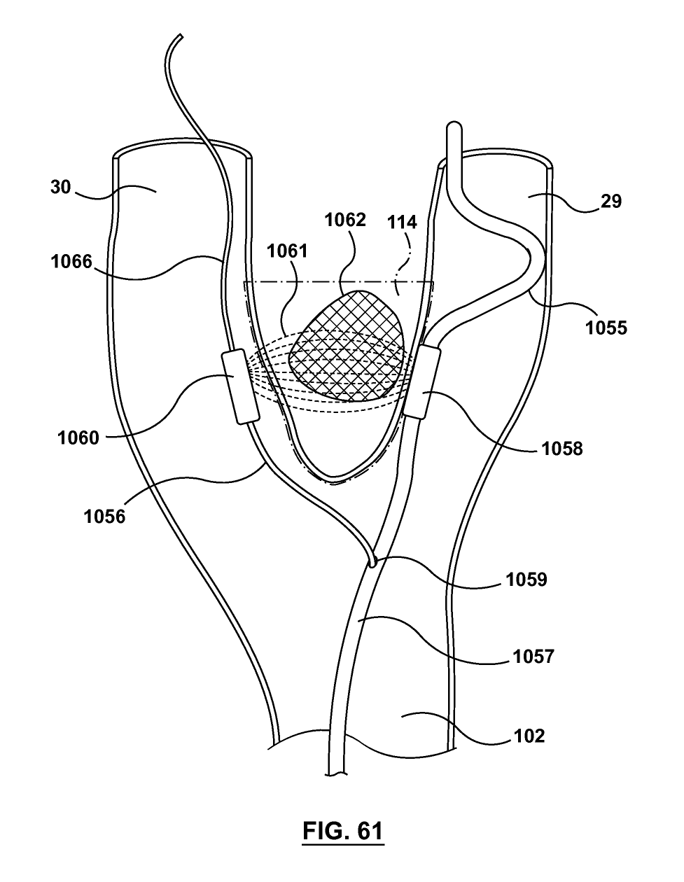

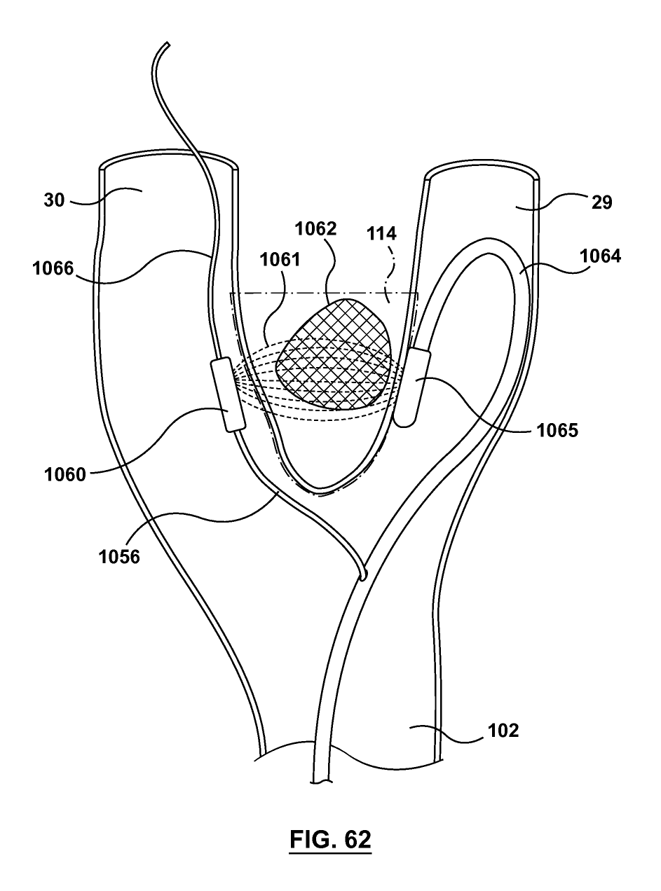

FIG. 61 and FIG. 62 show bifurcating catheters that include an external carotid prong and an internal carotid prong. The main resilient, load-bearing element of the system is, in this embodiment, the external carotid prong since external carotid intervention is not associated with a risk of brain embolization. The internal carotid prong can be telescoping out of the hollow shaft of the external prong. It is generally desired to make it less invasive and more atraumatic.

The embodiments in FIGS. 61 and 62 are similar conceptually to “keyed” embodiments herein, and can be modified in any manner with any of the components of the keyed embodiments to position and/or stabilize the active electrode in the external carotid artery and the reference electrode in the internal carotid artery. In the exemplary embodiment in FIG. 61, the ablation device includes elongate member 1057, on which active electrode 1058 is mounted. A distal region of elongate member 1057 on which ablation element 1058 is mounted is considered the first prong or arm 1055, and second arm or prong 1056 extends from the elongate member 1057. Elongate member 1057 includes a lumen therein configured to receive second arm 1056, and port 1059 in communication with the lumen, out of which second arm 1056 can pass from within elongate member 1057 to outside of elongate member 1057. Elongate member 1057 and second arm 1056 are configured such that when active electrode 1058 is in contact with external carotid artery 29 wall proximate the carotid septum 114, reference electrode 1060 is positioned in internal carotid artery 30 proximate the carotid septum 114. A region of elongate member 1057 distal to active electrode 1058 includes a stabilizing element 1055, configured to engage the external carotid artery wall and ensures pressure and apposition of the active electrode 1058 with the external carotid artery wall. Stabilizing element 1055 in this embodiment is a resilient element with a non-linear configuration configured to engage with an external carotid artery. Stabilizing element 1055 is configured so that it stabilizes the elongate member 1057 in a position so that port 1059 is oriented towards the internal carotid artery 30. When in the orientation, second arm 1056 can be advanced and the reference electrode 1060 is in position proximate the carotid septum 114 ready to direct the energy. Second arm 1056 can be any suitable elongate element configured to extend from elongate member 1057, such as a guide wire. Guide wire as used herein is not intended to be limited to a guide-wire as that term is commonly used in minimally invasive procedures, but rather it can be any suitable deployable elongate device. Alternatively, second arm 1056 can be secured to elongate member 1057, configured to be delivered within a delivery sheath substantially co-aligned with elongate member and have an at-rest extended configuration extending further radially away from elongate member 1057. In use, RF energy 1061 is passed from active electrode 1058 positioned in the external carotid artery 29 in contact with a vessel wall to reference electrode 1060 positioned in the internal carotid artery 30, which is not in contact with a vessel wall. The delivery of RF energy forms ablation region 1062 in the carotid septum 114.

FIG. 62 illustrates an exemplary carotid artery ablation catheter configured for energy-directed ablation. The primary difference between the embodiments in FIGS. 61 and 62 is the configuration of elongate member 1064 in FIG. 62. Other components in the two embodiments that have the same structure are labeled the same. In FIG. 62 elongate member 1064 includes a bend, wherein the configuration of the bend is sized such that it engages the external carotid artery and ensures pressure and apposition of the active electrode 1065 with the external carotid artery wall. The bend section in this embodiment bends back on itself, about 180 degrees.

One common element in the embodiment in FIGS. 13 and 14 is the atraumatic element 1066 at a distal end of the prong that resides in the internal carotid artery 30. This is the reference electrode prong that can be terminated in, for example, a J-tip wire or other wire, for example, an element that forms a soft curling leading edge to protect the vulnerable surface of the vessel when the element is advanced into the internal carotid artery.

An alternative embodiment of an ablation catheter 1070 shown in FIG. 15 employs a fluid filled balloon 1071 in an external carotid artery 29 intended to achieve apposition of the ablation electrode 1072 against the wall of the carotid septum 114. The electrode/balloon assembly can be similarly constructed as the balloon/electrode assembly in FIG. 42 above. Techniques are known how to mount electrodes on the surface of inflatable balloons. The fluid inside the balloon (e.g., cold saline) may be capable of absorbing the thermal energy conducted through the vessel wall from the resistive heating and cool the vessel wall sufficiently to maintain electrode temperature in the acceptable range. Alternatively the balloon may be perfused with a continuous flow of coolant for a duration of RF delivery. Like components are labeled the same as those from FIGS. 61 and 62.

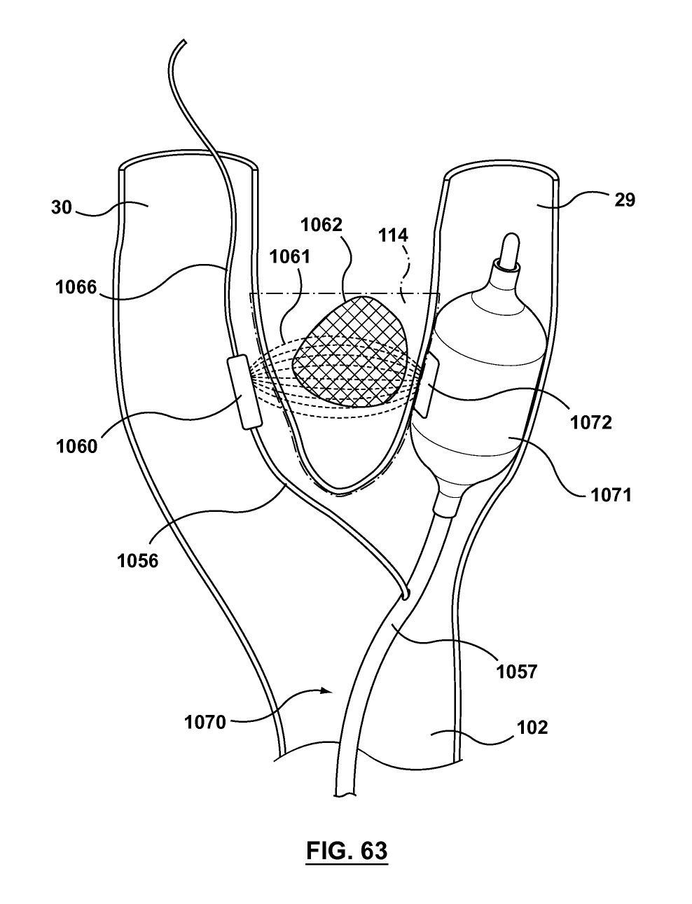

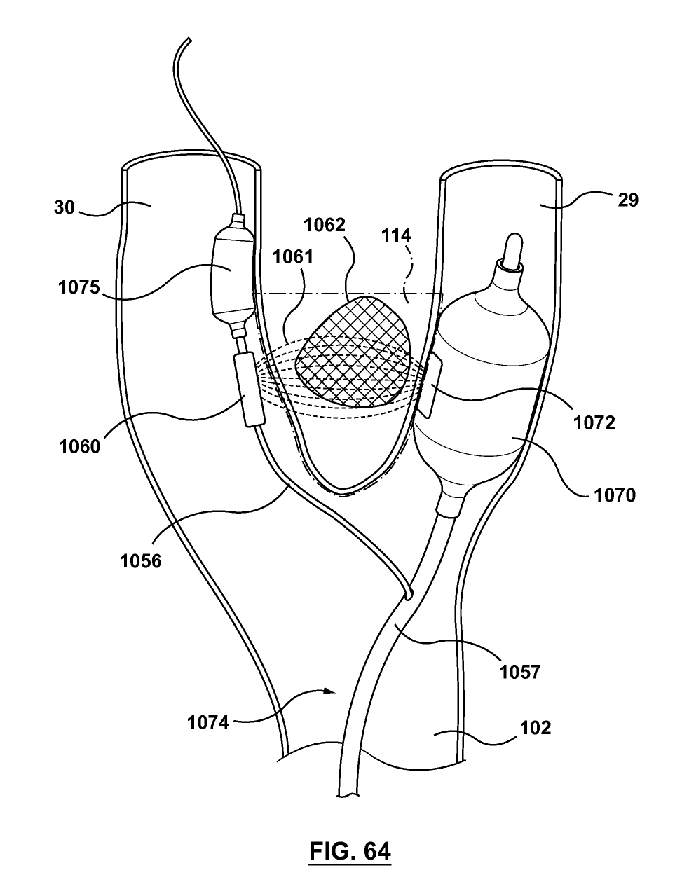

Another exemplary embodiment of a catheter configured for energy-directed ablation is shown in FIG. 64. Components similar to those in the embodiments in FIGS. 61 to 63 are labeled the same. As shown, catheter 1074 further comprises an atraumatic element 1075 in the form of a small floating balloon on the prong that comprises a reference electrode 1060. The atraumatic balloon may be relatively free floating in the blood stream inside the internal carotid artery 30 barely ever touching walls or significantly reducing blood flow. It may be made of a soft compliant material such as silicone or urethane. Its function is to center and align the reference electrode and prevent hard metal parts from coming in contact with the walls of internal carotid artery. Optionally a guide wire with soft tip can be threaded through both internal and external carotid prongs to facilitate advancement and placement of elements of the ablation system.

Another embodiment, not shown, comprises an active electrode, which may be placed in an external carotid artery, and an energy-directed reference electrode that is configured to be an embolic protection device such as a deployable net, which may be placed in an internal carotid artery and function both as a reference electrode and to catch any dislodged plaque in the blood stream flowing through the internal carotid artery, reducing embolic risk.

### Ablation Elements

Ablation elements may be electrodes configured for radiofrequency ablation. Embodiments of the present disclosure may comprise an active electrode, for example, with a surface area in a range of about 8 to 65 mm2 (e.g., about 12 to 17 mm2). For example, electrodes may be cylindrical with a hemispherical domed end having a circumference of about 0.8 to 2 mm (e.g., about 1.2 mm) and a length of about 3 to 10 mm (e.g., about 4 mm). A radiofrequency signal delivered to such electrodes may have a frequency in a range of about 300 to 500 kHz and a maximum power of about 12 W (e.g., a maximum power of about 5 W, 6 W, 7 W, 8 W, 9 W, 10 W, 11 W, or 12 W) and a duration of about 30 to 120 seconds (e.g., about 30 s). Electrodes may be made (e.g., machined) from an electrically conductive material such as stainless steel, copper, gold, platinum-iridium, or alloy such as 90% Au 10% Pt. For example, electrodes may be machined in a shape of a circular cylinder with hemispherical domed end with a hollow cavity, which may be used to position sensors (e.g., temperature sensor, impedance sensor), connect to structural segments of carotid prongs, or for cooling irrigation. Other shapes may be used for electrodes such as elliptical cylinder, cuboids, ribbon or complex shapes. Alternatively, any of the ablation elements described above can be incorporated into a catheter configured for energy-directed ablation.

### Methods of Therapy:

A method of using an ETAP catheter with having opening or closing, and deflection actuation may include the following steps:

1. Deliver a sheath (e.g., a 7 French compatible sheath) to a common carotid artery. An over the wire technique or fluoroscopic guidance may be used to deliver a sheath.

2. Deliver the ETAP catheter through the sheath to a common carotid artery. Optionally, the ETAP catheter may be connected to a console to test functionality of the catheter prior to delivering into the patient. For example, electrical current may be delivered through electrical conductors to check if all circuits are functioning properly and sensors, if any, are making reasonable measurements.

3. Deploy a distal working end of the ETAP catheter from the sheath in a closed configuration in the common carotid artery. If the ETAP catheter has a normally-open design the arms may be held in a closed configuration. For example an open/close actuator may be locked in a closed position.

4. Visualize position and rotational plane of the closed arms with respect to a carotid septum. Fluoroscopic techniques may be used to facilitate visualization. For example, contrast solution may be injected through the sheath into the common carotid artery to visualize the vasculature system and radiopaque markers may be placed on the catheter (e.g., on ablation elements and shaft).

5. Rotate/torque the ETAP catheter so arms are approximately in plane with a plane created by the axes of the internal and external carotid arteries.

6. Deflect the distal end of the ETAP catheter with a deflection actuator to aim the distal tip of the catheter at the carotid bifurcation. (note deflection plane is parallel with arms plane) An ETAP catheter configured without controllable deflection may be aimed at a carotid bifurcation using a deflectable sheath.

7. Open the arms with the open/close actuator. An ETAP catheter may be configured to open and close completely, that is, to its full range, upon actuation. Alternatively, an ETAP catheter may be configured to control variable position of the arms from fully open to fully closed. Variable position control may facilitate placement of electrodes, for example, in vasculature have a small bifurcation angle (e.g., less than about 15 degrees).

8. Advance open arms over a septum. The arms may be advanced until the bifurcation of the arms couples with the carotid bifurcation or carina. This may be indicated visually via fluoroscopy, through tactile feedback as a user feels the catheter meeting resistance, or by a contact or force sensor positioned on the distal end of the catheter. Alternatively, arms may be advanced partially, that is, before contact between the bifurcation of the arms and the carotid bifurcation made, for example as indicated visually via fluoroscopy. Partial advancement may be desired if a location of a carotid body or non-target nerves within a septum are known and a desired ablation zone is closer to the carina compared to an ablation zone created when arms are fully advanced. Furthermore, partial advancement may be desired to reduce risk of dislodging plaque that may exist at the carotid bifurcation.

9. Close the arms with the open/close actuator to bring ablation elements (e.g., RF electrodes, electroporation electrodes) into apposition with the septum. Actuation to close the arms may be fully actuated. Elasticity in elastic structural members of the arms may allow closed arms to adjust automatically to various septum thicknesses within a range (e.g., between 2 mm and 15 mm thick or between 4 mm and 10 mm thick) while applying approximately consistent electrode contact force. Alternatively, the degree of closing of the arms may variably controlled, for example, depending on septum thickness or electrode contact force, which may be indicated visually via fluoroscopy or with sensors (e.g., force or impedance sensors). An ETAP catheter may be configured to have arms that are substantially rigid, instead of elastic, so a closing force created by an open/close actuator causes the arms or ablation elements to squeeze an intercarotid septum. This may be advantageous, for example to decrease distance between ablation elements especially when a septum is thick (e.g., greater than 15 mm), which may improve the ability to create an effective ablation.

10. Run an ablation algorithm. For example, an ablation algorithm may be executed by a computerized console and may involve monitoring impedance and temperature, apply ablation energy (e.g., RF or irreversible electroporation) for a predetermined duration and at a predetermined power, shutting off ablation energy if an unwanted scenario occurs such as sudden rise in impedance, sudden large change in temperature, or physiological incidence.

11. Following ablation, open the arms with the open/close actuator to release electrode contact.

12. Retract the arms from the septum into the common carotid artery, for example by pulling the proximal end of the catheter out approximately 2 cm.

13. Close the arms with the open/close actuator. Alternatively, the arms may automatically close when the ETAP catheter is pulled into the sheath.

14. Collect the distal region of the ETAP catheter in the sheath.

15. Remove the sheath and ETAP catheter from the body. Alternatively or optionally, move the sheath and ETAP catheter to the patient's other side to perform a CBM procedure on the contralateral side. This may involve retracting the sheath into the aorta, optionally removing the ETAP catheter from the sheath, introducing a guide wire into the second common carotid artery, and repeating steps for placing the ETAP catheter and ablating.

An ablation energy source (e.g., energy field generator) may be located external to the patient. Various types of ablation energy generators or supplies, such as electrical frequency generators, ultrasonic generators, microwave generators, laser consoles, and heating or cryogenic fluid supplies, may be used to provide energy to the ablation element at the distal tip of the catheter. An electrode or other energy applicator at the distal tip of the catheter should conform to the type of energy generator coupled to the catheter. The generator may include computer controls to automatically or manually adjust frequency and strength of the energy applied to the catheter, timing and period during which energy is applied, and safety limits to the application of energy. It should be understood that embodiments of energy delivery electrodes described hereinafter may be electrically connected to the generator even though the generator is not explicitly shown or described with each embodiment.

An ablated tissue lesion at or near the carotid body may be created by the application of ablation energy from an ablation element in a vicinity of a distal end of the carotid body ablation device. The ablated tissue lesion may disable the carotid body or may suppress the activity of the carotid body or interrupt conduction of afferent nerve signals from a carotid body to sympathetic nervous system. The disabling or suppression of the carotid body reduces the responsiveness of the glomus cells to changes of blood gas composition and effectively reduces activity of afferent carotid body nerves or the chemoreflex gain of the patient.

A method in accordance with a particular embodiment includes ablating at least one of a patient's carotid bodies based at least in part on identifying the patient as having a sympathetically mediated disease such as cardiac, metabolic, or pulmonary disease such as hypertension, insulin resistance, diabetes, pulmonary hypertension, drug resistant hypertension (e.g., refractory hypertension), congestive heart failure (CHF), or dyspnea from heart failure or pulmonary disease causes.

A procedure may include diagnosis, selection based on diagnosis, further screening (e.g., baseline assessment of chemosensitivity), treating a patient based at least in part on diagnosis or further screening via a chemoreceptor (e.g., carotid body) ablation procedure such as one of the embodiments disclosed. Additionally, following ablation a method of therapy may involve conducting a post-ablation assessment to compare with the baseline assessment and making decisions based on the assessment (e.g., adjustment of drug therapy, re-treat in new position or with different parameters, or ablate a second chemoreceptor if only one was previously ablated).

A carotid body ablation procedure may comprise the following steps or a combination thereof: patient sedation, locating a target peripheral chemoreceptor, visualizing a target peripheral chemoreceptor (e.g., carotid body), confirming a target ablation site is or is proximate a peripheral chemoreceptor, confirming a target ablation site is safely distant from important non-target nerve structures that are preferably protected (e.g., hypoglossal, sympathetic and vagus nerves), providing stimulation (e.g., electrical, mechanical, chemical) to a target site or target peripheral chemoreceptor prior to, during or following an ablation step, monitoring physiological responses to said stimulation, providing temporary nerve block to a target site prior to an ablation step, monitoring physiological responses to said temporary nerve block, anesthetizing a target site, protecting the brain from potential embolism, thermally protecting an arterial or venous wall (e.g., carotid artery, jugular vein) or a medial aspect of an intercarotid septum or non-target nerve structures, ablating a target site (e.g., peripheral chemoreceptor), monitoring ablation parameters (e.g., temperature, pressure, duration, blood flow in a carotid artery), monitoring physiological responses during ablation and arresting ablation if unsafe or unwanted physiological responses occur before collateral nerve injury becomes permanent, confirming a reduction of chemoreceptor activity (e.g., chemosensitivity, HR, blood pressure, ventilation, sympathetic nerve activity) during or following an ablation step, removing a ablation device, conducting a post-ablation assessment, repeating any steps of the chemoreceptor ablation procedure on another peripheral chemoreceptor in the patient. Patient screening, as well as post-ablation assessment may include physiological tests or gathering of information, for example, chemoreflex sensitivity, central sympathetic nerve activity, heart rate, heart rate variability, blood pressure, ventilation, production of hormones, peripheral vascular resistance, blood pH, blood PCO2, degree of hyperventilation, peak VO2, VE/VCO2 slope. Directly measured maximum oxygen uptake (more correctly pVO2 in heart failure patients) and index of respiratory efficiency VE/VCO2 slope has been shown to be a reproducible marker of exercise tolerance in heart failure and provide objective and additional information regarding a patient's clinical status and prognosis.

A method of therapy may include electrical stimulation of a target region, using a stimulation electrode, to confirm proximity to a carotid body. For example, a stimulation signal having a 1-10 milliamps (mA) pulse train at about 20 to 40 Hz with a pulse duration of 50 to 500 microseconds (μs) that produces a positive carotid body stimulation effect may indicate that the stimulation electrode is within sufficient proximity to the carotid body or nerves of the carotid body to effectively ablate it. A positive carotid body stimulation effect could be increased blood pressure, heart rate, or ventilation concomitant with application of the stimulation. These variables could be monitored, recorded, or displayed to help assess confirmation of proximity to a carotid body. A catheter-based technique, for example, may have a stimulation electrode proximal to the ablation element used for ablation. Alternatively, the ablation element itself may also be used as a stimulation electrode. Alternatively, an energy delivery element that delivers a form of ablative energy that is not electrical, such as a cryogenic ablation applicator, may be configured to also deliver an electrical stimulation signal as described earlier. Yet another alternative embodiment comprises a stimulation electrode that is distinct from an ablation element. For example, during a surgical procedure a stimulation probe can be touched to a suspected carotid body that is surgically exposed. A positive carotid body stimulation effect could confirm that the suspected structure is a carotid body and ablation can commence. Physiological monitors (e.g., heart rate monitor, blood pressure monitor, blood flow monitor, MSNA monitor) may communicate with a computerized stimulation generator, which may also be an ablation generator, to provide feedback information in response to stimulation. If a physiological response correlates to a given stimulation the computerized generator may provide an indication of a positive confirmation.

Alternatively or in addition a drug known to excite the chemo sensitive cells of the carotid body can be injected directly into the carotid artery or given systemically into patients vein or artery in order to elicit hemodynamic or respiratory response. Examples of drugs that may excite a chemoreceptor include nicotine, atropine, Doxapram, Almitrine, hyperkalemia, Theophylline, adenosine, sulfides, Lobeline, Acetylcholine, ammonium chloride, methylamine, potassium chloride, anabasine, coniine, cytosine, acetaldehyde, acetyl ester and the ethyl ether of i-methylcholine, Succinylcholine, Piperidine, monophenol ester of homo-iso-muscarine and acetylsalicylamides, alkaloids of veratrum, sodium citrate, adenosinetriphosphate, dinitrophenol, caffeine, theobromine, ethyl alcohol, ether, chloroform, phenyldiguanide, sparteine, coramine(nikethamide), metrazol(pentylenetetrazol), iodomethylate of dimethylaminomethylenedioxypropane, ethyltrimethylammoniumpropane, trimethylammonium, hydroxytryptamine, papaverine, neostigmine, acidity.

A method of therapy may further comprise applying electrical or chemical stimulation to the target area or systemically following ablation to confirm a successful ablation. Heart rate, blood pressure or ventilation may be monitored for change or compared to the reaction to stimulation prior to ablation to assess if the targeted carotid body was ablated. Post-ablation stimulation may be done with the same apparatus used to conduct the pre-ablation stimulation. Physiological monitors (e.g., heart rate monitor, blood pressure monitor, blood flow monitor, MSNA monitor) may communicate with a computerized stimulation generator, which may also be an ablation generator, to provide feedback information in response to stimulation. If a physiological response correlated to a given stimulation is reduced following an ablation compared to a physiological response prior to the ablation, the computerized generator may provide an indication ablation efficacy or possible procedural suggestions such as repeating an ablation, adjusting ablation parameters, changing position, ablating another carotid body or chemosensor, or concluding the procedure.

The devices described herein may also be used to temporarily stun or block nerve conduction via electrical neural blockade. A temporary nerve block may be used to confirm position of an ablation element prior to ablation. For example, a temporary nerve block may block nerves associated with a carotid body, which may result in a physiological effect to confirm the position may be effective for ablation. Furthermore, a temporary nerve block may block important non-target nerves such as vagal, hypoglossal or sympathetic nerves that are preferably avoided, resulting in a physiological effect (e.g., physiological effects may be noted by observing the patient's eyes, tongue, throat or facial muscles or by monitoring patient's heart rate and respiration). This may alert a user that the position is not in a safe location. Likewise absence of a physiological effect indicating a temporary nerve block of such important non-target nerves in combination with a physiological effect indicating a temporary nerve block of carotid body nerves may indicate that the position is in a safe and effective location for carotid body ablation.

Important nerves may be located in proximity of the target site and may be inadvertently and unintentionally injured. Neural stimulation or blockade can help identify that these nerves are in the ablation zone before the irreversible ablation occurs. These nerves may include the following:

Vagus Nerve Bundle—The vagus is a bundle of nerves that carry separate functions, for example a) branchial motor neurons (efferent special visceral) which are responsible for swallowing and phonation and are distributed to pharyngeal branches, superior and inferior laryngeal nerves; b) visceral motor (efferent general visceral) which are responsible for involuntary muscle and gland control and are distributed to cardiac, pulmonary, esophageal, gastric, celiac plexuses, and muscles, and glands of the digestive tract; c) visceral sensory (afferent general visceral) which are responsible for visceral sensibility and are distributed to cervical, thoracic, abdominal fibers, and carotid and aortic bodies; d) visceral sensory (afferent special visceral) which are responsible for taste and are distributed to epiglottis and taste buds; e) general sensory (afferent general somatic) which are responsible for cutaneous sensibility and are distributed to auricular branch to external ear, meatus, and tympanic membrane. Dysfunction of the vagus may be detected by a) vocal changes caused by nerve damage (damage to the vagus nerve can result in trouble with moving the tongue while speaking, or hoarseness of the voice if the branch leading to the larynx is damaged); b) dysphagia due to nerve damage (the vagus nerve controls many muscles in the palate and tongue which, if damaged, can cause difficulty with swallowing); c) changes in gag reflex (the gag reflex is controlled by the vagus nerve and damage may cause this reflex to be lost, which can increase the risk of choking on saliva or food); d) hearing loss due to nerve damage (hearing loss may result from damage to the branch of the vagus nerve that innervates the concha of the ear): e) cardiovascular problems due to nerve damage (damage to the vagus nerve can cause cardiovascular side effects including irregular heartbeat and arrhythmia); or f) digestive problems due to nerve damage (damage to the vagus nerve may cause problems with contractions of the stomach and intestines, which can lead to constipation).

Superior Laryngeal Nerve—the superior laryngeal nerve is a branch of the vagus nerve bundle. Functionally, the superior laryngeal nerve function can be divided into sensory and motor components. The sensory function provides a variety of afferent signals from the supraglottic larynx. Motor function involves motor supply to the ipsilateral cricothyroid muscle. Contraction of the cricothyroid muscle tilts the cricoid lamina backward at the cricothyroid joint causing lengthening, tensing and adduction of vocal folds causing an increase in the pitch of the voice generated. Dysfunction of the superior laryngeal nerve may change the pitch of the voice and causes an inability to make explosive sounds. A bilateral palsy presents as a tiring and hoarse voice.

Cervical Sympathetic Nerve—The cervical sympathetic nerve provides efferent fibers to the internal carotid nerve, external carotid nerve, and superior cervical cardiac nerve. It provides sympathetic innervation of the head, neck and heart. Organs that are innervated by the sympathetic nerves include eyes, lacrimal gland and salivary glands. Dysfunction of the cervical sympathetic nerve includes Homer's syndrome, which is very identifiable and may include the following reactions: a) partial ptosis (drooping of the upper eyelid from loss of sympathetic innervation to the superior tarsal muscle, also known as Müller's muscle); b) upside-down ptosis (slight elevation of the lower lid); c) anhidrosis (decreased sweating on the affected side of the face); d) miosis (small pupils, for example small relative to what would be expected by the amount of light the pupil receives or constriction of the pupil to a diameter of less than two millimeters, or asymmetric, one-sided constriction of pupils); e) enophthalmos (an impression that an eye is sunken in); f) loss of ciliospinal reflex (the ciliospinal reflex, or pupillary-skin reflex, consists of dilation of the ipsilateral pupil in response to pain applied to the neck, face, and upper trunk. If the right side of the neck is subjected to a painful stimulus, the right pupil dilates about 1-2 mm from baseline. This reflex is absent in Horner's syndrome and lesions involving the cervical sympathetic fibers.)

### Visualization:

An optional step of visualizing internal structures (e.g., carotid body or surrounding structures) may be accomplished using one or more non-invasive imaging modalities, for example fluoroscopy, radiography, arteriography, computer tomography (CT), computer tomography angiography with contrast (CTA), magnetic resonance imaging (MRI), or sonography, or minimally invasive techniques (e.g., IVUS, endoscopy, optical coherence tomography, ICE). A visualization step may be performed as part of a patient assessment, prior to an ablation procedure to assess risks and location of anatomical structures, during an ablation procedure to help guide an ablation device, or following an ablation procedure to assess outcome (e.g., efficacy of the ablation). Visualization may be used to: (a) locate a carotid body, (b) locate important non-target nerve structures that may be adversely affected, or (c) locate, identify and measure arterial plaque.

Endovascular (for example transfemoral) arteriography of the common carotid and then selective arteriography of the internal and external carotids may be used to determine a position of a catheter tip at a carotid bifurcation. Additionally, ostia of glomic arteries (these arteries may be up to 4 mm long and arise directly from the main parent artery) can be identified by dragging the dye injection catheter and releasing small amounts (“puffs”) of dye. If a glomic artery is identified it can be cannulated by a guide wire and possibly further cannulated by small caliber catheter. Direct injection of dye into glomic arteries can further assist the interventionalist in the ablation procedure. It is appreciated that the feeding glomic arteries are small and microcatheters may be needed to cannulate them.

Alternatively, ultrasound visualization may allow a physician to see the carotid arteries and even the carotid body. Another method for visualization may consist of inserting a small needle (e.g., 22 Gauge) with sonography or computer tomography (CT) guidance into or toward the carotid body. A wire or needle can be left in place as a fiducial guide, or contrast can be injected into the carotid body. Runoff of contrast to the jugular vein may confirm that the target is achieved.

Computer Tomography (CT) and computer tomography angiography (CTA) may also be used to aid in identifying a carotid body. Such imaging could be used to help guide an ablation device to a carotid body.

Ultrasound visualization (e.g., sonography) is an ultrasound-based imaging technique used for visualizing subcutaneous body structures including blood vessels and surrounding tissues. Doppler ultrasound uses reflected ultrasound waves to identify and display blood flow through a vessel. Operators typically use a hand-held transducer/transceiver placed directly on a patient's skin and aimed inward directing ultrasound waves through the patient's tissue. Ultrasound may be used to visualize a patient's carotid body to help guide an ablation device. Ultrasound can be also used to identify atherosclerotic plaque in the carotid arteries and avoid disturbing and dislodging such plaque.

Visualization and navigation steps may comprise multiple imaging modalities (e.g., CT, fluoroscopy, ultrasound) superimposed digitally to use as a map for instrument positioning. Superimposing borders of great vessels such as carotid arteries can be done to combine images.

Responses to stimulation at different coordinate points can be stored digitally as a 3-dimensional or 2-dimensional orthogonal plane map. Such an electric map of the carotid bifurcation showing points, or point coordinates that are electrically excitable such as baroreceptors, baroreceptor nerves, chemoreceptors and chemoreceptor nerves can be superimposed with an image (e.g., CT, fluoroscopy, ultrasound) of vessels. This can be used to guide the procedure, and identify target areas and areas to avoid.

In addition, as noted above, it should be understood that a device providing therapy can also be used to locate a carotid body as well as to provide various stimuli (electrical, chemical, other) to test a baseline response of the carotid body chemoreflex (CBC) or carotid sinus baroreflex (CSB) and measure changes in these responses after therapy or a need for additional therapy to achieve the desired physiological and clinical effects.

### Patient Selection and Assessment:

In an embodiment, a procedure may comprise assessing a patient to be a plausible candidate for carotid body ablation. Such assessment may involve diagnosing a patient with a sympathetically mediated disease (e.g., MSNA microneurography, measure of cataclomines in blood or urine, heart rate, or low/high frequency analysis of heart rate variability may be used to assess sympathetic tone). Patient assessment may further comprise other patient selection criteria, for example indices of high carotid body activity (i.e. carotid body hypersensitivity or hyperactivity) such as a combination of hyperventilation and hypocarbia at rest, high carotid body nerve activity (e.g., measured directly), incidence of periodic breathing, dyspnea, central sleep apnea elevated brain natriuretic peptide, low exercise capacity, having cardiac resynchronization therapy, atrial fibrillation, ejection fraction of the left ventricle, using beta blockers or ACE inhibitors.

Patient selection may involve non-invasive visualization such as CTA or MRI to identify location of a carotid body. For example, if the patient does not have at least one carotid body that is sufficiently within an intercarotid septum the patient may be ineligible for a CBM procedure that targets an intercarotid septum. Another example of patient selection using non-invasive visualization may involve excluding patients having large risk of dislodging plaque into an internal carotid artery.

Patient assessment may further involve selecting patients with high peripheral chemosensitivity (e.g., a respiratory response to hypoxia normalized to the desaturation of oxygen greater than or equal to about 0.7 l/min/min SpO₂), which may involve characterizing a patient's chemoreceptor sensitivity, reaction to temporarily blocking carotid body chemoreflex, or a combination thereof.

Although there are many ways to measure chemosensitivity they can be divided into (a) active provoked response and (b) passive monitoring. Active tests can be done by inducing intermittent hypoxia (such as by taking breaths of nitrogen or CO₂ or combination of gases) or by rebreathing air into and from a 4 to 10 liter bag. For example: a hypersensitive response to a short period of hypoxia measured by increase of respiration or heart rate may provide an indication for therapy. Ablation or significant reduction of such response could be indicative of a successful procedure. Also, electrical stimulation, drugs and chemicals (e.g., dopamine, lidocaine) exist that can block or excite a carotid body when applied locally or intravenously.

The location and baseline function of the desired area of therapy (including the carotid and aortic chemoreceptors and baroreceptors and corresponding nerves) may be determined prior to therapy by application of stimuli to the carotid body or other organs that would result in an expected change in a physiological or clinical event such as an increase or decrease in SNS activity, heart rate or blood pressure. These stimuli may also be applied after the therapy to determine the effect of the therapy or to indicate the need for repeated application of therapy to achieve the desired physiological or clinical effect(s). The stimuli can be either electrical or chemical in nature and can be delivered via the same or another catheter or can be delivered separately (such as injection of a substance through a peripheral IV to affect the CBC that would be expected to cause a predicted physiological or clinical effect).

A baseline stimulation test may be performed to select patients that may benefit from a carotid body ablation procedure. For example, patients with a high peripheral chemosensitivity gain (e.g., greater than or equal to about two standard deviations above an age matched general population chemosensitivity, or alternatively above a threshold peripheral chemosensitivity to hypoxia of 0.5 or 0.7 ml/min % O2) may be selected for a carotid body ablation procedure. A prospective patient suffering from a cardiac, metabolic, or pulmonary disease (e.g., hypertension, CHF, diabetes) may be selected. The patient may then be tested to assess a baseline peripheral chemoreceptor sensitivity (e.g., minute ventilation, tidal volume, ventilator rate, heart rate, or other response to hypoxic or hypercapnic stimulus). Baseline peripheral chemosensitivity may be assessed using tests known in the art which involve inhalation of a gas mixture having reduced O₂ content (e.g., pure nitrogen, CO₂, helium, or breathable gas mixture with reduced amounts of O₂ and increased amounts of CO₂) or rebreathing of gas into a bag. Concurrently, the patient's minute ventilation or initial sympathetically mediated physiologic parameter such as minute ventilation or HR may be measured and compared to the O₂ level in the gas mixture. Tests like this may elucidate indices called chemoreceptor setpoint and gain. These indices are indicative of chemoreceptor sensitivity. If the patient's chemosensitivity is not assessed to be high (e.g., less than about two standard deviations of an age matched general population chemosensitivity, or other relevant numeric threshold) then the patient may not be a suitable candidate for a carotid body ablation procedure. Conversely, a patient with chemoreceptor hypersensitivity (e.g., greater than or equal to about two standard deviations above normal) may proceed to have a carotid body ablation procedure. Following a carotid body ablation procedure the patient's chemosensitivity may optionally be tested again and compared to the results of the baseline test. The second test or the comparison of the second test to the baseline test may provide an indication of treatment success or suggest further intervention such as possible adjustment of drug therapy, repeating the carotid body ablation procedure with adjusted parameters or location, or performing another carotid body ablation procedure on a second carotid body if the first procedure only targeted one carotid body. It may be expected that a patient having chemoreceptor hypersensitivity or hyperactivity may return to about a normal sensitivity or activity following a successful carotid body ablation procedure.

In an alternative protocol for selecting a patient for a carotid body ablation, patients with high peripheral chemosensitivity or carotid body activity (e.g., ≧about 2 standard deviations above normal) alone or in combination with other clinical and physiologic parameters may be particularly good candidates for carotid body ablation therapy if they further respond positively to temporary blocking of carotid body activity. A prospective patient suffering from a cardiac, metabolic, or pulmonary disease may be selected to be tested to assess the baseline peripheral chemoreceptor sensitivity. A patient without high chemosensitivity may not be a plausible candidate for a carotid body ablation procedure. A patient with a high chemosensitivity may be given a further assessment that temporarily blocks a carotid body chemoreflex. For example a temporary block may be done chemically, for example using a chemical such as intravascular dopamine or dopamine-like substances, intravascular alpha-2 adrenergic agonists, oxygen, in general alkalinity, or local or topical application of atropine externally to the carotid body. A patient having a negative response to the temporary carotid body block test (e.g., sympathetic activity index such as respiration, HR, heart rate variability, MSNA, vasculature resistance, etc. is not significantly altered) may be a less plausible candidate for a carotid body ablation procedure. Conversely, a patient with a positive response to the temporary carotid body block test (e.g., respiration or index of sympathetic activity is altered significantly) may be a more plausible candidate for a carotid body ablation procedure.

There are a number of potential ways to conduct a temporary carotid body block test. Hyperoxia (e.g., higher than normal levels of PO₂) for example, is known to partially block (about a 50%) or reduce afferent sympathetic response of the carotid body. Thus, if a patient's sympathetic activity indexes (e.g., respiration, HR, HRV, MSNA) are reduced by hyperoxia (e.g., inhalation of higher than normal levels of O₂) for 3-5 minutes, the patient may be a particularly plausible candidate for carotid body ablation therapy. A sympathetic response to hyperoxia may be achieved by monitoring minute ventilation (e.g., reduction of more than 20-30% may indicate that a patient has carotid body hyperactivity). To evoke a carotid body response, or compare it to carotid body response in normoxic conditions, CO₂ above 3-4% may be mixed into the gas inspired by the patient (nitrogen content will be reduced) or another pharmacological agent can be used to invoke a carotid body response to a change of CO₂, pH or glucose concentration. Alternatively, “withdrawal of hypoxic drive” to rest state respiration in response to breathing a high concentration O₂ gas mix may be used for a simpler test.

An alternative temporary carotid body block test involves administering a sub-anesthetic amount of anesthetic gas halothane, which is known to temporarily suppress carotid body activity. Furthermore, there are injectable substances such as dopamine that are known to reversibly inhibit the carotid body. However, any substance, whether inhaled, injected or delivered by another manner to the carotid body that affects carotid body function in the desired fashion may be used.

Another alternative temporary carotid body block test involves application of cryogenic energy to a carotid body (i.e. removal of heat). For example, a carotid body or its nerves may be cooled to a temperature range between about −15° C. to 0° C. to temporarily reduce nerve activity or blood flow to and from a carotid body thus reducing or inhibiting carotid body activity.

An alternative method of assessing a temporary carotid body block test may involve measuring pulse pressure. Noninvasive pulse pressure devices such as Nexfin (made by BMEYE, based in Amsterdam, The Netherlands) can be used to track beat-to-beat changes in peripheral vascular resistance. Patients with hypertension or CHF may be sensitive to temporary carotid body blocking with oxygen or injection of a blocking drug. The peripheral vascular resistance of such patients may be expected to reduce substantially in response to carotid body blocking. Such patients may be good candidates for carotid body ablation therapy.

Yet another index that may be used to assess if a patient may be a good candidate for carotid body ablation therapy is increase of baroreflex, or baroreceptor sensitivity, in response to carotid body blocking. It is known that hyperactive chemosensitivity suppresses baroreflex. If carotid body activity is temporarily reduced the carotid sinus baroreflex (baroreflex sensitivity (BRS) or baroreflex gain) may be expected to increase. Baroreflex contributes a beneficial parasympathetic component to autonomic drive. Depressed BRS is often associated with an increased incidence of death and malignant ventricular arrhythmias. Baroreflex is measurable using standard non-invasive methods. One example is spectral analysis of RR interval of ECG and systolic blood pressure variability in both the high- and low-frequency bands. An increase of baroreflex gain in response to temporary blockade of carotid body can be a good indication for permanent therapy. Baroreflex sensitivity can also be measured by heart rate response to a transient rise in blood pressure induced by injection of phenylephrine.

An alternative method involves using an index of glucose tolerance to select patients and determine the results of carotid body blocking or removal in diabetic patients. There is evidence that carotid body hyperactivity contributes to progression and severity of metabolic disease.

In general, a beneficial response can be seen as an increase of parasympathetic or decrease of sympathetic tone in the overall autonomic balance. For example, Power Spectral Density (PSD) curves of respiration or HR can be calculated using nonparametric Fast Fourier Transform algorithm (FFT). FFT parameters can be set to 256-64 k buffer size, Hamming window, 50% overlap, 0 to 0.5 or 0.1 to 1.0 Hz range. HR and respiratory signals can be analyzed for the same periods of time corresponding to (1) normal unblocked carotid body breathing and (2) breathing with blocked carotid body.

Power can be calculated for three bands: the very low frequency (VLF) between 0 and 0.04 Hz, the low frequency band (LF) between 0.04-0.15 Hz and the high frequency band (HF) between 0.15-0.4 Hz. Cumulative spectral power in LF and HF bands may also be calculated; normalized to total power between 0.04 and 0.4 Hz (TF=HF+LF) and expressed as % of total. Natural breathing rate of CHF patient, for example, can be rather high, in the 0.3-0.4 Hz range.

The VLF band may be assumed to reflect periodic breathing frequency (typically 0.016 Hz) that can be present in CHF patients. It can be excluded from the HF/LF power ratio calculations.

The powers of the LF and HF oscillations characterizing heart rate variability (HRV) appear to reflect, in their reciprocal relationship, changes in the state of the sympathovagal (sympathetic to parasympathetic) balance occurring during numerous physiological and pathophysiological conditions. Thus, increase of HF contribution in particular can be considered a positive response to carotid body blocking.

Another alternative method of assessing carotid body activity comprises nuclear medicine scanning, for example with ocreotide, somatostatin analogues, or other substances produced or bound by the carotid body.

Furthermore, artificially increasing blood flow may reduce carotid body activation. Conversely artificially reducing blood flow may stimulate carotid body activation. This may be achieved with drugs know in the art to alter blood flow.

There is a considerable amount of scientific evidence to demonstrate that hypertrophy of a carotid body often accompanies disease. A hypertrophied (i.e. enlarged) carotid body may further contribute to the disease. Thus identification of patients with enlarged carotid bodies may be instrumental in determining candidates for therapy. Imaging of a carotid body may be accomplished by angiography performed with radiographic, computer tomography, or magnetic resonance imaging.

It should be understood that the available measurements are not limited to those described above. It may be possible to use any single or a combination of measurements that reflect any clinical or physiological parameter effected or changed by either increases or decreases in carotid body function to evaluate the baseline state, or change in state, of a patient's chemosensitivity.

There is a considerable amount of scientific evidence to demonstrate that hypertrophy of a carotid body often accompanies disease. A hypertrophied or enlarged carotid body may further contribute to the disease. Thus identification of patients with enlarged carotid bodies may be instrumental in determining candidates for therapy.

Further, it is possible that although patients do not meet a preselected clinical or physiological definition of high peripheral chemosensitivity (e.g., greater than or equal to about two standard deviations above normal), administration of a substance that suppresses peripheral chemosensitivity may be an alternative method of identifying a patient who is a candidate for the proposed therapy. These patients may have a different physiology or co-morbid disease state that, in concert with a higher than normal peripheral chemosensitivity (e.g., greater than or equal to normal and less than or equal to about 2 standard deviations above normal), may still allow the patient to benefit from carotid body ablation. The proposed therapy may be at least in part based on an objective that carotid body ablation will result in a clinically significant or clinically beneficial change in the patient's physiological or clinical course. It is reasonable to believe that if the desired clinical or physiological changes occur even in the absence of meeting the predefined screening criteria, then therapy could be performed.

### Physiology:

Ablation of a target ablation site (e.g., peripheral chemoreceptor, carotid body) via an endovascular approach in patients having sympathetically mediated disease and augmented chemoreflex (e.g., high afferent nerve signaling from a carotid body to the central nervous system as in some cases indicated by high peripheral chemosensitivity) has been conceived to reduce peripheral chemosensitivity and reduce afferent signaling from peripheral chemoreceptors to the central nervous system. The expected reduction of chemoreflex activity and sensitivity to hypoxia and other stimuli such as blood flow, blood CO₂, glucose concentration or blood pH can directly reduce afferent signals from chemoreceptors and produce at least one beneficial effect such as the reduction of central sympathetic activation, reduction of the sensation of breathlessness (dyspnea), vasodilation, increase of exercise capacity, reduction of blood pressure, reduction of sodium and water retention, redistribution of blood volume to skeletal muscle, reduction of insulin resistance, reduction of hyperventilation, reduction of tachypnea, reduction of hypocapnia, increase of baroreflex and barosensitivity of baroreceptors, increase of vagal tone, or improve symptoms of a sympathetically mediated disease and may ultimately slow down the disease progression and extend life. It is understood that a sympathetically mediated disease that may be treated with carotid body ablation may comprise elevated sympathetic tone, an elevated sympathetic/parasympathetic activity ratio, autonomic imbalance primarily attributable to central sympathetic tone being abnormally or undesirably high, or heightened sympathetic tone at least partially attributable to afferent excitation traceable to hypersensitivity or hyperactivity of a peripheral chemoreceptor (e.g., carotid body). In some important clinical cases where baseline hypocapnia or tachypnea is present, reduction of hyperventilation and breathing rate may be expected. It is understood that hyperventilation in the context herein means respiration in excess of metabolic needs on the individual that generally leads to slight but significant hypocapnea (blood CO₂ partial pressure below normal of approximately 40 mmHg, for example in the range of 33 to 38 mmHg).

Patients having CHF or hypertension concurrent with heightened peripheral chemoreflex activity and sensitivity often react as if their system was hypercapnic even if it is not. The reaction is to hyperventilate, a maladaptive attempt to rid the system of CO₂, thus overcompensating and creating a hypocapnic and alkalotic system. Some researchers attribute this hypersensitivity/hyperactivity of the carotid body to the direct effect of catecholamines, hormones circulating in excessive quantities in the blood stream of CHF patients. The procedure may be particularly useful to treat such patients who are hypocapnic and possibly alkalotic resulting from high tonic output from carotid bodies. Such patients are particularly predisposed to periodic breathing and central apnea hypopnea type events that cause arousal, disrupt sleep, cause intermittent hypoxia and are by themselves detrimental and difficult to treat.

It is appreciated that periodic breathing of Cheyne Stokes pattern occurs in patients during sleep, exercise and even at rest as a combination of central hypersensitivity to CO₂, peripheral chemosensitivity to O₂ and CO₂ and prolonged circulatory delay. All these parameters are often present in CHF patients that are at high risk of death. Thus, patients with hypocapnea, CHF, high chemosensitivity and prolonged circulatory delay, and specifically ones that exhibit periodic breathing at rest or during exercise or induced by hypoxia are likely beneficiaries of the proposed therapy.

Hyperventilation is defined as breathing in excess of a person's metabolic need at a given time and level of activity. Hyperventilation is more specifically defined as minute ventilation in excess of that needed to remove CO2 from blood in order to maintain blood CO₂ in the normal range (e.g., around 40 mmHg partial pressure). For example, patients with arterial blood PCO₂ in the range of 32-37 mmHg can be considered hypocapnic and in hyperventilation.

For the purpose of this disclosure hyperventilation is equivalent to abnormally low levels of carbon dioxide in the blood (e.g., hypocapnia, hypocapnea, or hypocarbia) caused by overbreathing. Hyperventilation is the opposite of hypoventilation (e.g., underventilation) that often occurs in patients with lung disease and results in high levels of carbon dioxide in the blood (e.g., hypercapnia or hypercarbia).

A low partial pressure of carbon dioxide in the blood causes alkalosis, because CO2 is acidic in solution and reduced CO2 makes blood pH more basic, leading to lowered plasma calcium ions and nerve and muscle excitability. This condition is undesirable in cardiac patients since it can increase probability of cardiac arrhythmias.

Alkalemia may be defined as abnormal alkalinity, or increased pH of the blood. Respiratory alkalosis is a state due to excess loss of carbon dioxide from the body, usually as a result of hyperventilation. Compensated alkalosis is a form in which compensatory mechanisms have returned the pH toward normal. For example, compensation can be achieved by increased excretion of bicarbonate by the kidneys.

Compensated alkalosis at rest can become uncompensated during exercise or as a result of other changes of metabolic balance. Thus the invented method is applicable to treatment of both uncompensated and compensated respiratory alkalosis.

Tachypnea means rapid breathing. For the purpose of this disclosure a breathing rate of about 6 to 16 breaths per minute at rest is considered normal but there is a known benefit to lower rate of breathing in cardiac patients. Reduction of tachypnea can be expected to reduce respiratory dead space, increase breathing efficiency, and increase parasympathetic tone.

Therapy Example: Role of Chemoreflex and Central Sympathetic Nerve Activity in CHF

Chronic elevation in sympathetic nerve activity (SNA) is associated with the development and progression of certain types of hypertension and contributes to the progression of congestive heart failure (CHF). It is also known that sympathetic excitatory cardiac, somatic, and central/peripheral chemoreceptor reflexes are abnormally enhanced in CHF and hypertension (Ponikowski, 2011 and Giannoni, 2008 and 2009).

Arterial chemoreceptors serve an important regulatory role in the control of alveolar ventilation. They also exert a powerful influence on cardiovascular function.

Delivery of Oxygen (O₂) and removal of Carbon Dioxide (CO₂) in the human body is regulated by two control systems, behavioral control and metabolic control. The metabolic ventilatory control system drives our breathing at rest and ensures optimal cellular homeostasis with respect to pH, partial pressure of carbon dioxide (PCO₂), and partial pressure of oxygen (PO₂). Metabolic control uses two sets of chemoreceptors that provide a fine-tuning function: the central chemoreceptors located in the ventral medulla of the brain and the peripheral chemoreceptors such as the aortic chemoreceptors and the carotid body chemoreceptors. The carotid body, a small, ovoid-shaped (often described as a grain of rice), and highly vascularized organ is situated in or near the carotid bifurcation, where the common carotid artery branches in to an internal carotid artery (IC) and external carotid artery (EC). The central chemoreceptors are sensitive to hypercapnia (high PCO₂), and the peripheral chemoreceptors are sensitive to hypercapnia and hypoxia (low blood PO₂). Under normal conditions activation of the sensors by their respective stimuli results in quick ventilatory responses aimed at the restoration of cellular homeostasis.

As early as 1868, Pflüger recognized that hypoxia stimulated ventilation, which spurred a search for the location of oxygen-sensitive receptors both within the brain and at various sites in the peripheral circulation. When Corneille Heymans and his colleagues observed that ventilation increased when the oxygen content of the blood flowing through the bifurcation of the common carotid artery was reduced (winning him the Nobel Prize in 1938), the search for the oxygen chemosensor responsible for the ventilatory response to hypoxia was largely considered accomplished.

The persistence of stimulatory effects of hypoxia in the absence (after surgical removal) of the carotid chemoreceptors (e.g., the carotid bodies) led other investigators, among them Julius Comroe, to ascribe hypoxic chemosensitivity to other sites, including both peripheral sites (e.g., aortic bodies) and central brain sites (e.g., hypothalamus, pons and rostral ventrolateral medulla). The aortic chemoreceptor, located in the aortic body, may also be an important chemoreceptor in humans with significant influence on vascular tone and cardiac function.

Carotid Body Chemoreflex:

The carotid body is a small cluster of chemoreceptors (also known as glomus cells) and supporting cells located near, and in most cases directly at, the medial side of the bifurcation (fork) of the carotid artery, which runs along both sides of the throat.

These organs act as sensors detecting different chemical stimuli from arterial blood and triggering an action potential in the afferent fibers that communicate this information to the Central Nervous System (CNS). In response, the CNS activates reflexes that control heart rate (HR), renal function and peripheral blood circulation to maintain the desired homeostasis of blood gases, O₂ and CO₂, and blood pH. This closed loop control function that involves blood gas chemoreceptors is known as the carotid body chemoreflex (CBC). The carotid body chemoreflex is integrated in the CNS with the carotid sinus baroreflex (CSB) that maintains arterial blood pressure. In a healthy organism these two reflexes maintain blood pressure and blood gases within a narrow physiologic range. Chemosensors and barosensors in the aortic arch contribute redundancy and fine-tuning function to the closed loop chemoreflex and baroreflex. In addition to sensing blood gasses, the carotid body is now understood to be sensitive to blood flow and velocity, blood Ph and glucose concentration. Thus it is understood that in conditions such as hypertension, CHF, insulin resistance, diabetes and other metabolic derangements afferent signaling of carotid body nerves may be elevated. Carotid body hyperactivity may be present even in the absence of detectable hypersensitivity to hypoxia and hypercapnia that are traditionally used to index carotid body function. The purpose of the proposed therapy is therefore to remove or reduce afferent neural signals from a carotid body and reduce carotid body contribution to central sympathetic tone.

The carotid sinus baroreflex is accomplished by negative feedback systems incorporating pressure sensors (e.g., baroreceptors) that sense the arterial pressure. Baroreceptors also exist in other places, such as the aorta and coronary arteries. Important arterial baroreceptors are located in the carotid sinus, a slight dilatation of the internal carotid artery at its origin from the common carotid. The carotid sinus baroreceptors are close to but anatomically separate from the carotid body. Baroreceptors respond to stretching of the arterial wall and communicate blood pressure information to CNS. Baroreceptors are distributed in the arterial walls of the carotid sinus while the chemoreceptors (glomus cells) are clustered inside the carotid body. This makes the selective reduction of chemoreflex described in this application possible while substantially sparing the baroreflex.

The carotid body exhibits great sensitivity to hypoxia (low threshold and high gain). In chronic Congestive Heart Failure (CHF), the sympathetic nervous system activation that is directed to attenuate systemic hypoperfusion at the initial phases of CHF may ultimately exacerbate the progression of cardiac dysfunction that subsequently increases the extra-cardiac abnormalities, a positive feedback cycle of progressive deterioration, a vicious cycle with ominous consequences. It was thought that much of the increase in the sympathetic nerve activity (SNA) in CHF was based on an increase of sympathetic flow at a level of the CNS and on the depression of arterial baroreflex function. In the past several years, it has been demonstrated that an increase in the activity and sensitivity of peripheral chemoreceptors (heightened chemoreflex function) also plays an important role in the enhanced SNA that occurs in CHF.

Role of Altered Chemoreflex in CHF:

As often happens in chronic disease states, chemoreflexes that are dedicated under normal conditions to maintaining homeostasis and correcting hypoxia contribute to increase the sympathetic tone in patients with CHF, even under normoxic conditions. The understanding of how abnormally enhanced sensitivity of the peripheral chemosensors, particularly the carotid body, contributes to the tonic elevation in SNA in patients with CHF has come from several studies in animals. According to one theory, the local angiotensin receptor system plays a fundamental role in the enhanced carotid body chemoreceptor sensitivity in CHF. In addition, evidence in both CHF patients and animal models of CHF has clearly established that the carotid body chemoreflex is often hypersensitive in CHF patients and contributes to the tonic elevation in sympathetic function. This derangement derives from altered function at the level of both the afferent and central pathways of the reflex arc. The mechanisms responsible for elevated afferent activity from the carotid body in CHF are not yet fully understood.

Regardless of the exact mechanism behind the carotid body hypersensitivity, the chronic sympathetic activation driven from the carotid body and other autonomic pathways leads to further deterioration of cardiac function in a positive feedback cycle. As CHF ensues, the increasing severity of cardiac dysfunction leads to progressive escalation of these alterations in carotid body chemoreflex function to further elevate sympathetic activity and cardiac deterioration. The trigger or causative factors that occur in the development of CHF that sets this cascade of events in motion and the time course over which they occur remain obscure. Ultimately, however, causative factors are tied to the cardiac pump failure and reduced cardiac output. According to one theory, within the carotid body, a progressive and chronic reduction in blood flow may be the key to initiating the maladaptive changes that occur in carotid body chemoreflex function in CHF.

There is sufficient evidence that there is increased peripheral and central chemoreflex sensitivity in heart failure, which is likely to be correlated with the severity of the disease. There is also some evidence that the central chemoreflex is modulated by the peripheral chemoreflex. According to current theories, the carotid body is the predominant contributor to the peripheral chemoreflex in humans; the aortic body having a minor contribution.

Although the mechanisms responsible for altered central chemoreflex sensitivity remain obscure, the enhanced peripheral chemoreflex sensitivity can be linked to a depression of nitric oxide production in the carotid body affecting afferent sensitivity, and an elevation of central angiotensin II affecting central integration of chemoreceptor input. The enhanced chemoreflex may be responsible, in part, for the enhanced ventilatory response to exercise, dyspnea, Cheyne-Stokes breathing, and sympathetic activation observed in chronic heart failure patients. The enhanced chemoreflex may be also responsible for hyperventilation and tachypnea (e.g., fast breathing) at rest and exercise, periodic breathing during exercise, rest and sleep, hypocapnia, vasoconstriction, reduced peripheral organ perfusion and hypertension.

Dyspnea:

Shortness of breath, or dyspnea, is a feeling of difficult or labored breathing that is out of proportion to the patient's level of physical activity. It is a symptom of a variety of different diseases or disorders and may be either acute or chronic. Dyspnea is the most common complaint of patients with cardiopulmonary diseases.

Dyspnea is believed to result from complex interactions between neural signaling, the mechanics of breathing, and the related response of the central nervous system. A specific area has been identified in the mid-brain that may influence the perception of breathing difficulties.

The experience of dyspnea depends on its severity and underlying causes. The feeling itself results from a combination of impulses relayed to the brain from nerve endings in the lungs, rib cage, chest muscles, or diaphragm, combined with the perception and interpretation of the sensation by the patient. In some cases, the patient's sensation of breathlessness is intensified by anxiety about its cause. Patients describe dyspnea variously as unpleasant shortness of breath, a feeling of increased effort or tiredness in moving the chest muscles, a panicky feeling of being smothered, or a sense of tightness or cramping in the chest wall.

The four generally accepted categories of dyspnea are based on its causes: cardiac, pulmonary, mixed cardiac or pulmonary, and non-cardiac or non-pulmonary. The most common heart and lung diseases that produce dyspnea are asthma, pneumonia, COPD, and myocardial ischemia or heart attack (myocardial infarction). Foreign body inhalation, toxic damage to the airway, pulmonary embolism, congestive heart failure (CHF), anxiety with hyperventilation (panic disorder), anemia, and physical deconditioning because of sedentary lifestyle or obesity can produce dyspnea. In most cases, dyspnea occurs with exacerbation of the underlying disease. Dyspnea also can result from weakness or injury to the chest wall or chest muscles, decreased lung elasticity, obstruction of the airway, increased oxygen demand, or poor pumping action of the heart that results in increased pressure and fluid in the lungs, such as in CHF.

Acute dyspnea with sudden onset is a frequent cause of emergency room visits. Most cases of acute dyspnea involve pulmonary (lung and breathing) disorders, cardiovascular disease, or chest trauma. Sudden onset of dyspnea (acute dyspnea) is most typically associated with narrowing of the airways or airflow obstruction (bronchospasm), blockage of one of the arteries of the lung (pulmonary embolism), acute heart failure or myocardial infarction, pneumonia, or panic disorder.

Chronic dyspnea is different. Long-standing dyspnea (chronic dyspnea) is most often a manifestation of chronic or progressive diseases of the lung or heart, such as COPD, which includes chronic bronchitis and emphysema. The treatment of chronic dyspnea depends on the underlying disorder. Asthma can often be managed with a combination of medications to reduce airway spasms and removal of allergens from the patient's environment. COPD requires medication, lifestyle changes, and long-term physical rehabilitation. Anxiety disorders are usually treated with a combination of medication and psychotherapy.

Although the exact mechanism of dyspnea in different disease states is debated, there is no doubt that the CBC plays some role in most manifestations of this symptom. Dyspnea seems to occur most commonly when afferent input from peripheral receptors is enhanced or when cortical perception of respiratory work is excessive.

Surgical Removal of the Glomus and Resection of Carotid Body Nerves:

A surgical treatment for asthma, removal of the carotid body or glomus (glomectomy), was described by Japanese surgeon Komei Nakayama in 1940s. According to Nakayama in his study of 4,000 patients with asthma, approximately 80% were cured or improved six months after surgery and 58% allegedly maintained good results after five years. Komei Nakayama performed most of his surgeries while at the Chiba University during World War II. Later in the 1950's, a U.S. surgeon, Dr. Overholt, performed the Nakayama operation on 160 U.S. patients. He felt it necessary to remove both carotid bodies in only three cases. He reported that some patients feel relief the instant when the carotid body is removed, or even earlier, when it is inactivated by an injection of procaine (Novocain). Overholt, in his paper Glomectomy for Asthma published in Chest in 1961, described surgical glomectomy the following way: “A two-inch incision is placed in a crease line in the neck, one-third of the distance between the angle of the mandible and clavicle. The platysma muscle is divided and the sternocleidomastoid retracted laterally. The dissection is carried down to the carotid sheath exposing the bifurcation. The superior thyroid artery is ligated and divided near its take-off in order to facilitate rotation of the carotid bulb and expose the medial aspect of the bifurcation. The carotid body is about the size of a grain of rice and is hidden within the adventitia of the vessel and is of the same color. The perivascular adventitia is removed from one centimeter above to one centimeter below the bifurcation. This severs connections of the nerve plexus, which surrounds the carotid body. The dissection of the adventitia is necessary in order to locate and identify the body. It is usually located exactly at the point of bifurcation on its medial aspect. Rarely, it may be found either in the center of the crotch or on the lateral wall. The small artery entering the carotid body is clamped, divided, and ligated. The upper stalk of tissue above the carotid body is then clamped, divided, and ligated.”

In January 1965, the New England Journal of Medicine published a report of 15 cases in which there had been unilateral removal of the cervical glomus (carotid body) for the treatment of bronchial asthma, with no objective beneficial effect. This effectively stopped the practice of glomectomy to treat asthma in the U.S.

Winter developed a technique for separating nerves that contribute to the carotid sinus nerves into two bundles, carotid sinus (baroreflex) and carotid body (chemoreflex), and selectively cutting out the latter. The Winter technique is based on his discovery that carotid sinus (baroreflex) nerves are predominantly on the lateral side of the carotid bifurcation and carotid body (chemoreflex) nerves are predominantly on the medial side.

Neuromodulation of the Carotid Body Chemoreflex:

Hlavaka in U.S. Patent Application Publication 2010/0070004 filed Aug. 7, 2009, describes implanting an electrical stimulator to apply electrical signals, which block or inhibit chemoreceptor signals in a patient suffering dyspnea. Hlavaka teaches that “some patients may benefit from the ability to reactivate or modulate chemoreceptor functioning.” Hlavaka focuses on neuromodulation of the chemoreflex by selectively blocking conduction of nerves that connect the carotid body to the CNS. Hlavaka describes a traditional approach of neuromodulation with an implantable electric pulse generator that does not modify or alter tissue of the carotid body or chemoreceptors.

The central chemoreceptors are located in the brain and are difficult to access. The peripheral chemoreflex is modulated primarily by carotid bodies that are more accessible. Previous clinical practice had very limited clinical success with the surgical removal of carotid bodies to treat asthma in 1940s and 1960s.

While the invention has been described in connection with what is presently considered to be the best mode, it is to be understood that the invention is not to be limited to the disclosed embodiment(s). The invention covers various modifications and equivalent arrangements included within the spirit and scope of the appended claims.

1. An endovascular ablation catheter comprising a structure at a distal region, the structure comprising:
two arms configured to couple with a carotid bifurcation;
at least one ablation element on one of the arms positioned on the arm such that when the structure is coupled to a carotid bifurcation the at least one ablation element is placed on a target site for carotid body ablation.

2.-10. (canceled)

11. A system configured for endovascular transmural ablation of a carotid body including:
a catheter having two arms to facilitate positioning and apposition of ablation elements on an intercarotid septum.

12.-19. (canceled)

20. A device for ablating the function of a carotid body comprising:
an elongate tubular structure configured for endovascular access of a carotid bifurcation having a distal region and a proximal region,
a bifurcated structure at the distal region configured to abut the carotid bifurcation, the structure comprising diverging structures and at least one ablation element mounted on one of the diverging structures, and
a conveyor of energy to be applied to said ablation element from a source of ablation energy;
whereby, the bifurcated structure is configured to apply a contact force between the ablation element and a carotid artery wall.

21.-24. (canceled)

25. A system for ablating a function of a carotid body in a patient comprising:
a catheter configured for use in the vicinity of a carotid artery bifurcation comprising a distal region and a proximal region, a structure at the distal region configured for coupling with a carotid septum comprising at least one ablation element, a means for connecting said ablation element to a source of ablation energy;
a console comprising source of ablation energy and a means for controlling said energy, a user interface configured to provide the user with a selection of ablation parameters and to provide the user with indications of the status of the console and the status of ablation activity, and a means to activate and deactivate an ablation;
whereby, the catheter provides the means for user placement of said ablation element into an optimal position within a carotid artery for ablation, and the console provides the means for user selection of optimal ablation parameters.

26. An endovascular ablation catheter comprising:
a fixation structure configured to engage with a carotid bifurcation;
an arm configured to extend into a carotid artery when the fixation structure engages with the carotid bifurcation; and
at least one ablation element arranged on the arm such that it is spaced apart a fixed distance from a carotid bifurcation saddle when the fixation structure engages with the carotid bifurcation.

27.-33. (canceled)

34. An endovascular carotid septum ablation catheter comprising:
first and second diverging arms, the first arm comprising an ablation element and configured so that the ablation element is in contact with a carotid septal wall in one of an external carotid artery and an internal carotid artery when the catheter is coupled with a common carotid artery bifurcation, the second arm configured to be disposed in the other of the internal carotid artery and external carotid artery when the catheter is coupled with the bifurcation.

35.-37. (canceled)

38. The catheter of claim 34 wherein the second arm comprises a second ablation element, the second arm being configured so that the second ablation element is in contact with a carotid septal wall in the internal carotid artery between the bifurcation and about 10-15 mm cranial to the bifurcation when the catheter is coupled with the bifurcation.

39. The catheter of claim 34 wherein the first arm is configured such that substantially all contact that occurs between the first arm and the wall of the one of the internal carotid artery or the external carotid artery occurs between the ablation element and the wall.

40.-44. (canceled)

45. The catheter of claim 34 wherein the first arm comprises a clearance portion proximal the ablation element, the clearance portion configured to make less surface area contact with the wall of the one of the external carotid artery and internal carotid artery than the ablation element.

46. (canceled)

47. The catheter of claim 34 wherein the second arm comprises a second ablation element, the second arm configured so that the second ablation element is in contact with a carotid septal wall in the other of the external carotid artery and internal carotid artery when the catheter is coupled with a common carotid artery bifurcation, wherein the first and second arms are configured to self-align within the internal and external carotid arteries against the septum.

48.-49. (canceled)

50. The catheter of claim 47 wherein the first and second arms are in substantially the same plane in unstressed configurations.

51. The catheter of claim 50 wherein the first and second arms are flexible so that they are configured to be deflectable out of plane, and yet are resilient to allow them to return to the plane.

52. (canceled)

53. The catheter of claim 51 wherein the first and second arms have sufficient resiliency to allow them to move from one stress state to a lower stress state when positioned in contact with the walls of the internal and external carotid arteries.

54.-55. (canceled)

56. The catheter of claim 34 wherein the first and second arms have unstressed configurations in which the first and second ablation elements are less than about 4 mm apart measured along a line perpendicular to a longitudinal axis of a catheter axis.

57.-94. (canceled)

95. An endovascular carotid septum ablation catheter comprising:
first and second diverging arms, the first arm comprising a first ablation element and configured so that the first ablation element is in contact with an external carotid artery wall when the catheter is coupled with a common carotid artery bifurcation, the second arm comprising a second ablation element and configured so that the second ablation element is in contact with an internal carotid artery when the catheter is coupled with the bifurcation, wherein the first and second ablation elements are positioned on the first and second arms so that when the catheter is coupled with the bifurcation, a straight line passing through the first and second ablation elements passes through a carotid septum.

96. (canceled)

97. A method of ablating a carotid septum, comprising:
advancing a first diverging arm of an ablation catheter into an external carotid artery and a second diverging arm of the ablation catheter into an internal carotid artery so that a first ablation element on the first diverging arm is in apposition with a carotid septum wall in the external carotid artery and a second ablation element on the second diverging arm is positioned in the internal carotid artery; and
ablating carotid septal tissue by delivering ablation energy between the first and second ablation elements so that the ablation energy passes through a carotid septum.

98.-131. (canceled)

132. A method of ablating a carotid septum, comprising
advancing a first diverging arm of an ablation catheter into an external carotid artery and a second diverging arm of the ablation catheter into an internal carotid artery so that a first ablation element on the first diverging arm is in apposition with a carotid septum wall in the external carotid artery and a second ablation element on the second diverging arm is in apposition with a carotid septum wall in the internal carotid artery; and
ablating carotid septal tissue by delivering ablation energy between the first and second ablation elements so that the ablation energy passes through a carotid septum.

133. A method of endovascularly ablating a carotid septum, comprising
providing an elongate device comprising first and second diverging arms, the first diverging arm comprising a first ablation element and the second bifurcating arm comprising a second ablation element;
positioning the first ablation element in contact with an external carotid artery so as to create a surface contact area between the first ablation electrode and the external carotid artery that is between about 30% to about 70% of a total surface area of the first ablation element, and
positioning the second diverging arm in an internal carotid artery; and
ablating carotid septal tissue by delivering ablation energy between the first and second ablation elements through the carotid septum.

134. (canceled)

135. A method of endovascularly ablating a carotid septum, comprising
providing an elongate device comprising first and second diverging arms, the first diverging arm comprising a first ablation element and the second bifurcating arm comprising a second ablation element;
positioning the first ablation element in contact with an external carotid artery so as to create a surface contact area between the first ablation electrode and the external carotid artery that is between about 4.5 mm2 and about 21 mm2;
positioning the second diverging arm in an internal carotid artery; and
ablating carotid septal tissue by delivering ablation energy between the first and second ablation elements through the carotid septum.

136.-137. (canceled)

138. A method of endovascularly ablating a carotid body, comprising:
providing an elongate device comprising first and second diverging arms, the first diverging arm comprising a first electrode and the second diverging arm comprising a second electrode;
positioning the first diverging arm in an external carotid artery and the second diverging arm in an internal carotid artery;
delivering alternating electric current between the first and second electrodes;
forming an ablation zone within a carotid septum that does not extend to the internal or external carotid arteries, wherein the ablation zone includes a location midway along a line passing through the first and second electrodes; and
continuing to deliver alternating electric current energy to extend the ablation zone towards the internal and carotid arteries.

139.-141. (canceled)
